(19) United States
(12) Patent Application Publication
(10) Pub. No.: US 2025/0311621 A1
(43) Pub. Date: Oct. 2, 2025

(54) LIGHT EMITTING ELEMENT, AMINE COMPOUND FOR THE SAME, AND ELECTRONIC APPARATUS INCLUDING THE SAME

(71) Applicant: Samsung Display Co., Ltd., Yongin-si (KR)

(72) Inventors: Ichinori TAKADA, Yokohama (JP); Yusuke TAKITA, Yokohama (JP)

(21) Appl. No.: 19/060,109

(22) Filed: Feb. 21, 2025

(30) Foreign Application Priority Data
Apr. 1, 2024 (KR) 10-2024-0044340

(51) Int. Cl. H10K 85/60; C07C 211/61; C07D 307/91; C07D 333/76; C07D 405/12; H10K 50/15; H10K 59/38

(52) U.S. Cl. CPC H10K 85/636; C07C 211/61; C07D 307/91; C07D 333/76; C07D 405/12; H10K 85/633; C07C 2603/18; H10K 50/15; H10K 59/38; H10K 85/615; H10K 85/6572; H10K 85/6574; H10K 85/6576

(57) ABSTRACT

A light emitting element includes a first electrode, a second electrode on the first electrode, and at least one functional layer between the first electrode and the second electrode and including an amine compound represented by Formula 1.

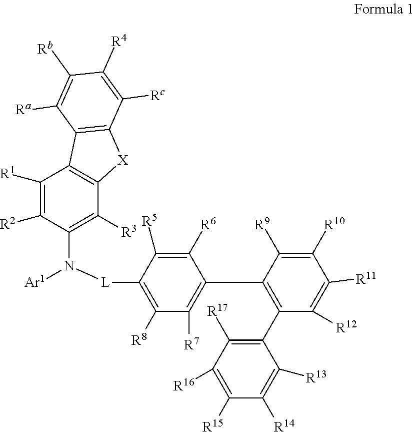

### CROSS-REFERENCE TO RELATED APPLICATION

The present application claims priority to and the benefit of Korean Patent Application No. 10-2024-0044340, filed on Apr. 1, 2024, in the Korean Intellectual Property Office, the entire disclosure of which is incorporated herein by reference.

### BACKGROUND

### 1. Field

One or more embodiments of the present disclosure herein relate to a light emitting element, an amine compound used therein, and an electronic apparatus including the light emitting element.

### 2. Description of the Related Art

An electronic apparatus includes a display device that displays an image. Recently, there has been significant research and development in image display devices, e.g., organic electroluminescence display devices and/or similar technologies. The organic electroluminescence display devices and/or the like are display devices including self-luminescent light emitting elements. In each of these light emitting elements, holes and electrons separately injected from a first electrode and a second electrode of the light emitting element, recombing in an emission layer. As a result, a luminescent material in the emission layer emits light due to the recombination of those holes and electrons, thereby achieving display (e.g., display of image).

For application of light emitting elements in display devices, there is a demand and/or desire for light emitting elements with relatively high luminous efficiency and long life. Consequently, the development and research of materials for light emitting elements that can stably achieves these desired characteristics are continuously pursued or required.

For example, to obtain light emitting elements with high efficiency and long service life, materials for hole transport regions having excellent or suitable hole transport properties and stability are actively researched and developed.

### SUMMARY

One or more aspects of embodiments of the present disclosure are directed toward a light emitting element having increased luminous efficiency and longer element service life.

One or more aspects of embodiments of the present disclosure are directed toward an amine compound capable of improving luminous efficiency and element service life of a light emitting element including the amine compound.

One or more aspects of embodiments of the present disclosure are directed toward an electronic apparatus having high display quality by including the light emitting element having improved luminous efficiency and lifetime.

Additional aspects will be set forth in part in the description which follows and, in part, will be apparent from the description, or may be learned by practice of the presented embodiments.

According to one or more embodiments of the present disclosure, a light emitting element includes a first electrode, a second electrode on (e.g., arranged on) the first electrode, and at least one functional layer between the first electrode and the second electrode and including an amine compound represented by Formula 1.

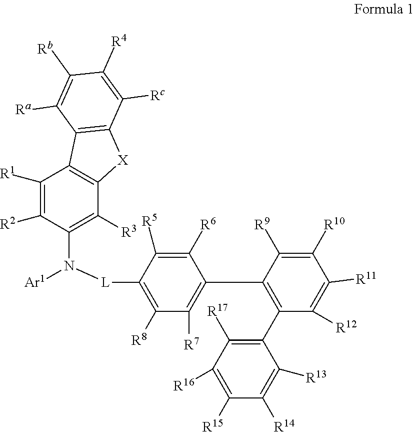

In Formula 1, X may be O, S, NAr², or CAr³Ar⁴, R^{a} to R^{c} may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, at least one selected from among R^{a} to R^{c} is a substituted or unsubstituted aryl group having 6 to 30 ring-forming carbon atoms, R¹ to R⁴ may each independently be hydrogen, deuterium, a substituted or unsubstituted alkyl group having 1 to 20 carbon atoms, or a substituted or unsubstituted aryl group having 6 to 30 ring-forming carbon atoms, Ar¹ may be a substituted or unsubstituted aryl group having 6 to 30 ring-forming carbon atoms or a substituted or unsubstituted heteroaryl group having 2 to 30 ring-forming carbon atoms, Ar² to Ar⁴ may each independently be a substituted or unsubstituted aryl group having 6 to 30 ring-forming carbon atoms, L may be a direct linkage, a substituted or unsubstituted arylene group having 6 to 30 ring-forming carbon atoms, or a substituted or unsubstituted heteroarylene group having 2 to 30 ring-forming carbon atoms, R⁵ to R¹⁷ may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring, in Formula 1, a case in which L is a direct linkage and R⁹ and R¹⁰, and R¹¹ and R¹² are each bonded together to form a ring is excluded, in Formula 1, a case in which L is a direct linkage and any of the pairs R¹² and R¹³, and R¹² and R¹⁷ are bonded together to form a ring is excluded, in Formula 1, a case in which L is a direct linkage and R¹¹ and R¹² are bonded together to form a nitrogen-containing heterocycle is excluded, in Formula 1, a case in which if (e.g., when) X is CAr³Ar⁴, Ar¹ is an unsubstituted phenyl group is excluded, a case in which the amine compound represented by Formula 1 contains a fluorene moiety substituted with at least one alkyl group at position 9 or unsubstituted, a substituted or unsubstituted 9-fluorene moiety, a substituted or unsubstituted spirobifluorene moiety, or a substituted or unsubstituted N,N-bis(4-(2-naphthyl)phenyl)amine moiety is excluded, and in one or more embodiments, the amine compound represented by Formula 1 may include a structure in which at least one hydrogen is substituted with deuterium.

In one or more embodiments, the at least one functional layer may include an emission layer, a hole transport region between the first electrode and the emission layer, and an electron transport region between the emission layer and the second electrode, and the hole transport region may include the amine compound represented by Formula 1.

In one or more embodiments, the hole transport region may include a hole injection layer on (e.g., arranged on) the first electrode, and a hole transport layer on (e.g., arranged on) the hole injection layer, and the hole transport layer may include the amine compound represented by Formula 1.

In one or more embodiments, the amine compound represented by Formula 1 may be represented by any one selected from among Formulas 2-1 to 2-3.

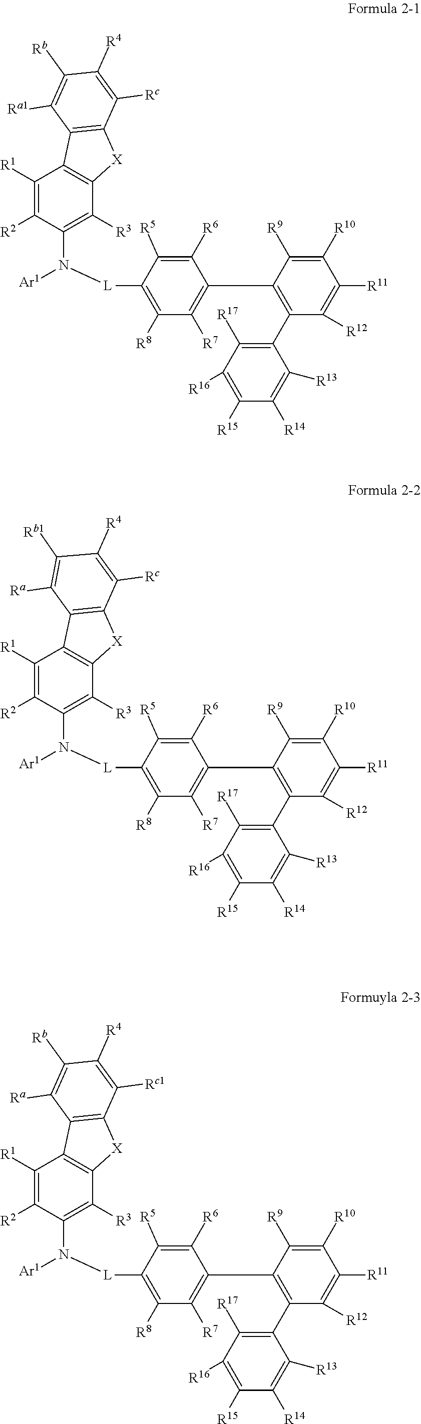

In Formulas 2-1 to 2-3, R^{a1}, R^{b1}, and R^{c1} may each independently be a substituted or unsubstituted aryl group having 6 to 30 ring-forming carbon atoms.

In Formulas 2-1 to 2-3, Ar¹, L, X, R^{a}, R^{b}, R^{c}, and R¹ to R¹⁷ may each be the same as defined in Formula 1.

In one or more embodiments, the amine compound represented by Formula 1 may be represented by Formula 3.

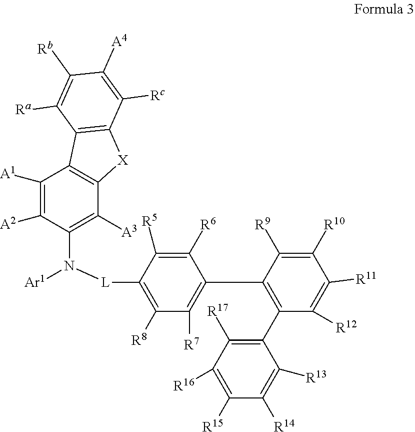

In Formula 3, A¹ to A⁴ may each independently be hydrogen or deuterium.

In Formulas 3, Ar¹, L, X, R^{a}, R^{b}, R^{c}, and R⁵ to R¹⁷ may each be the same as defined in Formula 1.

In one or more embodiments, Ar¹ may be a substituted or unsubstituted phenyl group, a substituted or unsubstituted biphenylyl group, a substituted or unsubstituted naphthyl group, a substituted or unsubstituted naphthylphenyl group, a substituted or unsubstituted phenanthrenyl group, a substituted or unsubstituted dibenzofuranyl group, or a substituted or unsubstituted dibenzothiophenyl group.

In one or more embodiments, if (e.g., when) Ar¹ is substituted, a substituent thereof may be deuterium, a substituted or unsubstituted phenyl group, or a substituted or unsubstituted naphthyl group.

In one or more embodiments, the amine compound represented by Formula 1 may be represented by Formula 4-1 or Formula 4-2.

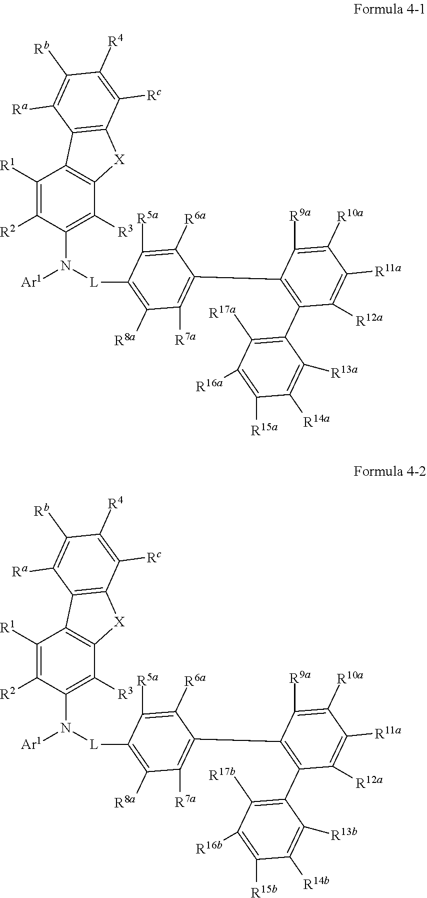

In Formula 4-1, R^{5a} to R^{17a} may each independently be hydrogen, deuterium, a substituted or unsubstituted alkyl group having 1 to 20 carbon atoms, or a substituted or unsubstituted aryl group having 6 to 30 ring-forming carbon atoms; in Formula 4-2, R^{5a} to R^{12a} may each independently be hydrogen, deuterium, a substituted or unsubstituted alkyl group having 1 to 20 carbon atoms, or a substituted or unsubstituted aryl group having 6 to 30 ring-forming carbon atoms, and R^{13b} to R^{17b} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring.

In Formulas 4-1 and 4-2, Ar¹, L, X, R^{a}, R^{b}, R^{c}, and R¹ to R⁴ may each be the same as defined in Formula 1.

In one or more embodiments, the amine compound represented by Formula 1 may be represented by Formula 5.

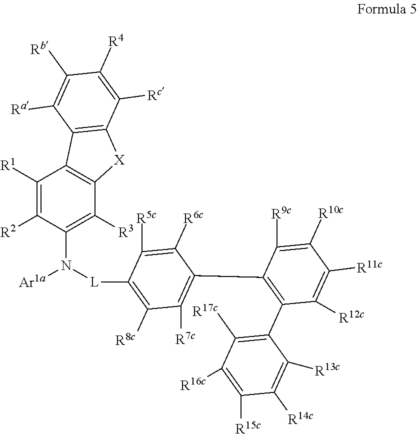

In Formula 5, R^{a}′ to R^{c}′ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, at least one selected from among R^{a}′ to R^{c}′ may be a substituted or unsubstituted phenyl group, Aria may be a substituted or unsubstituted aryl group having 6 to 30 ring-forming carbon atoms, R^{5c} to R^{17c} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring, in Formula 5, a case in which if (e.g., when) R^{11c} and R^{12c} are bonded together to form a ring, Aria is a substituted or unsubstituted naphthylphenyl group may be excluded, in Formula 5, a case in which L is a direct linkage and R^{9c} and R^{10c}, and R^{11c} and R^{12c} are each bonded together to form a ring may be excluded, in Formula 5, a case in which L is a direct linkage and any of the pairs R^{12c} and R^{13c}, and R^{12c} and R^{17c} are bonded together to form a ring may be excluded, in Formula 5, a case in which L is a direct linkage and R^{11c} and R^{12c} are bonded together to form a nitrogen-containing heterocycle may be excluded, in Formula 5, a case in which L is a direct linkage and R^{6c} and R^{9c} are bonded together to form a ring may be excluded, and, in one or more embodiments, if (e.g., when) Ar^{1a} is substituted, a substituent may be deuterium or a substituted or unsubstituted aryl group having 6 to 15 ring-forming carbon atoms.

In Formula 5, R¹ to R⁴, X, and L may each be the same as defined in Formula 1.

In one or more embodiments, the amine compound represented by Formula 1 may be represented by Formula 6.

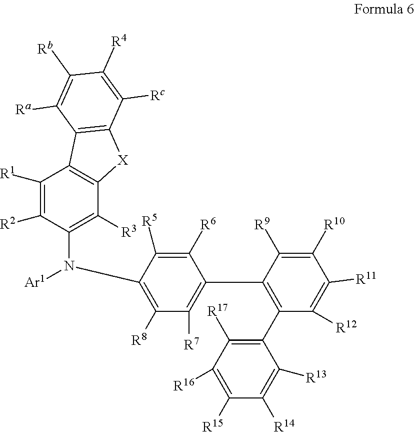

In Formulas 6, Ar¹, X, R^{a}, R^{b}, R^{c}, and R¹ to R¹⁷ may each be the same as defined in Formula 1.

In one or more embodiments, the amine compound represented by Formula 1 may be represented by any one selected from among Formulas 7-1 to 7-3.

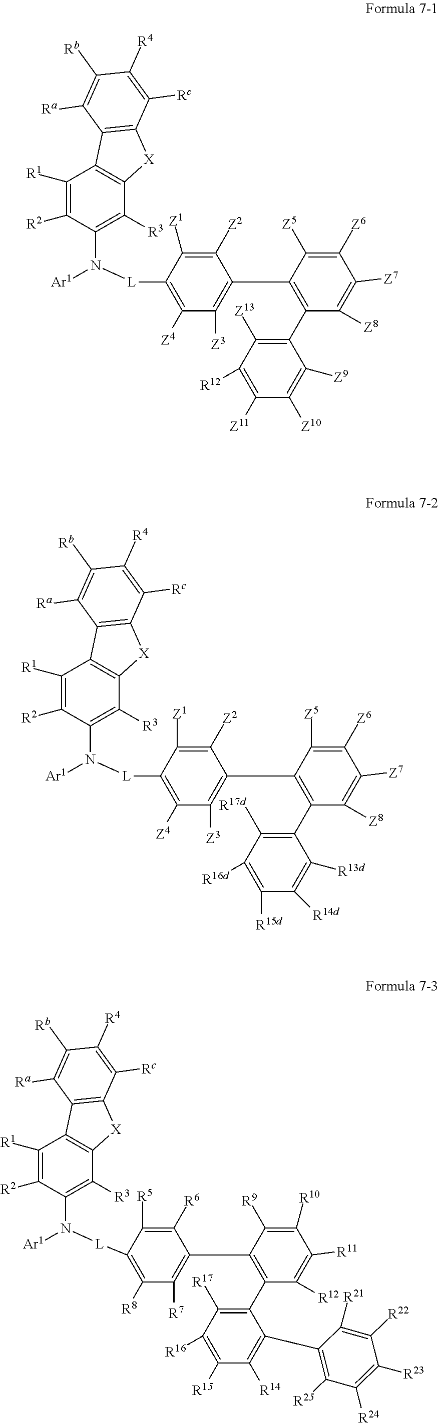

In Formula 7-1, Z¹ to Z¹³ may each independently be hydrogen or deuterium; in Formula 7-2, Z¹ to Z⁸ may each independently be hydrogen or deuterium, R^{13d} to R^{17d} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring, one adjacent pair among R^{13d} to R^{17d} may be bonded together to form a ring; in Formula 7-3, R²¹ to R²⁵ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring.

In Formulas 7-1 to 7-3, Ar¹, L, X, R^{a}, R^{b}, R^{c}, R¹ to R¹², and R¹⁴ to R¹⁷ may be the same as defined in Formula 1.

In one or more embodiments, the amine compound represented by Formula 1 may be represented by Formula 8, and the amine compound may be a compound satisfying any one selected from among the combinations shown in Compound Combination Table 1.

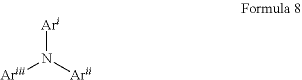

In Formula 8, Arⁱ may be any one selected from Substituent Group A, Arⁱⁱ may be any one selected from Substituent Group B, and Arⁱⁱⁱ may be any one selected from Substituent Group C.

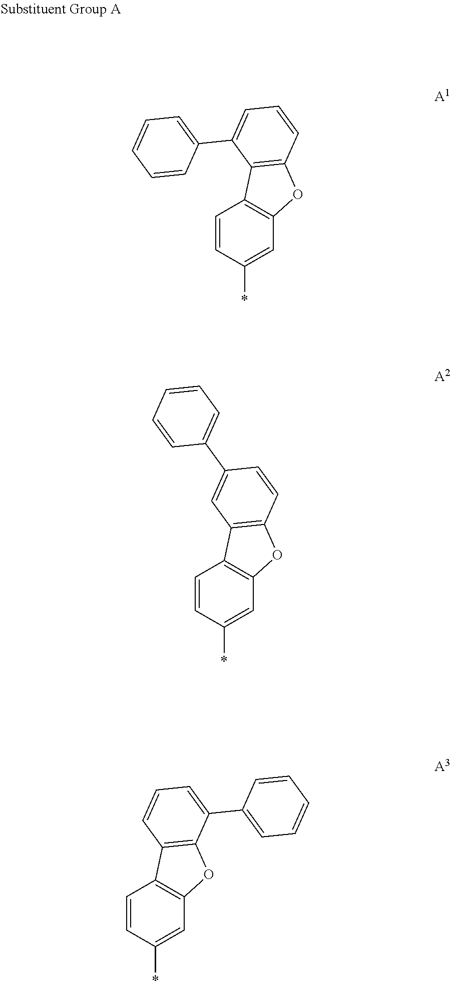 [SMILES: *c1ccc2c(c1)oc1cccc(-c3ccccc3)c12 ; *c1ccc2c(c1)oc1ccc(-c3ccccc3)cc12 ; *c1ccc2c(c1)oc1c(-c3ccccc3)cccc12]

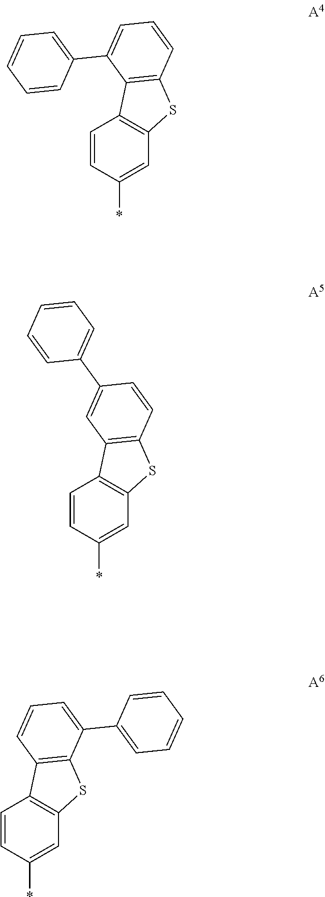 [SMILES: *c1ccc2c(c1)sc1cccc(-c3ccccc3)c12 ; *c1ccc2c(c1)sc1ccc(-c3ccccc3)cc12 ; *c1ccc2c(c1)sc1c(-c3ccccc3)cccc12]

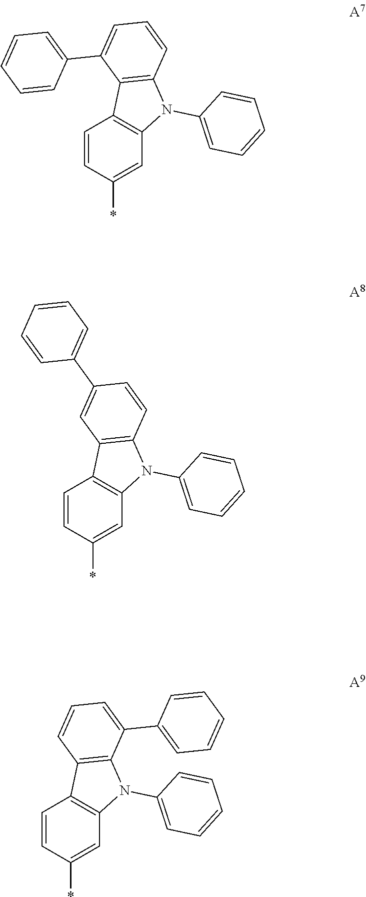 [SMILES: *c1ccc2c3c(-c4ccccc4)cccc3n(-c3ccccc3)c2c1 ; *c1ccc2c3cc(-c4ccccc4)ccc3n(-c3ccccc3)c2c1 ; *c1ccc2c3cccc(-c4ccccc4)c3n(-c3ccccc3)c2c1]

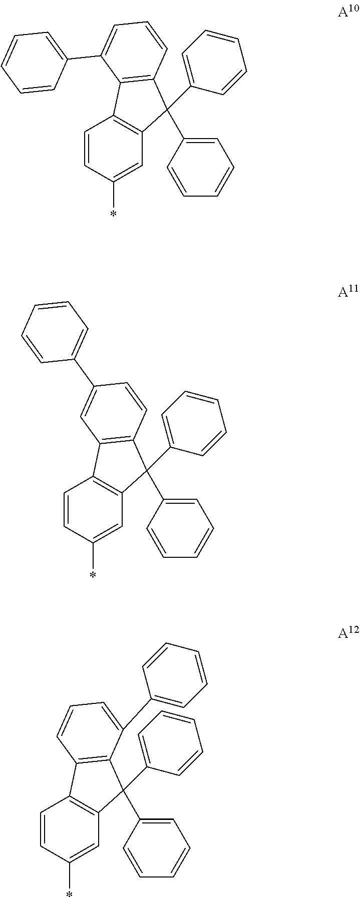 [SMILES: *c1ccc2c(c1)C(c1ccccc1)(c1ccccc1)c1cccc(-c3ccccc3)c1-2 ; *c1ccc2c(c1)C(c1ccccc1)(c1ccccc1)c1ccc(-c3ccccc3)cc1-2 ; *c1ccc2c(c1)C(c1ccccc1)(c1ccccc1)c1c(-c3ccccc3)cccc1-2]

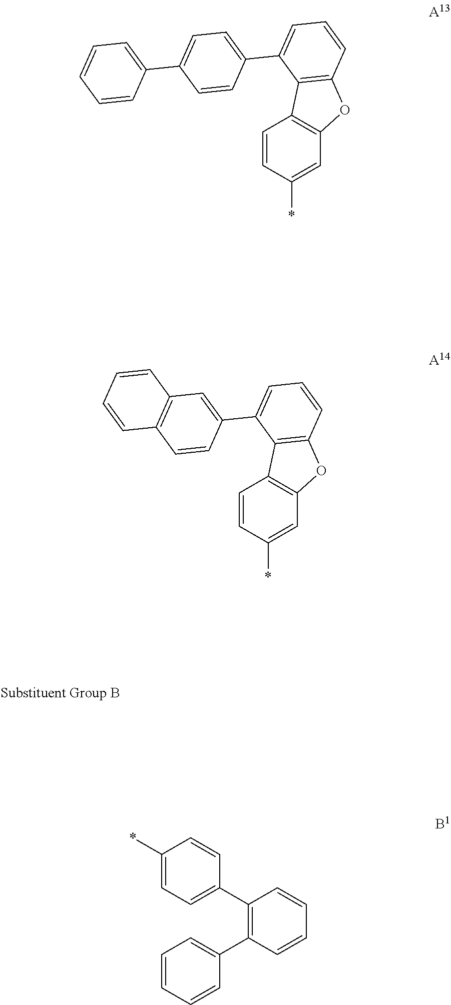 [SMILES: *c1ccc2c(c1)oc1cccc(-c3ccc(-c4ccccc4)cc3)c12 ; *c1ccc2c(c1)oc1cccc(-c3ccc4ccccc4c3)c12 ; *c1ccc(-c2ccccc2-c2ccccc2)cc1]

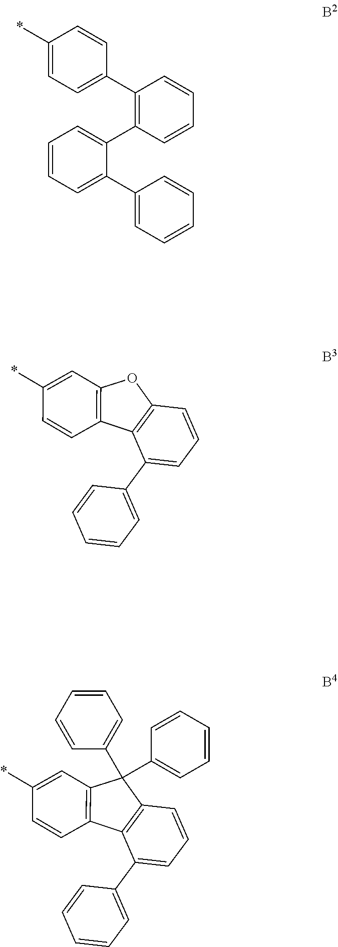 [SMILES: *c1ccc(-c2ccccc2-c2ccccc2-c2ccccc2)cc1 ; *c1ccc2c(c1)oc1cccc(-c3ccccc3)c12 ; *c1ccc2c(c1)C(c1ccccc1)(c1ccccc1)c1cccc(-c3ccccc3)c1-2]

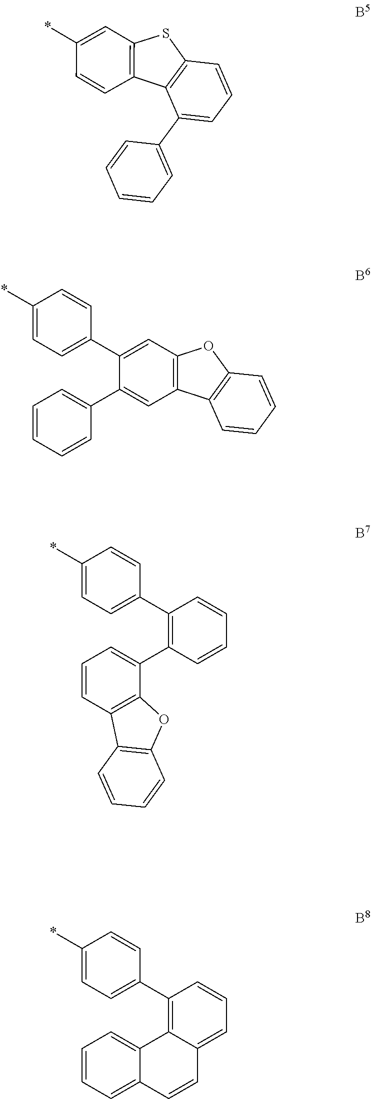 [SMILES: *c1ccc2c(c1)sc1cccc(-c3ccccc3)c12 ; *c1ccc(-c2cc3oc4ccccc4c3cc2-c2ccccc2)cc1 ; *c1ccc(-c2ccccc2-c2cccc3c2oc2ccccc23)cc1 ; *c1ccc(-c2cccc3ccc4ccccc4c23)cc1]

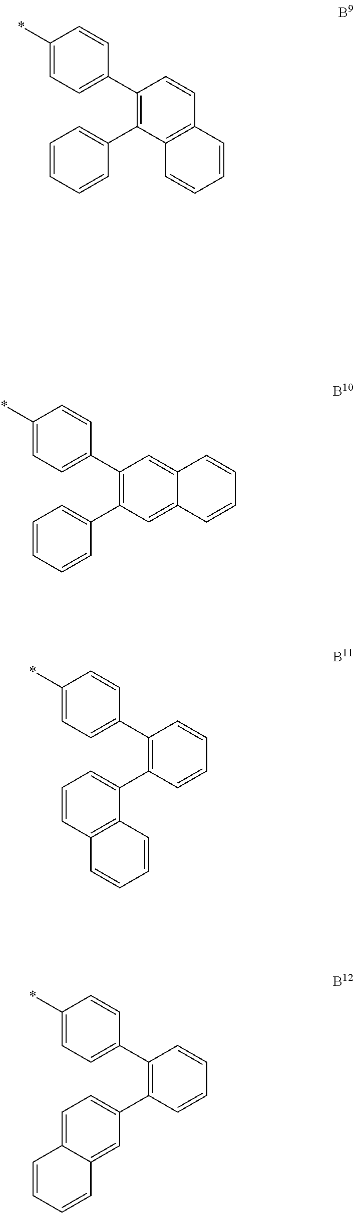 [SMILES: *c1ccc(-c2ccc3ccccc3c2-c2ccccc2)cc1 ; *c1ccc(-c2cc3ccccc3cc2-c2ccccc2)cc1 ; *c1ccc(-c2ccccc2-c2cccc3ccccc23)cc1 ; *c1ccc(-c2ccccc2-c2ccc3ccccc3c2)cc1]

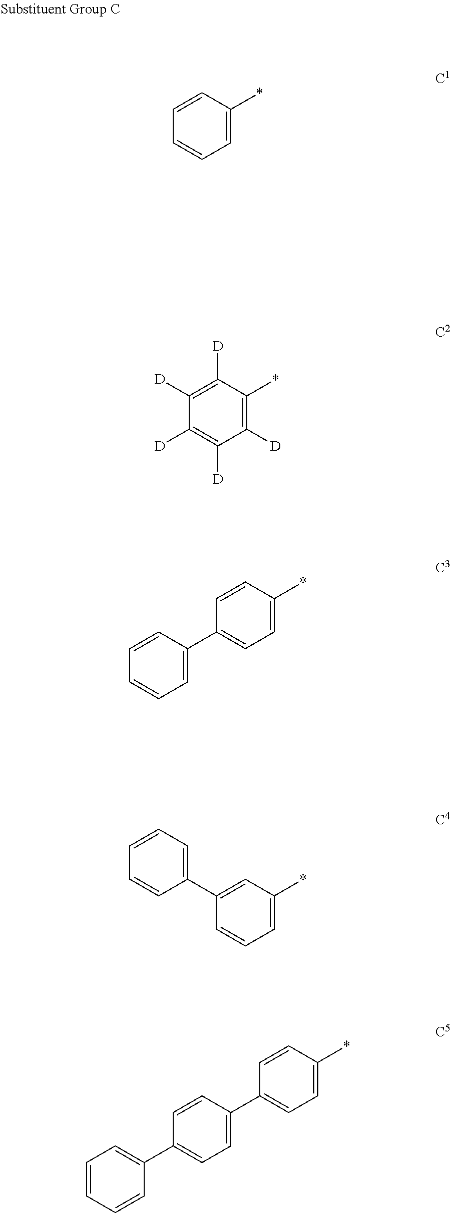 [SMILES: *c1ccccc1 ; *c1c([2H])c([2H])c([2H])c([2H])c1[2H] ; *c1ccc(-c2ccccc2)cc1 ; *c1cccc(-c2ccccc2)c1 ; *c1ccc(-c2ccc(-c3ccccc3)cc2)cc1]

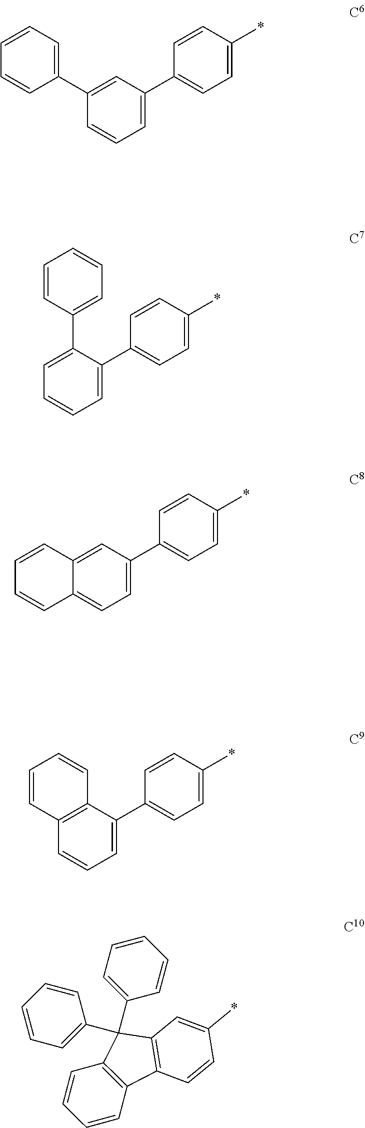 [SMILES: *c1ccc(-c2cccc(-c3ccccc3)c2)cc1 ; *c1ccc(-c2ccccc2-c2ccccc2)cc1 ; *c1ccc(-c2ccc3ccccc3c2)cc1 ; *c1ccc(-c2cccc3ccccc23)cc1 ; *c1ccc2c(c1)C(c1ccccc1)(c1ccccc1)c1ccccc1-2]

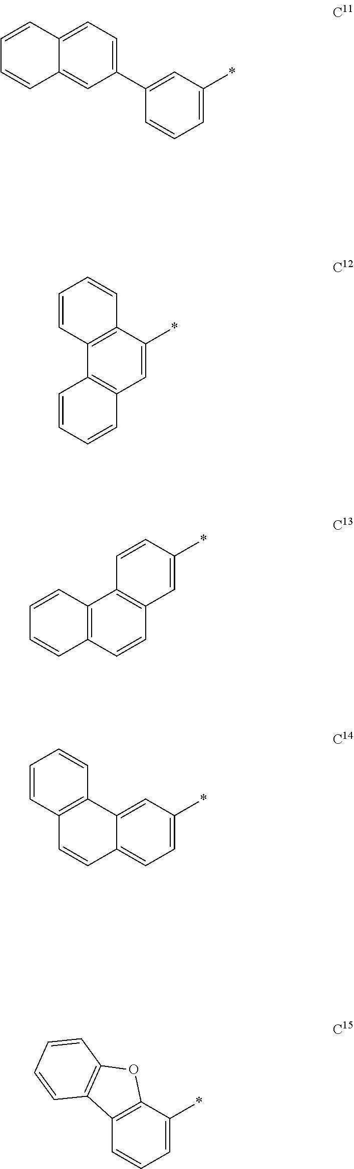 [SMILES: *c1cccc(-c2ccc3ccccc3c2)c1 ; *c1cc2ccccc2c2ccccc12 ; *c1ccc2c(ccc3ccccc32)c1 ; *c1ccc2ccc3ccccc3c2c1 ; *c1cccc2c1oc1ccccc12]

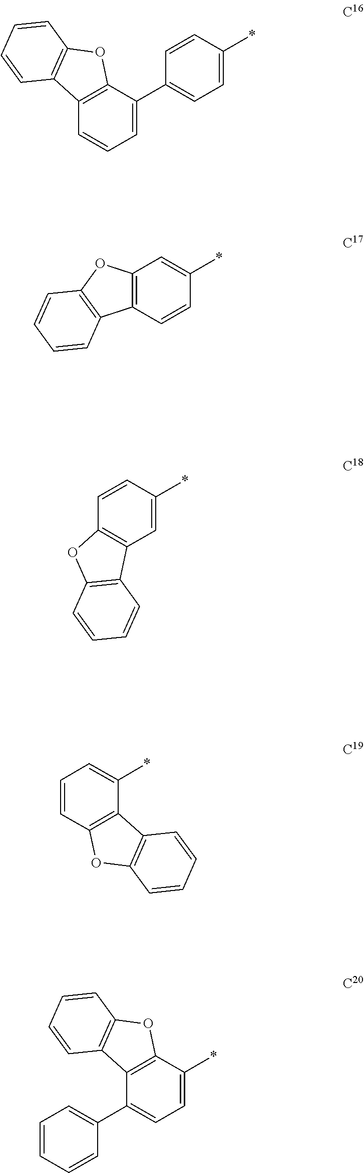 [SMILES: *c1ccc(-c2cccc3c2oc2ccccc23)cc1 ; *c1ccc2c(c1)oc1ccccc12 ; *c1ccc2oc3ccccc3c2c1 ; *c1cccc2oc3ccccc3c12 ; *c1ccc(-c2ccccc2)c2c1oc1ccccc12]

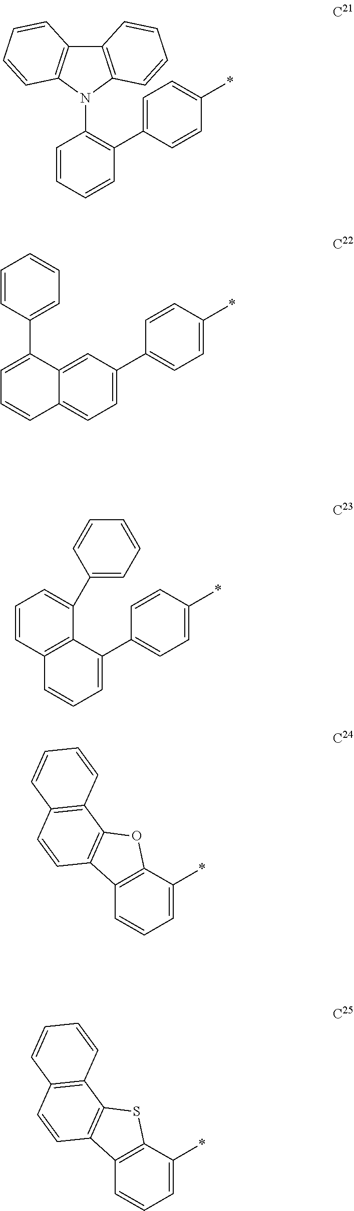

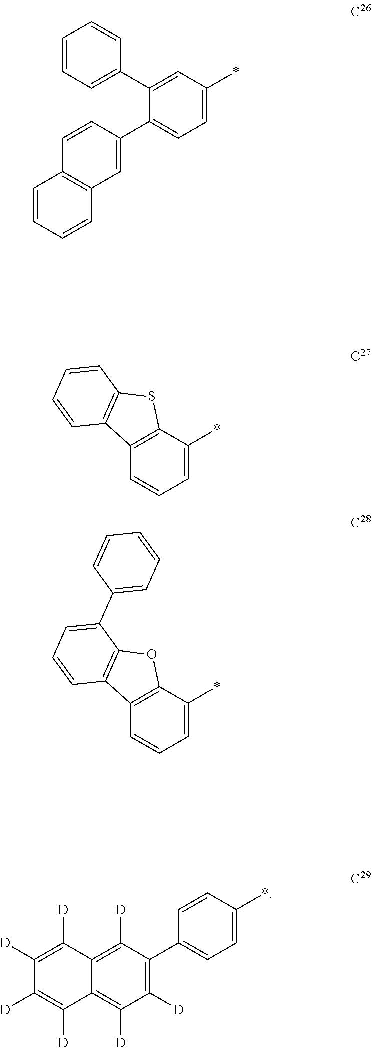

In one or more embodiments of the present disclosure, a display device includes a base layer, a circuit layer on (e.g., arranged on) the base layer, and a display element layer on (e.g., arranged on) the circuit layer and including a light emitting element, wherein the light emitting element includes a first electrode, a second electrode on (e.g., arranged on) the first electrode, and a functional layer between the first electrode and the second electrode and containing an amine compound represented by Formula 1.

In one or more embodiments, the light emitting element may further include a capping layer on (e.g., arranged on) the second electrode, wherein the capping layer may have a refractive index of about 1.6 or greater in a wavelength range of about 550 nanometers (nm) to about 660 nm.

In one or more embodiments, the display device may further include a light control layer on (e.g., arranged on) the display element layer and including quantum dots, wherein the light emitting element may be to emit first color light, and the light control layer may include a first light control portion including a first quantum dot that converts the first color light into second color light having a longer wavelength than the first color light, a second light control portion including a second quantum dot that converts the first color light into third color light having a longer wavelength than both the first color light and the second color light, and a third light control portion that transmits the first color light.

In one or more embodiments, the display device may further include a color filter layer on (e.g., arranged on) the light control layer, wherein the color filter layer may include a first filter that transmits the second color light, a second filter that transmits the third color light, and a third filter that transmits the first color light.

In one or more embodiments of the present disclosure, provided is an amine compound represented by Formula 1.

### BRIEF DESCRIPTION OF DRAWINGS

The accompanying drawings are included to provide a further understanding of the present disclosure, and are incorporated in and constitute a part of this disclosure. The drawings illustrate example embodiments of the disclosure and, together with the description, serve to explain principles of the present disclosure. Above and/or other aspects of the disclosure should become apparent and appreciated from the following description of embodiments taken in conjunction with the accompanying drawings. In the drawings:

FIG. 1 is a plan view showing a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display apparatus according to one or more embodiments of the present disclosure;

FIG. 8 is a cross-sectional view of a display apparatus according to one or more embodiments;

FIG. 9 is a cross-sectional view showing a display apparatus according to one or more embodiments of the present disclosure;

FIG. 10 is a cross-sectional view showing a display apparatus according to one or more embodiments of the present disclosure;

FIG. 11 is a view showing a vehicle in which display apparatuses according to one or more embodiments of the present disclosure are arranged;

FIG. 12 is a perspective view showing an electronic apparatus according to one or more embodiments of the present disclosure;

FIG. 13 is an exploded perspective view showing an electronic apparatus according to one or more embodiments of the present disclosure;

FIG. 14 is a block diagram of an electronic apparatus according to one or more embodiments of the present disclosure;

FIG. 15 is a diagram showing electronic apparatuses according to one or more embodiments of the present disclosure; and

FIG. 16 is a diagram showing electronic apparatuses according to one or more embodiments of the present disclosure.

### DETAILED DESCRIPTION

The present disclosure may be modified and implemented in one or more suitable manners and have many forms, and thus specific/example embodiments will be illustrated in the drawings and described in more detail in the detailed description of present disclosure. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/comprising,” “include(s)/including,” “have(has)/having” and/or the like specify the presence of features, numbers, steps, operations, component, parts, and/or one or more (e.g., any suitable) combinations thereof disclosed in the disclosure, but do not exclude the possibility of presence or addition of one or more other features, numbers, steps, operations, component, parts, and/or one or more (e.g., any suitable) combinations thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, if (e.g., when) a layer, a film, a region, or a plate is referred to as being “on” or “in an upper portion of” another layer, film, region, or plate, it may be not only “directly on” the layer, film, region, or plate, but one or more intervening layers, films, regions, or plates may also be present therebetween. Opposite this, if (e.g., when) a layer, a film, a region, or a plate is referred to as being “below”, “in a lower portion of” another layer, film, region, or plate, it can be not only directly under the layer, film, region, or plate, but one or more intervening layers, films, regions, or plates may also be present therebetween. In addition, it will be understood that if (e.g., when) a part is referred to as being “on” another part, it can be arranged above the other part, or arranged under the other part as well. In the present disclosure, “directly on” may refer to that there are no additional layers, films, regions, plates, and/or like, between a layer, a film, a region, a plate, etc. and the other part. For example, “directly on” may refer to two layers or two members are arranged without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents exemplified above may be substituted or unsubstituted. For example, a biphenylyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may be monocyclic or polycyclic. In addition, the rings formed by adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In one or more embodiments, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of the present disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but embodiments of the present disclosure are not limited thereto.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

In the present disclosure, an alkynyl group refers to a hydrocarbon group including at least one carbon-carbon triple bond in the middle or terminal of an alkyl group having 2 or more carbon atoms. The alkynyl group may be linear or branched. Although the number of carbon atoms is not specifically limited, it may be 2 to 30, 2 to 20, or 2 to 10. Examples of the alkynyl group may include an ethynyl group, a propynyl group, and/or the like, but embodiments of the present disclosure are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 50, 6 to 40, 6 to 30, 6 to 20, or 6 to 15. Examples of the aryl group may include a phenyl group, a naphthyl group, a fluorenyl group, an anthracenyl group, a phenanthrenyl group, a biphenylyl group, a terphenylyl group, a quaterphenylyl group, a quinquephenylyl group, a sexiphenylyl group, a triphenylenyl group, a pyrenyl group, a benzofluoranthenyl group, a chrysenyl group, and/or the like, but embodiments of the present disclosure are not limited thereto.

In the present disclosure, a fluorenyl group may be substituted, and two substituents may be bonded to each other to form a spiro structure. Examples of the substituted fluorenyl group are as follows. However, embodiments of the present disclosure are not limited thereto.

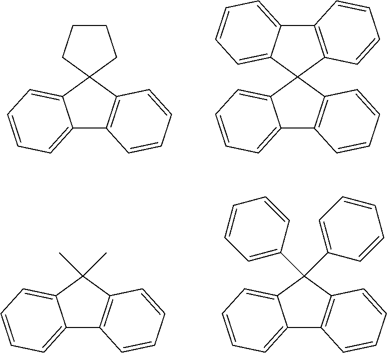

A heterocyclic group as used herein refers to any functional group or substituent derived from a ring containing at least one of B, O, N, P, Si, or Se as a heteroatom. The heterocyclic group includes an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heterocyclic group may contain at least one of B, O, N, P, Si, or S as a heteroatom. If (e.g., when) the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 60, 2 to 50, 2 to 40, 2 to 30, 2 to 20, or 2 to 10.

In the present disclosure, an aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but embodiments of the present disclosure are not limited thereto.

In the present disclosure, a heteroaryl group may contain at least one of B, O, N, P, Si, or S as a heteroatom. If (e.g., when) the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 50, 2 to 40, 2 to 30, 2 to 20, or 2 to 10. Examples of the heteroaryl group may include a thienyl group, a furyl group, a pyrrolyl group, an imidazolyl group, a pyridyl group, a bipyridinyl group, a triazolyl group, an acridinyl group, a pyridazinyl group, a pyazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, an N-arylcarbazolyl group, an N-heteroarylcarbazolyl group, an N-alkylcarbazolyl group, a benzoxazolyl group, a benzimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, an isoxazolyl group, an oxazolyl group, an oxadiazolyl group, a thiadiazolyl group, a phenothiazinyl group, a dibenzosilolyl group, a dibenzofuranyl group, and/or the like, but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but embodiments of the present disclosure are not limited thereto.

In the present disclosure, the number of carbon atoms in a carbonyl group is not specifically limited, but may be 1 to 40, 1 to 30, or 1 to 20. For example, the carbonyl group may have the following structures, but embodiments of the present disclosure are not limited thereto.

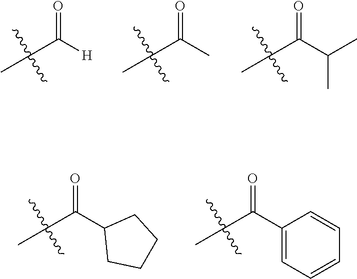

In the present disclosure, the number of carbon atoms in a sulfinyl group or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

A boron group as used herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, and/or the like, but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, and/or the like, but embodiments of the present disclosure are not limited thereto.

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, and an arylamine group may be the same as the examples of the aryl group described above.

As used herein, a dibenzofuranyl group may be substituted, and adjacent substituents may be bonded together to form a fused ring structure. For example, a dibenzofuranyl group may be substituted and two adjacent substituents may be bonded together to form a fused ring structure as shown herein. However, embodiments of the present disclosure are not limited thereto.

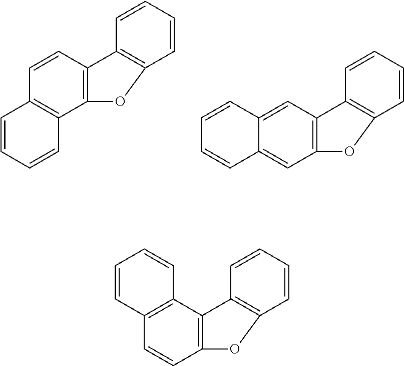 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

As used herein, a dibenzothiophenyl group may be substituted, and adjacent substituents may be bonded together to form a fused ring structure. For example, a dibenzothiophenyl group may be substituted and two adjacent substituents may be bonded together to form a fused ring structure as shown herein. However, embodiments of the present disclosure are not limited thereto.

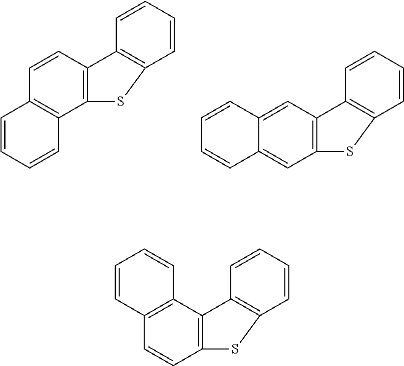 [SMILES: c1ccc2c(c1)ccc1c3ccccc3sc21 ; c1ccc2cc3c(cc2c1)sc1ccccc13 ; c1ccc2c(c1)ccc1sc3ccccc3c12]

As used herein, a carbazolyl group may be substituted, and adjacent substituents may be bonded together to form a fused ring structure. An example that a carbazolyl group is substituted is as follows. However, embodiments of the present disclosure are not limited thereto.

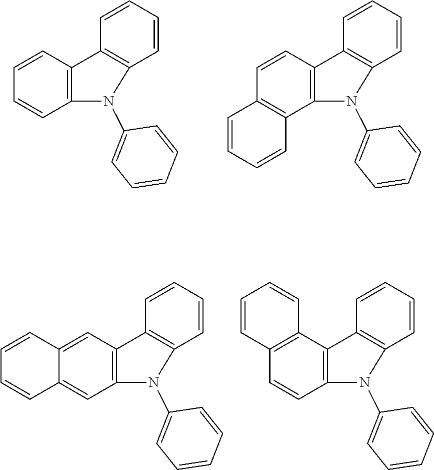

In the present disclosure, a direct linkage may refer to a single bond.

In the present disclosure, “ [symbol] ” and “ [symbol] ” each refer to a position to be connected.

Hereinafter, embodiments of the disclosure will be described in more detail with reference to the accompanying drawings. In the present disclosure, the terms “display device” and “display apparatus” may be used interchangeably.

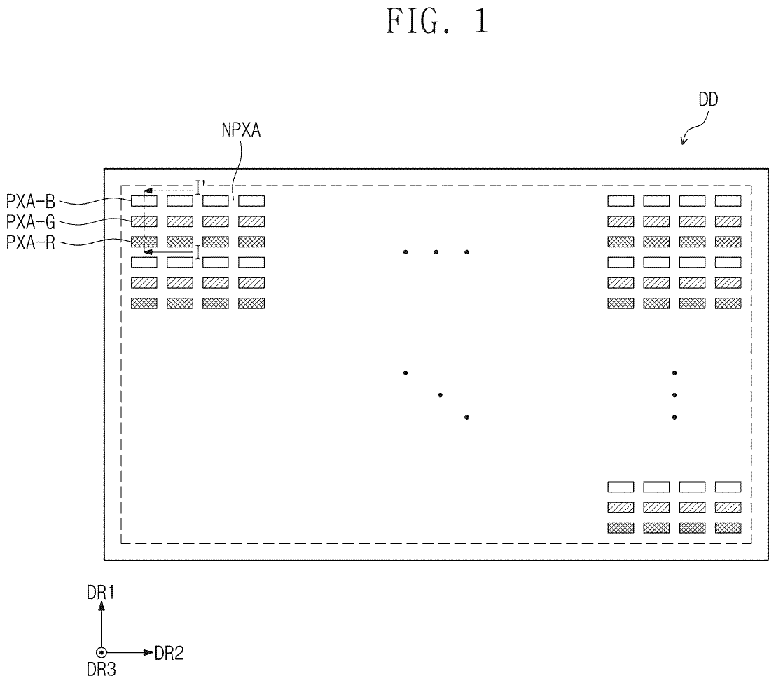

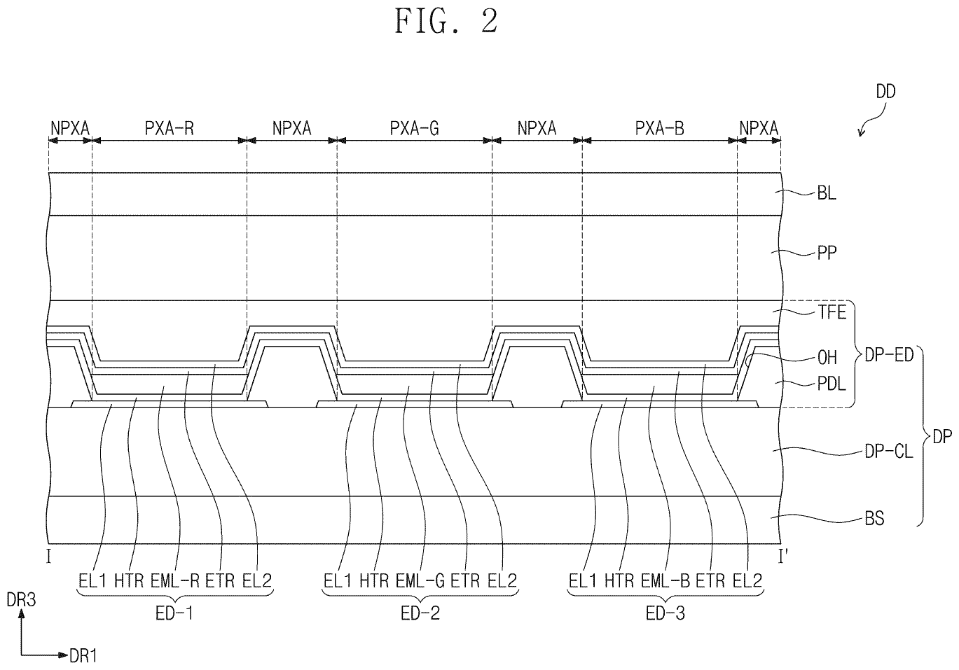

FIG. 1 is a plan view illustrating a display device DD according to one or more embodiments of the present disclosure. FIG. 2 is a cross-sectional view of the display device DD. FIG. 2 is a cross-sectional view illustrating a part taken along the line I-I′ of the display device DD of FIG. 1.

The display device DD may include a display panel DP and an optical layer PP arranged on the display panel DP. The display panel DP may include light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light. The optical layer PP may include, for example, a polarization layer and/or a color filter layer. In one or more embodiments, the optical layer PP may not be provided in the display device DD.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

The display device DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting elements ED-1, ED-2, and ED-3 arranged between respective portions of the pixel defining film PDL, and an encapsulation layer TFE arranged on the light emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, embodiments of the present disclosure are not limited thereto, and the base layer BS may be an inorganic layer, an organic layer, or a composite material layer.

In one or more embodiments, the circuit layer DP-CL may be arranged on the base layer BS, and the circuit layer DP-CL may include a plurality of transistors. Each of the transistors may include a control electrode, an input electrode, and an output electrode. For example, in some embodiments, the circuit layer DP-CL may include switching transistor(s) and driving transistor(s) for driving the light emitting elements ED-1, ED-2, and ED-3 of the display device layer DP-ED.

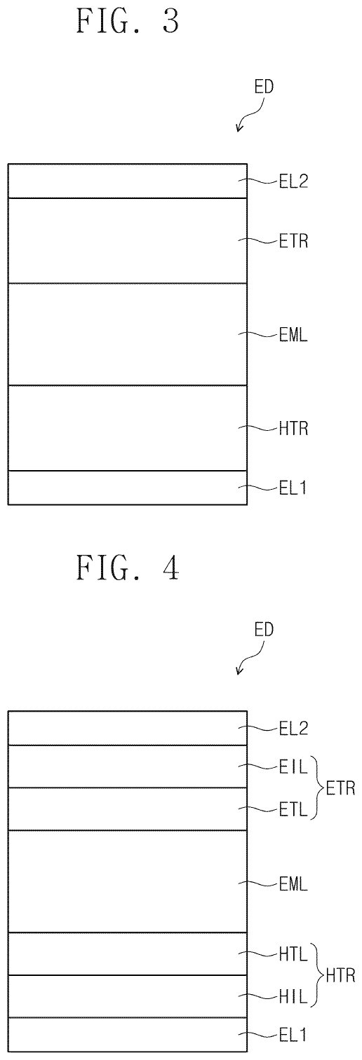

Each of the light emitting elements ED-1, ED-2, and ED-3 may have a structure of one of light emitting elements ED of embodiments according to FIGS. 3 to 6, which will be described in more detail later. Each of the light emitting elements ED-1, ED-2, and ED-3 may include a first electrode EL1, a hole transport region HTR, respective emission layers EML-R, EML-G, and EML-B, an electron transport region ETR, and a second electrode EL2.

FIG. 2 illustrates one or more embodiments in which the respective emission layers EML-R, EML-G, and EML-B of the light emitting elements ED-1, ED-2, and ED-3 are arranged in openings OH defined in the pixel defining film PDL, and the hole transport region HTR, the electron transport region ETR, and the second electrode EL2 are each provided as a common layer in the entire light emitting elements ED-1, ED-2, and ED-3. However, embodiments of the present disclosure are not limited thereto, for example, in one or more embodiments, the hole transport region HTR and the electron transport region ETR may each be provided by being patterned inside the openings OH defined in the pixel defining film PDL. For example, the hole transport region HTR, the respective emission layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2, and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to one or more embodiments may include at least one inorganic film (hereinafter, an encapsulation-inorganic film). The encapsulation layer TFE according to one or more embodiments may include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. In one or more embodiments, the encapsulation-organic film may include an acrylic-based compound, an epoxy-based compound, and/or the like. In one or more embodiments, the encapsulation-organic film may include a photopolymerizable organic material, but embodiments of the present disclosure are not particularly limited thereto.

The encapsulation layer TFE may be arranged on the second electrode EL2 and may be arranged filling the opening OH.

Referring to FIG. 1 and FIG. 2, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart (e.g., spaced apart or separated) from each other on a plane (e.g., in a plan view).

Each of the light emitting regions PXA-R, PXA-G, and PXA-B may be a region divided by the pixel defining film PDL. The non-light emitting areas NPXA may be areas between adjacent light emitting areas PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide the light emitting elements ED-1, ED-2, and ED-3. The respective emission layers EML-R, EML-G, and EML-B of the light emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display device DD of one or more embodiments illustrated in FIG. 1 and FIG. 2, three light emitting regions PXA-R, PXA-G, and PXA-B, which emit red light, green light, and blue light, respectively, are exemplarily illustrated. For example, the display device DD of one or more embodiments may include a red light emitting region PXA-R, a green light emitting region PXA-G, and a blue light emitting region PXA-B that are separated from each other.

In the display device DD according to one or more embodiments, the plurality of light emitting elements ED-1, ED-2 and ED-3 may be to emit light beams having wavelengths different from each other. For example, in one or more embodiments, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may be to emit light beams in substantially the same wavelength range or at least one light emitting element may be to emit a light beam in a wavelength range different from the others. For example, in one or more embodiments, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In addition, the red light emitting region PXA-R, the green light emitting region PXA-G, and the blue light emitting region PXA-B may be alternately arranged in this order along a first direction axis DR1.

FIG. 1 and FIG. 2 illustrate that all the light emitting regions PXA-R, PXA-G, and PXA-B have similar area, but embodiments of the present disclosure are not limited thereto. Thus, the light emitting regions PXA-R, PXA-G, and PXA-B may have different areas from each other according to the wavelength range of the emitted light. The areas of the light emitting regions PXA-R, PXA-G, and PXA-B may refer to areas when viewed on a plane defined by the first direction axis DR1 and the second direction axis DR2 (e.g., the areas in a plan view of the light emitting regions PXA-R, PXA-G, and PXA-B).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, in one or more embodiments, the arrangement form of the light emitting regions PXA-R, PXA-G, and PXA-B may be a pentile (PENTILE®) arrangement form (for example, an RGBG matrix, an RGBG structure, or an RGBG matrix structure) or a diamond (Diamond Pixel™) arrangement form (e.g., a display (e.g., an OLED display) containing red, blue, and green (RGB) light-emitting regions arranged in the shape of diamonds). PENTILE® is a duly registered trademark of Samsung Display Co., Ltd. Diamond Pixel™ is a trademark of Samsung Display Co., Ltd.

In one or more embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but embodiments of the present disclosure are not limited thereto.

Hereinafter, FIG. 3 to FIG. 6 are each a cross-sectional view schematically showing a light emitting element according to one or more embodiments of the present disclosure. The light emitting element ED according to one or more embodiments may include a first electrode EL1, a second electrode EL2 oppositely arranged to the first electrode EL1, and at least one functional layer arranged between the first electrode EL1 and the second electrode EL2. The light emitting element ED of one or more embodiments may include an amine compound of one or more embodiments of the present disclosure, which will be explained later, in the at least one functional layer.

In one or more embodiments, the light emitting element ED may include a hole transport region HTR, an emission layer EML, an electron transport region ETR, and/or the like, stacked in order (e.g., in the stated order), as the at least one functional layer. Referring to FIG. 3, the light emitting element ED of one or more embodiments may include a first electrode EL1, a hole transport region HTR, an emission layer EML, an electron transport region ETR, and a second electrode EL2, stacked in the stated order.

The light emitting element ED according to one or more embodiments may include an amine compound of one or more embodiments of the present disclosure, which will be explained later, in the hole transport region HTR between the first electrode EL1 and the second electrode EL2. However, embodiments of the present disclosure are not limited thereto. In addition to the hole transport region HTR, the light emitting element ED according to one or more embodiments may include an amine compound of one or more embodiments of the present disclosure, which will be explained later, in the emission layer EML or the electron transport region ETR each of which is a functional layer between the first electrode EL1 and the second electrode EL2, or may include an amine compound of one or more embodiments of the present disclosure, which will be explained later, in a capping layer CPL on the second electrode EL2.

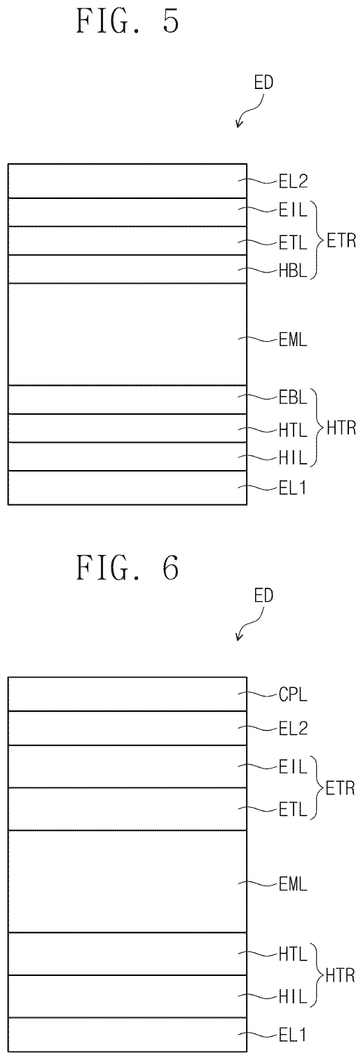

Compared with FIG. 3, FIG. 4 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In addition, compared with FIG. 3, FIG. 5 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light emitting element ED of one or more embodiments including a capping layer CPL arranged on a second electrode EL2.

The light emitting element ED according to one or more embodiments may include an amine compound of one or more embodiments of the present disclosure, which will be explained layer, in the hole transport region HTR. In the light emitting element ED according to one or more embodiments, at least one selected from among the hole injection layer HIL, the hole transport layer HTL, and the electron blocking layer EBL may include an amine compound of one or more embodiments of the present disclosure. In one or more embodiments, a layer adjacent to the emission layer selected from among the plurality of layers included in the hole transport region HTR may contain an amine compound of one or more embodiments represented by Formula 1. For example, in the light emitting element ED according to one or more embodiments, the hole transport layer HTL may include an amine compound of one or more embodiments represented by Formula 1. That is, in one or more embodiments, the light-emitting element ED may include the disclosed amine compound in the hole transport region HTR. This amine compound can be present in at least one of the hole injection layer HIL, the hole transport layer HTL, or the electron blocking layer EBL. Specifically, for example, the layer adjacent to the emission layer within the hole transport region HTR may contain an amine compound represented by Formula 1. For instance, the hole transport layer HTL in the light-emitting element ED may include this amine compound.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), a compound of two or more selected therefrom, a mixture of two or more selected therefrom, and/or an oxide thereof.

If (e.g., when) the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). If (e.g., when) the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In one or more embodiments, the first electrode EL1 may have a multilayer structure including a reflective film or a transflective film formed of one or more of the above-described materials, and a transparent conductive film formed of ITO, IZO, ZnO, ITZO, and/or the like. For example, in one or more embodiments, the first electrode EL1 may have a three-layer structure of ITO/Ag/ITO, but embodiments of the present disclosure are not limited thereto. The first electrode EL1 may include one of the above-described metal materials, one or more combinations of at least two metal materials of the above-described metal materials, any oxide of the above-described metal materials, and/or the like. A thickness of the first electrode EL1 may be from about 700 ångströms (Å) to about 10,000 Å. For example, in one or more embodiments, the thickness of the first electrode EL1 may be from about 1,000 Å to about 3,000 Å.

The hole transport region HTR may be provided on the first electrode EL1. The hole transport region HTR may have a single layer formed of a single material, a single layer formed of a plurality of different materials, or a multilayer structure including a plurality of layers formed of a plurality of different materials.

In one or more embodiments, the hole transport region HTR may include at least one of a hole injection layer HIL, a hole transport layer HTL, or an electron blocking layer EBL. In one or more embodiments, the hole transport region HTR may include multiple hole transport layers stacked.

For example, in one or more embodiments, the hole transport region HTR may have a single layer structure of a hole injection layer HIL or a hole transport layer HTL, or may have a single layer structure formed of a hole injection material and a hole transport material. In one or more embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in the stated order) from the first electrode EL1, but embodiments of the present disclosure are not limited thereto.

A thickness of the hole transport region HTR may be, for example, in a range of 50 Å to about 15,000 Å. The hole transport region HTR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

The light emitting element ED of one or more embodiments may include an amine compound of one or more embodiments of the present disclosure in the hole transport region HTR. In the light emitting element ED of one or more embodiments, the hole transport region HTR may include an hole injection layer HIL and a hole transport layer HTL, and the hole transport layer HTL may include the amine compound of one or more embodiments. In one or more embodiments, the amine compound of one or more embodiments may be included in a layer adjacent to the emission layer EML among the layers included in the hole transport region HTR.

The amine compound of one or more embodiments includes an amine group and further includes a first substituent, a second substituent, and a third substituent, each of which is linked to the amine group. For example, the amine compound of one or more embodiments may include an amine group, that is, a structure including a core nitrogen atom and having the first substituent, the second substituent, and the third substituent all bonded to the core nitrogen atom.

The first substituent may include a dibenzofuran moiety, a dibenzothiophene moiety, a carbazole moiety, or a fluorene moiety. In the amine compound of one or more embodiments, the dibenzofuran moiety, the dibenzothiophene moiety, the carbazole moiety, or the fluorene moiety is bonded to the core nitrogen atom, and an oxygen atom of the dibenzofuran moiety, a sulfur atom of the dibenzothiophene moiety, a nitrogen atom of the carbazole moiety, or a 9th carbon atom of the fluorene moiety may be positioned meta with respect to the core nitrogen atom. For example, in the amine compound of one or more embodiments, the core nitrogen atom may be linked to a 3rd carbon of the dibenzofuran moiety, a 3rd carbon of the dibenzothiophene moiety, a 3rd carbon of the carbazole moiety, or a 3rd carbon of the fluorene moiety. The carbon numbering of the dibenzofuran moiety, the dibenzothiophene moiety, the carbazole moiety, or the fluorene moiety in the first substituent will be further described with reference to Formula S1 later.

The first substituent is directly bonded to the core nitrogen atom. The first substituent may further include a first sub-substituent linked to a carbon at a specific position among the carbon atoms constituting the dibenzofuran moiety, the dibenzothiophene moiety, the carbazole moiety, and the fluorene moiety. The first sub-substituent may be directly bonded to the dibenzofuran moiety, the dibenzothiophene moiety, the carbazole moiety, and the fluorene moiety. In one or more embodiments, the first sub-substituent may be a substituted or unsubstituted aryl group having 6 to 30 ring-forming carbon atoms.

In the amine compound of one or more embodiments, three first sub-substituents or less may be provided. The first sub-substituent may be linked to at least one of a 5th carbon, a 7th carbon, or a 8th carbon of the dibenzofuran moiety, the dibenzothiophene moiety, the carbazole moiety, and the fluorene moiety. For example, the 3rd carbon of the dibenzofuran moiety, the 3rd carbon of the dibenzothiophene moiety, the 3rd carbon of the carbazole moiety, and the 3rd carbon of the fluorene moiety in the first substituent may be linked to the core nitrogen atom, and at least one of the 5th carbon, the 7th carbon, or the 8th carbon among the other carbons may be linked to the first sub-substituent. For example, in one or more embodiments, the first sub-substituent may be linked to the 5th carbon of the dibenzofuran moiety, the 5th carbon of the dibenzothiophene moiety, the 5th carbon of the carbazole moiety, and the 5th carbon of the fluorene moiety. Alternatively, in one or more embodiments, the first sub-substituent may be linked to the 7th carbon of the dibenzofuran moiety, the 7th carbon of the dibenzothiophene moiety, the 7th carbon of the carbazole moiety, and the 7th carbon of the fluorene moiety. Alternatively, in one or more embodiments, the first sub-substituent may be linked to the 8th carbon of the dibenzofuran moiety, the 8th carbon of the dibenzothiophene moiety, the 8th carbon of the carbazole moiety, and the 8th carbon of the fluorene moiety. However, embodiments of the present disclosure are not limited thereto. The amine compound of one or more embodiments has a structure in which the first sub-substituent is linked to a specific position of the dibenzofuran moiety, the dibenzothiophene moiety, the carbazole moiety, and the fluorene moiety, and may thus exhibit high thermal properties, and accordingly, if (e.g., when) applied to the light emitting element ED of one or more embodiments, may contribute to improving element service life and efficiency. In other words, the amine compound in one or more embodiments may have up to three first sub-substituents. These sub-substituents may be attached to specific carbon positions (5th, 7th, or 8th) on the dibenzofuran, dibenzothiophene, carbazole, or fluorene moieties. For instance, the 3rd carbon of these moieties is linked to the core nitrogen atom, while the 5th, 7th, or 8th carbon can be linked to the first sub-substituent. This specific structure enhances the thermal properties of the amine compound, which, when used in the light-emitting element ED, may improve its efficiency and lifespan.

In present disclosure, the carbon numbers of the dibenzofuran moiety, the dibenzothiophene moiety, the carbazole moiety, and the fluorene moiety in the first substituent are defined as shown in Formula S1.

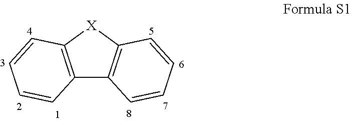

In Formula S1, X is O, S, NAr², or CAr³Ar⁴. Ar² to Ar⁴ may each independently be a substituted or unsubstituted aryl group having 6 to 30 ring-forming carbon atoms. In Formula S1, if (e.g., when) X is O, the first substituent may include a dibenzofuran moiety. In Formula S1, if (e.g., when) X is S, the first substituent may include a dibenzothiophene moiety. In Formula S1, if (e.g., when) X is NAr², the first substituent may include a carbazole moiety. In Formula S1, if (e.g., when) X is CAr³Ar⁴, the first substituent may include a fluorene moiety.

In the carbon numbering of the dibenzofuran moiety, the dibenzothiophene moiety, the carbazole moiety, and the fluorene moiety, if (e.g., when) the first substituent is placed such that X is placed on an upper side as shown in Formula S1, it is shown that carbons are numbered clockwise from a carbon atom placed on a lower side and positioned meta to X among the carbon atoms forming a left benzene ring, and carbons at fused positions are not numbered. In Formula S1, if (e.g., when) X is CAr³Ar⁴, C included in X may be a 9th carbon. Here, for convenience of description, substituents linked to both (e.g., simultaneously) benzene rings are not given. Unlike Formula S1, in one or more embodiments of the present disclosure, the dibenzofuran moiety, the dibenzothiophene moiety, the carbazole moiety, and the fluorene moiety in the first substituent may each have at least one substituent in addition to hydrogen atoms.

The second substituent may include an ortho-terphenyl moiety. The second substituent may be bonded to the amine group of the amine compound of one or more embodiments through a linker. In one or more embodiments, the second substituent may be linked to the core nitrogen atom through a linker. The ortho-terphenyl moiety includes three benzene rings and a structure in which the three benzene rings are linked to be positioned ortho. For example, herein, the ortho-terphenyl may be represented by Formula S2.

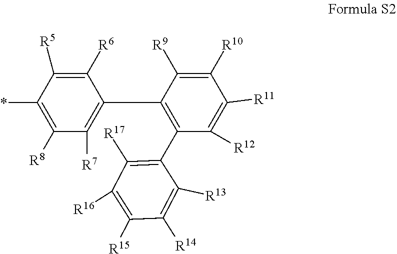

In Formula S2, the descriptions of R⁵ to R¹⁷ in Formula 1, which will be described in more detail later, may apply to R⁵ to R¹⁷. In Formula S2, [symbol] may be a position linked to the linker of the amine compound of one or more embodiments.

The third substituent is directly bonded to the core nitrogen atom. For example, the third substituent may be directly linked to the core nitrogen atom without a separate linker. In one or more embodiments, the third substituent may be a substituted or unsubstituted aryl group having 6 to 30 ring-forming carbon atoms or a substituted or unsubstituted heteroaryl group having 2 to 30 ring-forming carbon atoms. In one or more embodiments, the third substituent may indicate a substituent represented by Ar¹ in Formula 1, which will be described in more detail later.

The amine compound of one or more embodiments may be a monoamine compound including one amine group. The amine compound of one or more embodiments may be a monoamine compound having only one amine group present in a molecular structure without forming a ring.

In one or more embodiments, the amine compound may be represented by Formula 1.

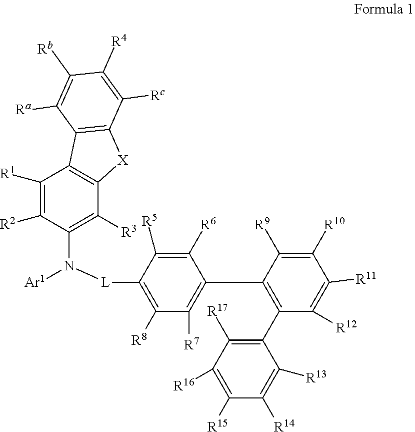

In Formula 1, X may be O, S, NAr², or CAr³Ar⁴.

In Formula 1, R^{a} to R^{c} may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In one or more embodiments, R^{a} to R^{c} may each independently be hydrogen, deuterium, or a substituted or unsubstituted aryl group having 6 to 30 ring-forming carbon atoms. For example, in one or more embodiments, R^{a} to R^{c} may each independently be hydrogen, deuterium, or a substituted or unsubstituted phenyl group.

In Formula 1, at least one selected from among R^{a} to R^{c} is a substituted or unsubstituted aryl group having 6 to 30 ring-forming carbon atoms. For example, in one or more embodiments, one selected from among R^{a} to R^{c} may be a substituted or unsubstituted aryl group having 6 to 30 ring-forming carbon atoms, and the other two may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, in one or more embodiments, two selected from among R^{a} to R^{c} may each independently be a substituted or unsubstituted aryl group having 6 to 30 ring-forming carbon atoms, and the remaining one may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, in one or more embodiments, R^{a} to R^{c} may all be a substituted or unsubstituted aryl group having 6 to 30 ring-forming carbon atoms.

In one or more embodiments, at least one selected from among R^{a} to R^{c} may be a substituted or unsubstituted phenyl group. For example, in one or more embodiments, one selected from among R^{a} to R^{c} may be a substituted or unsubstituted phenyl group, and the other two may each independently be hydrogen or deuterium. Alternatively, in one or more embodiments, two selected from among R^{a} to R^{c} may each independently be a substituted or unsubstituted phenyl group, and the remaining one may be hydrogen or deuterium. Alternatively, in one or more embodiments, R^{a} to R^{c} may all be substituted or unsubstituted phenyl groups.

In Formula 1, R¹ to R⁴ may each independently be hydrogen, deuterium, a substituted or unsubstituted alkyl group having 1 to 20 carbon atoms, or a substituted or unsubstituted aryl group having 6 to 30 ring-forming carbon atoms, For example, in one or more embodiments, R¹ to R⁴ may each independently be hydrogen or deuterium.

In Formula 1, Ar¹ may be a substituted or unsubstituted aryl group having 6 to 30 ring-forming carbon atoms or a substituted or unsubstituted heteroaryl group having 2 to 30 ring-forming carbon atoms.

In one or more embodiments, Ar¹ may be selected from among Substituent Group C, which will be described in more detail later. Ar¹ may be any one selected from among Substituent Group C.

In one or more embodiments, Ar¹ may be a substituted or unsubstituted phenyl group, a substituted or unsubstituted biphenylyl group, a substituted or unsubstituted naphthyl group, a substituted or unsubstituted naphthylphenyl group, a substituted or unsubstituted phenanthrenyl group, a substituted or unsubstituted dibenzofuranyl group, or a substituted or unsubstituted dibenzothiophenyl group.

In one or more embodiments, if (e.g., when) Ar¹ is substituted, the substituent of Ar¹ may be a substituted or unsubstituted aryl group having 6 to 15 ring-forming carbon atoms. For example, in one or more embodiments, if (e.g., when) Ar¹ is substituted, the substituent of Ar¹ may be deuterium, a substituted or unsubstituted phenyl group, or a substituted or unsubstituted naphthyl group.

In Formula 1, Ar² to Ar⁴ may each independently be a substituted or unsubstituted aryl group having 6 to 30 ring-forming carbon atoms, For example, in one or more embodiments, Ar² to Ar⁴ may each independently be a substituted or unsubstituted phenyl group.

In Formula 1, L may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, L may be a direct linkage or a substituted or unsubstituted arylene group having 6 to 30 ring-forming carbon atoms. For example, in one or more embodiments, L may be a direct linkage or a substituted or unsubstituted phenylene group.

In Formula 1, R⁵ to R¹⁷ may each independently be hydrogen, deuterium, a substituted or unsubstituted alkyl group having 1 to 20 carbon atoms, a substituted or unsubstituted alkenyl group having 2 to 30 carbon atoms, or a substituted or unsubstituted aryl group having 6 to 30 ring-forming carbon atoms. Alternatively, in one or more embodiments, R⁵ to R¹⁷ may each independently be bonded to an adjacent group to form a ring. For example, in one or more embodiments, R⁵ to R¹⁷ may each independently be hydrogen, deuterium, or a substituted or unsubstituted phenyl group. Alternatively, in one or more embodiments, at least one pair of adjacent pairs among R⁵ to R¹⁷ may be bonded together to form an aromatic hydrocarbon ring or an aromatic hetero ring. For example, R¹⁴ and R¹⁵, or R¹⁵ and R¹⁶ may be bonded together to form an aromatic hydrocarbon ring.

In the amine compound of one or more embodiments represented by Formula 1, a case in which L is a direct linkage and R⁹ and R¹⁰, and R¹¹ and R¹² are each bonded together to form a ring is excluded. In one or more embodiments, a case in which a pair of R⁹ and R¹⁰ and a pair of R¹¹ and R¹² are each bonded together to form an aromatic hydrocarbon ring if (e.g., when) L is a direct linkage in Formula 1 may be excluded. For example, a case in which a pair of R⁹ and R¹⁰ and a pair of R¹¹ and R¹² are each bonded together to form an aromatic hydrocarbon ring such as

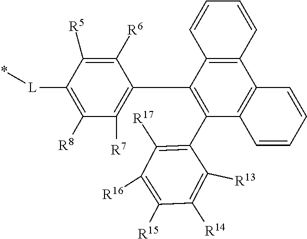

if (e.g., when) L is a direct linkage in Formula 1 may be excluded. In the amine compound represented by Formula 1, if (e.g., when) L is a direct linkage, and both (e.g., simultaneously) a pair of R⁹ and R¹⁰ and a pair of R¹¹ and R¹² are subjected to fused cyclization, intramolecular distortion may be caused and thus the thermal stability of the amine compound may be reduced. According to one or more embodiments of the present disclosure, the thermal stability of the amine compound may be improved as the case in which L is a direct linkage, and a pair of R⁹ and R¹⁰ and a pair of R¹¹ and R¹² are each bonded together to form a ring is excluded in the amine compound of one or more embodiments represented by Formula 1, and accordingly, if (e.g., when) the amine compound of one or more embodiments is applied to a light emitting element, high efficiency and long life of the light emitting element may be achieved.

In the amine compound of one or more embodiments represented by Formula 1, a case in which L is a direct linkage and any of the pairs R¹² and R¹³, and R¹² and R¹⁷ are bonded together to form a heterocycle is excluded. In one or more embodiments, a case in which L is a direct linkage and any of the pairs R¹² and R¹³, and R¹² and R¹⁷ are bonded together to form an aromatic heterocycle may be excluded. For example, a case in which R¹² and R¹³ are bonded together to form a dibenzofuran ring such as

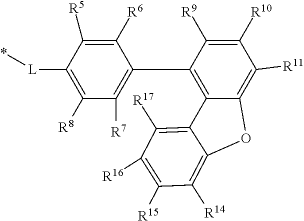

or a dibenzothiophene ring such as

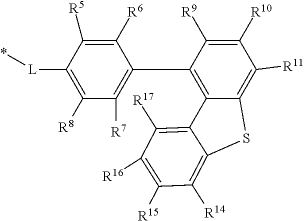

if (e.g., when) L is a direct linkage in Formula 1 may be excluded. Alternatively, in one or more embodiments, a case in which R¹² and R¹⁷ are bonded together to form a dibenzofuran ring such as

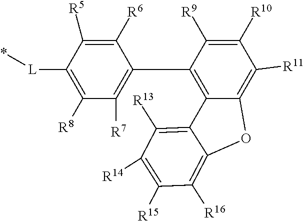

or a dibenzothiophene ring such as

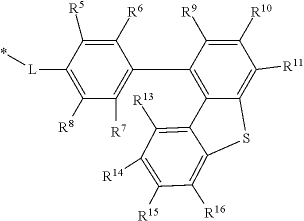

if (e.g., when) L is a direct linkage in Formula 1 may be excluded. In the amine compound represented by Formula 1, if (e.g., when) L is a direct linkage and any of the pairs R¹² and R¹³, and R¹² and R¹⁷ are bonded together to form a heterocycle, a benzene ring substituted by R⁵ to R⁸ and the heterocycle may be greatly distorted sterically, resulting in reduced thermal stability of the amine compound. According to one or more embodiments of the present disclosure, the thermal stability of the amine compound may be improved as the case in which L is a direct linkage, and any of the pairs R¹² and R¹³, and R¹² and R¹⁷ are bonded together to form a heterocycle is excluded in the amine compound of one or more embodiments represented by Formula 1, and accordingly, if (e.g., when) the amine compound of one or more embodiments is applied to a light emitting element, high efficiency and long life of the light emitting element may be achieved.

In the amine compound of one or more embodiments represented by Formula 1, a case in which L is a direct linkage and R¹¹ and R¹² are bonded together to form a nitrogen-containing heterocycle is excluded. For example, a case in which L is a direct linkage, and R¹¹ and R¹² are bonded together to form a carbazole ring such as

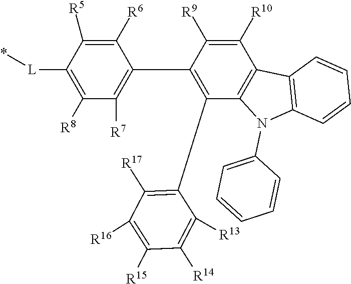

may be excluded. In the amine compound represented by Formula 1, if (e.g., when) L is a direct linkage and R¹¹ and R¹² are bonded together to form a nitrogen-containing heterocycle, a benzene ring substituted by R¹³ to R¹⁷ and the nitrogen-containing heterocycle may be greatly distorted sterically, resulting in reduced thermal stability of the amine compound. According to one or more embodiments of the present disclosure, the stability of the amine compound may be improved as the case in which L is a direct linkage, and R¹¹ and R¹² are bonded together to form a heterocycle is excluded in the amine compound of one or more embodiments represented by Formula 1, and accordingly, if (e.g., when) the amine compound of one or more embodiments is applied to a light emitting element, high efficiency and long life of the light emitting element may be achieved.

In the amine compound of one or more embodiments represented by Formula 1, a case in which Ar¹ is an unsubstituted phenyl group if (e.g., when) X is CAr³Ar⁴ is excluded.

In one or more embodiments, a case in which the amine compound represented by Formula 1 includes a substituted or unsubstituted fluorene moiety, or an N,N-bis(4-(2-naphthyl)phenyl)amine moiety is excluded.

In one or more embodiments, a case in which the amine compound represented by Formula 1 includes a fluorene moiety substituted with at least one alkyl group at position 9 or unsubstituted, a substituted or unsubstituted 9-fluorene moiety, or a substituted or unsubstituted spirobifluorene moiety is excluded. For example, a case in which the amine compound represented by Formula 1 includes a substituted or unsubstituted 9-fluorene moiety, a spirobifluorene moiety such as

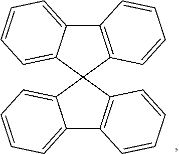 [SMILES: c1ccc2c(c1)-c1ccccc1C21c2ccccc2-c2ccccc21]

or a 9,9-dimethylfluorene moiety such as

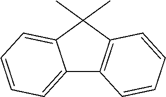

may be excluded. When the fluorene moiety having the structure described above in included in the amine compound, the amine compound may be thermally and chemically unstable, which may cause a decrease in element service life. According to one or more embodiments of the present disclosure, the amine compound of one or more embodiments represented by Formula 1 does not include the fluorene moiety having the structure described above, and thus the amine compound may have improved thermal and chemical stability, resulting in improved element service life. The 9-fluorene moiety is represented by Formula A.

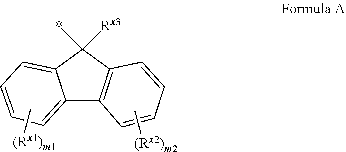

In Formula A, R^{x1} to R^{x3} may each independently be hydrogen, deuterium, a halogen, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula A, m1 and m2 may each independently be an integer of 0 to 4.

In Formula A, [symbol] may be a position at which the moiety represented by [symbol] Formula A is linked to the structure of Formula 1. For example, “ [symbol] ” may be a position at which the moiety represented by Formula A is linked to a central nitrogen atom in Formula 1, or linked to the first substituent or the second substituent described above.

In one or more embodiments, a case in which the amine compound represented by Formula 1 includes an N,N-bis(4-(2-naphthyl)phenyl)amine moiety is excluded. In one or more embodiments, a case in which R¹¹ and R¹² are bonded together to form an aromatic hydrocarbon ring and Ar¹ is a naphthyl phenyl group such as

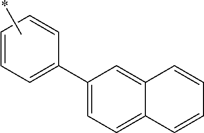

in the amine compound represented by Formula 1 may be excluded. For example, a case in which the amine compound represented by Formula 1 is represented by Formula 1-a may be excluded.

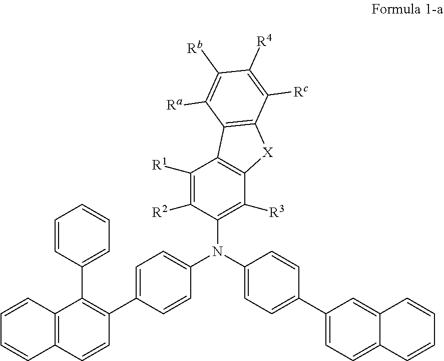

Formula 1-a corresponds to a case in which R¹¹ and R¹² are bonded together to form an additional fused benzene ring, and Ar¹ is an unsubstituted naphthylphenyl group in Formula 1. When the amine compound includes an N,N-bis(4-(2-naphthyl)phenyl)amine moiety, deposition temperature may increase, causing a concern or issue over thermal decomposition of the material upon deposition. According to one or more embodiments of the present disclosure, the thermal stability of the amine compound may be improved as the amine compound of one or more embodiments represented by Formula 1 does not include an N,N-bis(4-(2-naphthyl)phenyl)amine moiety. Accordingly, if (e.g., when) the amine compound of one or more embodiments is applied to a light emitting element, improved element service life of the light emitting element may be achieved.

In one or more embodiments, the amine compound represented by Formula 1 may be represented by any one selected from among Formulas 2-1 to 2-3.

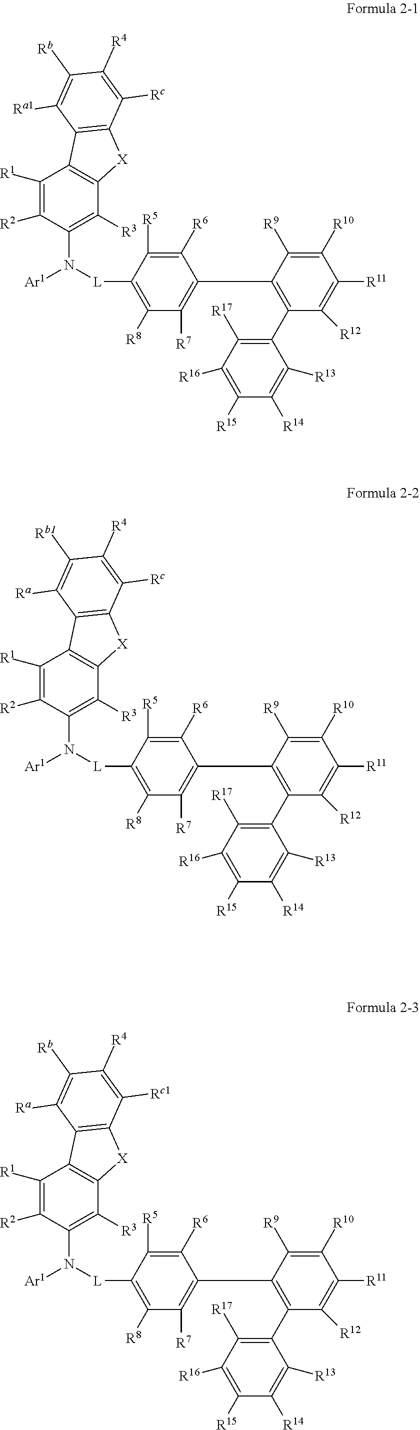

Formulas 2-1 and 2-3 show embodiments in which the types (kinds) of R^{a} to R^{c} in Formula 1 are specified. Formula 2-1 corresponds to an embodiment in which R^{a} is a substituted or unsubstituted aryl group having 6 to 30 ring-forming carbon atoms in Formula 1. Formula 2-2 corresponds to an embodiments in which R^{b} is a substituted or unsubstituted aryl group having 6 to 30 ring-forming carbon atoms in Formula 1. Formula 2-3 corresponds to an embodiment in which R^{c} is a substituted or unsubstituted aryl group having 6 to 30 ring-forming carbon atoms in Formula 1.

In Formulas 2-1 to 2-3, R^{a1}, R^{b1}, and R^{c1} may each independently be a substituted or unsubstituted aryl group having 6 to 30 ring-forming carbon atoms. For example, in one or more embodiments, R^{a1}, R^{b1}, and R^{c1} may each independently be a substituted or unsubstituted phenyl group.

In Formulas 2-1 to 2-3, Ar¹, L, X, R^{a}, R^{b}, R^{c}, and R¹ to R¹⁷ may each be the same as described in Formula 1.

In one or more embodiments, the amine compound represented by Formula 1 may be represented by Formula 3.

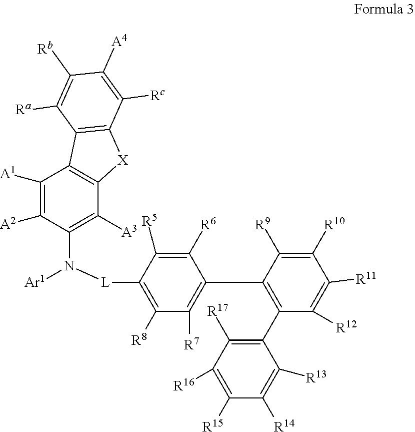

Formula 3 shows embodiments in which the types (kinds) of R¹ to R⁴ in Formula 1 are specified.

In Formula 3, A¹ to A⁴ may each independently be hydrogen or deuterium.

In Formulas 3, Ar¹, L, X, R^{a}, R^{b}, R^{c}, and R⁵ to R¹⁷ may each be the same as described in Formula 1.

In one or more embodiments, the amine compound represented by Formula 1 may be represented by Formula 4-1 or Formula 4-2.

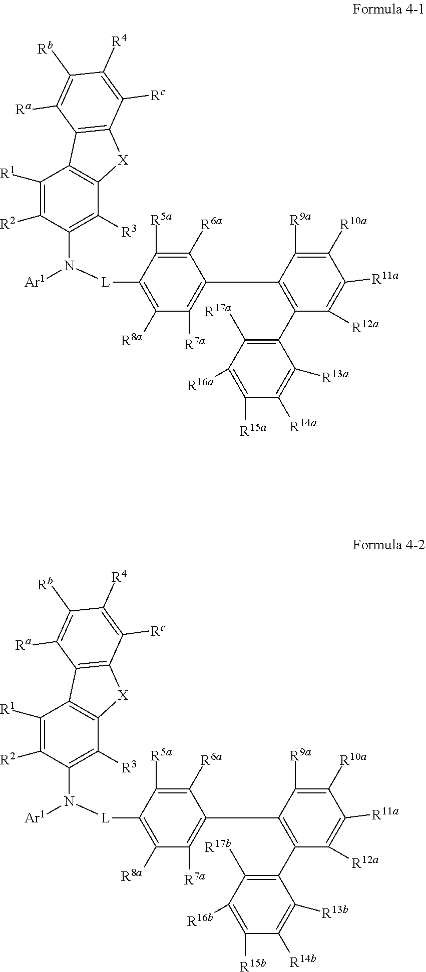

In Formula 4-1, R^{5a} to R^{17a} may each independently be hydrogen, deuterium, a substituted or unsubstituted alkyl group having 1 to 20 carbon atoms, or a substituted or unsubstituted aryl group having 6 to 30 ring-forming carbon atoms. In one or more embodiments, R^{5a} to R^{17a} may each independently be hydrogen, deuterium, or a substituted or unsubstituted aryl group having 6 to 30 ring-forming carbon atoms. For example, in one or more embodiments, R^{5a} to R^{17a} may each independently be hydrogen, deuterium, or a substituted or unsubstituted phenyl group.

In Formula 4-2, R^{5a} to R^{12a} may each independently be hydrogen, deuterium, a substituted or unsubstituted alkyl group having 1 to 20 carbon atoms, or a substituted or unsubstituted aryl group having 6 to 30 ring-forming carbon atoms. For example, in one or more embodiments, R^{5a} to R^{12a} may each independently be hydrogen or deuterium.

In Formula 4-2, R^{13b} to R^{17b} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms. Alternatively, in one or more embodiments, R^{13b} to R^{17b} may each independently be bonded to an adjacent group to form a ring. Alternatively, in one or more embodiments, at least one pair selected from among adjacent pairs among R^{13b} to R^{17b} may be bonded together to form an aromatic hydrocarbon ring.

In Formulas 4-1 and 4-2, Ar¹, L, X, R^{a}, R^{b}, R^{c}, and R¹ to R⁴ may each be the same as described in Formula 1.

In one or more embodiments, the amine compound represented by Formula 1 may be represented by Formula 5.

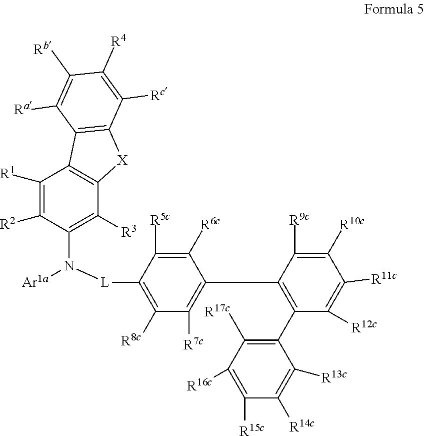

In Formula 5, R^{a}′ to R^{c}′ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula 5, at least one selected from among R^{a}′ to R^{c}′ may be a substituted or unsubstituted phenyl group. For example, in one or more embodiments, one selected from among R^{a}′ to R^{c}′ may be a substituted or unsubstituted phenyl group, and the other two may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, in one or more embodiments, two selected from among R^{a}′ to R^{c}′ may be a substituted or unsubstituted phenyl group, and the remaining one may be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, in one or more embodiments, R^{a}′ to R^{c}′ may all be a substituted or unsubstituted phenyl group.

In one or more embodiments, at least one selected from among R^{a}′ to R^{c}′ may be a substituted or unsubstituted phenyl group, and the others may each independently be hydrogen or deuterium. For example, in one or more embodiments, one selected from among R^{a}′ to R^{c}′ may be a substituted or unsubstituted phenyl group, and the other two may each independently be hydrogen or deuterium. Alternatively, in one or more embodiments, two selected from among R^{a}′ to R^{c}′ may each independently be a substituted or unsubstituted phenyl group, and the remaining one may be hydrogen or deuterium. Alternatively, in one or more embodiments, R^{a}′ to R^{c}′ may all be a substituted or unsubstituted phenyl group.

In Formula 5, Ar^{1a} may be a substituted or unsubstituted aryl group having 6 to 30 ring-forming carbon atoms.

In one or more embodiments, Ar^{1a} may be selected from Substituent Group C described herein. In one or more embodiments, Ar^{1a} may be any one selected from Substituent Group C described herein.

In one or more embodiments, Ar^{1a} may be a substituted or unsubstituted phenyl group, a substituted or unsubstituted biphenylyl group, a substituted or unsubstituted naphthyl group, a substituted or unsubstituted naphthylphenyl group, a substituted or unsubstituted phenanthrenyl group, a substituted or unsubstituted dibenzofuranyl group, or a substituted or unsubstituted dibenzothiophenyl group.

In one or more embodiments, if (e.g., when) Ar^{1a} is substituted, the substituent of Ar^{1a} may be deuterium or a substituted or unsubstituted aryl group having 6 to 15 ring-forming carbon atoms. For example, in one or more embodiments, if (e.g., when) Ar^{1a} is substituted, the substituent of Ar^{1a} may be deuterium, a substituted or unsubstituted phenyl group, or a substituted or unsubstituted naphthyl group.

In Formula 5, R^{5c} to R^{17c} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms. Alternatively, in one or more embodiments, R^{5c} to R^{17c} may each independently be bonded to an adjacent group to form a ring. For example, in one or more embodiments, R^{5c} to R^{17c} may each independently be hydrogen, deuterium, or a substituted or unsubstituted phenyl group. Alternatively, in one or more embodiments, at least one pair selected from among adjacent pairs among R^{5c} to R^{17c} may be bonded together to form an aromatic hydrocarbon ring or an aromatic hetero ring. For example, R^{14c} and R^{15c}, or R^{15c} and R^{16c} may be bonded together to form an aromatic hydrocarbon ring.

In Formula 5, a case in which if (e.g., when) R^{11c} and R^{12c} are bonded together to form a ring, Ar^{1a} is a substituted or unsubstituted naphthylphenyl group may be excluded. For example, in Formula 5, a case in which R^{11c} and R^{12c} are bonded together to form an aromatic hydrocarbon ring and Ar^{1a} is a naphthylphenyl group such as

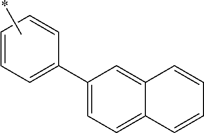

may be excluded.

In Formula 5, a case in which L is a direct linkage and R^{9c} and R^{10c}, and R^{11c} and R^{12c} are each bonded together to form a ring may be excluded.

In Formula 5, a case in which L is a direct linkage and any of the pairs R^{12c} and R^{13c}, and R^{12c} and R^{17c} are bonded together to form a heterocycle may be excluded. For example, in Formula 5, a case in which L is a direct linkage and any of the pairs R^{12c} and R^{13c}, and R^{12c} and R^{17c} are bonded together to form a dibenzofuran ring or a dibenzothiophene ring may be excluded.

In Formula 5, a case in which L is a direct linkage and R^{11c} and R^{12c} are bonded together to form a nitrogen-containing heterocycle may be excluded. For example, in Formula 5, a case in which L is a direct linkage and R^{11c} and R^{12c} are bonded together to form a carbazole ring may be excluded.

In Formula 5, a case in which L is a direct linkage and R^{6c} and R^{9c} are bonded together to form a ring may be excluded. In one or more embodiments, in Formula 5, a case in which L is a direct linkage and R^{6c} and R^{9c} are bonded together to form an aromatic hydrocarbon ring may be excluded. For example, in Formula 5, a case in which L is a direct linkage and R^{6c} and R^{9c} are bonded together to form a fluorene ring such as

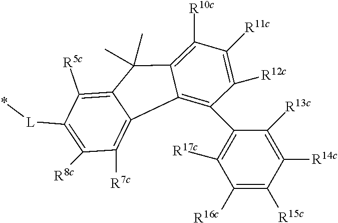

may be excluded.

In Formula 5, if (e.g., when) Ar^{1a} is substituted, the substituent of Ar^{1a} may be deuterium or a substituted or unsubstituted aryl group having 6 to 15 ring-forming carbon atoms. For example, in one or more embodiments, if (e.g., when) Ar^{1a} is substituted, the substituent of Ar^{1a} may be deuterium, a substituted or unsubstituted phenyl group, or a substituted or unsubstituted naphthyl group.

In Formula 5, the same descriptions as those described in Formula 1 may apply to R¹ to R⁴, X, and L.

In one or more embodiments, the amine compound represented by Formula 1 may be represented by Formula 6.

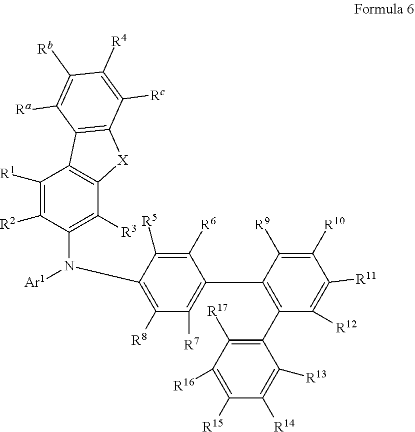

Formula 6 shows an embodiment in which the type or kind of L in Formula 1 is specified.

In Formulas 6, the same descriptions as those described in Formula 1 may apply to Ar¹, X, R^{a}, R^{b}, R^{c}, and R¹ to R¹⁷.

In one or more embodiments, the amine compound represented by Formula 1 may be represented by any one selected from among Formulas 7-1 to 7-3.

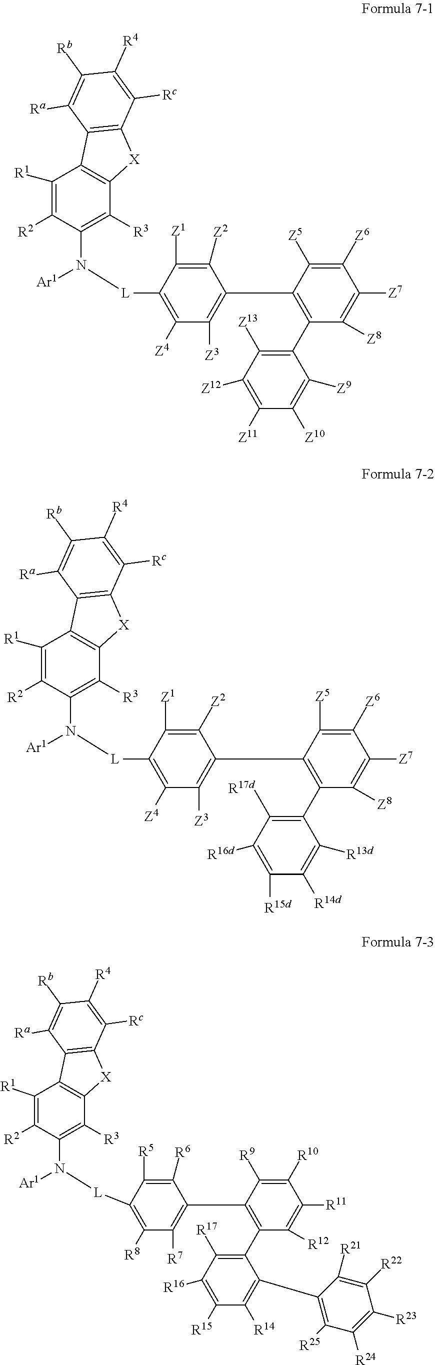

In Formula 7-1, Z¹ to Z¹³ may each independently be hydrogen or deuterium. Alternatively, in one or more embodiments, in Formula 7-1, Z¹ to Z¹³ may each be hydrogen.

In Formula 7-2, Z¹ to Z⁸ may each independently be hydrogen or deuterium. Alternatively, in one or more embodiments, in Formula 7-2, Z¹ to Z⁸ may each be hydrogen.

In Formula 7-2, R^{13d} to R^{17d} may each independently be hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms. Alternatively, in one or more embodiments, R^{13d} to R^{17d} may each independently be bonded to an adjacent group to form a ring. Alternatively, in one or more embodiments, at least one pair selected from among adjacent pairs among R^{13d} to R^{17d} may be bonded together to form an aromatic hydrocarbon ring.

In Formula 7-3, R²¹ to R²⁵ may each independently be hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Alternatively, in one or more embodiments, R²¹ to R²⁵ may each independently be bonded to an adjacent group to form a ring. For example, in one or more embodiments, R²¹ to R²⁵ may each independently be hydrogen or deuterium.

In Formulas 7-1 to 7-3, Ar¹, L, X, R^{a}, R^{b}, R^{c}, R¹ to R¹², and R¹⁴ to R¹⁷ may each be the same as described in Formula 1.

In one or more embodiments, the amine compound of one or more embodiments represented by Formula 1 may include at least one deuterium as a substituent. The amine compound of one or more embodiments represented by Formula 1 may include a structure in which at least one hydrogen is substituted with deuterium.

In one or more embodiments, the amine compound represented by Formula 1 may be represented by Formula 8, and the amine compound may be a compound satisfying any one selected from among the combinations shown in Compound Combination Table 1.

The hole transport region HTR of the light emitting element ED of one or more embodiments may include at least one selected from among the amine compounds satisfying the combinations shown in Compound Combination Table 1. For example, in one or more embodiments, the hole transport layer HTL of the light emitting element ED may include at least one selected from among the amine compounds satisfying the combinations shown in Compound Combination Table 1.

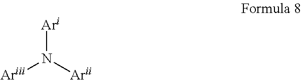

In Formula 8, Arⁱ may be any one selected from Substituent Group A, Arⁱⁱ may be any one selected from Substituent Group B, and Arⁱⁱⁱ may be any one selected from Substituent Group C. The superscript utilized in Substituent Group A, Substituent Group B, and Substituent Group C corresponds to the reference numeral used in Compound Combination Table 1 for each corresponding substituent moiety.

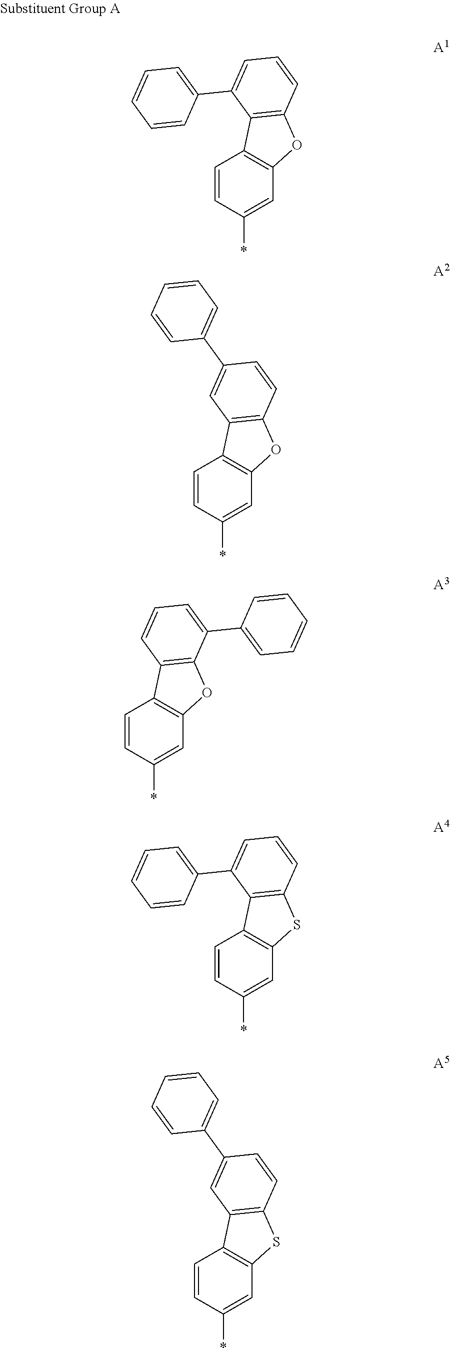 [SMILES: *c1ccc2c(c1)oc1cccc(-c3ccccc3)c12 ; *c1ccc2c(c1)oc1ccc(-c3ccccc3)cc12 ; *c1ccc2c(c1)oc1c(-c3ccccc3)cccc12 ; *c1ccc2c(c1)sc1cccc(-c3ccccc3)c12 ; *c1ccc2c(c1)sc1ccc(-c3ccccc3)cc12]

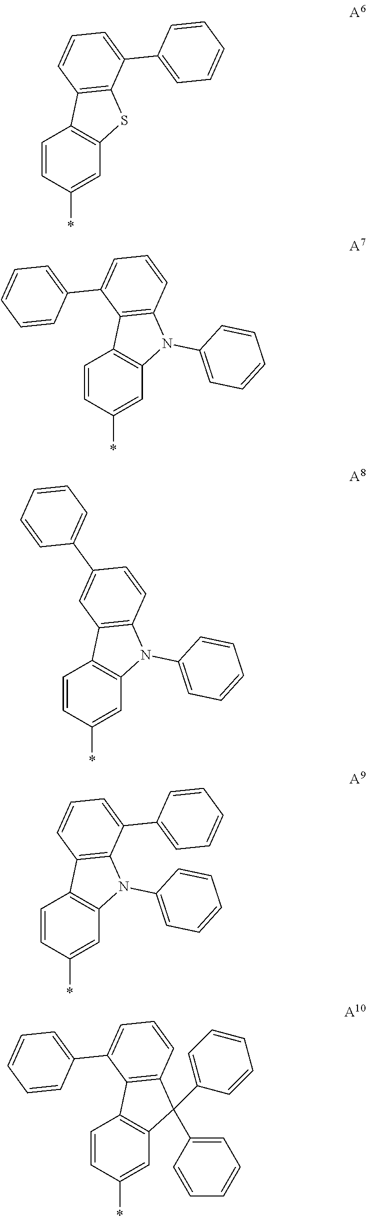

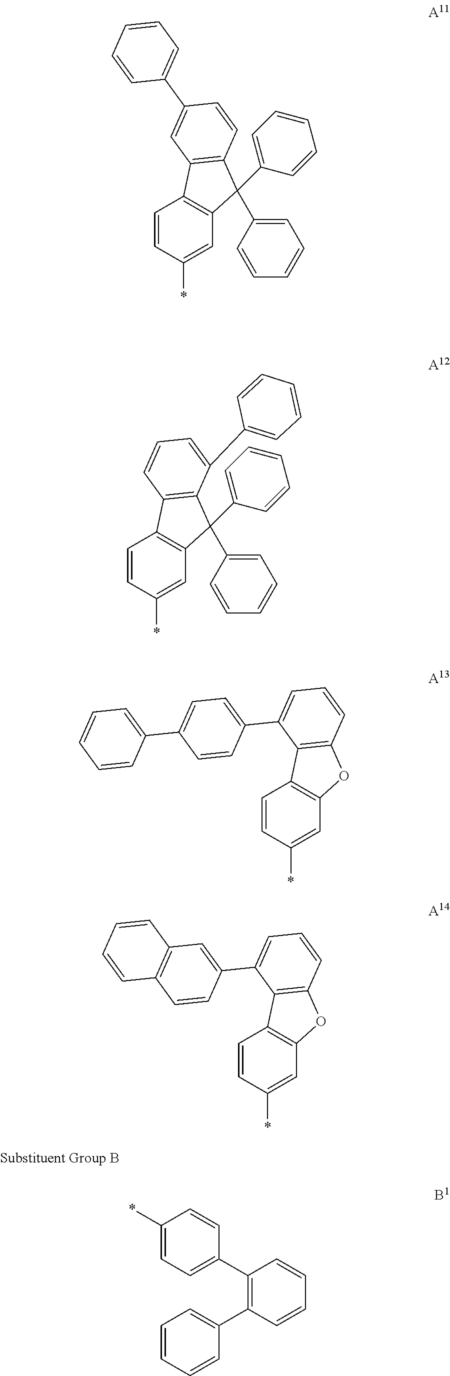 [SMILES: *c1ccc2c(c1)C(c1ccccc1)(c1ccccc1)c1ccc(-c3ccccc3)cc1-2 ; *c1ccc2c(c1)C(c1ccccc1)(c1ccccc1)c1c(-c3ccccc3)cccc1-2 ; *c1ccc2c(c1)oc1cccc(-c3ccc(-c4ccccc4)cc3)c12 ; *c1ccc2c(c1)oc1cccc(-c3ccc4ccccc4c3)c12 ; *c1ccc(-c2ccccc2-c2ccccc2)cc1]

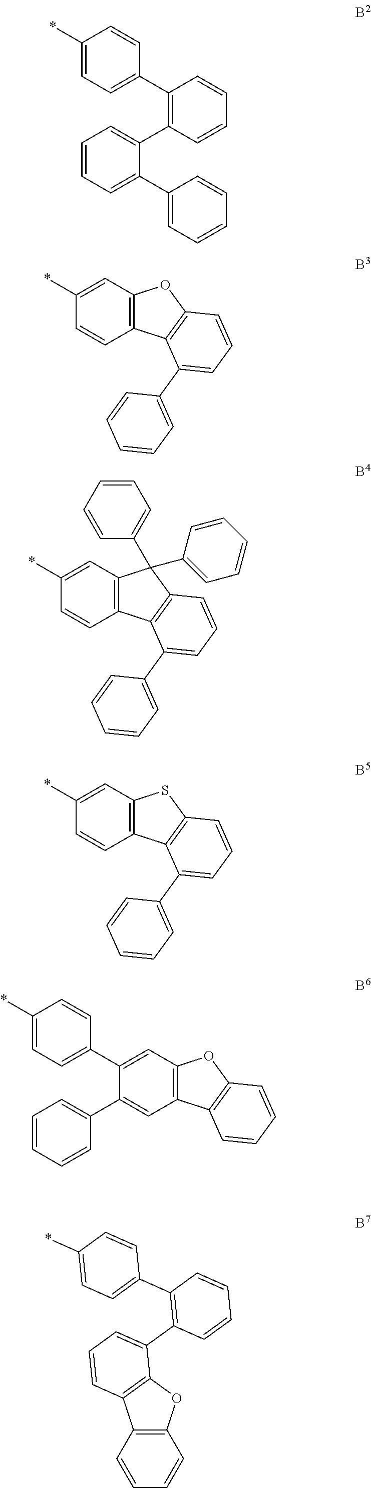

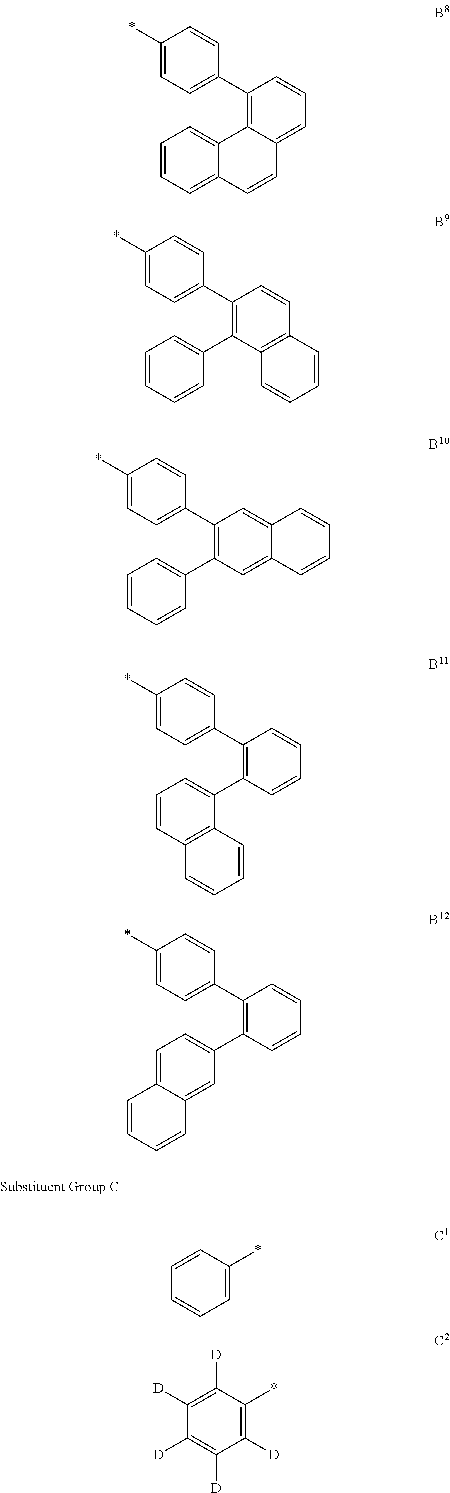

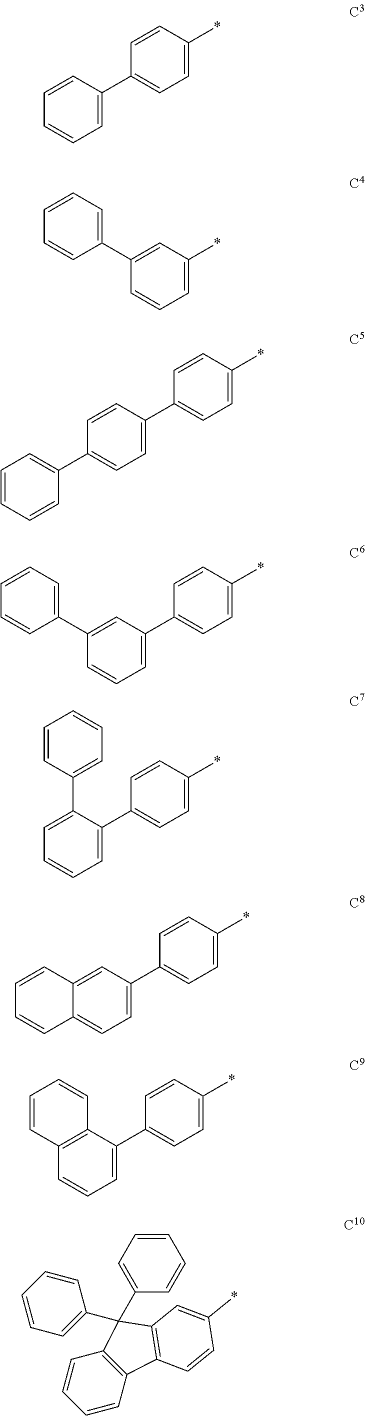

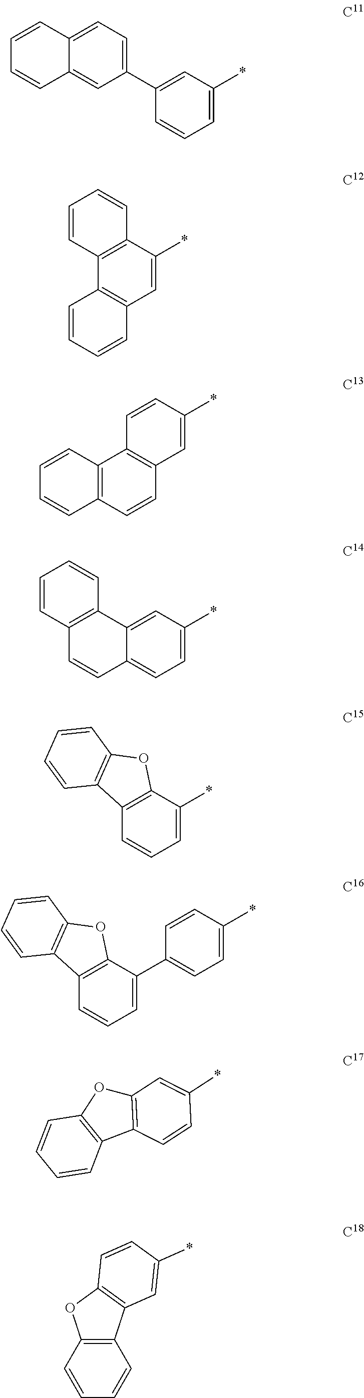

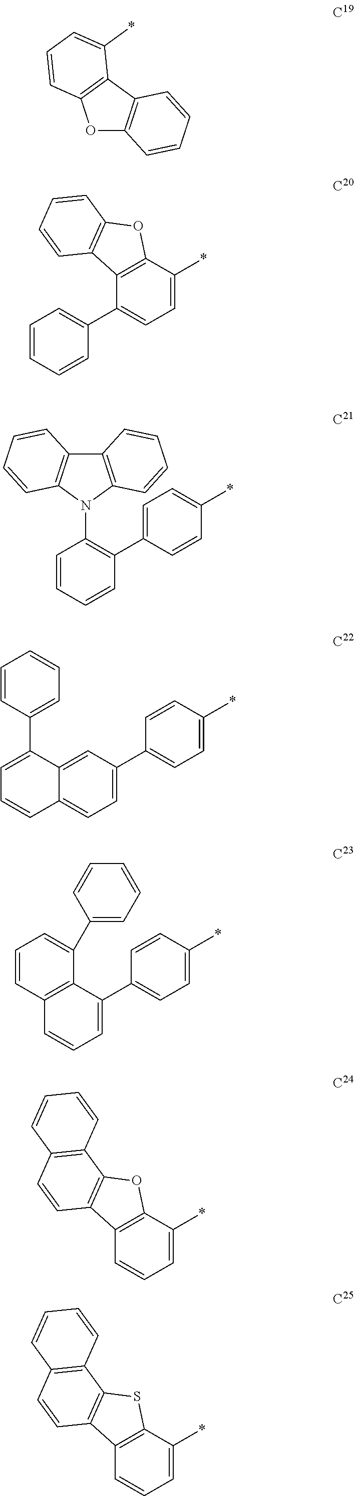

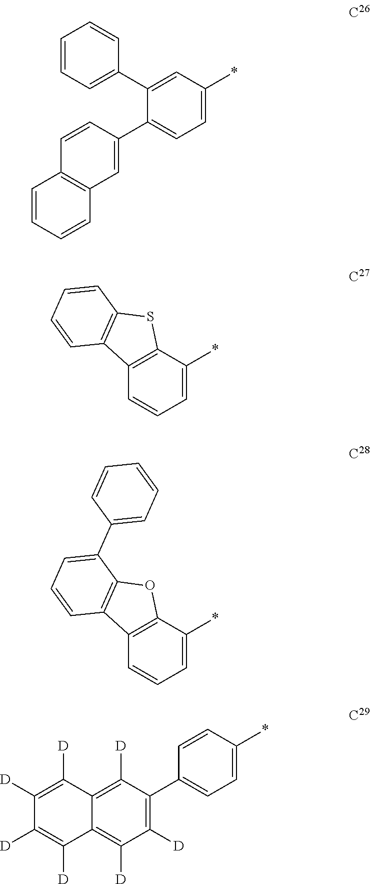 [SMILES: *c1ccc(-c2ccc3ccccc3c2)c(-c2ccccc2)c1 ; *c1cccc2c1sc1ccccc12 ; *c1cccc2c1oc1c(-c3ccccc3)cccc12 ; *c1ccc(-c2c([2H])c([2H])c3c([2H])c([2H])c([2H])c([2H])c3c2[2H])cc1]

TABLE 1
Compound Combination

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 5 | 1 | 1 |
| 6 | 6 | 1 | 1 |
| 7 | 7 | 1 | 1 |
| 8 | 8 | 1 | 1 |
| 9 | 9 | 1 | 1 |
| 10 | 1 | 1 | 28 |
| 11 | 1 | 2 | 28 |
| 12 | 12 | 1 | 1 |
| 13 | 13 | 1 | 1 |
| 14 | 14 | 1 | 1 |
| 15 | 1 | 1 | 2 |
| 16 | 2 | 1 | 2 |
| 17 | 3 | 1 | 2 |
| 18 | 4 | 1 | 2 |
| 19 | 5 | 1 | 2 |
| 20 | 6 | 1 | 2 |
| 21 | 7 | 1 | 2 |
| 22 | 8 | 1 | 2 |
| 23 | 9 | 1 | 2 |
| 24 | 10 | 1 | 2 |
| 25 | 11 | 1 | 2 |
| 26 | 12 | 1 | 2 |
| 27 | 13 | 1 | 2 |
| 28 | 14 | 1 | 2 |
| 29 | 1 | 1 | 3 |
| 30 | 2 | 1 | 3 |
| 31 | 3 | 1 | 3 |
| 32 | 4 | 1 | 3 |
| 33 | 5 | 1 | 3 |
| 34 | 6 | 1 | 3 |
| 35 | 7 | 1 | 3 |
| 36 | 8 | 1 | 3 |
| 37 | 9 | 1 | 3 |
| 38 | 10 | 1 | 3 |
| 39 | 11 | 1 | 3 |
| 40 | 12 | 1 | 3 |
| 41 | 13 | 1 | 3 |
| 42 | 14 | 1 | 3 |
| 43 | 1 | 1 | 8 |
| 44 | 2 | 1 | 8 |
| 45 | 3 | 1 | 8 |
| 46 | 4 | 1 | 8 |
| 47 | 5 | 1 | 8 |
| 48 | 6 | 1 | 8 |
| 49 | 7 | 1 | 8 |
| 50 | 8 | 1 | 8 |
| 51 | 9 | 1 | 8 |
| 52 | 10 | 1 | 8 |
| 53 | 11 | 1 | 8 |
| 54 | 12 | 1 | 8 |
| 55 | 13 | 1 | 8 |
| 56 | 14 | 1 | 8 |
| 57 | 1 | 2 | 1 |
| 58 | 2 | 2 | 1 |
| 59 | 3 | 2 | 1 |
| 60 | 4 | 2 | 1 |
| 61 | 5 | 2 | 1 |
| 62 | 6 | 2 | 1 |
| 63 | 7 | 2 | 1 |
| 64 | 8 | 2 | 1 |
| 65 | 9 | 2 | 1 |
| 66 | 10 | 2 | 1 |
| 67 | 11 | 2 | 1 |
| 68 | 12 | 2 | 1 |
| 69 | 13 | 2 | 1 |
| 70 | 14 | 2 | 1 |
| 71 | 1 | 2 | 2 |
| 72 | 2 | 2 | 2 |
| 73 | 3 | 2 | 2 |
| 74 | 4 | 2 | 2 |
| 75 | 5 | 2 | 2 |
| 76 | 6 | 2 | 2 |
| 77 | 7 | 2 | 2 |
| 78 | 8 | 2 | 2 |
| 79 | 9 | 2 | 2 |
| 80 | 10 | 2 | 2 |
| 81 | 11 | 2 | 2 |
| 82 | 12 | 2 | 2 |
| 83 | 13 | 2 | 2 |
| 84 | 14 | 2 | 2 |
| 85 | 1 | 2 | 3 |
| 86 | 2 | 2 | 3 |
| 87 | 3 | 2 | 3 |
| 88 | 4 | 2 | 3 |
| 89 | 5 | 2 | 3 |
| 90 | 6 | 2 | 3 |
| 91 | 7 | 2 | 3 |
| 92 | 8 | 2 | 3 |
| 93 | 9 | 2 | 3 |
| 94 | 10 | 2 | 3 |
| 95 | 11 | 2 | 3 |
| 96 | 12 | 2 | 3 |
| 97 | 13 | 2 | 3 |
| 98 | 14 | 2 | 3 |
| 99 | 1 | 2 | 8 |
| 100 | 2 | 2 | 8 |
| 101 | 3 | 2 | 8 |
| 102 | 4 | 2 | 8 |
| 103 | 5 | 2 | 8 |
| 104 | 6 | 2 | 8 |
| 105 | 7 | 2 | 8 |
| 106 | 8 | 2 | 8 |
| 107 | 9 | 2 | 8 |
| 108 | 10 | 2 | 8 |
| 109 | 11 | 2 | 8 |
| 110 | 12 | 2 | 8 |
| 111 | 13 | 2 | 8 |
| 112 | 14 | 2 | 8 |
| 113 | 1 | 3 | 8 |
| 114 | 1 | 4 | 8 |
| 115 | 1 | 5 | 8 |
| 116 | 1 | 6 | 8 |
| 117 | 1 | 7 | 8 |
| 118 | 1 | 8 | 8 |
| 119 | 1 | 11 | 8 |
| 120 | 1 | 12 | 8 |
| 121 | 1 | 1 | 13 |
| 122 | 1 | 2 | 13 |
| 123 | 1 | 3 | 13 |
| 124 | 1 | 4 | 13 |
| 125 | 1 | 5 | 13 |
| 126 | 1 | 6 | 13 |
| 127 | 1 | 7 | 13 |
| 128 | 1 | 8 | 13 |
| 129 | 1 | 9 | 13 |
| 130 | 1 | 10 | 13 |
| 131 | 1 | 11 | 13 |
| 132 | 1 | 12 | 13 |
| 133 | 3 | 3 | 8 |
| 134 | 3 | 4 | 8 |
| 135 | 3 | 5 | 8 |
| 136 | 3 | 6 | 8 |
| 137 | 3 | 7 | 8 |
| 138 | 3 | 8 | 8 |
| 139 | 3 | 11 | 8 |
| 140 | 3 | 12 | 8 |
| 141 | 3 | 1 | 13 |
| 142 | 3 | 2 | 13 |
| 143 | 3 | 3 | 13 |
| 144 | 3 | 4 | 13 |
| 145 | 3 | 5 | 13 |
| 146 | 3 | 6 | 13 |
| 147 | 3 | 7 | 13 |
| 148 | 3 | 8 | 13 |
| 149 | 3 | 9 | 13 |
| 150 | 3 | 10 | 13 |
| 151 | 3 | 11 | 13 |
| 152 | 3 | 12 | 13 |
| 153 | 1 | 1 | 4 |
| 154 | 1 | 1 | 5 |
| 155 | 1 | 1 | 6 |
| 156 | 1 | 1 | 7 |
| 157 | 1 | 1 | 9 |
| 158 | 1 | 1 | 10 |
| 159 | 1 | 1 | 11 |
| 160 | 1 | 1 | 12 |
| 161 | 1 | 1 | 14 |
| 162 | 1 | 1 | 15 |
| 163 | 1 | 1 | 16 |
| 164 | 1 | 1 | 17 |
| 165 | 1 | 1 | 18 |
| 166 | 1 | 1 | 19 |
| 167 | 1 | 1 | 20 |
| 168 | 1 | 1 | 21 |
| 169 | 1 | 1 | 22 |
| 170 | 1 | 1 | 23 |
| 171 | 1 | 1 | 24 |
| 172 | 1 | 1 | 25 |
| 173 | 1 | 1 | 26 |
| 174 | 1 | 1 | 27 |
| 175 | 1 | 2 | 4 |
| 176 | 1 | 2 | 5 |
| 177 | 1 | 2 | 6 |
| 178 | 1 | 2 | 7 |
| 179 | 1 | 2 | 9 |
| 180 | 1 | 2 | 10 |
| 181 | 1 | 2 | 11 |
| 182 | 1 | 2 | 12 |
| 183 | 1 | 2 | 14 |
| 184 | 1 | 2 | 15 |
| 185 | 1 | 2 | 16 |
| 186 | 1 | 2 | 17 |
| 187 | 1 | 2 | 18 |
| 188 | 1 | 2 | 19 |
| 189 | 1 | 2 | 20 |
| 190 | 1 | 2 | 21 |
| 191 | 1 | 2 | 22 |
| 192 | 1 | 2 | 23 |
| 193 | 1 | 2 | 24 |
| 194 | 1 | 2 | 25 |
| 195 | 1 | 2 | 26 |
| 196 | 1 | 2 | 27 |
| 197 | 1 | 7 | 1 |
| 198 | 1 | 7 | 2 |
| 199 | 1 | 7 | 3 |
| 200 | 1 | 7 | 4 |
| 201 | 1 | 7 | 5 |
| 202 | 1 | 7 | 6 |
| 203 | 1 | 7 | 7 |
| 204 | 1 | 7 | 9 |
| 205 | 1 | 7 | 10 |
| 206 | 1 | 7 | 11 |
| 207 | 1 | 7 | 12 |
| 208 | 1 | 7 | 14 |
| 209 | 1 | 7 | 15 |
| 210 | 1 | 7 | 16 |
| 211 | 1 | 7 | 17 |
| 212 | 1 | 7 | 18 |
| 213 | 1 | 7 | 19 |
| 214 | 1 | 7 | 20 |
| 215 | 1 | 7 | 21 |
| 216 | 1 | 7 | 22 |
| 217 | 1 | 7 | 23 |
| 218 | 1 | 7 | 24 |
| 219 | 1 | 7 | 25 |
| 220 | 1 | 7 | 26 |
| 221 | 1 | 7 | 27 |
| 222 | 1 | 11 | 1 |
| 223 | 1 | 11 | 2 |
| 224 | 1 | 11 | 3 |
| 225 | 1 | 11 | 4 |
| 226 | 1 | 11 | 5 |
| 227 | 1 | 11 | 6 |
| 228 | 1 | 11 | 7 |
| 229 | 1 | 11 | 9 |
| 230 | 1 | 11 | 10 |
| 231 | 1 | 11 | 11 |
| 232 | 1 | 11 | 12 |
| 233 | 1 | 11 | 14 |
| 234 | 1 | 11 | 15 |
| 235 | 1 | 11 | 16 |
| 236 | 1 | 11 | 17 |
| 237 | 1 | 11 | 18 |
| 238 | 1 | 11 | 19 |
| 239 | 1 | 11 | 20 |
| 240 | 1 | 11 | 21 |
| 241 | 1 | 11 | 22 |
| 242 | 1 | 11 | 23 |
| 243 | 1 | 11 | 24 |
| 244 | 1 | 11 | 25 |
| 245 | 1 | 11 | 26 |
| 246 | 1 | 11 | 27 |
| 247 | 3 | 1 | 4 |
| 248 | 3 | 1 | 5 |
| 249 | 3 | 1 | 6 |
| 250 | 3 | 1 | 7 |
| 251 | 3 | 1 | 9 |
| 252 | 3 | 1 | 10 |
| 253 | 3 | 1 | 11 |
| 254 | 3 | 1 | 12 |
| 255 | 3 | 1 | 14 |
| 256 | 3 | 1 | 15 |
| 257 | 3 | 1 | 16 |
| 258 | 3 | 1 | 17 |
| 259 | 3 | 1 | 18 |
| 260 | 3 | 1 | 19 |
| 261 | 3 | 1 | 20 |
| 262 | 3 | 1 | 21 |
| 263 | 3 | 1 | 22 |
| 264 | 3 | 1 | 23 |
| 265 | 3 | 1 | 24 |
| 266 | 3 | 1 | 25 |
| 267 | 3 | 1 | 26 |
| 268 | 3 | 1 | 27 |
| 269 | 3 | 2 | 4 |
| 270 | 3 | 2 | 5 |
| 271 | 3 | 2 | 6 |
| 272 | 3 | 2 | 7 |
| 273 | 3 | 2 | 9 |
| 274 | 3 | 2 | 10 |
| 275 | 3 | 2 | 11 |
| 276 | 3 | 2 | 12 |
| 277 | 3 | 2 | 14 |
| 278 | 3 | 2 | 15 |
| 279 | 3 | 2 | 16 |
| 280 | 3 | 2 | 17 |
| 281 | 3 | 2 | 18 |
| 282 | 3 | 2 | 19 |
| 283 | 3 | 2 | 20 |
| 284 | 3 | 2 | 21 |
| 285 | 3 | 2 | 22 |
| 286 | 3 | 2 | 23 |
| 287 | 3 | 2 | 24 |
| 288 | 3 | 2 | 25 |
| 289 | 3 | 2 | 26 |
| 290 | 3 | 2 | 27 |
| 291 | 3 | 7 | 1 |
| 292 | 3 | 7 | 2 |
| 293 | 3 | 7 | 3 |
| 294 | 3 | 7 | 4 |
| 295 | 3 | 7 | 5 |
| 296 | 3 | 7 | 6 |
| 297 | 3 | 7 | 7 |
| 298 | 3 | 7 | 9 |
| 299 | 3 | 7 | 10 |
| 300 | 3 | 7 | 11 |
| 301 | 3 | 7 | 12 |
| 302 | 3 | 7 | 14 |
| 303 | 3 | 7 | 15 |
| 304 | 3 | 7 | 16 |
| 305 | 3 | 7 | 17 |
| 306 | 3 | 7 | 18 |
| 307 | 3 | 7 | 19 |
| 308 | 3 | 7 | 20 |
| 309 | 3 | 7 | 21 |
| 310 | 3 | 7 | 22 |
| 311 | 3 | 7 | 23 |
| 312 | 3 | 7 | 24 |
| 313 | 3 | 7 | 25 |
| 314 | 3 | 7 | 26 |
| 315 | 3 | 7 | 27 |
| 316 | 3 | 11 | 1 |
| 317 | 3 | 11 | 2 |
| 318 | 3 | 11 | 3 |
| 319 | 3 | 11 | 4 |
| 320 | 3 | 11 | 5 |
| 321 | 3 | 11 | 6 |
| 322 | 3 | 11 | 7 |
| 323 | 3 | 11 | 9 |
| 324 | 3 | 11 | 10 |
| 325 | 3 | 11 | 11 |
| 326 | 3 | 11 | 12 |
| 327 | 3 | 11 | 14 |
| 328 | 3 | 11 | 15 |
| 329 | 3 | 11 | 16 |
| 330 | 3 | 11 | 17 |
| 331 | 3 | 11 | 18 |
| 332 | 3 | 11 | 19 |
| 333 | 3 | 11 | 20 |
| 334 | 3 | 11 | 21 |
| 335 | 3 | 11 | 22 |
| 336 | 3 | 11 | 23 |
| 337 | 3 | 11 | 24 |
| 338 | 3 | 11 | 25 |
| 339 | 3 | 11 | 26 |
| 340 | 3 | 11 | 27 |
| 341 | 4 | 1 | 4 |
| 342 | 4 | 1 | 5 |
| 343 | 4 | 1 | 6 |
| 344 | 4 | 1 | 7 |
| 345 | 4 | 1 | 9 |
| 346 | 4 | 1 | 10 |
| 347 | 4 | 1 | 11 |
| 348 | 4 | 1 | 12 |
| 349 | 4 | 1 | 13 |
| 350 | 4 | 1 | 14 |
| 351 | 4 | 1 | 15 |
| 352 | 4 | 1 | 16 |
| 353 | 4 | 1 | 17 |
| 354 | 4 | 1 | 18 |
| 355 | 4 | 1 | 19 |
| 356 | 4 | 1 | 20 |
| 357 | 4 | 1 | 21 |
| 358 | 4 | 1 | 22 |
| 359 | 4 | 1 | 23 |
| 360 | 4 | 1 | 24 |
| 361 | 4 | 1 | 25 |
| 362 | 4 | 1 | 26 |
| 363 | 4 | 1 | 27 |
| 364 | 4 | 2 | 4 |
| 365 | 4 | 2 | 5 |
| 366 | 4 | 2 | 6 |
| 367 | 4 | 2 | 7 |
| 368 | 4 | 2 | 9 |
| 369 | 4 | 2 | 10 |
| 370 | 4 | 2 | 11 |
| 371 | 4 | 2 | 12 |
| 372 | 4 | 2 | 13 |
| 373 | 4 | 2 | 14 |
| 374 | 4 | 2 | 15 |
| 375 | 4 | 2 | 16 |
| 376 | 4 | 2 | 17 |
| 377 | 4 | 2 | 18 |
| 378 | 4 | 2 | 19 |
| 379 | 4 | 2 | 20 |
| 380 | 4 | 2 | 21 |
| 381 | 4 | 2 | 22 |
| 382 | 4 | 2 | 23 |
| 383 | 4 | 2 | 24 |
| 384 | 4 | 2 | 25 |
| 385 | 4 | 2 | 26 |
| 386 | 4 | 2 | 27 |
| 387 | 4 | 7 | 1 |
| 388 | 4 | 7 | 2 |
| 389 | 4 | 7 | 3 |
| 390 | 4 | 7 | 4 |
| 391 | 4 | 7 | 5 |
| 392 | 4 | 7 | 6 |
| 393 | 4 | 7 | 7 |
| 394 | 4 | 7 | 8 |
| 395 | 4 | 7 | 9 |
| 396 | 4 | 7 | 10 |
| 397 | 4 | 7 | 11 |
| 398 | 4 | 7 | 12 |
| 399 | 4 | 7 | 13 |
| 400 | 4 | 7 | 14 |
| 401 | 4 | 7 | 15 |
| 402 | 4 | 7 | 16 |
| 403 | 4 | 7 | 17 |
| 404 | 4 | 7 | 18 |
| 405 | 4 | 7 | 19 |
| 406 | 4 | 7 | 20 |
| 407 | 4 | 7 | 21 |
| 408 | 4 | 7 | 22 |
| 409 | 4 | 7 | 23 |
| 410 | 4 | 7 | 24 |
| 411 | 4 | 7 | 25 |
| 412 | 4 | 7 | 26 |
| 413 | 4 | 7 | 27 |
| 414 | 4 | 11 | 1 |
| 415 | 4 | 11 | 2 |
| 416 | 4 | 11 | 3 |
| 417 | 4 | 11 | 4 |
| 418 | 4 | 11 | 5 |
| 419 | 4 | 11 | 6 |
| 420 | 4 | 11 | 7 |
| 421 | 4 | 11 | 8 |
| 422 | 4 | 11 | 9 |
| 423 | 4 | 11 | 10 |
| 424 | 4 | 11 | 11 |
| 425 | 4 | 11 | 12 |
| 426 | 4 | 11 | 13 |
| 427 | 4 | 11 | 14 |
| 428 | 4 | 11 | 15 |
| 429 | 4 | 11 | 16 |
| 430 | 4 | 11 | 17 |
| 431 | 4 | 11 | 18 |
| 432 | 4 | 11 | 19 |
| 433 | 4 | 11 | 20 |
| 434 | 4 | 11 | 21 |
| 435 | 4 | 11 | 22 |
| 436 | 4 | 11 | 23 |
| 437 | 4 | 11 | 24 |
| 438 | 4 | 11 | 25 |
| 439 | 4 | 11 | 26 |
| 440 | 4 | 11 | 27 |
| 441 | 7 | 1 | 4 |
| 442 | 7 | 1 | 5 |
| 443 | 7 | 1 | 6 |
| 444 | 7 | 1 | 7 |
| 445 | 7 | 1 | 9 |
| 446 | 7 | 1 | 10 |
| 447 | 7 | 1 | 11 |
| 448 | 7 | 1 | 12 |
| 449 | 7 | 1 | 13 |
| 450 | 7 | 1 | 14 |
| 451 | 7 | 1 | 15 |
| 452 | 7 | 1 | 16 |
| 453 | 7 | 1 | 17 |
| 454 | 7 | 1 | 18 |
| 455 | 7 | 1 | 19 |
| 456 | 7 | 1 | 20 |
| 457 | 7 | 1 | 21 |
| 458 | 7 | 1 | 22 |
| 459 | 7 | 1 | 23 |
| 460 | 7 | 1 | 24 |
| 461 | 7 | 1 | 25 |
| 462 | 7 | 1 | 26 |
| 463 | 7 | 1 | 27 |
| 464 | 7 | 2 | 4 |
| 465 | 7 | 2 | 5 |
| 466 | 7 | 2 | 6 |
| 467 | 7 | 2 | 7 |
| 468 | 7 | 2 | 9 |
| 469 | 7 | 2 | 10 |
| 470 | 7 | 2 | 11 |
| 471 | 7 | 2 | 12 |
| 472 | 7 | 2 | 13 |
| 473 | 7 | 2 | 14 |
| 474 | 7 | 2 | 15 |
| 475 | 7 | 2 | 16 |
| 476 | 7 | 2 | 17 |
| 477 | 7 | 2 | 18 |
| 478 | 7 | 2 | 19 |
| 479 | 7 | 2 | 20 |
| 480 | 7 | 2 | 21 |
| 481 | 7 | 2 | 22 |
| 482 | 7 | 2 | 23 |
| 483 | 7 | 2 | 24 |
| 484 | 7 | 2 | 25 |
| 485 | 7 | 2 | 26 |
| 486 | 7 | 2 | 27 |
| 487 | 7 | 7 | 1 |
| 488 | 7 | 7 | 2 |
| 489 | 7 | 7 | 3 |
| 490 | 7 | 7 | 4 |
| 491 | 7 | 7 | 5 |
| 492 | 7 | 7 | 6 |
| 493 | 7 | 7 | 7 |
| 494 | 7 | 7 | 8 |
| 495 | 7 | 7 | 9 |
| 496 | 7 | 7 | 10 |
| 497 | 7 | 7 | 11 |
| 498 | 7 | 7 | 12 |
| 499 | 7 | 7 | 13 |
| 500 | 7 | 7 | 14 |
| 501 | 7 | 7 | 15 |
| 502 | 7 | 7 | 16 |
| 503 | 7 | 7 | 17 |
| 504 | 7 | 7 | 18 |
| 505 | 7 | 7 | 19 |
| 506 | 7 | 7 | 20 |
| 507 | 7 | 7 | 21 |
| 508 | 7 | 7 | 22 |
| 509 | 7 | 7 | 23 |
| 510 | 7 | 7 | 24 |
| 511 | 7 | 7 | 25 |
| 512 | 7 | 7 | 26 |
| 513 | 7 | 7 | 27 |
| 514 | 7 | 11 | 1 |
| 515 | 7 | 11 | 2 |
| 516 | 7 | 11 | 3 |
| 517 | 7 | 11 | 4 |
| 518 | 7 | 11 | 5 |
| 519 | 7 | 11 | 6 |
| 520 | 7 | 11 | 7 |
| 521 | 7 | 11 | 8 |
| 522 | 7 | 11 | 9 |
| 523 | 7 | 11 | 10 |
| 524 | 7 | 11 | 11 |
| 525 | 7 | 11 | 12 |
| 526 | 7 | 11 | 13 |
| 527 | 7 | 11 | 14 |
| 528 | 7 | 11 | 15 |
| 529 | 7 | 11 | 16 |
| 530 | 7 | 11 | 17 |
| 531 | 7 | 11 | 18 |
| 532 | 7 | 11 | 19 |
| 533 | 7 | 11 | 20 |
| 534 | 7 | 11 | 21 |
| 535 | 7 | 11 | 22 |
| 536 | 7 | 11 | 23 |
| 537 | 7 | 11 | 24 |
| 538 | 7 | 11 | 25 |
| 539 | 7 | 11 | 26 |
| 540 | 7 | 11 | 27 |
| 541 | 10 | 1 | 4 |
| 542 | 10 | 1 | 5 |
| 543 | 10 | 1 | 6 |
| 544 | 10 | 1 | 7 |
| 545 | 10 | 1 | 9 |
| 546 | 10 | 1 | 10 |
| 547 | 10 | 1 | 11 |
| 548 | 10 | 1 | 12 |
| 549 | 10 | 1 | 13 |
| 550 | 10 | 1 | 14 |
| 551 | 10 | 1 | 15 |
| 552 | 10 | 1 | 16 |
| 553 | 10 | 1 | 17 |
| 554 | 10 | 1 | 18 |
| 555 | 10 | 1 | 19 |
| 556 | 10 | 1 | 20 |
| 557 | 10 | 1 | 21 |
| 558 | 10 | 1 | 22 |
| 559 | 10 | 1 | 23 |
| 560 | 10 | 1 | 24 |
| 561 | 10 | 1 | 25 |
| 562 | 10 | 1 | 26 |
| 563 | 10 | 1 | 27 |
| 564 | 10 | 2 | 4 |
| 565 | 10 | 2 | 5 |
| 566 | 10 | 2 | 6 |
| 567 | 10 | 2 | 7 |
| 568 | 10 | 2 | 9 |
| 569 | 10 | 2 | 10 |
| 570 | 10 | 2 | 11 |
| 571 | 10 | 2 | 12 |
| 572 | 10 | 2 | 13 |
| 573 | 10 | 2 | 14 |
| 574 | 10 | 2 | 15 |
| 575 | 10 | 2 | 16 |
| 576 | 10 | 2 | 17 |
| 577 | 10 | 2 | 18 |
| 578 | 10 | 2 | 19 |
| 579 | 10 | 2 | 20 |
| 580 | 10 | 2 | 21 |
| 581 | 10 | 2 | 22 |
| 582 | 10 | 2 | 23 |
| 583 | 10 | 2 | 24 |
| 584 | 10 | 2 | 25 |
| 585 | 10 | 2 | 26 |
| 586 | 10 | 2 | 27 |
| 587 | 10 | 7 | 1 |
| 588 | 10 | 7 | 2 |
| 589 | 10 | 7 | 3 |
| 590 | 10 | 7 | 4 |
| 591 | 10 | 7 | 5 |
| 592 | 10 | 7 | 6 |
| 593 | 10 | 7 | 7 |
| 594 | 10 | 7 | 8 |
| 595 | 10 | 7 | 9 |
| 596 | 10 | 7 | 10 |
| 597 | 10 | 7 | 11 |
| 598 | 10 | 7 | 12 |
| 599 | 10 | 7 | 13 |
| 600 | 10 | 7 | 14 |
| 601 | 10 | 7 | 15 |
| 602 | 10 | 7 | 16 |
| 603 | 10 | 7 | 17 |
| 604 | 10 | 7 | 18 |
| 605 | 10 | 7 | 19 |
| 606 | 10 | 7 | 20 |
| 607 | 10 | 7 | 21 |
| 608 | 10 | 7 | 22 |
| 609 | 10 | 7 | 23 |
| 610 | 10 | 7 | 24 |
| 611 | 10 | 7 | 25 |
| 612 | 10 | 7 | 26 |
| 613 | 10 | 7 | 27 |
| 614 | 10 | 11 | 1 |
| 615 | 10 | 11 | 2 |
| 616 | 10 | 11 | 3 |
| 617 | 10 | 11 | 4 |
| 618 | 10 | 11 | 5 |
| 619 | 10 | 11 | 6 |
| 620 | 10 | 11 | 7 |
| 621 | 10 | 11 | 8 |
| 622 | 10 | 11 | 9 |
| 623 | 10 | 11 | 10 |
| 624 | 10 | 11 | 11 |
| 625 | 10 | 11 | 12 |
| 626 | 10 | 11 | 13 |
| 627 | 10 | 11 | 14 |
| 628 | 10 | 11 | 15 |
| 629 | 10 | 11 | 16 |
| 630 | 10 | 11 | 17 |
| 631 | 10 | 11 | 18 |
| 632 | 10 | 11 | 19 |
| 633 | 10 | 11 | 20 |
| 634 | 10 | 11 | 21 |
| 635 | 10 | 11 | 22 |
| 636 | 10 | 11 | 23 |
| 637 | 10 | 11 | 24 |
| 638 | 10 | 11 | 25 |
| 639 | 10 | 11 | 26 |
| 640 | 10 | 11 | 27 |
| 641 | 3 | 1 | 29. |

The amine compound according to one or more embodiments includes the first substituent, the second substituent, and the third substituent linked to the core nitrogen atom, and thus allow a light emitting element to obtain high efficiency and long service life.

The amine compound of one or more embodiments includes an amine group and has a structure in which the first substituent, the second substituent, and the third substituent are bonded to the amine group of one or more embodiments.

In one or more embodiments, the first substituent may include a dibenzofuran moiety, a dibenzothiophene moiety, a carbazole moiety, or a fluorene moiety in which the first substituent is substituted at a specific position. The 3rd carbon of the dibenzofuran moiety, the 3rd carbon of the dibenzothiophene moiety, the 3rd carbon of the carbazole moiety, or the 3rd carbon of the fluorene moiety in the first substituent may be linked to the core nitrogen atom, and at least one of the 5th carbon, the 7th carbon, or the 8th carbon among the other carbons may be linked to a first sub-substituent. The second substituent may include an ortho-terphenyl moiety. The second substituent may be bonded to the core nitrogen atom through an arylene linker or a heteroarylene linker, or may be bonded directly to the core nitrogen atom without a separate linker. The third substituent may be an aryl group or a heteroaryl group and is directly bonded to the core nitrogen atom.

The amine compound of one or more embodiments may have excellent or suitable electrical and thermal stability and high charge transport properties due to the introduction of such substituents and the specific substitution positions. Accordingly, the amine compound of one or more embodiments may have improved lifetime. In addition, the light emitting element of one or more embodiments including the amine compound of one or more embodiments may have improved luminous efficiency and lifetime.

Referring back to FIGS. 3 to 6, the light emitting element according to one or more embodiments of the disclosure will be further described.

As described above, the hole transport region HTR may include the amine compound according to one or more embodiments of the present disclosure described herein. For example, the hole transport region HTR may include an amine compound represented by Formula 1.

When the hole transport region HTR has a multilayer structure having a plurality of layers, any one selected from among the plurality of layers may include the amine compound represented by Formula 1. For example, in one or more embodiments, the hole transport region HTR may include a hole injection layer HIL arranged on the first electrode EL1, and a hole transport layer HTL arranged on the hole injection layer HIL, and the hole transport layer HTL may include the amine compound represented by Formula 1. However, embodiments of the present disclosure are not limited thereto, and for example, in one or more embodiments, the hole injection layer HIL may include the amine compound represented by Formula 1.

In one or more embodiments, the hole transport region HTR may include one or two or more amine compounds represented by Formula 1. For example, in one or more embodiments, the hole transport region HTR may include at least one selected from among amine compounds satisfying the combinations shown in Compound Combination Table 1 described herein.

In one or more embodiments, the hole transport region HTR may further include a compound represented by Formula H-1:

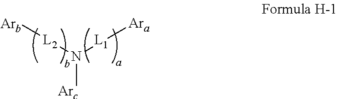

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a and/or b are each an integer of 2 or greater, a plurality of L₁'s and/or a plurality of L₂'s may each independently be a substituted or unsubstituted arylene group having 6 to 30 ring-forming carbon atoms or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms or a substituted or unsubstituted heteroaryl group having 2 to 30 ring-forming carbon atoms. In addition, in Formula H-1, Ar_{c} may be a substituted or unsubstituted aryl group having 6 to 30 ring-forming carbon atoms or a substituted or unsubstituted heteroaryl group having 2 to 30 ring-forming carbon atoms.

In one or more embodiments, the compound represented by Formula H-1 may be a monoamine compound. Alternatively, in one or more embodiments, the compound represented by Formula H-1 may be a diamine compound in which at least one selected from among Arₐ to Ar_{c} includes an amine group as a substituent. In one or more embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be any one selected from among compounds in Compound Group H. However, the compounds listed in Compound Group H are mere examples, and the compounds represented by Formula H-1 are not limited to those represented in Compound Group H:

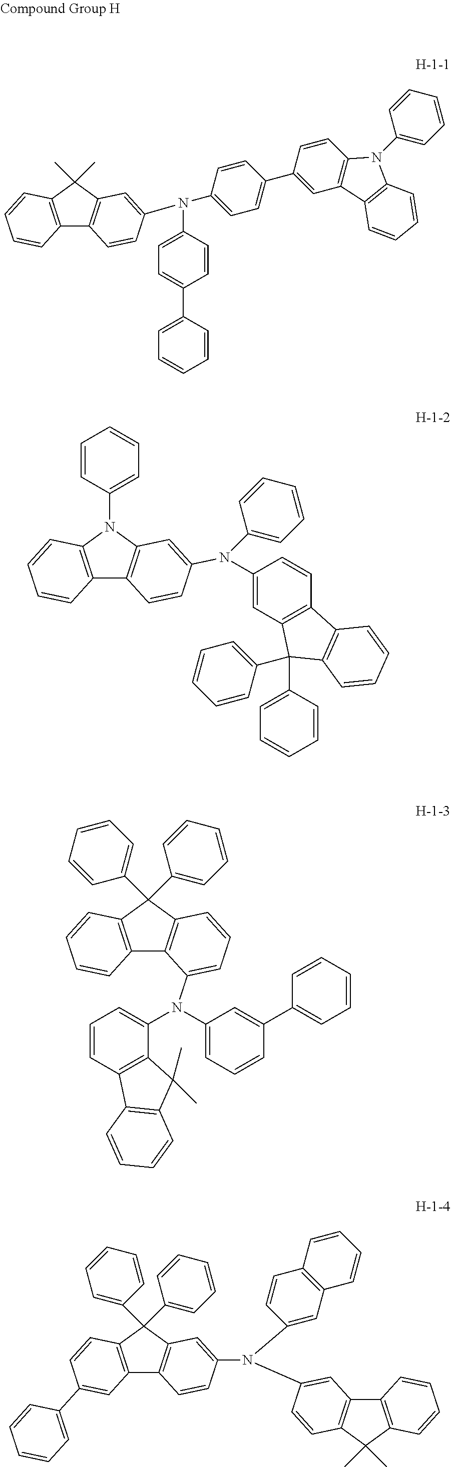 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

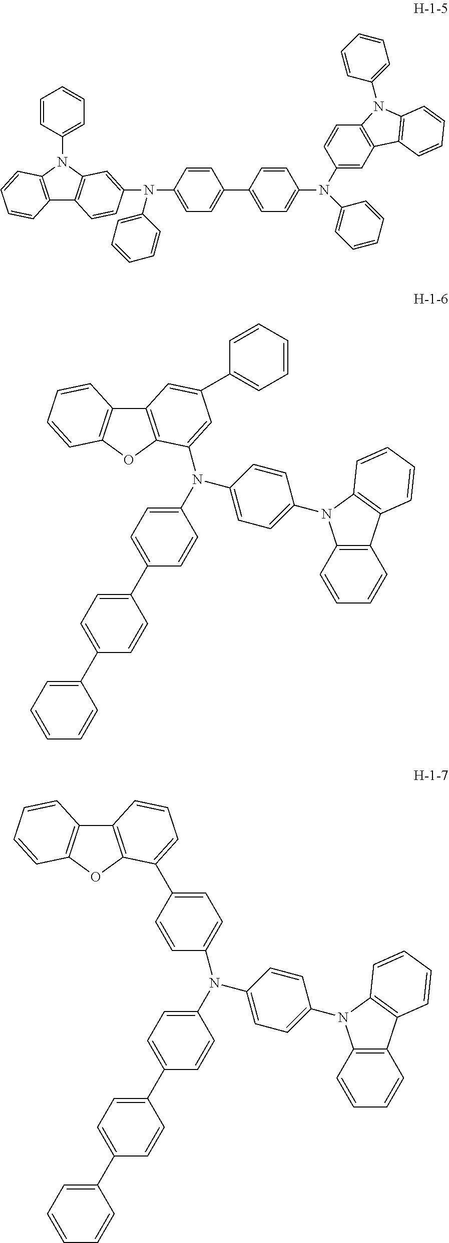 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

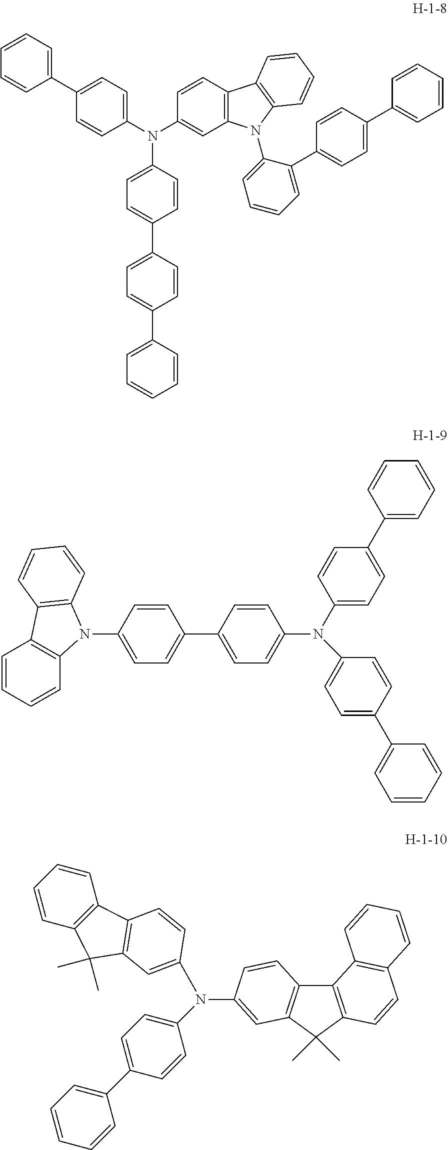 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

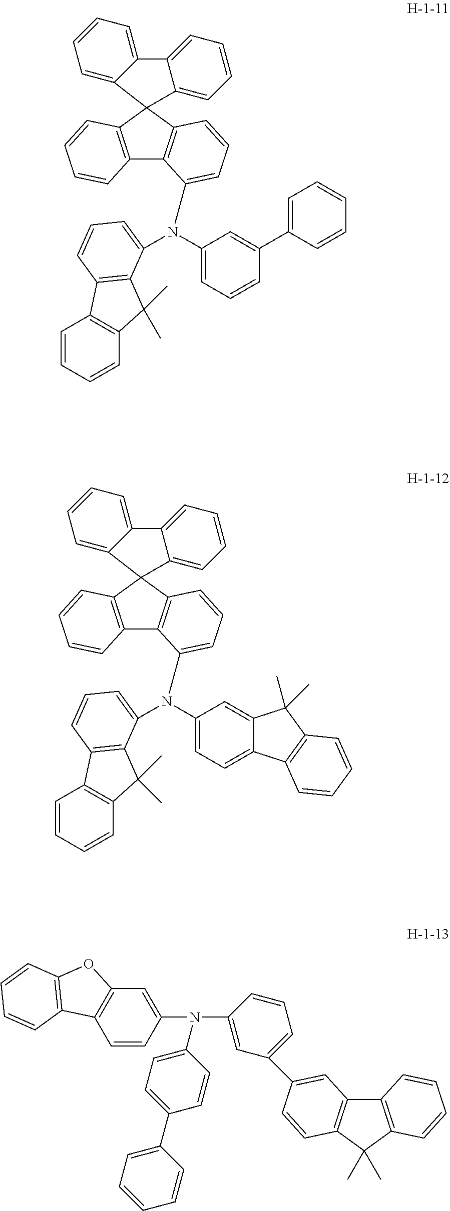 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

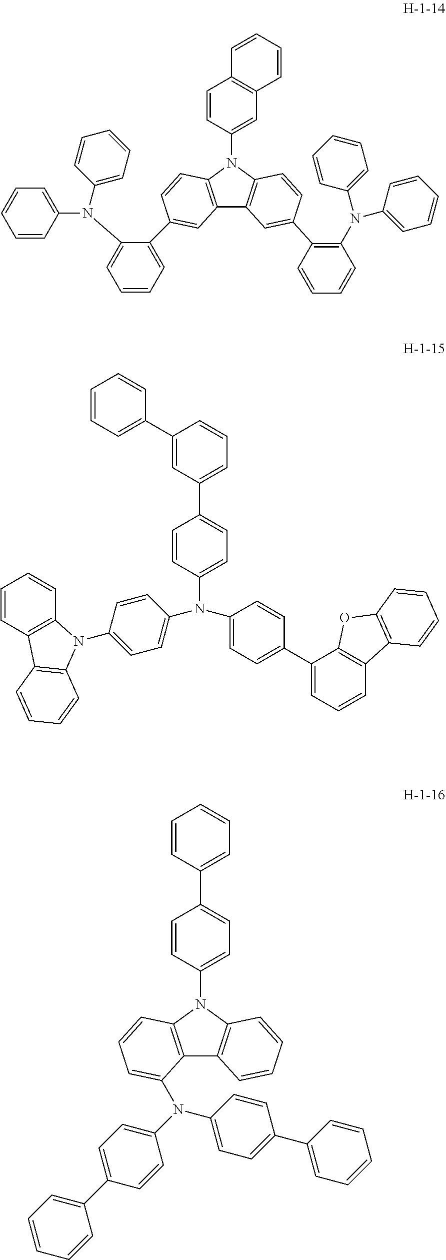 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

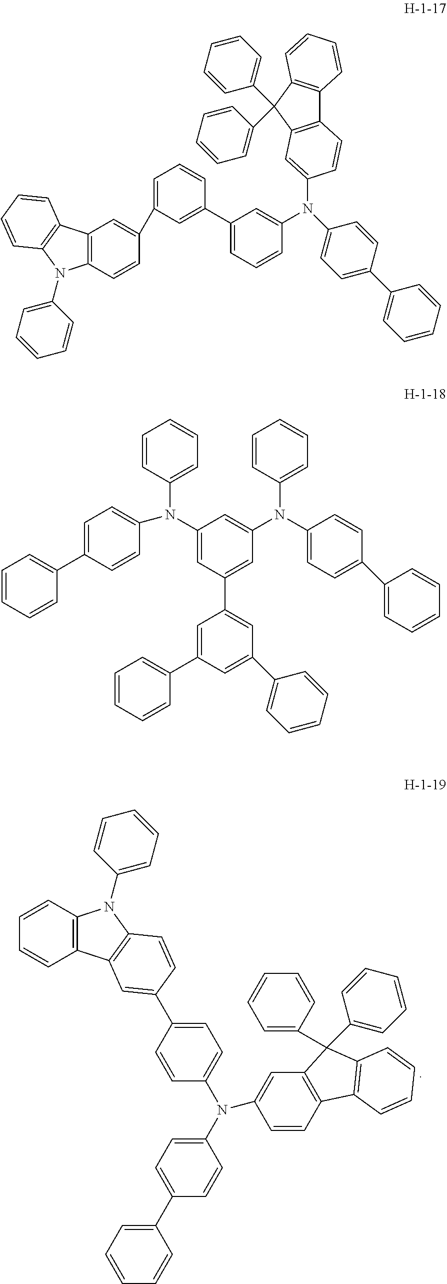 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may further include a suitable hole transport material.

For example, in one or more embodiments, the hole transport region HTR may include at least one selected from among a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

In one or more embodiments, the hole transport region HTR may include at least one selected from among carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalen-1-yl)-N,N′-diphenyl-benzidine (NPB), 4,4′-cyclohexylidene bis[N,N-bis(4-methylphenyl)benzeneamine](TAPC), 4,4′-bis[N,N′-(3-tolyl)amino]-3,3′-dimethylbiphenyl (HMTPD), 1,3-bis(N-carbazolyl)benzene (mCP), and/or the like.

In one or more embodiments, the hole transport region HTR may include at least one selected from among 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include one or more of the above-described compounds of the hole transport region in at least one of a hole injection layer HIL, a hole transport layer HTL, or an electron blocking layer EBL.

A thickness of the hole transport region HTR may be from about 100 Å to about 10,000 Å, for example, from about 100 Å to about 5,000 Å. When the hole transport region HTR includes a hole injection layer HIL, the hole injection layer HIL may have, for example, a thickness of about 30 Å to about 1,000 Å. When the hole transport region HTR includes a hole transport layer HTL, the hole transport layer HTL may have a thickness of about 250 Å to about 1,000 Å. For example, if (e.g., when) the hole transport region HTR includes an electron blocking layer EBL, the electron blocking layer EBL may have a thickness of about 10 Å to about 1,000 Å. If (e.g., when) the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

In one or more embodiments, the hole transport region HTR may further include a charge generating material to increase conductivity (e.g., electric conductivity) in addition to the above-described materials. The charge generating material may be dispersed uniformly (e.g., substantially uniformly) or non-uniformly in the hole transport region HTR. The charge generating material may be, for example, a p-dopant. The p-dopant may include at least one of a halogenated metal compound, a quinone derivative, a metal oxide, or a cyano group-containing compound, but embodiments of the present disclosure are not limited thereto. For example, in one or more embodiments, the p-dopant may include a metal halide compound such as CuI and/or RbI, a quinone derivative such as tetracyanoquinodimethane (TCNQ) and/or 2,3,5,6-tetrafluoro-7,7,8,8-tetracyanoquinodimethane (F₄-TCNQ), a metal oxide such as tungsten oxide and/or molybdenum oxide, a cyano group-containing compound such as dipyrazino[2,3-f: 2′,3′-h]quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The emission layer EML may be provided on the hole transport region HTR. The emission layer EML may have a thickness of, for example, about 100 Å to about 1,000 Å, or about 100 Å to about 300 Å. The emission layer EML may have a single layer formed of a single material, a single layer formed of multiple different materials, or a multi-layered structure having a plurality of layers formed of multiple different materials.

In the light emitting element ED of one or more embodiments, the emission layer EML may be to emit blue light. The light emitting element ED of one or more embodiments may include the amine compound of one or more embodiments in a hole transport region HTR and may thus show high efficiency and long-life characteristics in a blue emission region. However, embodiments of the present disclosure are not limited thereto.

In the light emitting element ED of one or more embodiments, the emission layer EML may include at least one of anthracene derivatives, pyrene derivatives, fluoranthene derivatives, chrysene derivatives, dihydrobenzanthracene derivatives, or triphenylene derivatives. For example, in one or more embodiments, the emission layer EML may include one or more anthracene derivatives or one or more pyrene derivatives.

In the light emitting elements ED of embodiments, shown in FIG. 3 to FIG. 6, the emission layer EML may include a host and a dopant, and the emission layer EML may include a compound represented by Formula E-1. The compound represented by Formula E-1 may be used as a fluorescence host material.

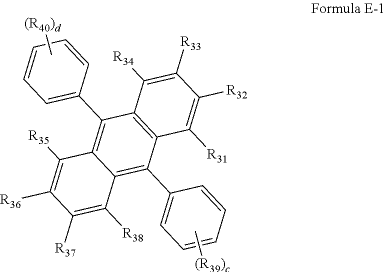

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In one or more embodiments, one or more selected from among R₃₁ to R₄₀ may be each independently combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among Compound E1 to Compound E19.

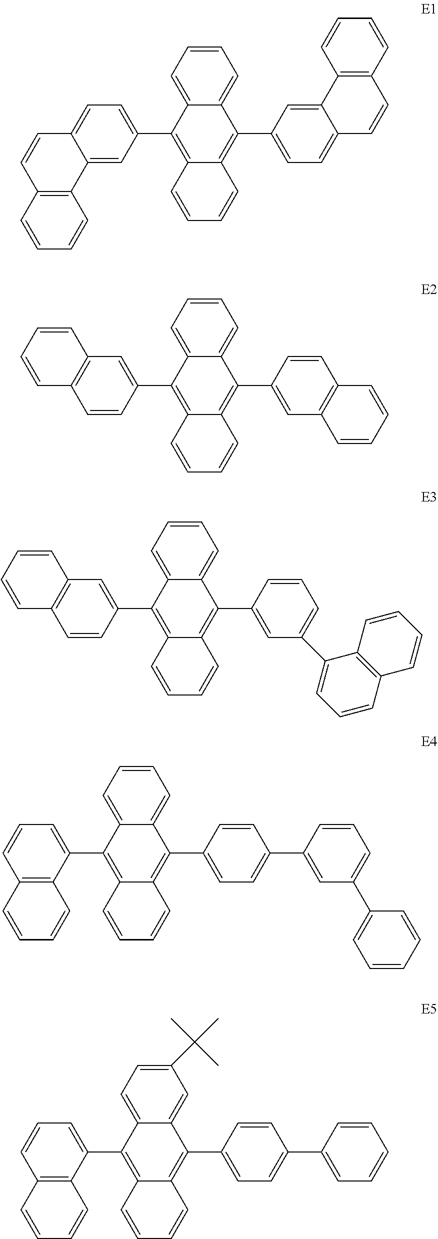

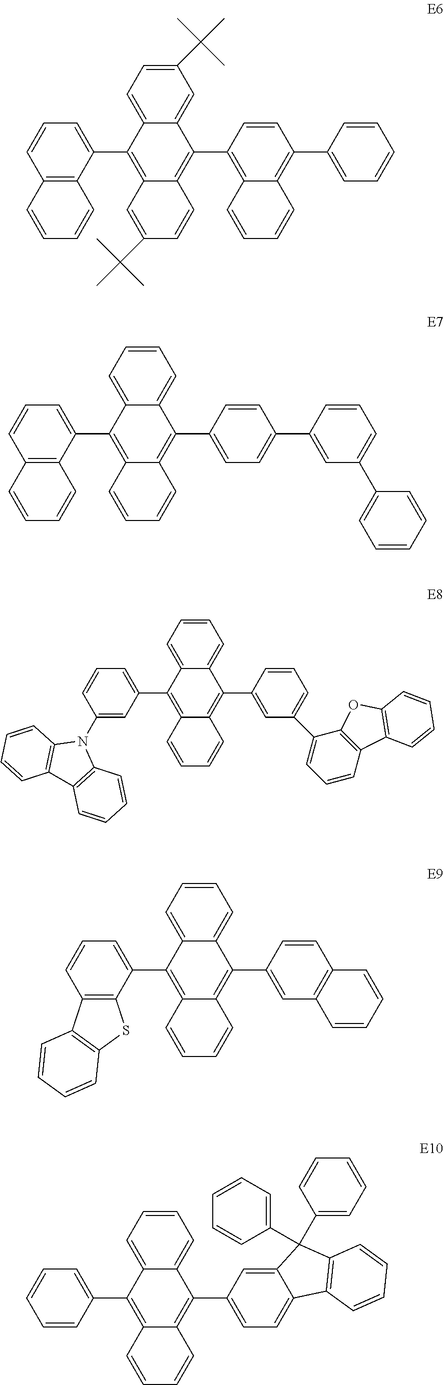

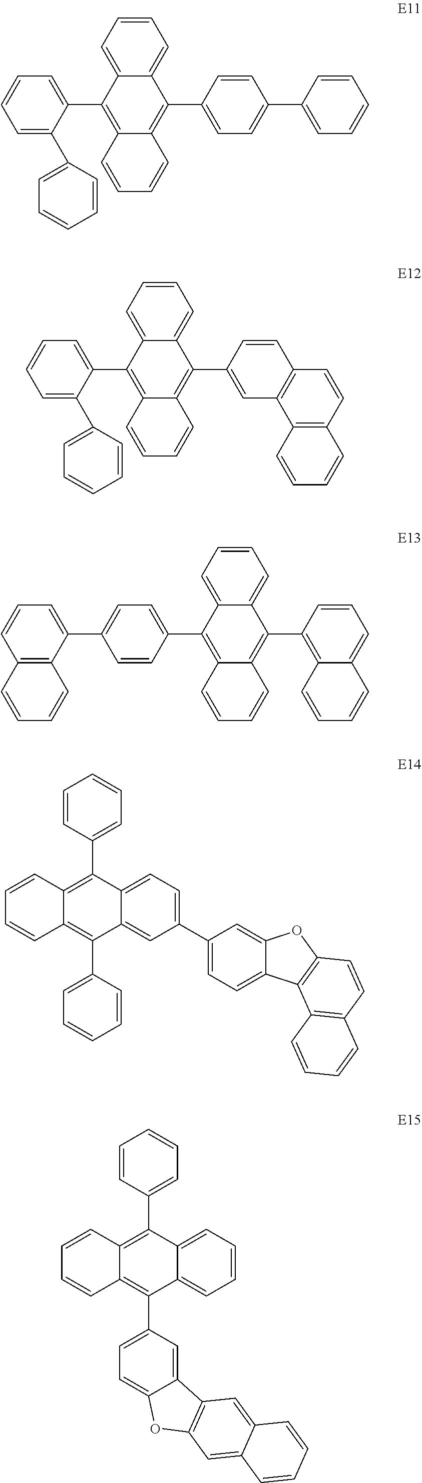

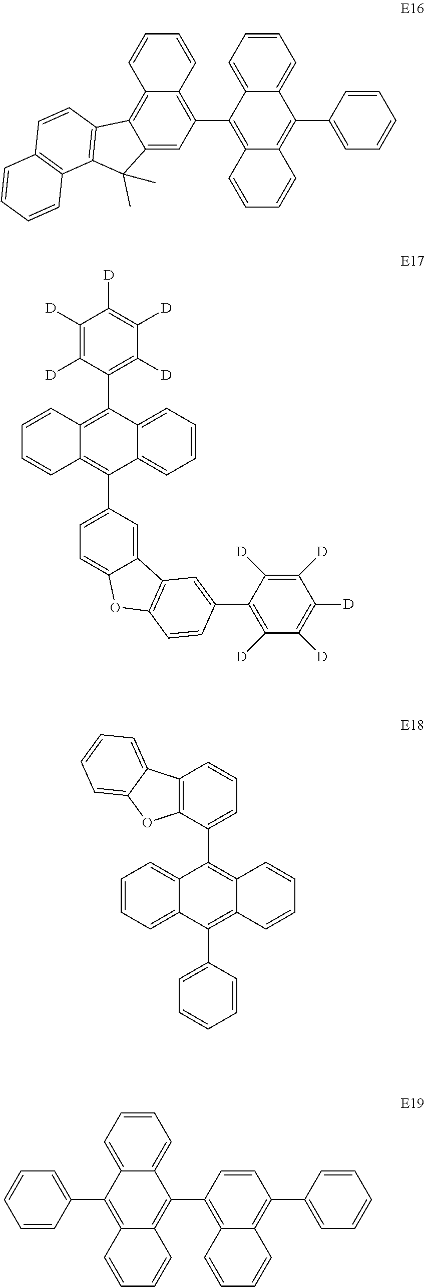

In one or more embodiments, the emission layer EML may include a compound represented by Formula E-2a or Formula E-2b. The compound represented by Formula E-2a or Formula E-2b may be used as a phosphorescence host material.

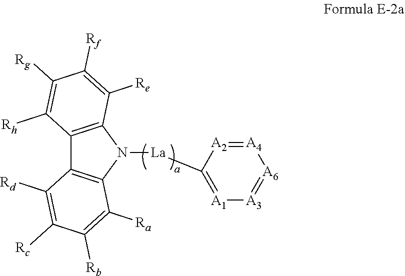

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or more, multiple La's may each independently be a substituted or unsubstituted arylene group of 6 to 30 ring-forming carbon atoms or a substituted or unsubstituted heteroarylene group of 2 to 30 ring-forming carbon atoms.

In addition, in Formula E-2a, A₁ to A₅ may each independently be N or CRi. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In one or more embodiments, one or more selected from among Rₐ to Rᵢ may be independently combined with an adjacent group to form a hydrocarbon ring or a heterocycle including N, O, S, and/or the like, as a ring-forming atom.

In one or more embodiments, in Formula E-2a, two or three selected from among A₁ to A₅ may be N, and the remainder may be CRi.

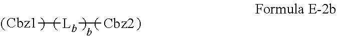

In Formula E-2b, Cbz1 and Cbz2 may each independently be an unsubstituted carbazolyl group or a carbazolyl group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if (e.g., when) “b” is an integer of 2 or more, multiple L_{b}'s may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any one selected from among compounds in Compound Group E-2. However, the compounds listed in Compound Group E-2 are mere examples, and the compound represented by Formula E-2a or Formula E-2b is not limited to the compounds represented in Compound Group E-2.

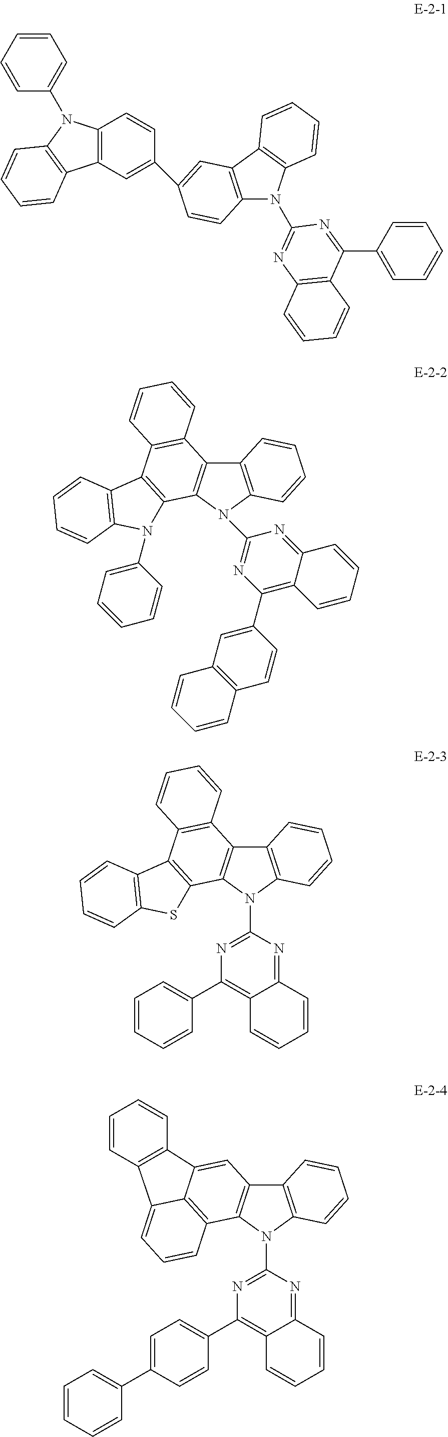

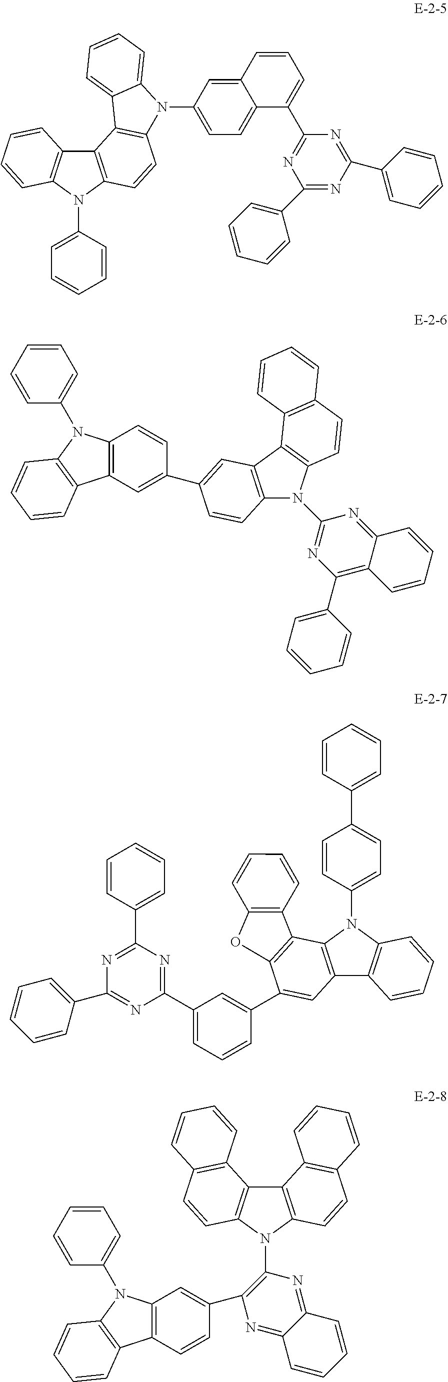 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

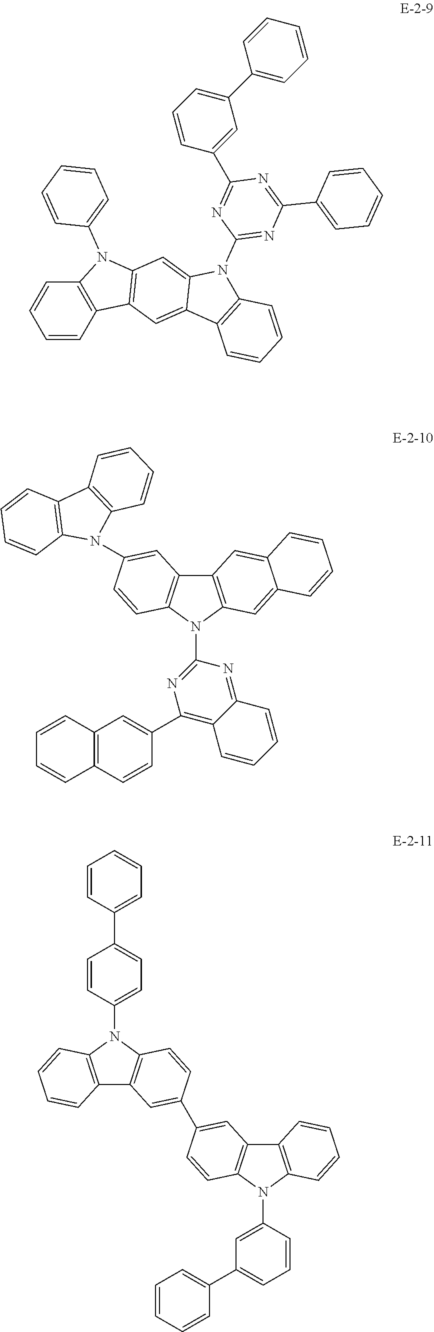 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

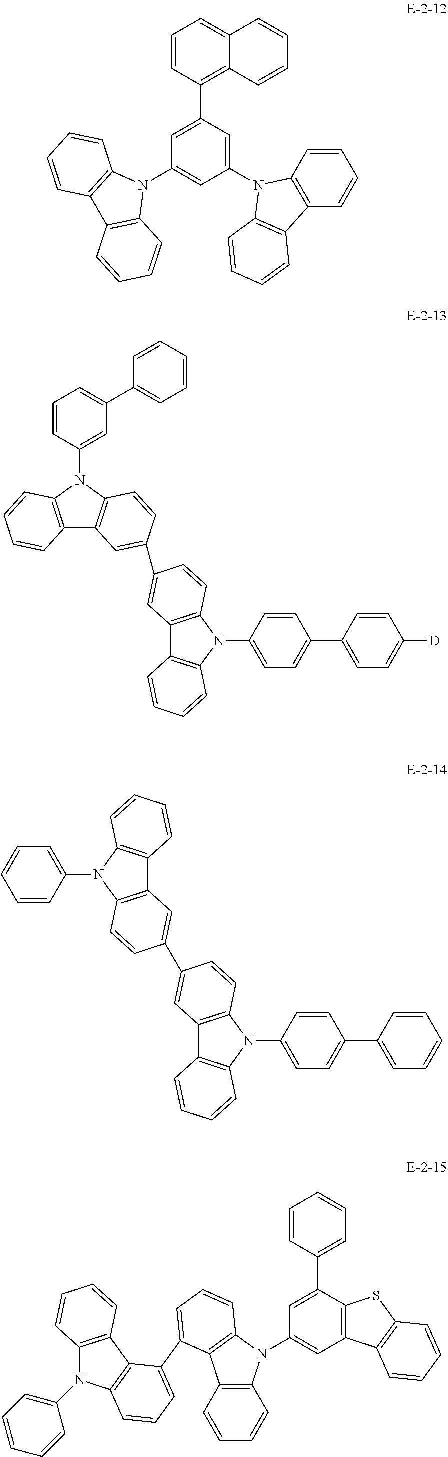 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

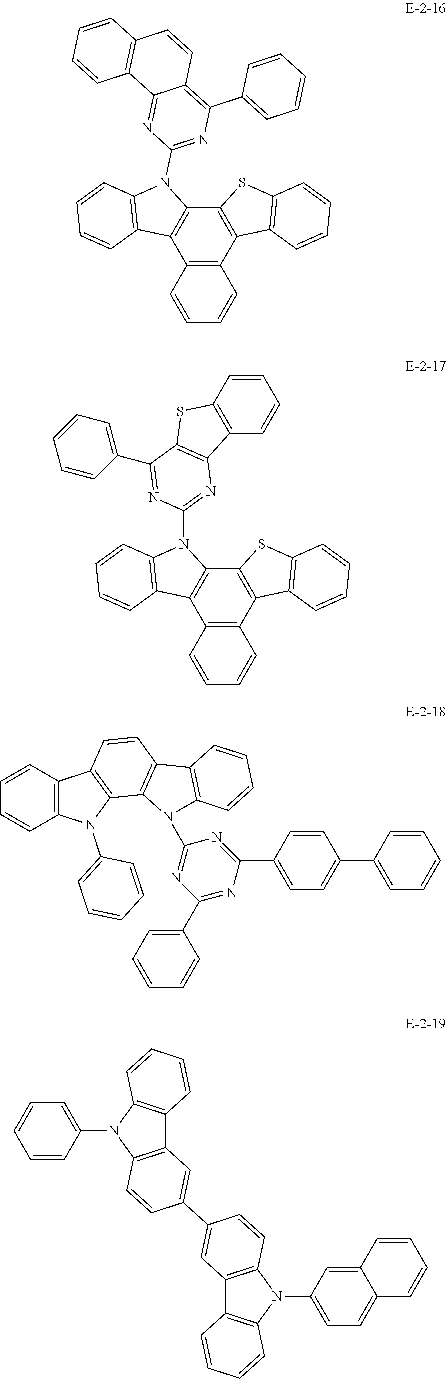 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

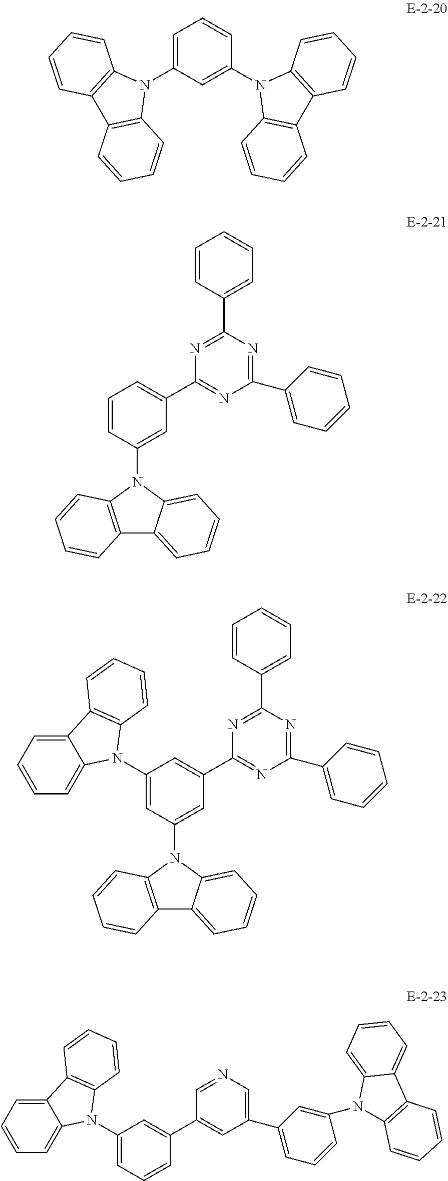 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

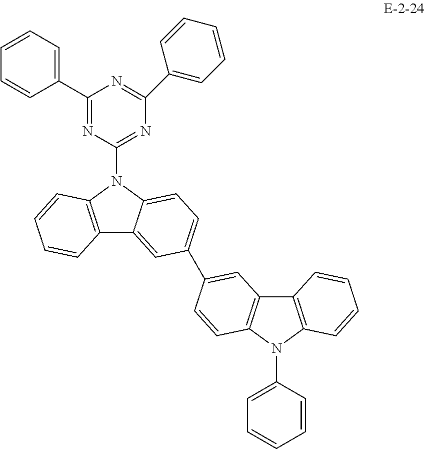 [SMILES: c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the emission layer EML may further include a material well-suitable in the art as a host material. For example, the emission layer EML may include, as a host material, at least one of bis(4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto. For example, tris(8-hydroxyquinolinato)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and/or the like, may be used as the host material.

In one or more embodiments, the emission layer EML may include a compound represented by Formula M-a or Formula M-b. The compound represented by Formula M-a or Formula M-b may be used as a phosphorescence dopant material. In addition, in one or more embodiments, the compound represented by Formula M-a or Formula M-b may be used as an auxiliary dopant material.

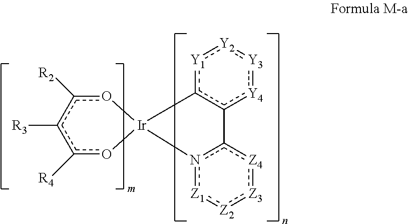

In Formula M-a, Y₁ to Y₄, and Z₁ to Z₄ may each independently be CR₁ or N, and R₁ to R₄ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In Formula M-a, “m” is 0 or 1, and “n” is 2 or 3. In Formula M-a, if (e.g., when) “m” is 0, “n” is 3, and if (e.g., when) “m” is 1, “n” is 2.

The compound represented by Formula M-a may be used as a phosphorescence dopant.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

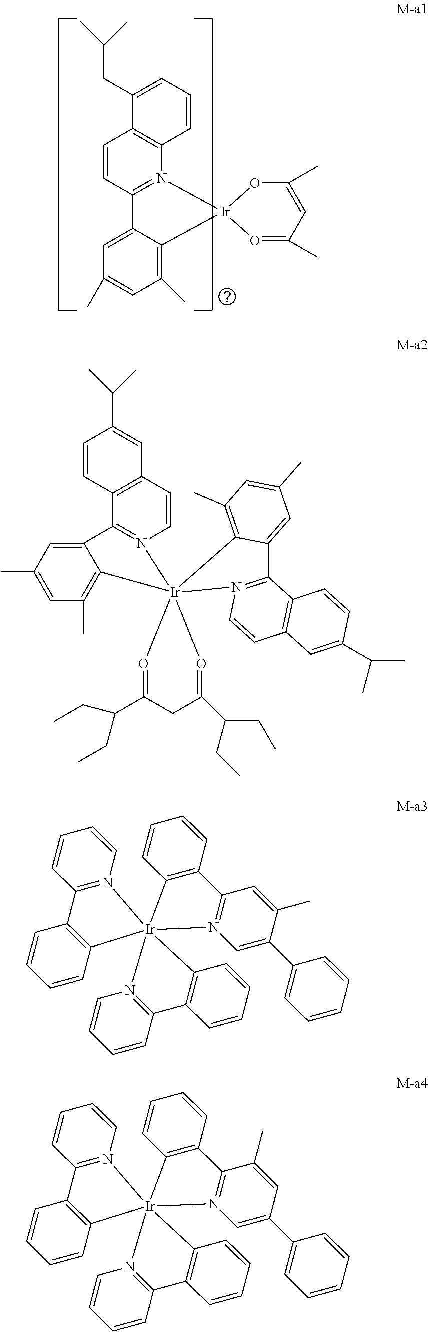

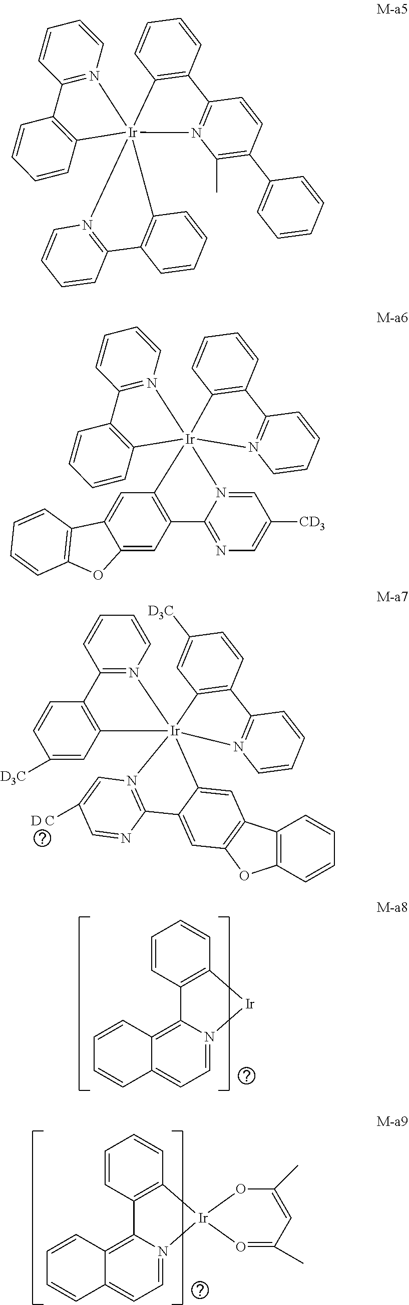

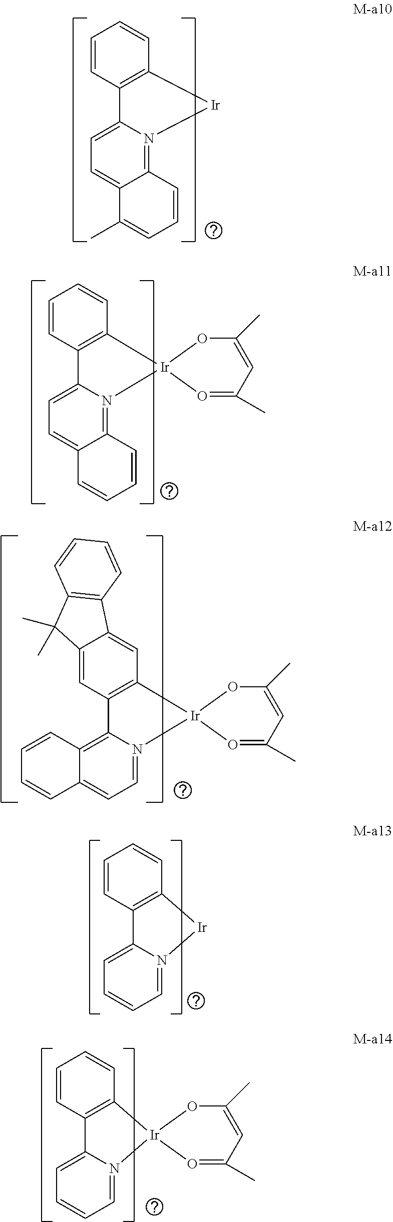

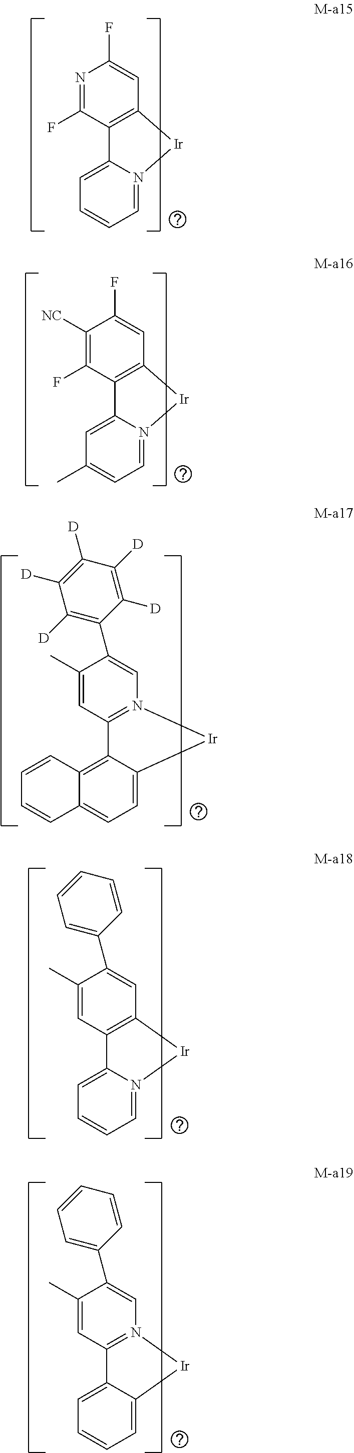

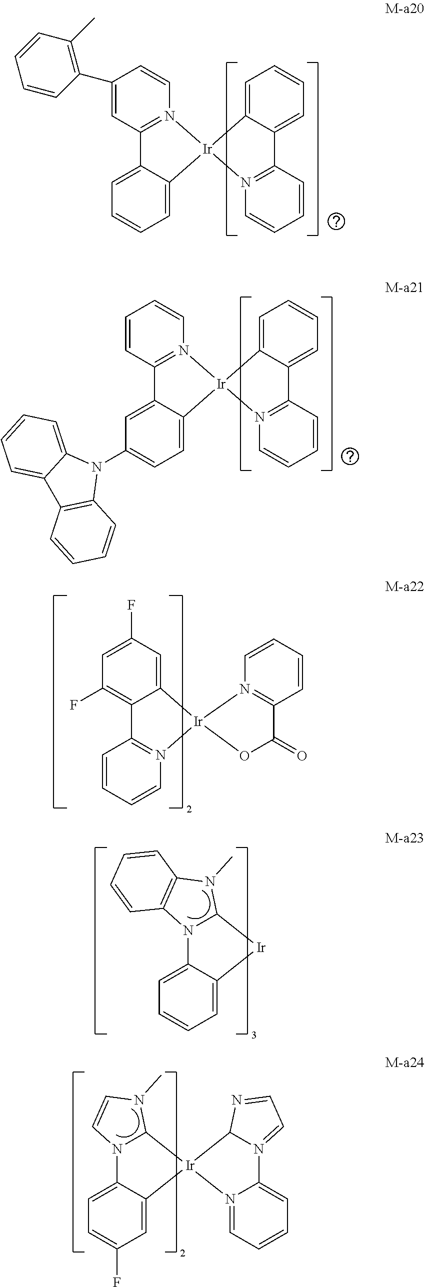

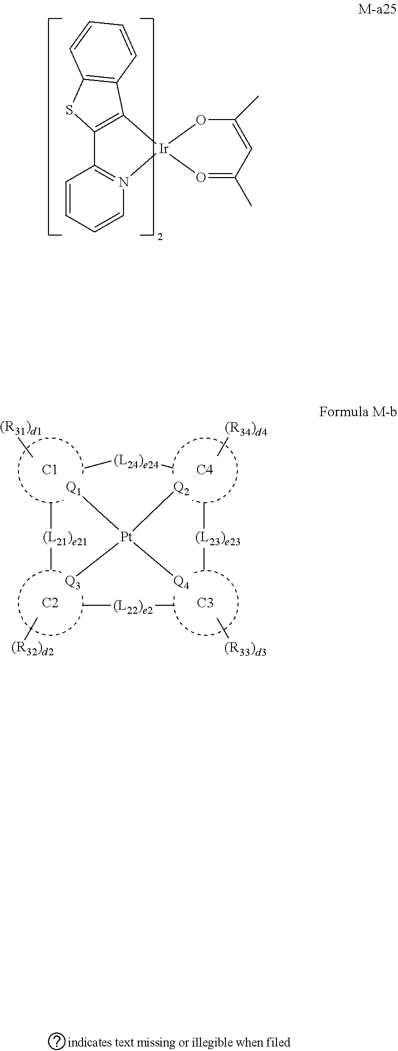

In Formula M-b, Q₁ to Q₄ may each independently be C or N, C1 to C4 may each independently be a substituted or unsubstituted hydrocarbon ring of 5 to 30 ring-forming carbon atoms or a substituted or unsubstituted heterocycle of 2 to 30 ring-forming carbon atoms. L₂₁ to L₂₄ may each independently be a direct linkage,

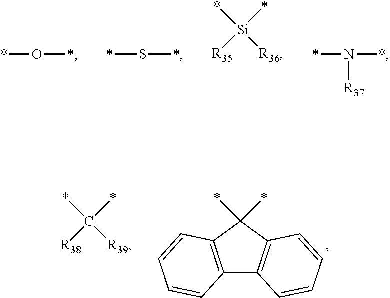

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be used as a blue phosphorescence dopant or a green phosphorescence dopant. In one or more embodiments, the compound represented by Formula M-b may be an auxiliary dopant and may be further included in the emission layer EML.

The compound represented by Formula M-b may be any one selected from among Compound M-b-1 to Compound M-b-11. However, the Compound M-b-1 to Compound M-b-11 are mere examples, and the compound represented by Formula M-b is not limited to Compound M-b-1 to Compound M-b-11.

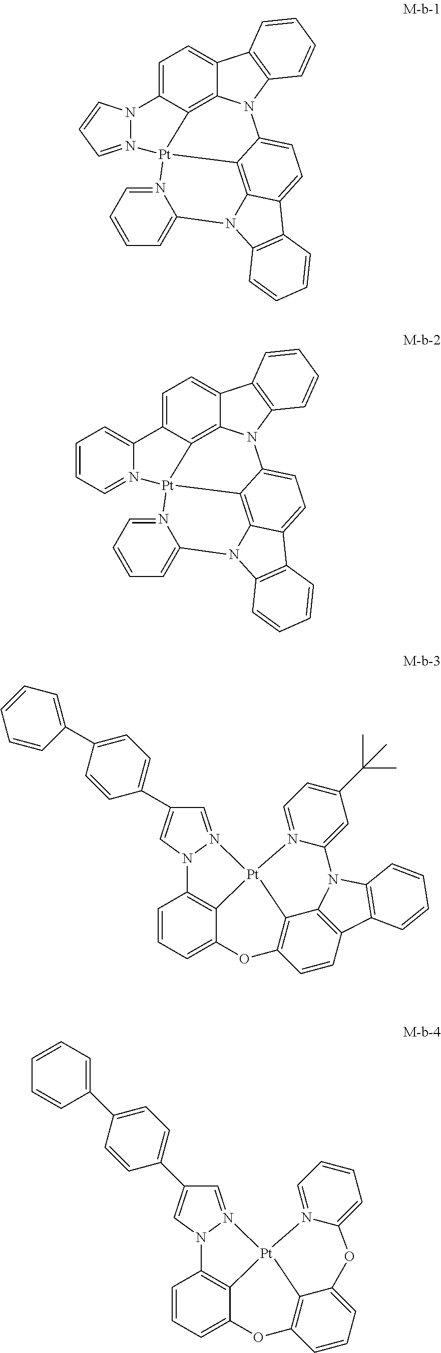

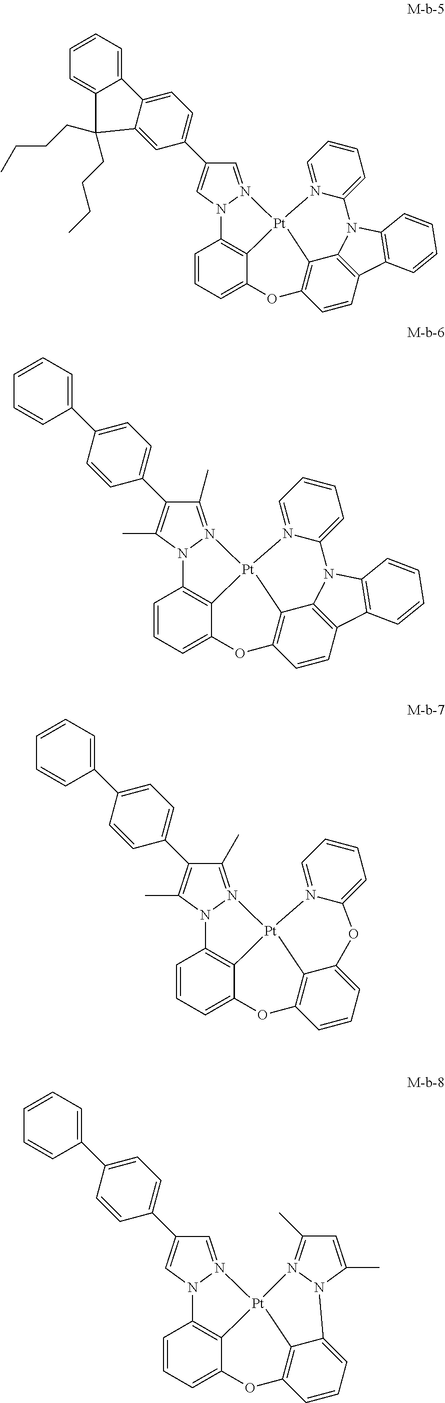

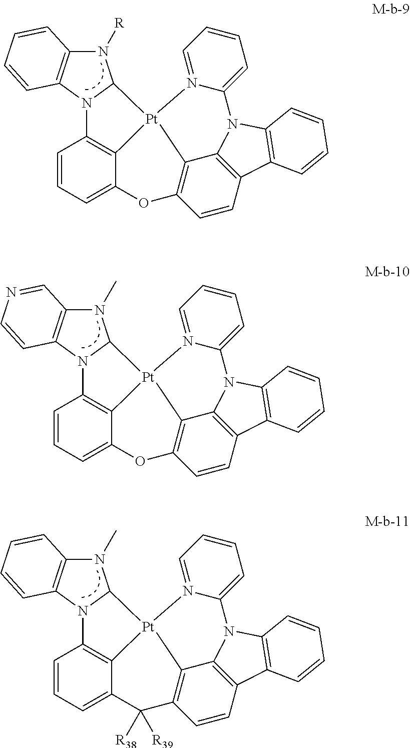

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the emission layer EML may contain a first compound represented by any one selected from among Formulas F-a to F-c, a second compound represented by Formula HT-1, a third compound represented by Formula ET-1, and a fourth compound represented by Formula D-1.

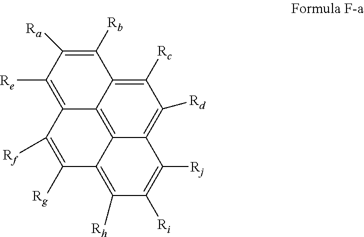

In Formula F-a, two selected from among Rₐ to Rⱼ may each independently be substituted with

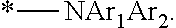

The remainder not substituted with

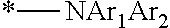

among Rₐ to Rⱼ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In

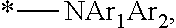

Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, in some embodiments, at least one selected from among Ar₁ and Ar₂ may be a heteroaryl group including O or S as a ring-forming atom.

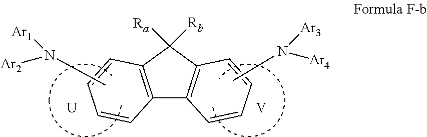

In Formula F-b, Rₐ and Rb may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring.

In Formula F-b, Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms.

In Formula F-b, the number of rings represented by U and V may each independently be 0 or 1. For example, in Formula F-b, if (e.g., when) the number of U or V is 1, one ring forms a part of a fused ring at the designated part by U or V, and if (e.g., when) the number of U or V is 0, a ring is not present at the designated part by U or V. for example, if (e.g., when) the number of U is 0, and the number of V is 1, or if (e.g., when) the number of U is 1, and the number of V is 0, a fused ring having the fluorene core of Formula F-b may be a ring compound with four rings. In one or more embodiments, if (e.g., when) the number of both (e.g., simultaneously) U and V is 0, the fused ring of Formula F-b may be a ring compound with three rings. In one or more embodiments, if (e.g., when) the number of both (e.g., simultaneously) U and V is 1, a fused ring having the fluorene core of Formula F-b may be a ring compound with five rings.

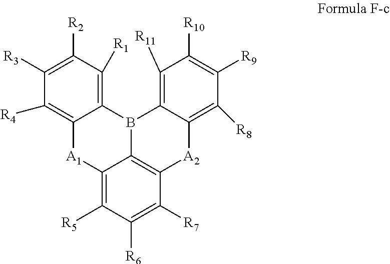

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or independently combined with an adjacent group to form a ring.

In Formula F-c, A₁ and A₂ may each independently be combined with the substituents of an adjacent ring to form a fused ring. For example, if (e.g., when) A₁ and A₂ may each independently be NRₘ, A₁ may be combined with R₄ or R₅ to form a ring. In addition, A₂ may be combined with R₇ or R₈ to form a ring.

In one or more embodiments, the second compound may be used as a hole transporting host material of the emission layer EML.

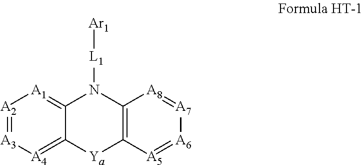

In Formula HT-1, A₁ to A₈ may each independently be N or CR₅₁. For example, in one or more embodiments, all A₁ to A₈ may be CR₅₁. In one or more embodiments, any one selected from among A₁ to A₈ may be N, and the remainder may be CR₅₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, in one or more embodiments, L₁ may be a direct linkage, a substituted or unsubstituted phenylene group, a substituted or unsubstituted divalent biphenylyl group, a substituted or unsubstituted divalent carbazolyl group, and/or the like, but embodiments of the present disclosure are not limited thereto.

In Formula HT-1, Yₐ may be a direct linkage, CR₅₂R₅₃, or SiR₅₄R₅₅. For example, it may refer to that two six-membered rings (e.g., two benzene rings) connected with the nitrogen atom of Formula HT-1 may be connected via a direct linkage,

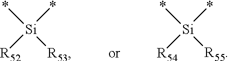

In Formula HT-1, if (e.g.,) Yₐ is a direct linkage, the substituent represented by Formula HT-1 may include a carbazolyl moiety.

In Formula HT-1, Ar₁ may be a substituted or unsubstituted aryl group of 6 to 30 ring-forming carbon atoms or a substituted or unsubstituted heteroaryl group of 2 to 30 ring-forming carbon atoms. For example, in one or more embodiments, Ar₁ may be a substituted or unsubstituted carbazolyl group, a substituted or unsubstituted dibenzofuran group, a substituted or unsubstituted dibenzothiophene group, a substituted or unsubstituted biphenylyl group, and/or the like, but embodiments of the present disclosure are not limited thereto.

In Formula HT-1, R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R₅₁ to R₅₅ may be each independently combined with an adjacent group to form a ring. For example, in one or more embodiments, R₅₁ to R₅₅ may each independently be hydrogen or deuterium. In one or more embodiments, R₅₁ to R₅₅ may each independently be an unsubstituted methyl group or an unsubstituted phenyl group.

In one or more embodiments, the second compound represented by Formula HT-1 may be any one selected from among compounds represented in Compound Group 2. The emission layer EML may include at least one selected from among the compounds represented in Compound Group 2 as a hole transporting host material.

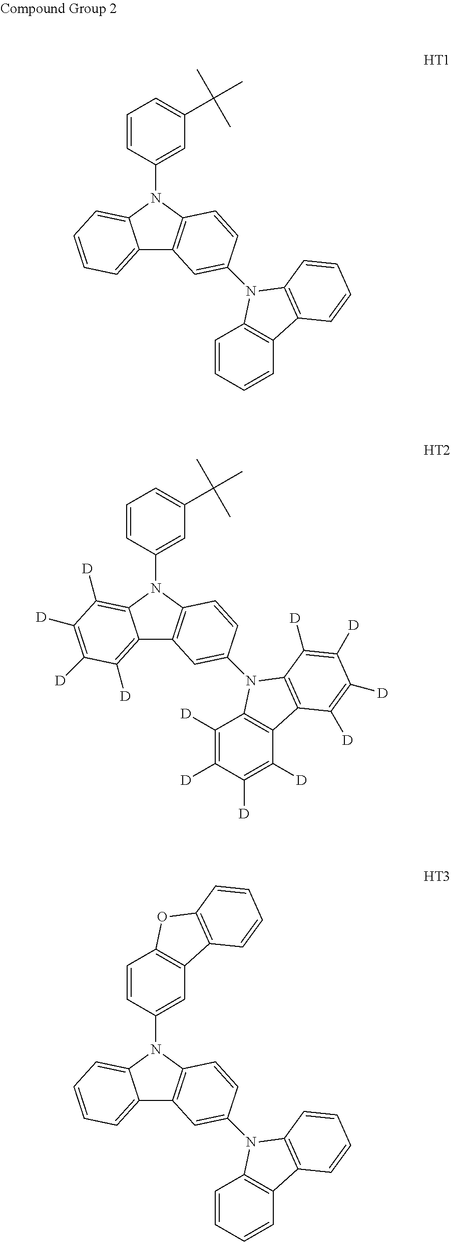 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

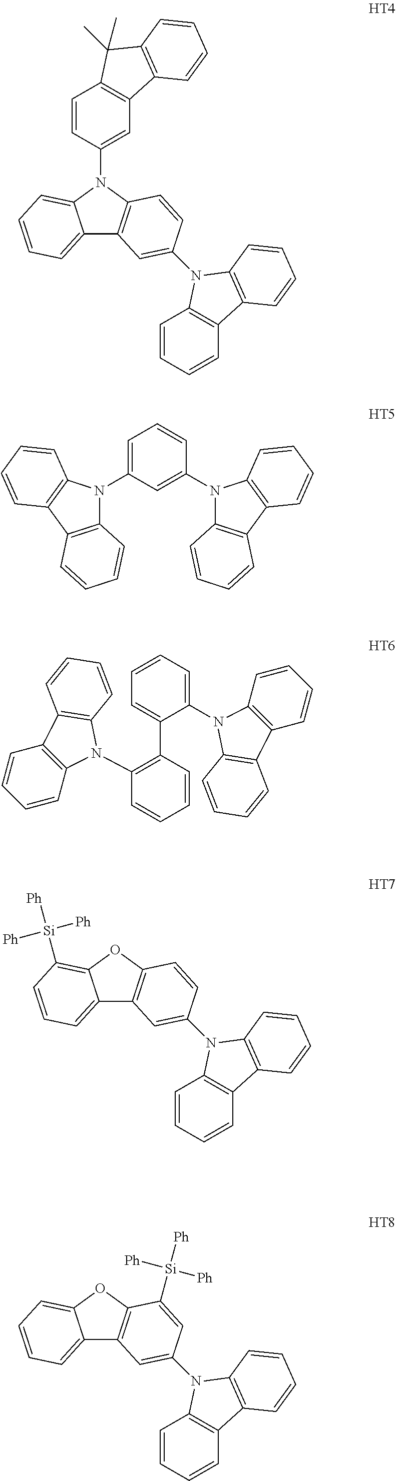 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

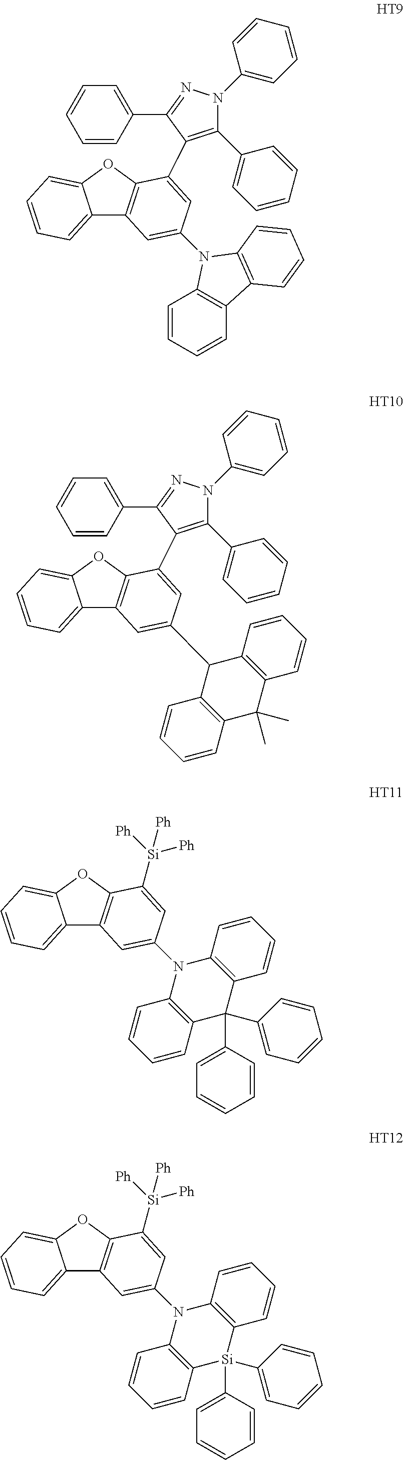 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2C(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

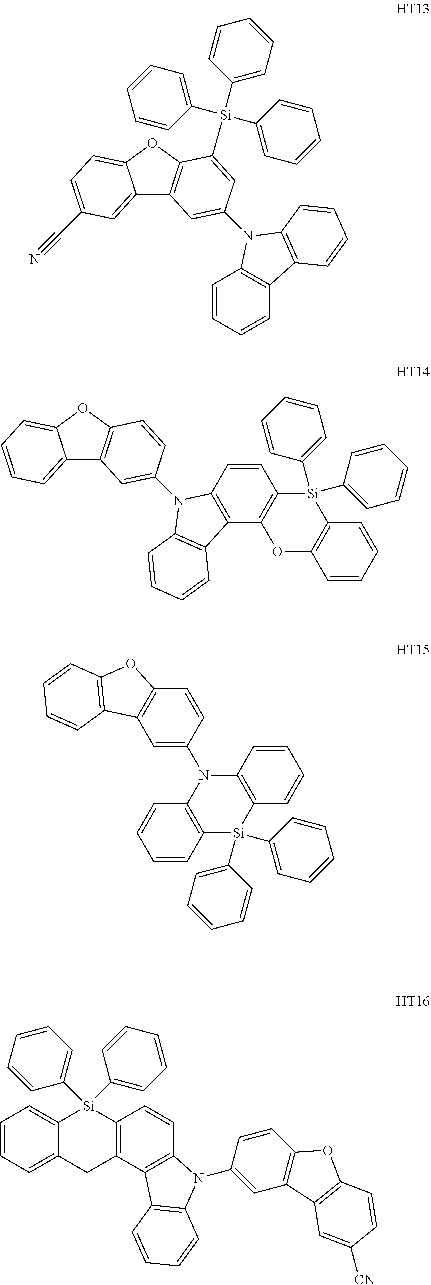 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4C6)cc3c2c1]

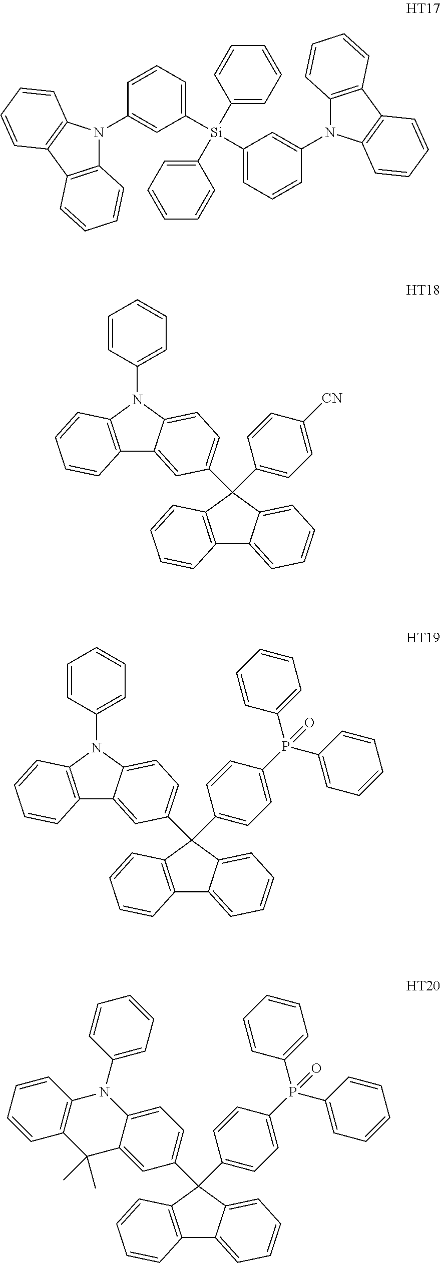 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

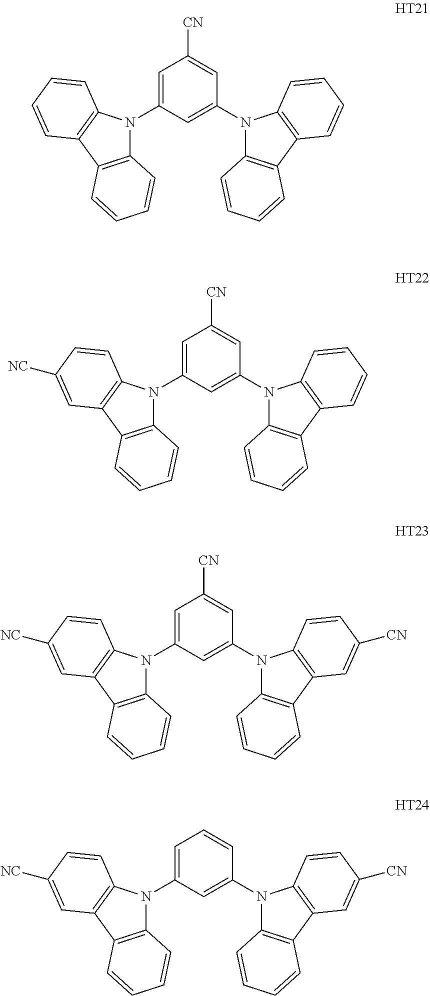 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)c1]

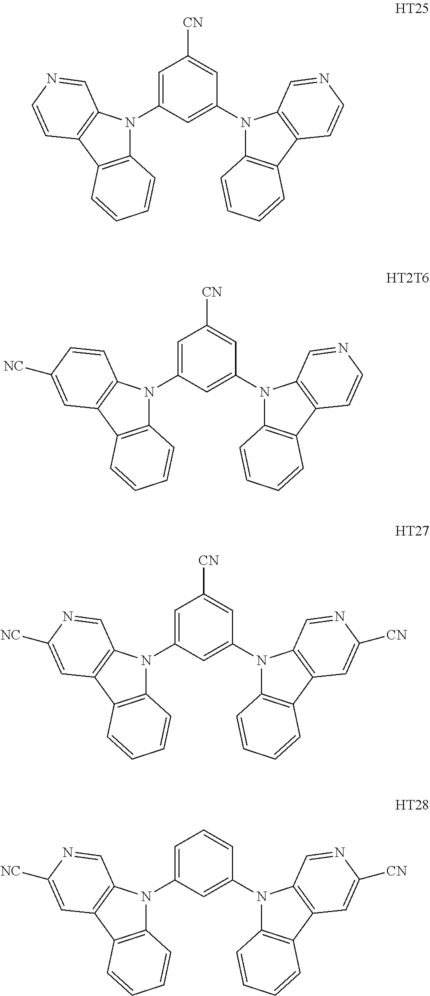 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)c3)c2cn1]

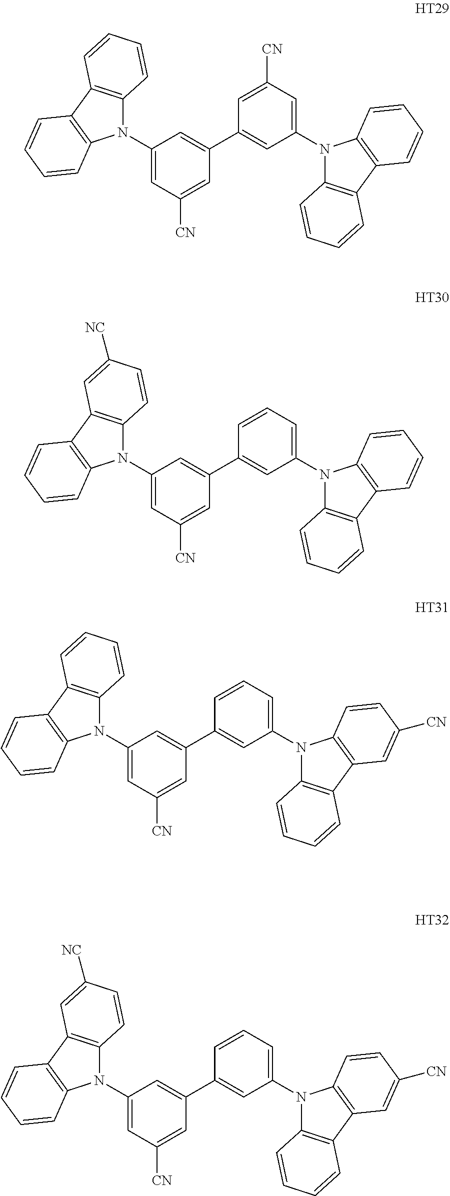 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

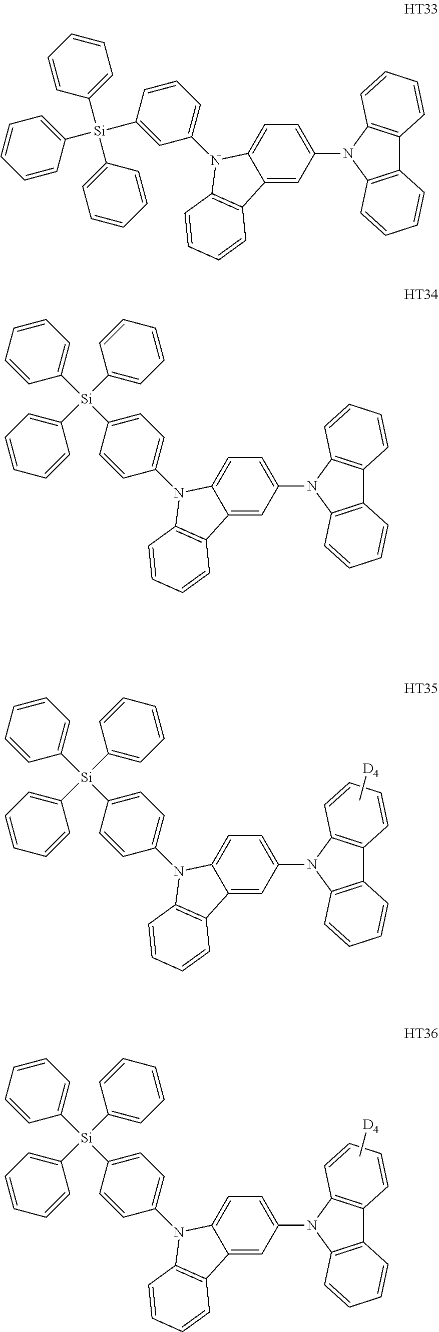

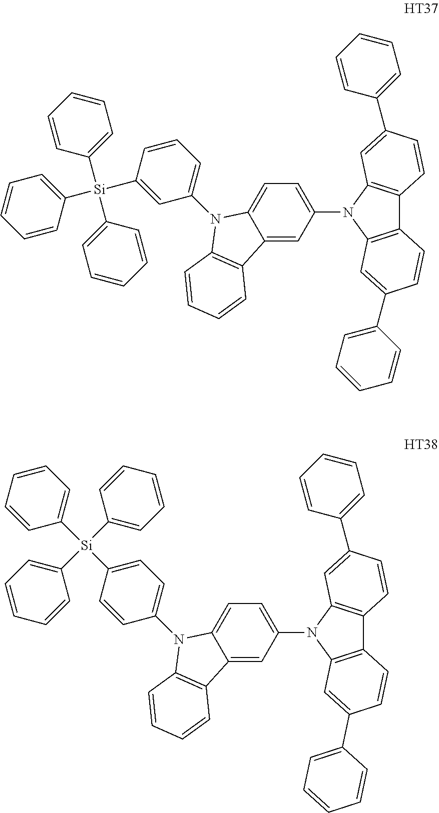 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1]

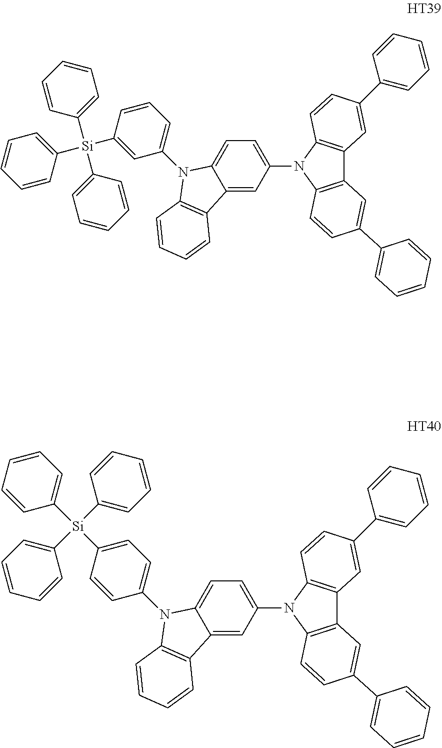 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1]

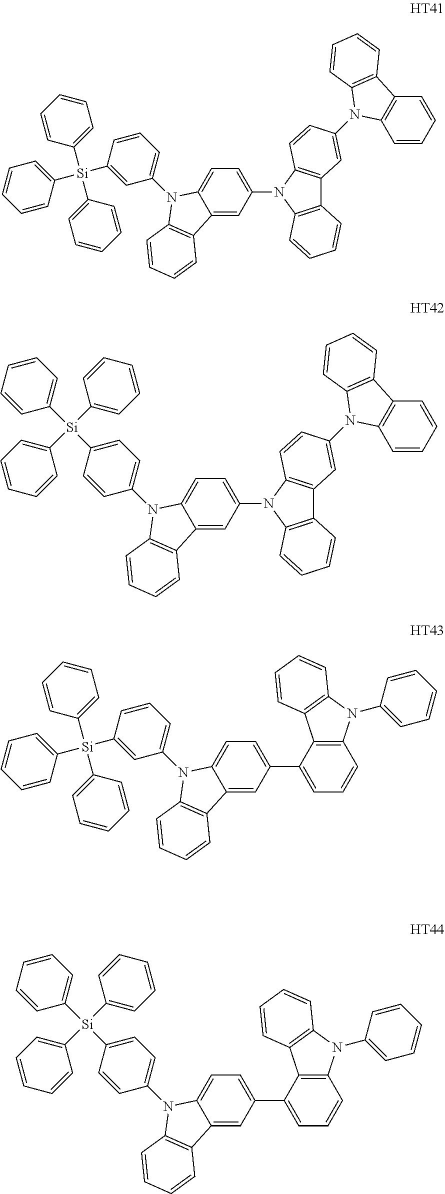 [SMILES: c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1 ; c1ccc(-n2c3ccccc3c3c(-c4ccc5c(c4)c4ccccc4n5-c4ccc([Si](c5ccccc5)(c5ccccc5)c5ccccc5)cc4)cccc32)cc1]

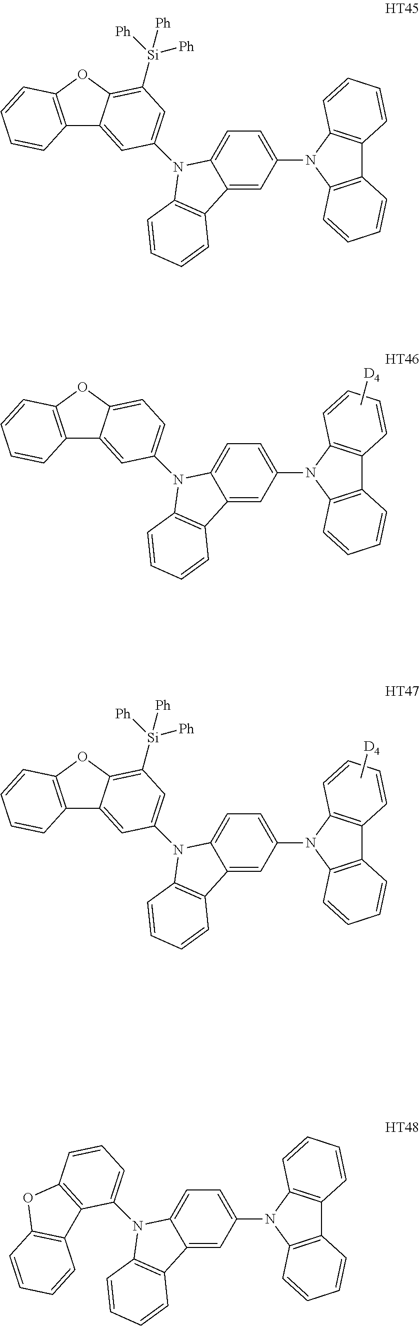

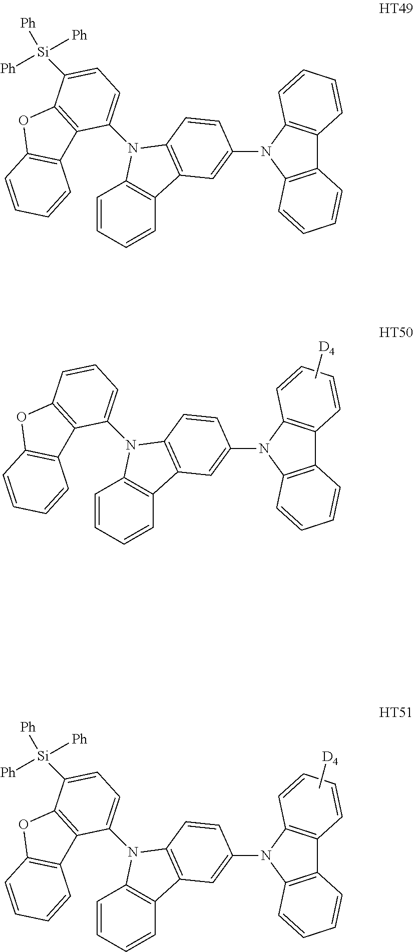

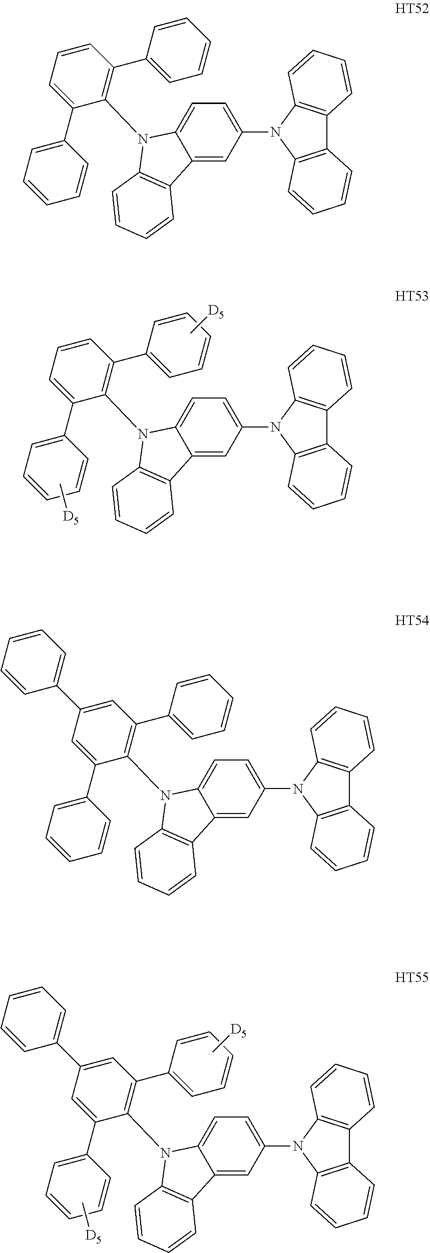

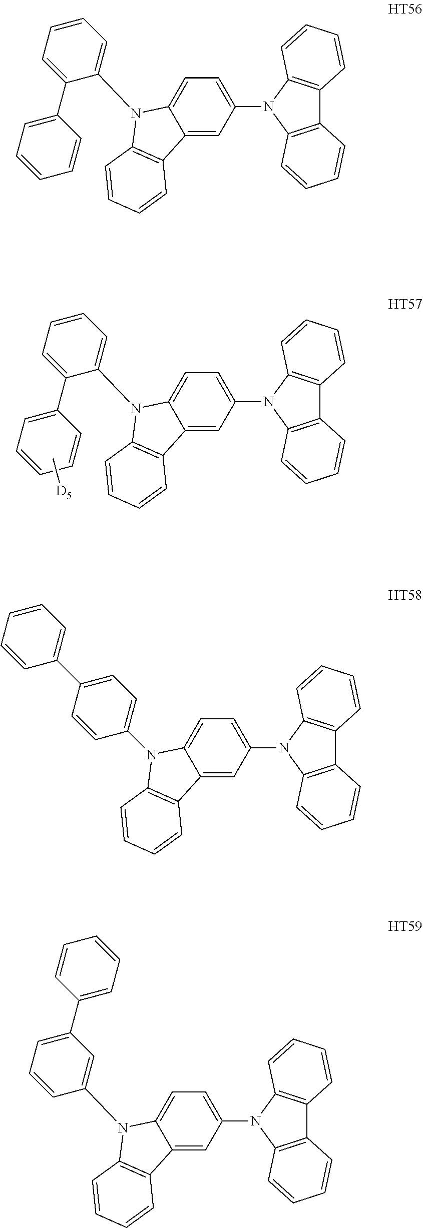

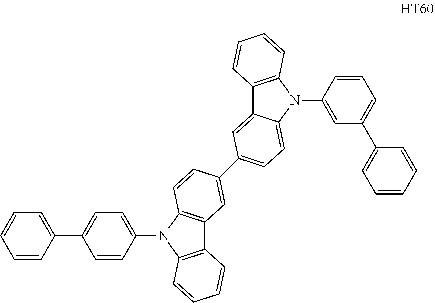 [SMILES: c1ccc(-c2ccc(-n3c4ccccc4c4cc(-c5ccc6c(c5)c5ccccc5n6-c5cccc(-c6ccccc6)c5)ccc43)cc2)cc1]

In the example compounds suggested in Compound Group 2, “D” refers to deuterium, and “Ph” may refer to a substituted or unsubstituted phenyl group. For example, in the example compounds suggested in Compound Group 2, “Ph” may be an unsubstituted phenyl group.

In one or more embodiments, the emission layer EML may contain the third compound represented by Formula ET-1. For example, the third compound may be used as an electron transporting host material of the emission layer EML.

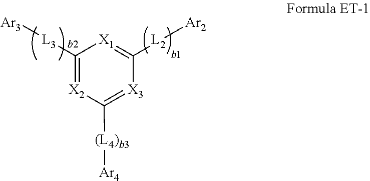

In Formula ET-1, at least one selected from among X₁ to X₃ may be N, and the remainder may be CR₅₆. For example, in one or more embodiments, one selected from among X₁ to X₃ may be N, and the remainder two may each independently be CR₅₆. In these embodiments, the third compound represented by Formula ET-1 may include a pyridine moiety. In one or more embodiments, two selected from among X₁ to X₃ may be N, and the remainder may be CR₅₆. In these embodiments, the third compound represented by Formula ET-1 may include a pyrimidine moiety. In one or more embodiments, X₁ to X₃ may be all N. In these embodiments, the third compound represented by Formula ET-1 may include a triazine moiety.

In Formula ET-1, R₅₆ may be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer of 0 to 10.

In Formula ET-1, Ar₂ to Ar₄ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, Ar₂ to Ar₄ may each independently be substituted or unsubstituted phenyl groups or substituted or unsubstituted carbazolyl groups.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) each of b1 to b3 is an integer of 2 or more, L₂'s to L₄'s may each independently be a substituted or unsubstituted arylene group of 6 to 30 ring-forming carbon atoms or a substituted or unsubstituted heteroarylene group of 2 to 30 ring-forming carbon atoms.

In one or more embodiments, the third compound may be any one selected from among compounds in Compound Group 3. The light emitting element ED of one or more embodiments may include any one selected from among the compounds in Compound Group 3.

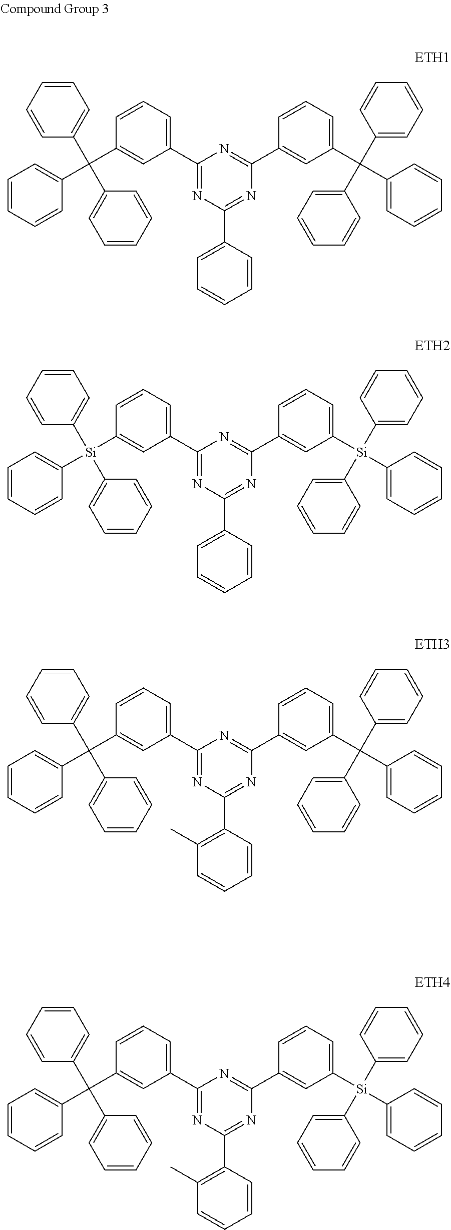 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc(C(c3ccccc3)(c3ccccc3)c3ccccc3)c2)nc(-c2cccc([Si](c3ccccc3)(c3ccccc3)c3ccccc3)c2)n1]

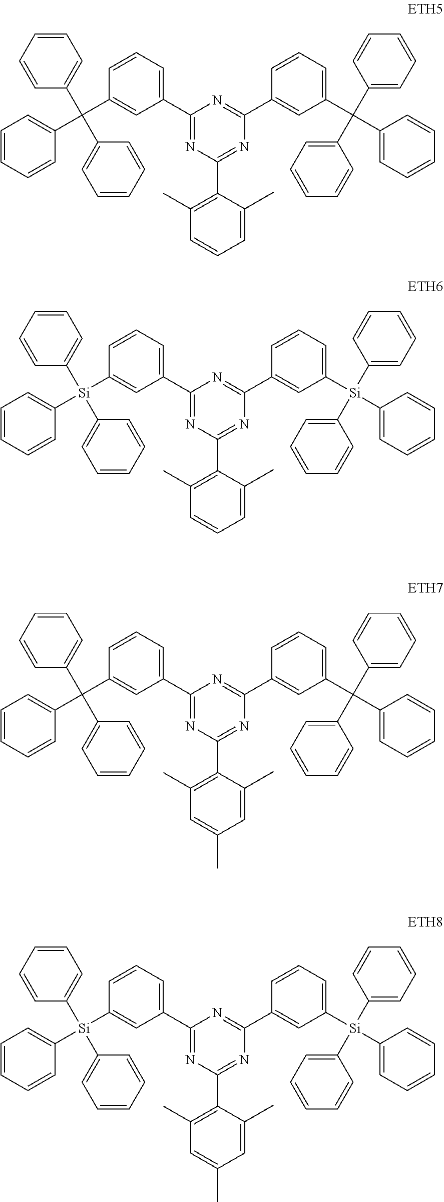 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

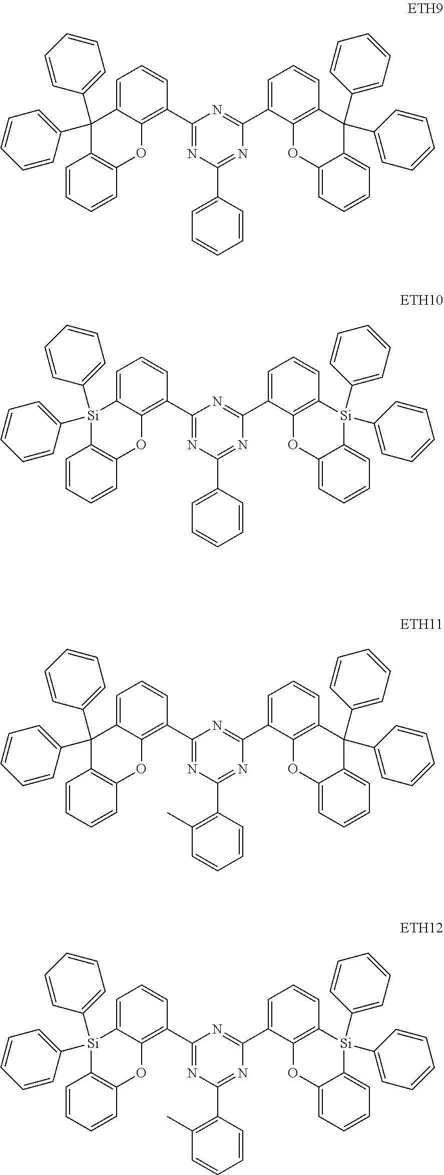 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

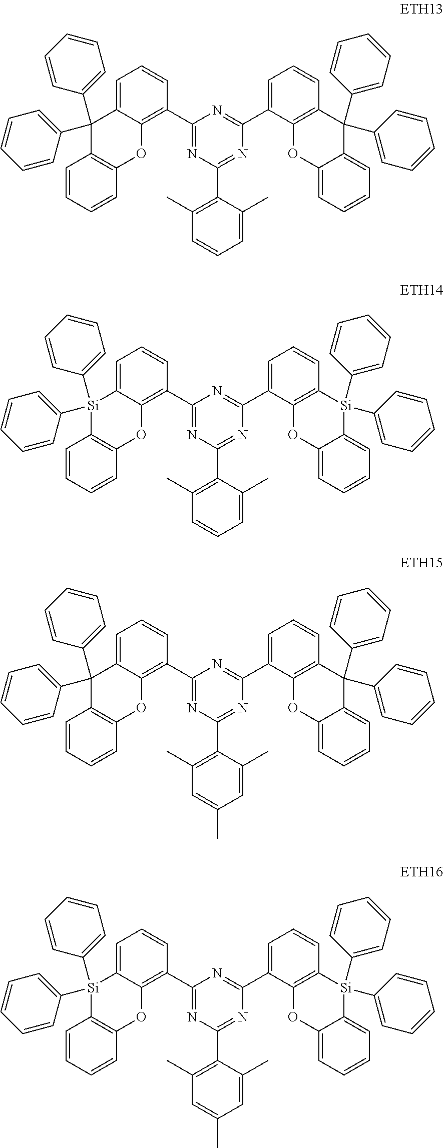 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

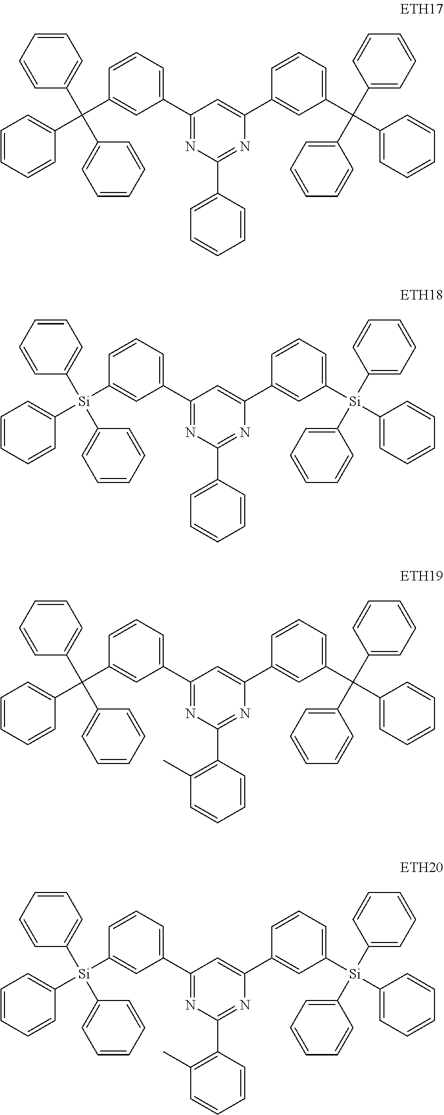 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

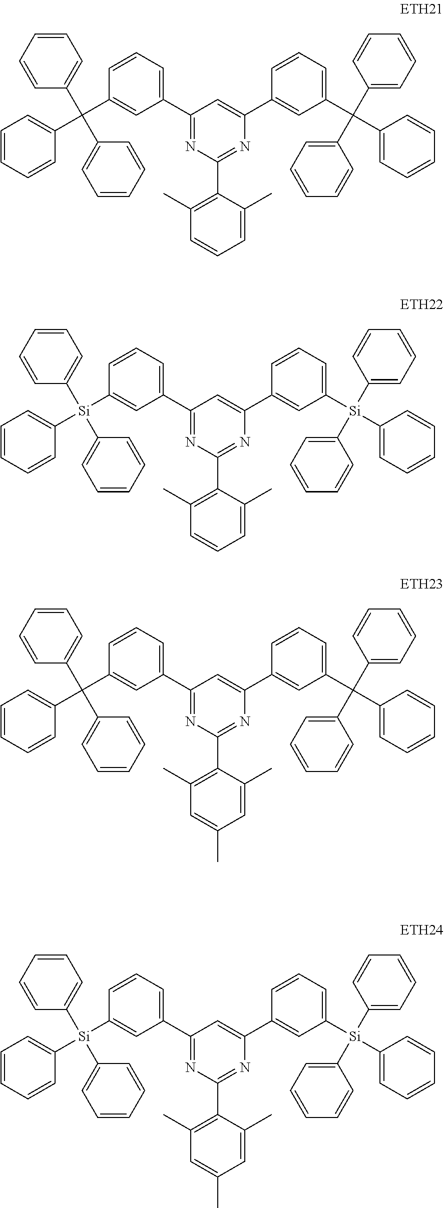 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

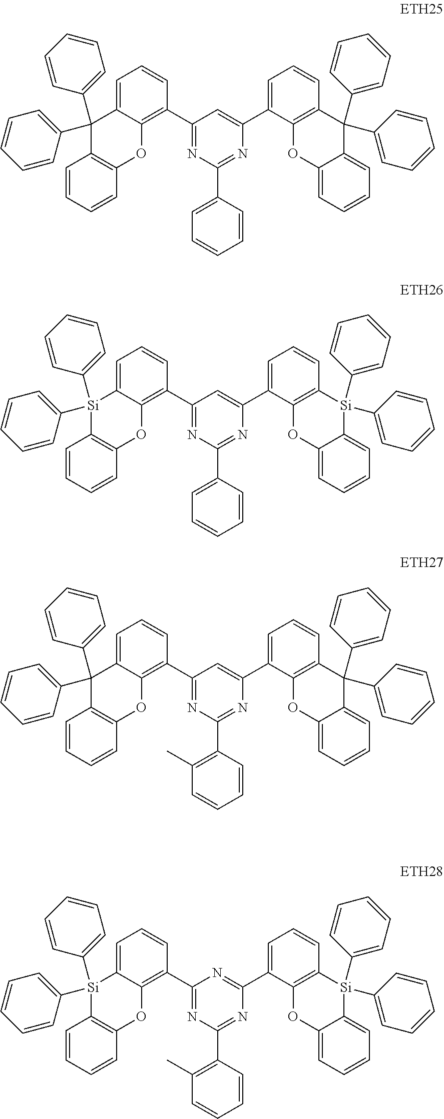 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

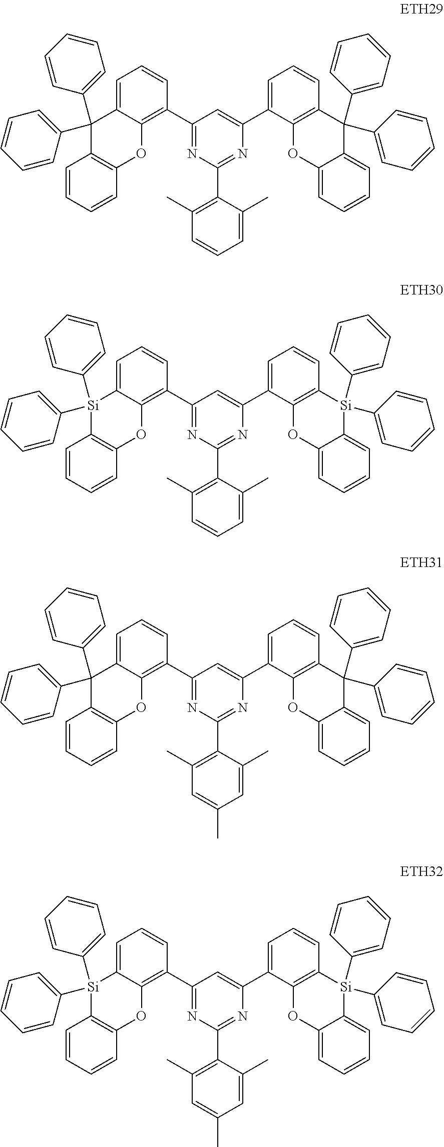 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

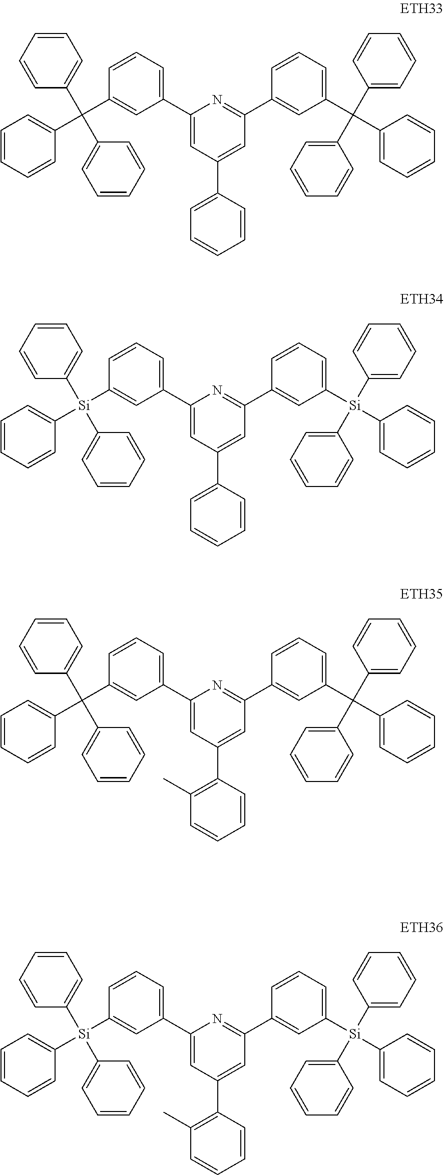 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

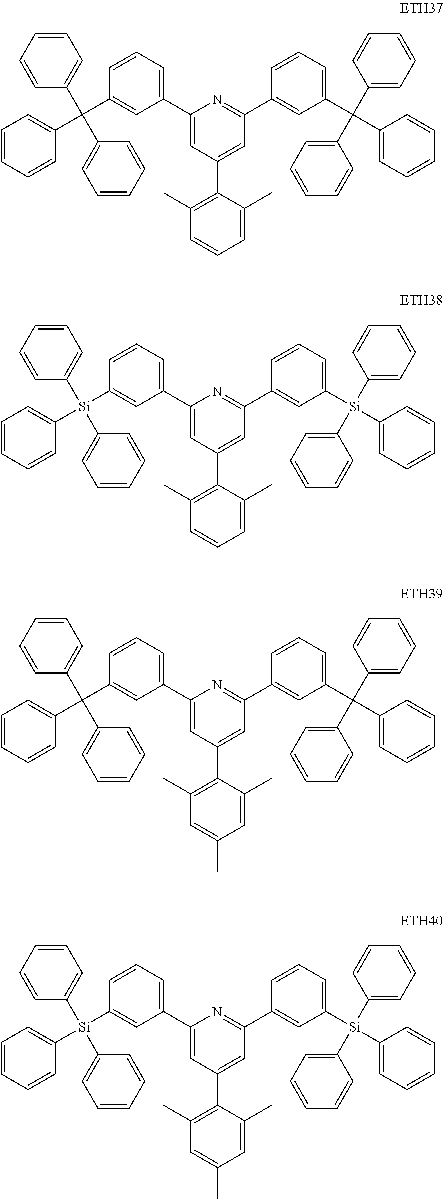 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

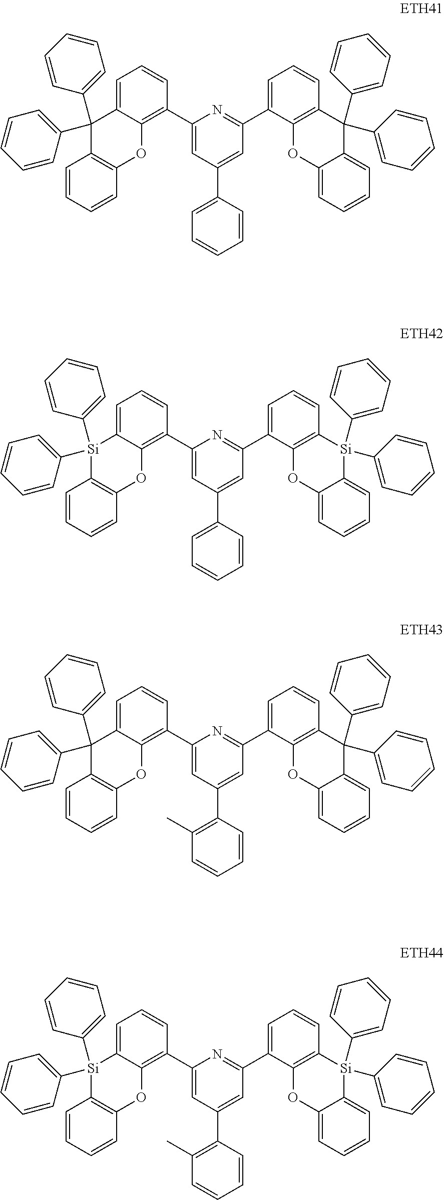 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

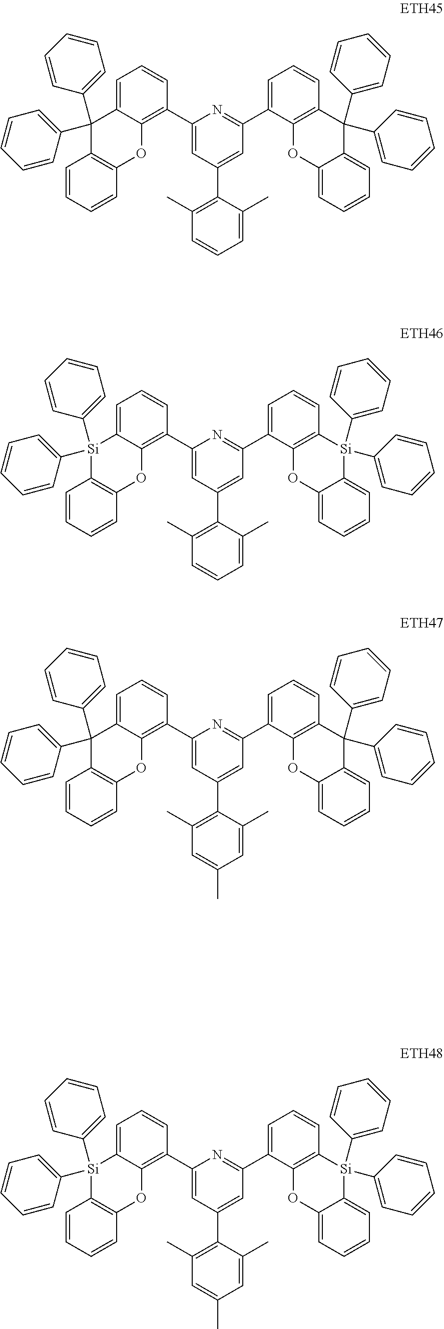 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

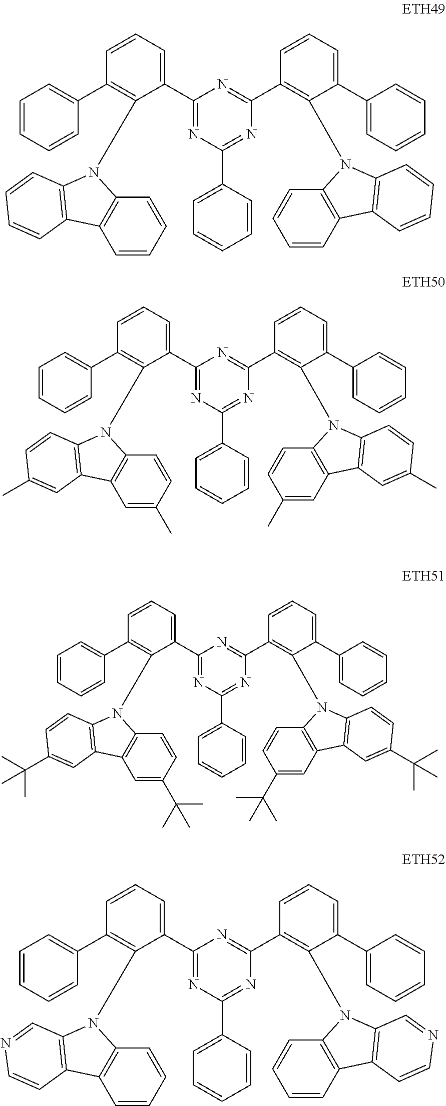

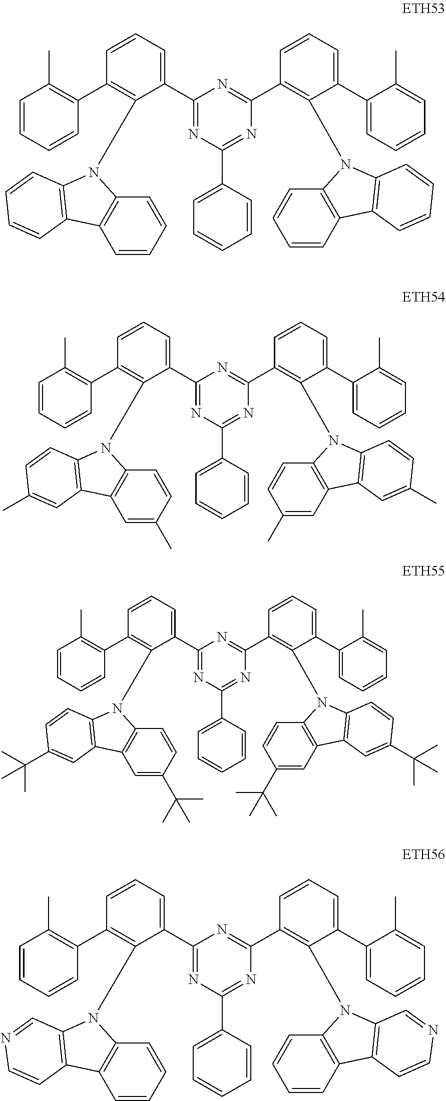

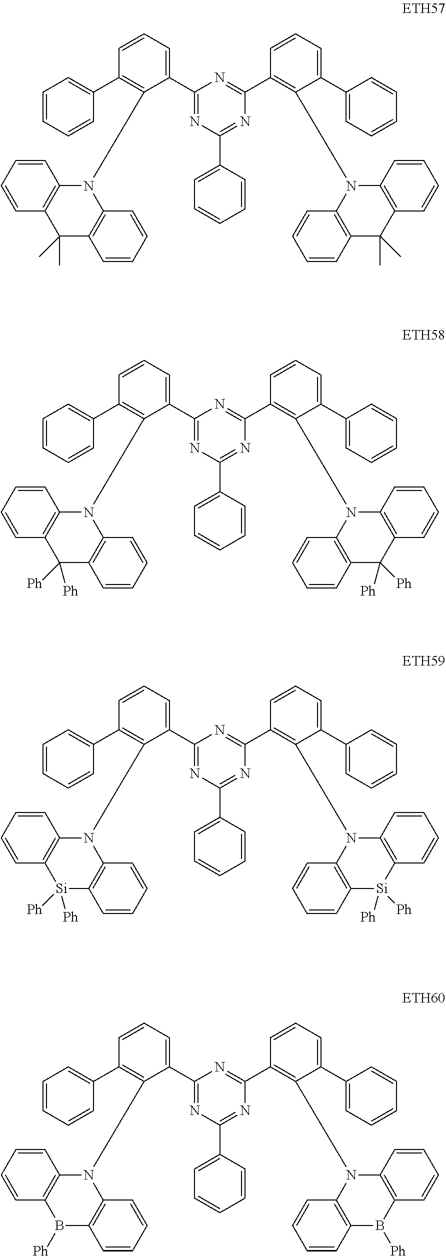

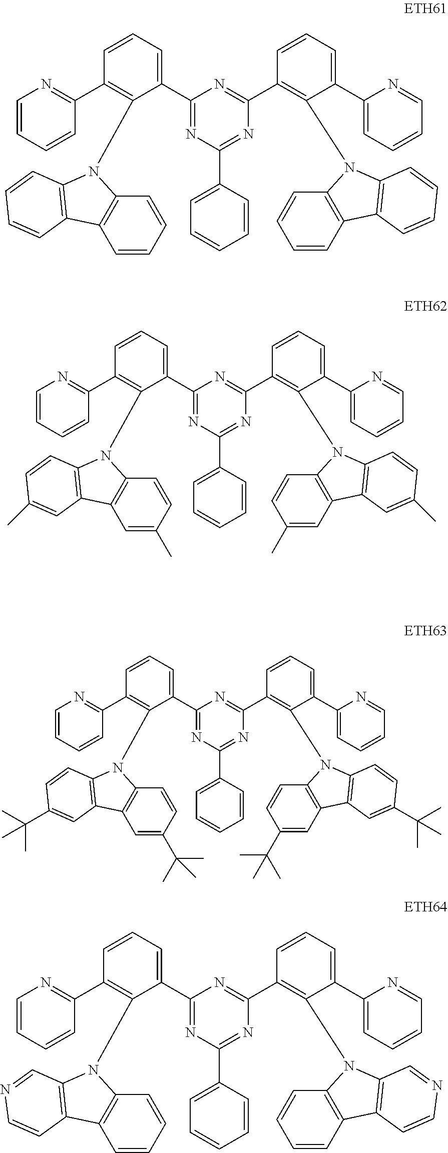

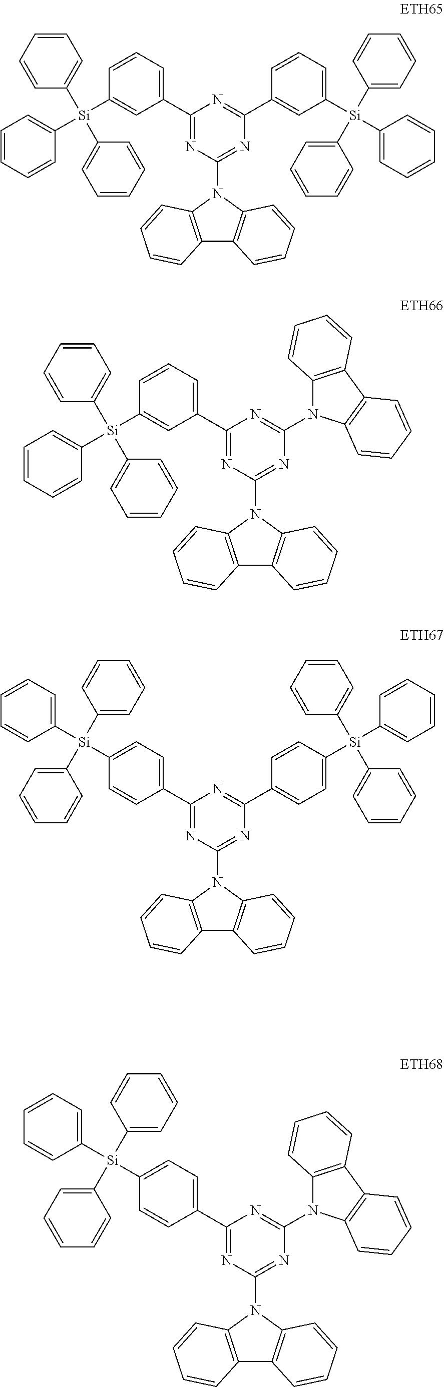 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

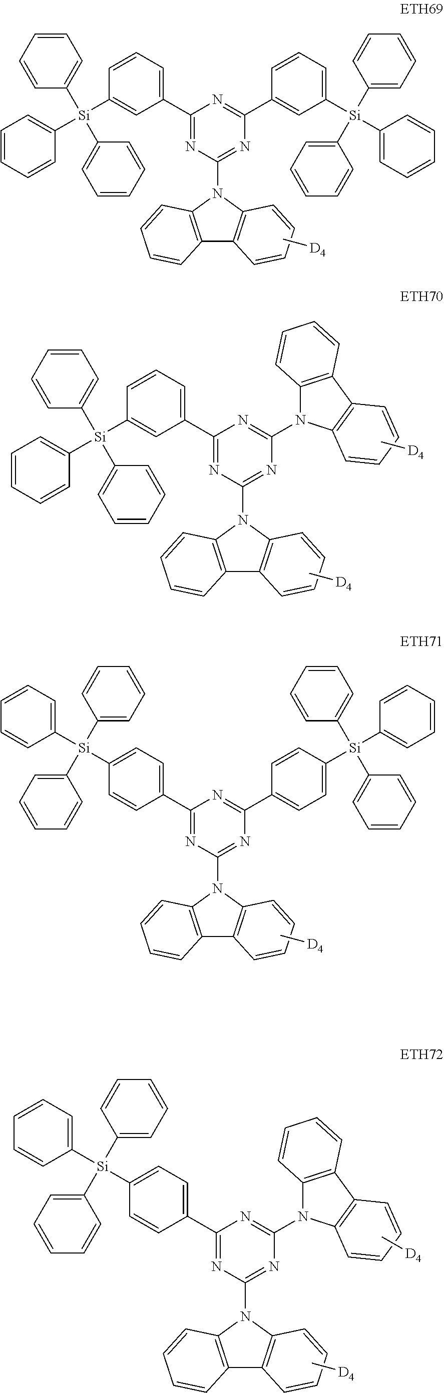

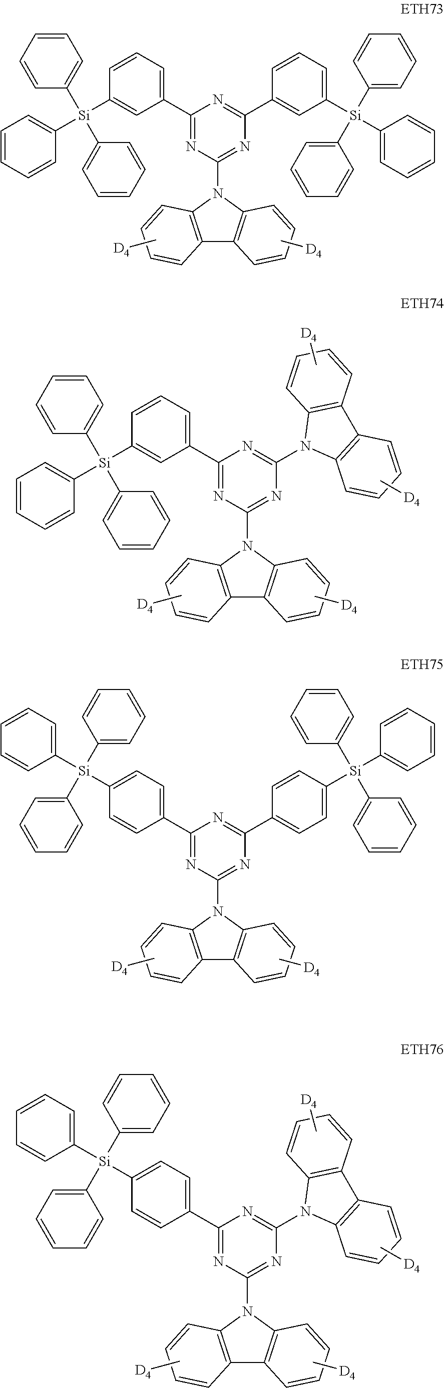

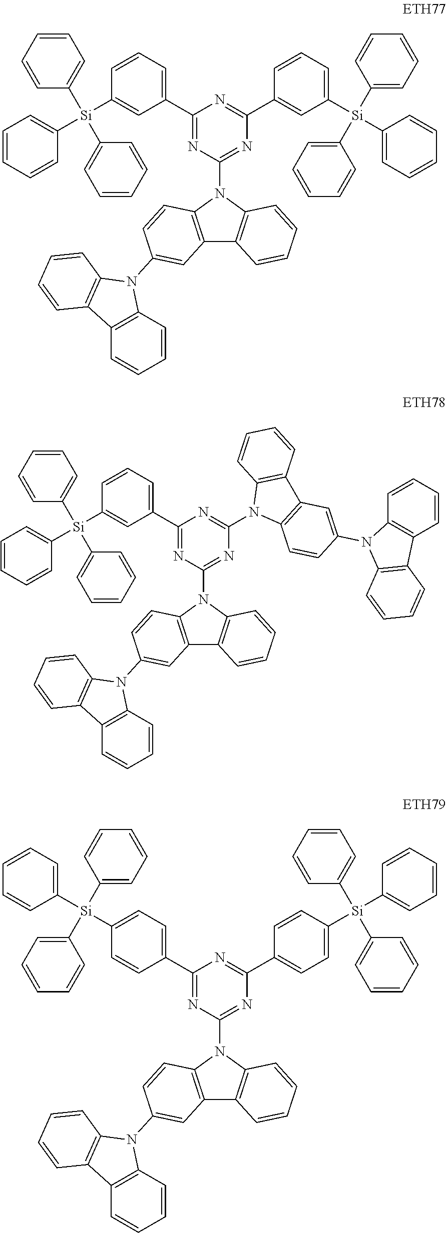 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

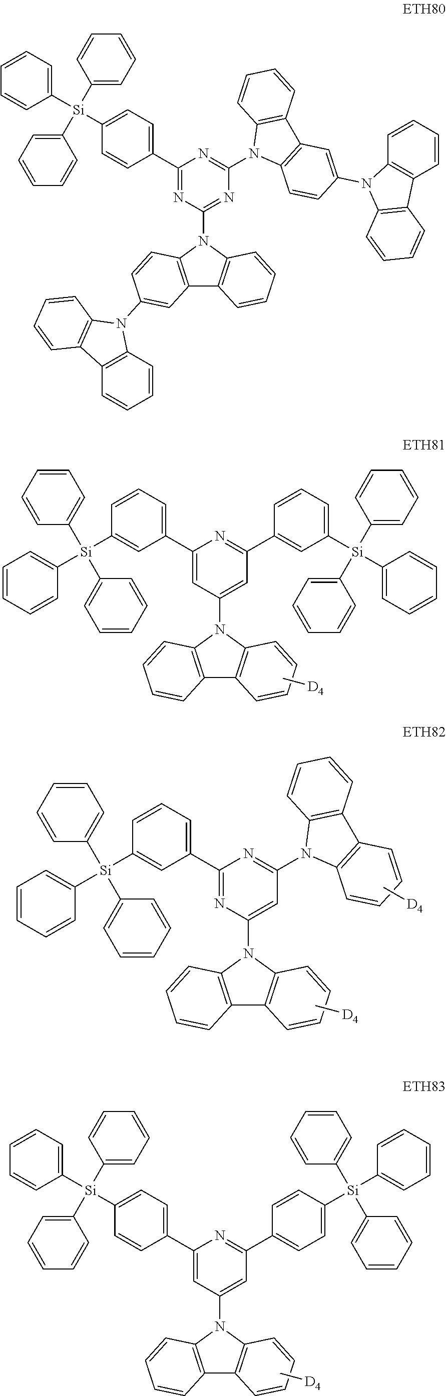

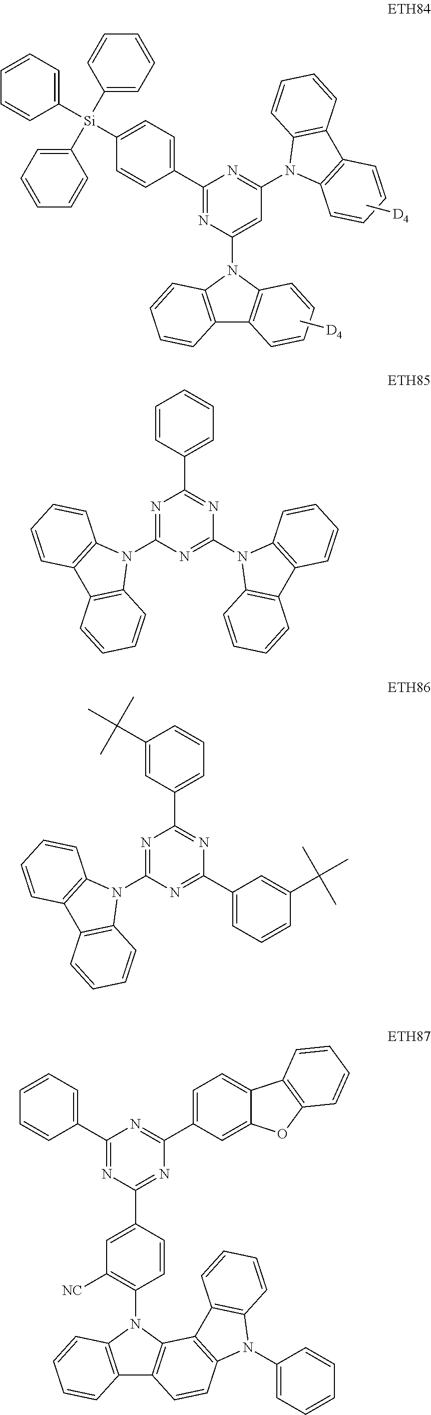

In the example compounds suggested in Compound Group 3, “D” refers to deuterium, and “Ph” refers to an unsubstituted phenyl group.

The emission layer EML may include the second compound and the third compound, and the second compound and the third compound may form an exciplex. In the emission layer EML, the exciplex may be formed by a hole transporting host and an electron transporting host. In this case, the triplet energy of the exciplex formed by the hole transporting host and the electron transporting host may correspond to a difference between the lowest unoccupied molecular orbital (LUMO) energy level of the electron transporting host and the highest occupied molecular orbital (HOMO) energy level of the hole transporting host.

For example, in one or more embodiments, an absolute value of the triplet energy level (T1) of the exciplex formed by the hole transporting host and the electron transporting host may be about 2.4 eV to about 3.0 eV. In addition, the triplet energy of the exciplex may be a smaller value than the energy gap of each host material. in one or more embodiments, the exciplex may have a triplet energy of about 3.0 eV or less, that is the energy gap between the hole transporting host and the electron transporting host.

In one or more embodiments, the emission layer EML may include a fourth compound in addition to the first compound to the third compound. The fourth compound may be used as a phosphorescence sensitizer of the emission layer EML. Because energy may transfer from the fourth compound to the first compound, light emission may arise.

For example, in one or more embodiments, the emission layer EML may include an organometallic complex which contains platinum (Pt) as a center metal atom and contains ligands bonded to the center metal atom, as the fourth compound. In the light emitting element ED according to one or more embodiments, the emission layer EML may contain a compound represented by Formula D-1 as the fourth compound.

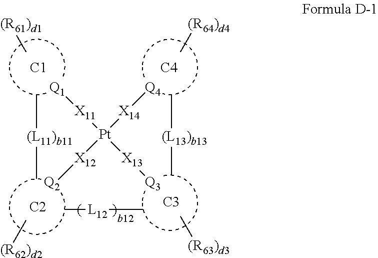

In Formula D-1, Q₁ to Q₄ may each independently be C or N.

In Formula D-1, rings C1 to C4 may each independently be a substituted or unsubstituted hydrocarbon ring of 5 to 30 ring-forming carbon atoms or a substituted or unsubstituted heterocycle of 2 to 30 ring-forming carbon atoms.

In Formula D-1, X₁₁ to X₁₄ may each independently be a direct linkage, or

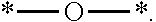

For example, any one of the X₁₁ to X₁₄ may be

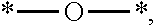

and the remainder may be a direct linkage.

In Formula D-1, L₁₁ to L₁₃ may each independently be a direct linkage,

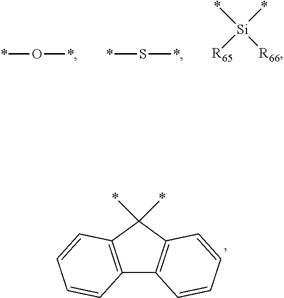

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” refers to a part connected with C1 to C4.

In Formula D-1, b11 to b13 may each independently be 0 or 1. If (e.g., when) b11 is 0, C1 and C2 may be unconnected. If (e.g., when) b12 is 0, C2 and C3 may be unconnected. If (e.g., when) b13 is 0, C3 and C4 may be unconnected.

In Formula D-1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R₆₁ to R₆₆ may be independently combined with an adjacent group to form a ring. In one or more embodiments, R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, if (e.g., when) d1 to d4 are 0, the fourth compound may be unsubstituted with R₆₁ to R₆₄, respectively. An embodiment in which d1 to d4 are 4, and R₆₁ to R₆₄ are all hydrogens, may be the same as an embodiment in which d1 to d4 each are 0. If (when) d1 to d4 are integers of 2 or more, each of multiple R₆₁'s to R₆₄'s may be all the same, or at least one selected from among multiple R₆₁'s to R₆₄'s may be different.

In Formula D-1, C₁ to C₄ may each independently be a substituted or unsubstituted hydrocarbon ring or a substituted or unsubstituted heterocycle, represented by any one selected from among C-1 to C-5.

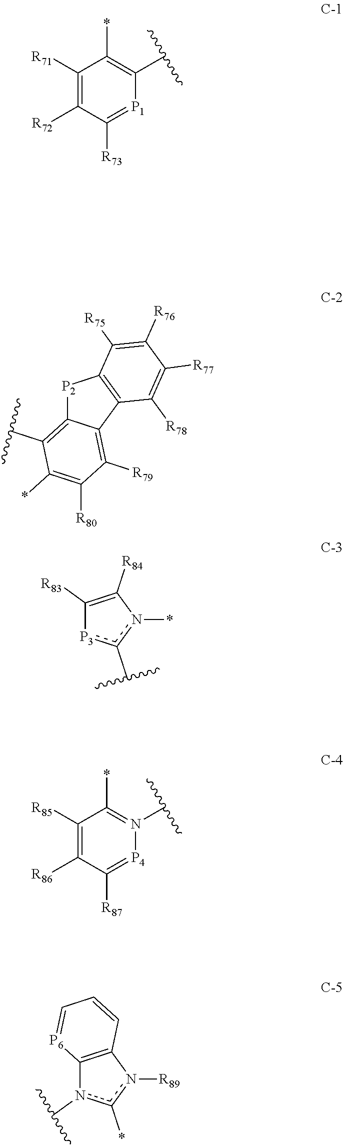

In C-1 to C-5, P₁ may be [symbol] or CR₇₄, P₂ may be [symbol] or NR₈₁, P₃ may be [symbol] or NR₈₂, and P₄ may be [symbol] or CR₈₈, P₆ may be [symbol] or CR₉₀. R₇₁ to R₉₀ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In addition, in C-1 to C-5, “ [symbol] ” is a part connected with a central metal atom of Pt, and “ [symbol] ” corresponds to a part connected with an adjacent ring group (C1 to C4) or an adjacent linker (L₁₁ to L₁₃).

The emission layer EML of one or more embodiments may include the first compound that is a fused polycyclic compound, and at least one selected from among the second to fourth compounds. For example, in one or more embodiments, the emission layer EML may include the first compound, the second compound, and the third compound. In the emission layer EML, the second compound and the third compound may form an exciplex, and via the exciplex, energy transfer to the first compound may arise, and light emission may then arise.

In one or more embodiments, the emission layer EML may include the first compound, the second compound, the third compound, and the fourth compound. In the emission layer EML, the second compound and the third compound may form an exciplex, and via the exciplex, energy transfer to the fourth compound and the first compound may arise, and light emission may then arise. In one or more embodiments, the fourth compound may be a sensitizer. In the light emitting element ED of one or more embodiments, the fourth compound included in the emission layer EML may act as a sensitizer and may play the role of transferring energy from a host to the first compound that is a light-emitting dopant. For example, the fourth compound that plays the role of an auxiliary dopant may accelerate energy transfer to the first compound that is a light emitting dopant and increase the light emitting ratio and efficiency of the first compound. Accordingly, the emission efficiency of the emission layer EML of one or more embodiments may be improved. In addition, if (e.g., when) the energy transfer to the first compound increases, excitons formed in the emission layer EML may not be accumulated but rapidly emit light, and the deterioration of a device may be reduced. Accordingly, the lifetime of the light emitting element ED of one or more embodiments may increase.

The light emitting element ED of one or more embodiments includes all of the first compound, the second compound, the third compound, and the fourth compound, and the emission layer EML may include the combination of two host materials and two dopant materials. In the light emitting element ED of one or more embodiments, the emission layer EML may include the second compound and the third compound, which are two different hosts, the first compound which emits delayed fluorescence, and the fourth compound including an organometallic complex, concurrently (e.g., simultaneously), and may show excellent or suitable emission efficiency properties.

In one or more embodiments, the fourth compound represented by Formula D-1 may be at least one selected from among compounds represented in Compound Group 4. The emission layer EML may include at least one selected from among the compounds represented in Compound Group 4 as a sensitizer material.

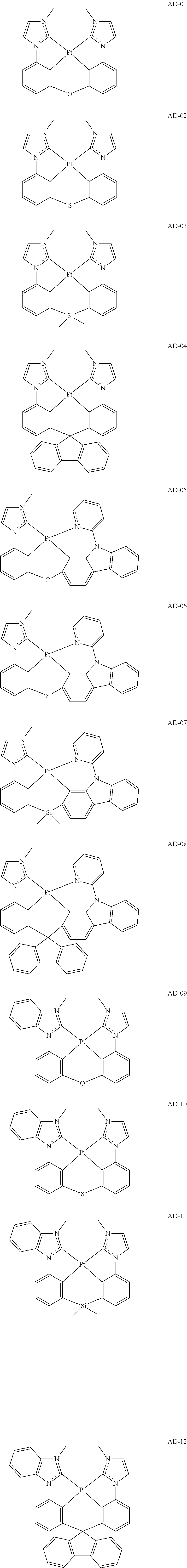

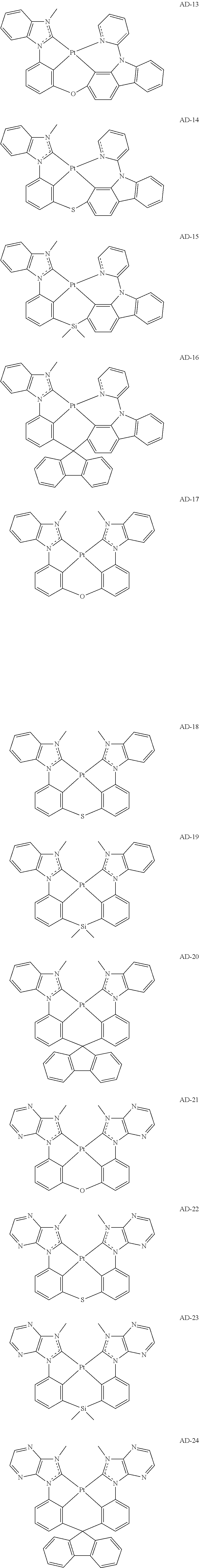

[chemical structure]

In the example compounds suggested in Compound Group 4, “D” refers to deuterium.

In the light emitting element ED of one or more embodiments, if (e.g., when) the emission layer EML includes all of the first compound, the second compound, the third compound, and the fourth compound, an amount of the first compound may be about 0.1 wt % to about 5 wt % based on a total weight, 100 wt %, of the first compound, the second compound, the third compound, and the fourth compound. However, embodiments of the present disclosure are not limited thereto. If (e.g., when) the amount of the first compound satisfies the above-described ratio, energy transfer from the second compound and the third compound to the first compound may increase, and accordingly, the emission efficiency and device lifetime may increase.

In the emission layer EML, an total amount of the second compound and the third compound may be the remaining amount excluding the amount of the first compound and the fourth compound. For example, in one or more embodiments, the total amount of the second compound and the third compound may be about 65 wt % to about 95 wt % based on the total weight of the first compound, the second compound, the third compound, and the fourth compound.

In the total amount of the second compound and the third compound, a weight ratio of the second compound to the third compound may be about 3:7 to about 7:3.

If (e.g., when) the total amount of the second compound and the third compound satisfies the above-described ratio, charge balance properties in the emission layer EML may be improved, and emission efficiency and device lifetime may be improved. If (e.g., when) the total amount of the second compound and the third compound deviates from the above-described ratio range, charge balance in the emission layer EML may be broken, emission efficiency may be degraded, and the device may be easily deteriorated.

If (e.g., when) the emission layer EML includes the fourth compound, an amount of the fourth compound may be about 4 wt % to 30 wt % based on the total weight of the first compound, the second compound, the third compound, and the fourth compound in the emission layer EML. However, embodiments of the present disclosure are not limited thereto. If (e.g., when) the amount of the fourth compound satisfies the above-described amount, energy transfer from a host to the first compound that is a light emitting dopant may increase, and emission ratio and efficiency may be improved. Accordingly, the emission efficiency of the emission layer EML may be improved. If (e.g., when) the amount ratios of the first compound, the second compound, the third compound, and the fourth compound, included in the emission layer EML, satisfies the above-described amount ratio, excellent or suitable emission efficiency and long lifetime may be achieved.

In one or more embodiments, the emission layer EML may include as a suitable dopant material, one or more selected from among styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like.

In one or more embodiments, the emission layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), or thulium (Tm). for example, in one or more embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments of the present disclosure are not limited thereto.

In one or more embodiments, the emission layer EML may include a hole transporting host and an electron transporting host. In addition, the emission layer EML may include an auxiliary dopant and a light emitting dopant. In one or more embodiments, the auxiliary dopant may include a phosphorescence dopant material or a thermally activated delayed fluorescence dopant. For example, in one or more embodiments, the emission layer EML may include a hole transporting host, an electron transporting host, an auxiliary dopant, and a light emitting dopant.

In one or more embodiments, the emission layer may include a quantum dot.

In the present disclosure, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling an element ratio in the quantum dot compound.

A diameter of the quantum dot may be, for example, about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a chemical bath deposition, a metal organic chemical vapor deposition, a molecular beam epitaxy, or a similar process therewith.

The chemical bath deposition is a method of mixing an organic solvent and a precursor material of a quantum dot and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous and beneficial if (e.g., when) compared to a vapor deposition method including a metal organic chemical vapor deposition (MOCVD) and/or a molecular beam epitaxy (MBE), and the growth of the quantum dot particle may be controlled or selected through a low-cost process.

In one or more embodiments, the emission layer EML may include a quantum dot material. In one or more embodiments, the quantum dot may have a core/shell structure. The core of the quantum dot may be selected from among a II-VI group compound, a III-VI group compound, a I-III-VI group compound, a III-V group compound, a Ill-II-V group compound, a IV-VI group compound, a IV group element, a IV group compound, and/or one or more (e.g., any suitable) combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a (e.g., any suitable) mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a (e.g., any suitable) mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a (e.g., any suitable) mixture thereof. In one or more embodiments, the Group II-VI compound may further include a Group I metal and/or a Group IV element. The Group I-II-VI compound may be selected from among CuSnS and CuZnS, and the Group II-IV-VI compound may be selected from among ZnSnS and/or the like. The Group I-II-IV-VI compound may be selected from among quaternary compounds selected from the group consisting of Cu₂ZnSnS₂, Cu₂ZnSnS₄, Cu₂ZnSnSe₄, Ag₂ZnSnS₂, and a (e.g., any suitable) mixture thereof.

The III-VI group compound may include a binary compound such as In₂S₃ and/or In₂Se₃, a ternary compound such as InGaS₃ and/or InGaSes, or arbitrary combinations thereof.

The I-III-VI group compound may be selected from among a ternary compound selected from the group consisting of AgInS, AgInS₂, CuInS, CulnS₂, AgGaS₂, CuGaS₂, CuGaO₂, AgGaO₂, AgAIO₂, and mixtures thereof, and/or a quaternary compound such as AgInGaS₂ and CulnGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In one or more embodiments, the Ill-V group compound may further include a II group metal. For example, InZnP, and/or the like may be selected as a III-II-V group compound.

The Group IV-VI compound may be selected from the group consisting of a binary compound selected from the group consisting of SnS, SnSe, SnTe, PbS, PbSe, PbTe, and a (e.g., any suitable) mixture thereof, a ternary compound selected from the group consisting of SnSeS, SnSeTe, SnSTe, PbSeS, PbSeTe, PbSTe, SnPbS, SnPbSe, SnPbTe, and a (e.g., any suitable) mixture thereof, and a quaternary compound selected from the group consisting of SnPbSSe, SnPbSeTe, SnPbSTe, and a (e.g., any suitable) mixture thereof.

The Group II-IV-V compound may be a ternary compound selected from the group consisting of ZnSnP, ZnSnP₂, ZnSnAs₂, ZnGeP₂, ZnGeAs₂, CdSnP₂, CdGeP₂, and a (e.g., any suitable) mixture thereof.

The Group IV element may be selected from the group consisting of Si, Ge, and a (e.g., any suitable) mixture thereof. The Group IV compound may be a binary compound selected from the group consisting of SiC, SiGe, and a (e.g., any suitable) mixture thereof.

Each element included in a multi-element compound such as the binary compound, ternary compound, or quaternary compound may be present in a particle at a substantially uniform concentration or a non-uniform concentration. For example, Formula above indicates the types (kinds) of elements included in a compound, and element ratios in the compound may be different. For example, AgInGaS₂ may indicate AgInₓGa₁₋ₓS₂ (x is a real number between 0 and 1).

In one or more embodiments, the binary compound, the ternary compound, or the quaternary compound may be present at substantially uniform concentration in a particle or may be present at a partially different concentration distribution state in the same particle. In one or more embodiments, a core/shell structure in which one quantum dot wraps another quantum dot may be desired. The interface of the core and the shell may have a concentration gradient in which the concentration of an element present in the shell is decreased toward the center.

In one or more embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, and/or one or more (e.g., any suitable) combinations thereof.

For example, the metal or non-metal oxide for the shell may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, and/or a ternary compound such as MgAl₂O₄, CoFe₂O₄, NiFe₂O₄, and/or CoMn₂O₄, but embodiments of the present disclosure are not limited thereto.

Also, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but embodiments of the present disclosure are not limited thereto.

The quantum dot may have a full width of half maximum (FWHM) of emission spectrum of about 45 nm or less, about 40 nm or less, or about 30 nm or less. Within this range, color purity or color reproducibility of the quantum dot may be improved. In addition, light emitted via such quantum dot is emitted in all directions, and light view angle properties may be improved.

In addition, the shape of the quantum dot may be a generally used shape in the art, without specific limitation. For example, spherical nanoparticles, pyramidal nanoparticles, multi-arm nanoparticles, cubic nanoparticle, nanotubes, nanowires, nanofibers, nanoplates, and/or the like, may be used.

As the size of the quantum dot or the ratio of elements in the quantum dot compound is regulated, the energy band gap of the quantum dot may be accordingly controlled or selected to obtain light of one or more suitable wavelengths from a quantum dot emission layer. Therefore, by using the quantum dots as described above (using quantum dots of different sizes or having different element ratios in the quantum dot compound), a light emitting element emitting light of one or more suitable wavelengths may be obtained. For example, the size of the quantum dots or the ratio of elements in the quantum dot compound may be regulated to enable the quantum dots to emit red, green, and/or blue light. In one or more embodiments, the quantum dots may be configured to emit white light by combining light of one or more suitable colors.

In each of the light emitting elements ED of embodiments, as shown in FIG. 3 to FIG. 6, the electron transport region ETR may be provided on the emission layer EML. The electron transport region ETR may include at least one of an electron blocking layer HBL, an electron transport layer ETL, or an electron injection layer EIL. However, embodiments of the present disclosure are not limited thereto.

The electron transport region ETR may have a single layer formed using a single material, a single layer formed using multiple different materials, or a multilayer structure having multiple layers formed using multiple different materials.

For example, in one or more embodiments, the electron transport region ETR may have a single layer structure of an electron injection layer EIL or an electron transport layer ETL, or a single layer structure formed using an electron injection material and/or an electron transport material. Further, in one or more embodiments, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. A thickness of the electron transport region ETR may be, for example, from about 1,000 Å to about 1,500 Å.

The electron transport region ETR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

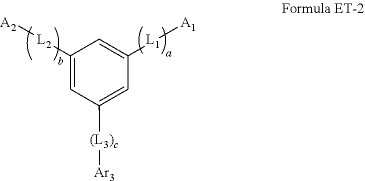

In Formula ET-2, at least one selected from among X₁ to X₃ may be N, and the remainder are CRₐ. Rₐ may be hydrogen, deuterium, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) “a” to “c” are integers of 2 or more, L₁'s to L₃'s may each independently be a substituted or unsubstituted arylene group of 6 to 30 ring-forming carbon atoms or a substituted or unsubstituted heteroarylene group of 2 to 30 ring-forming carbon atoms.

In one or more embodiments, the electron transport region ETR may include an anthracene-based compound. However, embodiments of the present disclosure are not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAIq), berylliumbis(benzoquinolin-10-olate (Bebq₂), 9,10-di(naphthalen-2-yl)anthracene (ADN), 1,3-bis[3,5-di(pyridin-3-yl)phenyl]benzene (BmPyPhB), CNNPTRZ (4′-(4-(4-(4,6-diphenyl-1,3,5-triazin-2-yl)phenyl)naphthalen-1-yl)-[1,1′-biphenyl]-4-carbonitrile), and/or a (e.g., any suitable) mixture thereof, without limitation.

In one or more embodiments, the electron transport region ETR may include at least one selected from among Compounds ET1 to ET36.

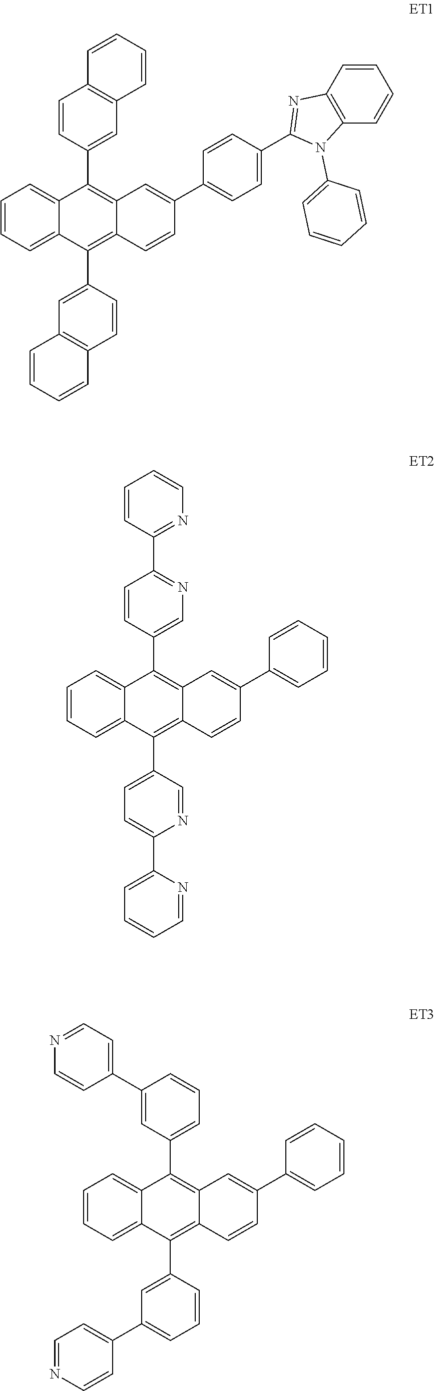 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

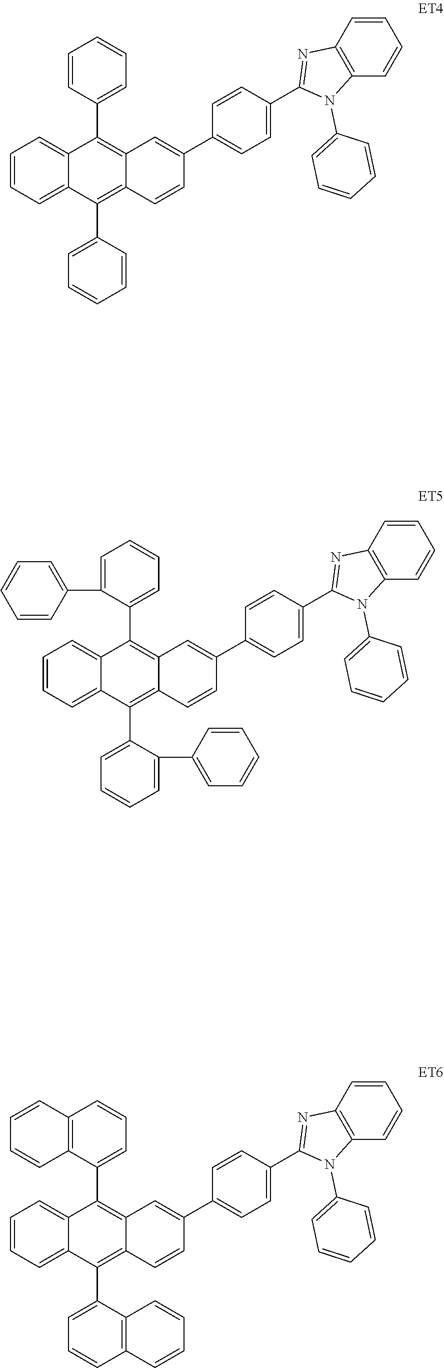 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

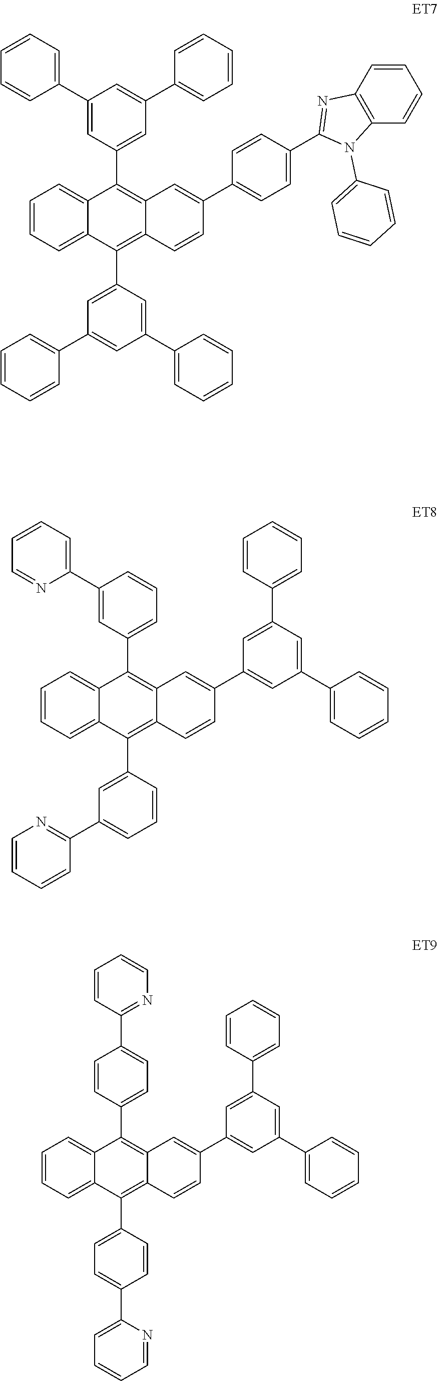 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

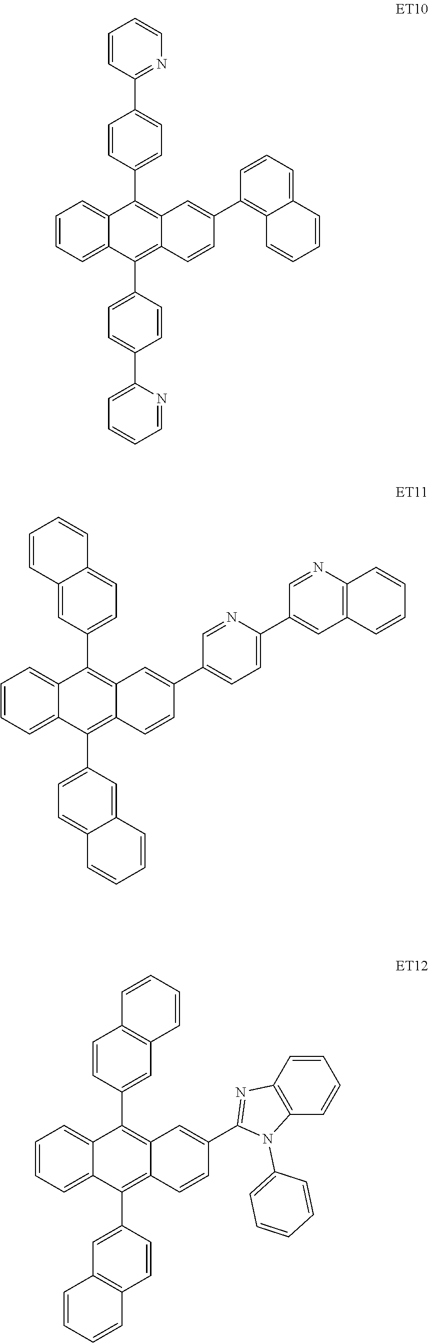 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

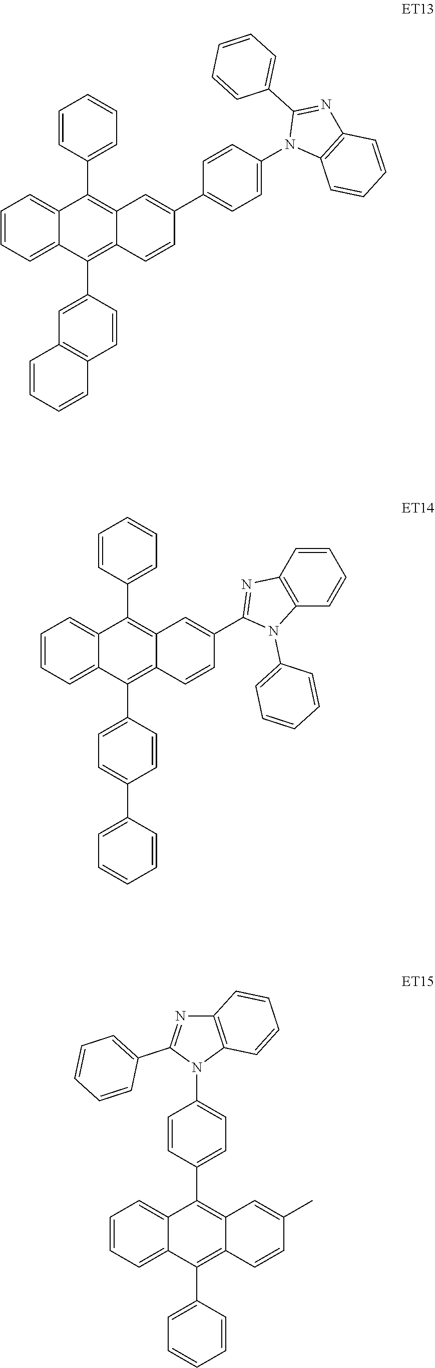 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

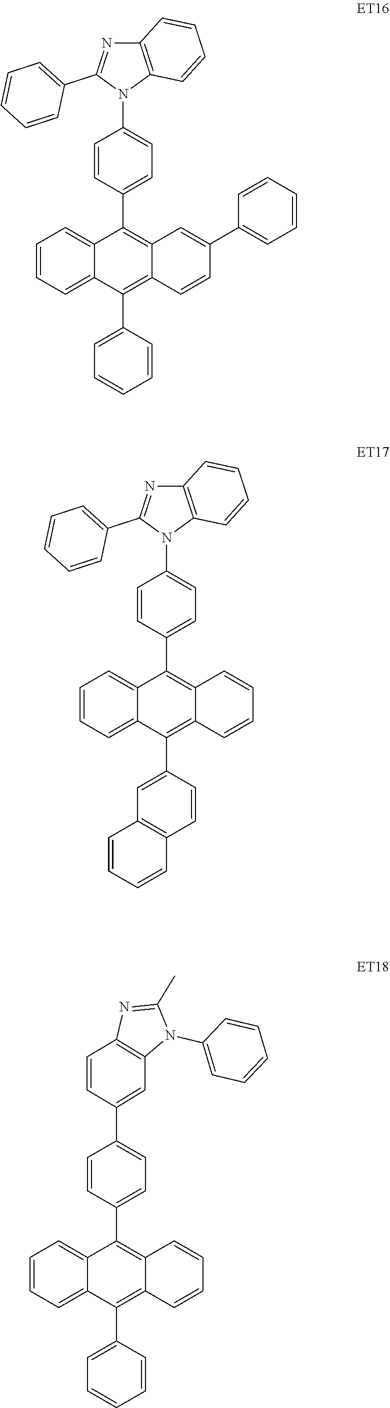 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

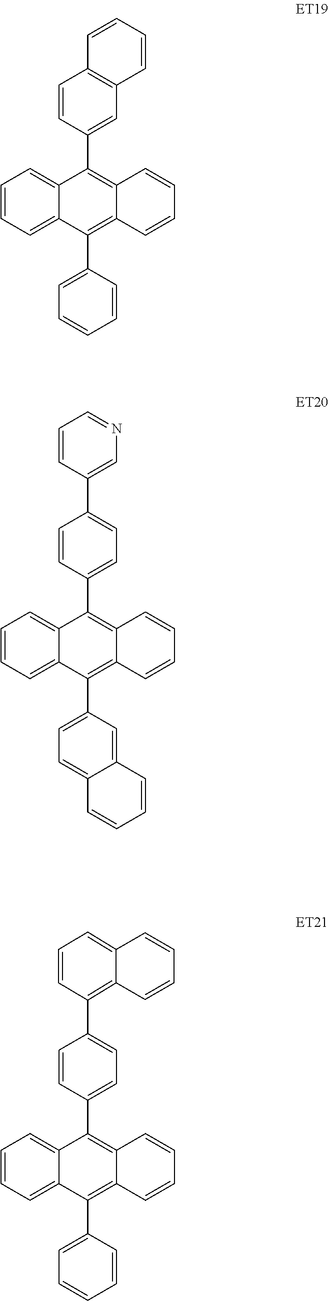

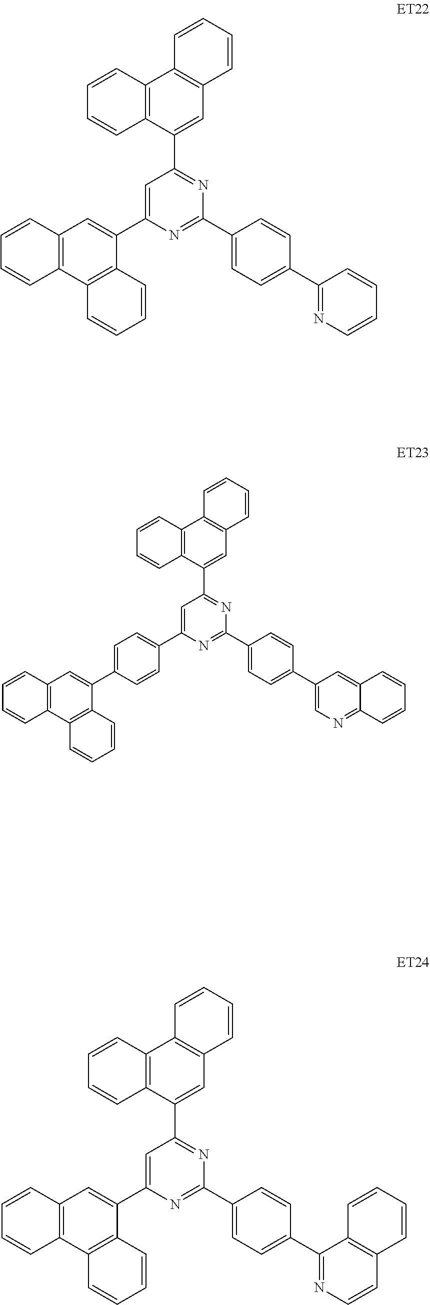 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

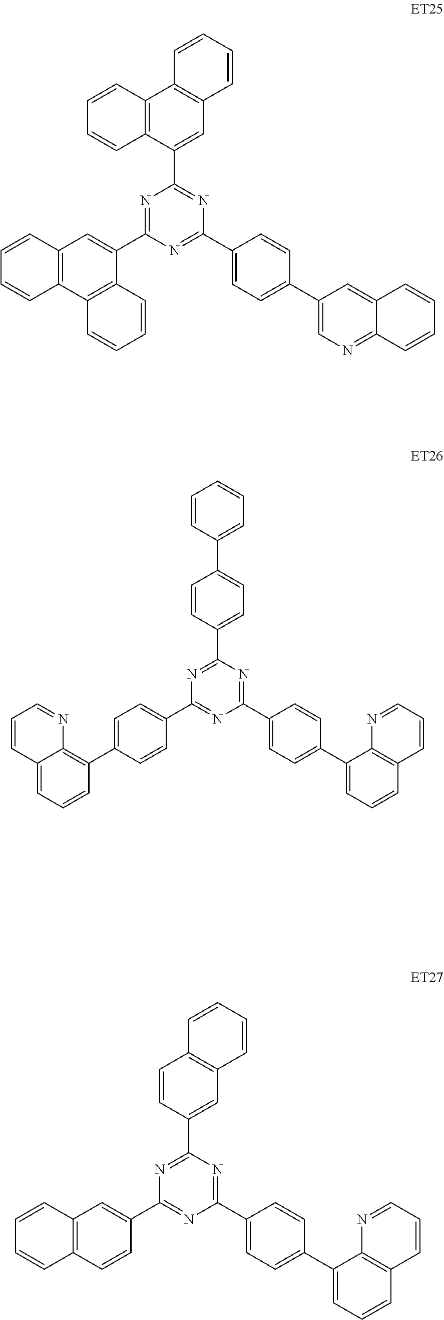 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

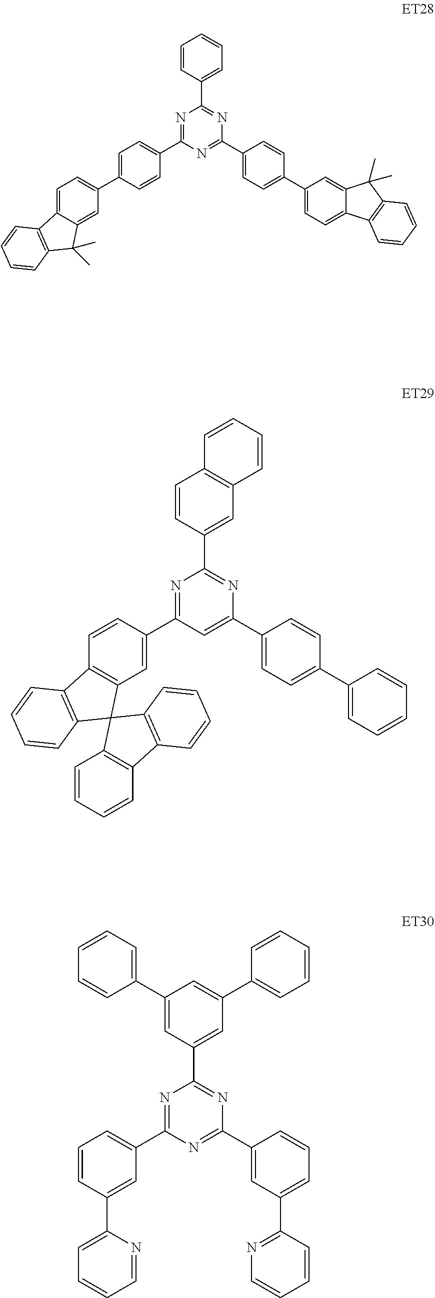 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

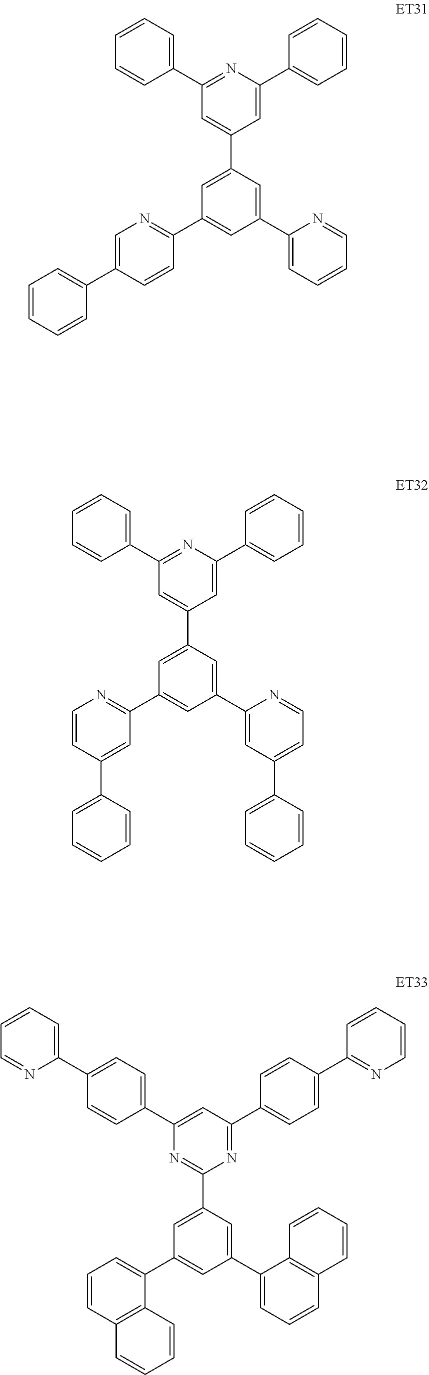 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

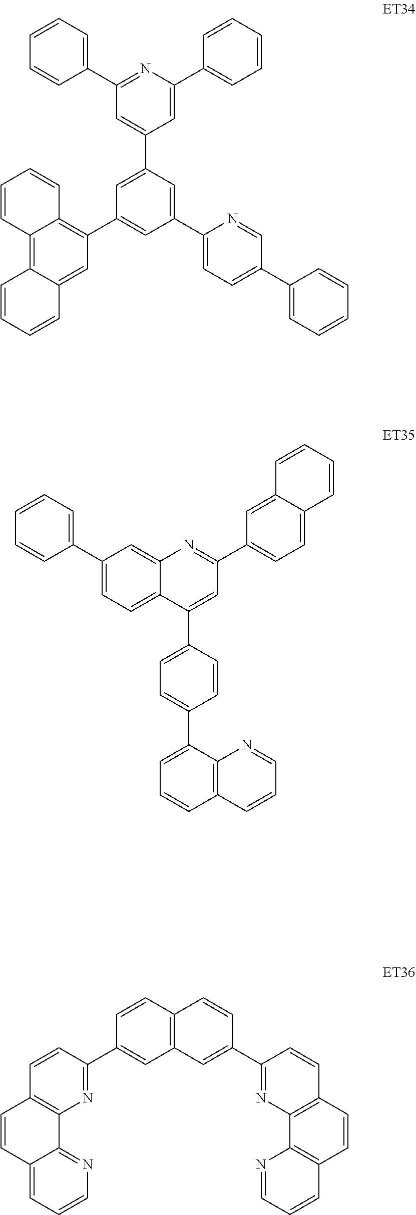 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region ETR may include a metal halide such as LiF, NaCl, CsF, RbCl, RbI, CuI, and/or KI, a lanthanide metal such as Yb, or a co-depositing material of the metal halide and the lanthanide metal. For example, in some embodiments, the electron transport region ETR may include KI:Yb, RbI:Yb, LiF:Yb, and/or the like, as the co-depositing material. In one or more embodiments, the electron transport region ETR may use a metal oxide such as Li₂O and/or BaO, or 8-hydroxy-lithium quinolate (Liq). However, embodiments of the present disclosure are not limited thereto. The electron transport region ETR also may be formed using a mixture material of an electron transport material and an insulating organo metal salt. The insulating organo metal salt may be a material having an energy band gap of about 4 eV or more. For example, the insulating organo metal salt may include one or more of, for example, metal acetates, metal benzoates, metal acetoacetates, metal acetylacetonates, or metal stearates.

In one or more embodiments, the electron transport region ETR may include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to one or more of the aforementioned materials. However, embodiments of the present disclosure are not limited thereto.

The electron transport region ETR may include one or more of the compounds of the electron transport region in at least one selected from among an electron injection layer EIL, an electron transport layer ETL, and a hole blocking layer HBL.

If (e.g., when) the electron transport region ETR includes an electron transport layer ETL, the thickness of the electron transport layer ETL may be from about 100 Å to about 1,000 Å, for example, from about 150 Å to about 500 Å. If (e.g., when) the thickness of the electron transport layer ETL satisfies the above-described range, satisfactory electron transport properties may be obtained without a substantial increase in driving voltage. If (e.g., when) the electron transport region ETR includes an electron injection layer EIL, the thickness of the electron injection layer EIL may be from about 1 Å to about 100 Å, or from about 3 Å to about 90 Å. If (e.g., when) the thickness of the electron injection layer EIL satisfies the above described range, satisfactory electron injection properties may be obtained without inducing a substantial increase in driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, if (e.g., when) the first electrode EL1 is an anode, the second cathode EL2 may be a cathode, and if (e.g., when) the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, a (e.g., any suitable) compound of two or more selected therefrom, a (e.g., any suitable) mixture of two or more selected therefrom, or an oxide thereof.

The second electrode EL2 may be a transmissive electrode, a transflective electrode, or a reflective electrode. If (e.g., when) the second electrode EL2 is the transmissive electrode, the second electrode EL2 may include a transparent metal oxide, for example, ITO, IZO, ZnO, ITZO, and/or the like.

If (e.g., when) the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, a (e.g., any suitable) compound thereof, or a (e.g., any suitable) mixture thereof (for example, AgMg, AgYb, or MgAg). In one or more embodiments, the second electrode EL2 may have a multilayered structure including a reflective layer or a transflective layer formed using one or more of the above-described materials and a transparent conductive layer formed using ITO, IZO, ZnO, ITZO, and/or the like. For example, in one or more embodiments, the second electrode EL2 may include one of the aforementioned metal materials, a (e.g., any suitable) combination of two or more metal materials selected therefrom, or an oxide of the aforementioned metal materials.

In one or more embodiments, the second electrode EL2 may be connected with an auxiliary electrode. If (e.g., when) the second electrode EL2 is connected with the auxiliary electrode, the resistance of the second electrode EL2 may decrease.

In one or more embodiments, on the second electrode EL2 in the light emitting element ED, a capping layer CPL may be further arranged. The capping layer CPL may include a multilayer or a single layer.

In one or more embodiments, the capping layer CPL may be an organic layer or an inorganic layer. For example, if (e.g., when) the capping layer CPL includes an inorganic material, the inorganic material may include an alkali metal compound such as LiF, an alkaline earth metal compound, SiON, SiNₓ, SiO_{y}, and/or the like.

For example, in one or more embodiments, if the capping layer CPL includes an organic material, the organic material may include 2,2′-dimethyl-N,N′-di-[(1-naphthyl)-N,N′-diphenyl]-1,1′-biphenyl-4,4′-diamine (α-NPD), NPB, TPD, m-MTDATA, Alq3, CuPc, N4,N4,N4′,N4′-tetra(biphenyl-4-yl) biphenyl-4,4′-diamine (TPD15), 4,4′,4″-tris(carbazol-9-yl) triphenylamine (TCTA), and/or the like, or may include an epoxy resin, or acrylate such as methacrylate. In one or more embodiments, the capping layer CPL may include at least one selected from among Compounds P1 to P5, but embodiments of the present disclosure are not limited thereto.

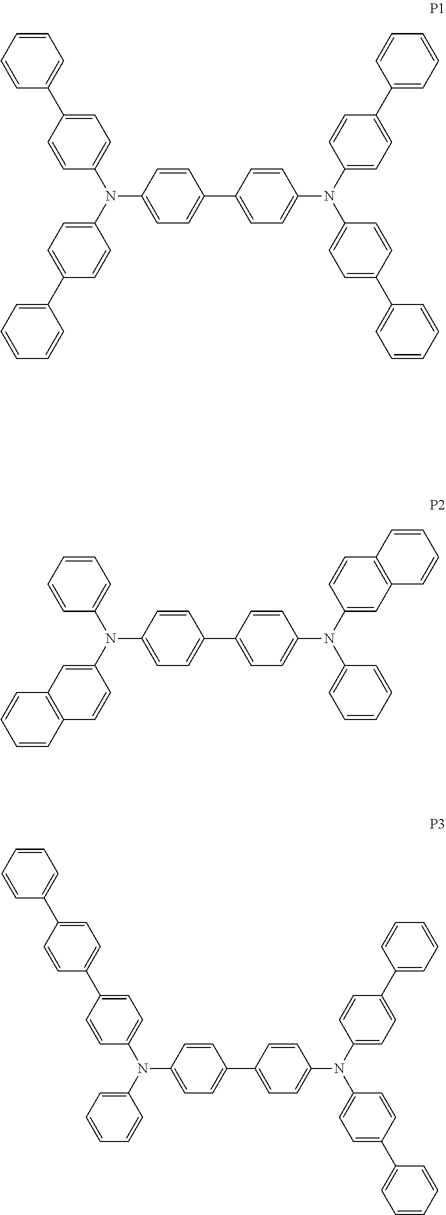 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

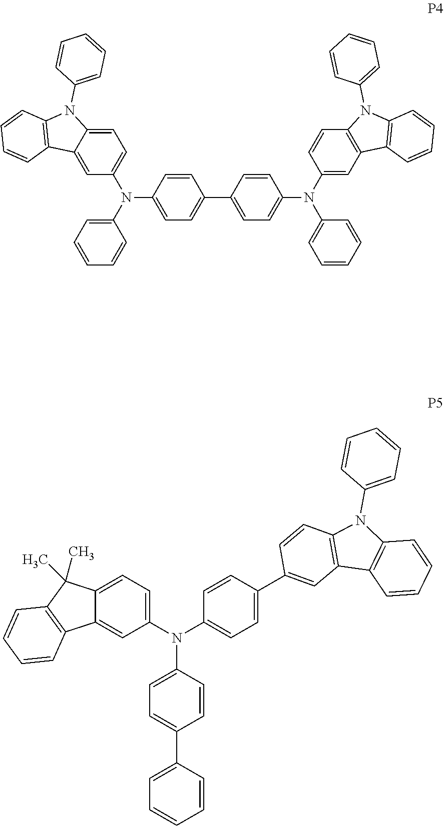 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. for example, in one or more embodiments, the refractive index of the capping layer CPL with respect to light in a wavelength range of about 550 nm to about 660 nm may be about 1.6 or more.

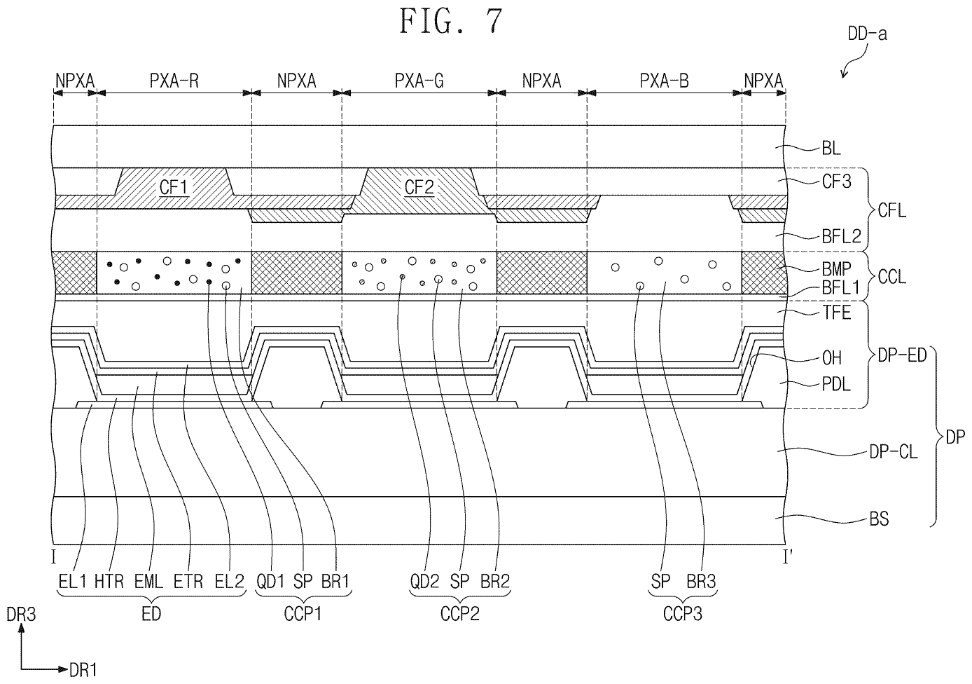

FIG. 7 to FIG. 10 are each a cross-sectional view of a display apparatus according to one or more embodiments. In the explanation on the display apparatuses of embodiments referring to FIG. 7 to FIG. 10, the overlapping parts with the explanation on FIG. 1 to FIG. 6 will not be explained again, and only different features will be explained chiefly.

Referring to FIG. 7, the display apparatus DD-a according to one or more embodiments may include a display panel DP including a display device layer DP-ED, a light controlling layer CCL arranged on the display panel DP, and a color filter layer CFL.

In one or more embodiments, as shown in FIG. 7, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. In one or more embodiments, the same structure as any one of the light emitting elements of FIG. 3 to FIG. 6 may be applied to the structure of the light emitting element ED shown in FIG. 7.

The emission layer EML of the light emitting element ED included in the display device DD-a according to one or more embodiments may include a fused polycyclic compound of one or more embodiments described above.

Referring to FIG. 7, the emission layer EML may be arranged in an opening part OH defined in a pixel definition layer PDL. For example, the emission layer EML divided by the pixel definition layer PDL and correspondingly provided to each of luminous areas PXA-R, PXA-G, and PXA-B may be to emit light in substantially the same wavelength range. In the display apparatus DD-a of one or more embodiments, the emission layer EML may be to emit blue light. In one or more embodiments, the emission layer EML may be provided as a common layer across all luminous areas PXA-R, PXA-G, and PXA-B.

The light controlling layer CCL may be arranged on the display panel DP. The light controlling layer CCL may include a light converter. The light converter may be a quantum dot or a phosphor. The light converter may transform the wavelength of light provided and then emit the transformed light. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated from one another.

Referring to FIG. 7, a partition pattern BMP may be arranged between the separated light controlling parts CCP1, CCP2, and CCP3, but embodiments of the present disclosure are not limited thereto. In FIG. 7, the partition pattern BMP is shown not to be overlapped with the light controlling parts CCP1, CCP2, and CCP3, but, in one or more embodiments, at least a portion of the edges of the light controlling parts CCP1, CCP2, and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting the first color light into third color light, and a third light controlling part CCP3 transmitting the first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, in one or more embodiments, the first quantum dot QD1 may be a red quantum dot to emit red light, and the second quantum dot QD2 may be a green quantum dot to emit green light. On the quantum dots QD1 and QD2, the same contents as those described above on quantum dots may be applied.

In one or more embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot but include the scatterer SP.

The scatterer SP may be an inorganic particle. For example, the scatterer SP may include at least one selected from among TiO₂, ZnO, Al₂O₃, SiO₂, and hollow silica. In one or more embodiments, the scatterer SP may include one selected from among TiO₂, ZnO, Al₂O₃, SiO₂, and hollow silica, or may be a mixture of two or more materials selected among TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 in which the quantum dots QD1 and QD2 and the scatterer SP are dispersed accordingly. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3.

The base resins BR1, BR2, and BR3 are media in which the quantum dots QD1 and QD2 and the scatterer SP are dispersed accordingly, and may each be composed of one or more suitable resin compositions which may be generally referred to as a binder. For example, the base resins BR1, BR2, and BR3 may each independently be an acrylic-based resin, a urethane-based resin, a silicone-based resin, an epoxy-based resin, and/or the like. The base resins BR1, BR2, and BR3 may each be a transparent resin. In one or more embodiments, the first base resin BR1, the second base resin BR2, and the third base resin BR3 may be the same or different from one another.

In one or more embodiments, the light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block the exposure of the light controlling parts CCP1, CCP2, and CCP3 to humidity/oxygen. In one or more embodiments, the barrier layer BFL1 may cover the light controlling parts CCP1, CCP2, and CCP3. In one or more embodiments, a color filter layer CFL, which will be explained later, may include a barrier layer BFL2 arranged on the light controlling parts CCP1, CCP2, and CCP3.

The barrier layers BFL1 and BFL2 may each include at least one inorganic layer. For example, in one or more embodiments, the barrier layers BFL1 and BFL2 may each be formed by including an inorganic material. For example, the barrier layers BFL1 and BFL2 may be each independently formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. In one or more embodiments, the barrier layers BFL1 and BFL2 may each independently further include an organic layer. The barrier layers BFL1 and BFL2 may each independently be composed of a single layer of multiple layers.

In the display apparatus DD-a of one or more embodiments, the color filter layer CFL may be arranged on the light controlling layer CCL. For example, in one or more embodiments, the color filter layer CFL may be arranged directly on the light controlling layer CCL. In these embodiments, the barrier layer BFL2 may not be provided.

The color filter layer CFL may include filters CF1, CF2, and CF3. The first to third filters CF1, CF2, and CF3 may be arranged corresponding to a red luminous area PXA-R, a green luminous area PXA-G, and a blue luminous area PXA-B, respectively.

The color filter layer CFL may include a first filter CF1 configured to transmit the second color light, a second filter CF2 configured to transmit the third color light, and a third filter CF3 configured to transmit the first color light. For example, in one or more embodiments, the first filter CF1 may be a red filter, the second filter CF2 may be a green filter, and the third filter CF3 may be a blue filter. Each of the filters CF1, CF2 and CF3 may include a polymer photosensitive resin and a pigment and/or a dye. For example, the first filter CF1 may include a red pigment and/or a red dye, the second filter CF2 may include a green pigment and/or a green dye, and the third filter CF3 may include a blue pigment and/or a blue dye.

Embodiments of the present disclosure are not limited thereto, for example, the third filter CF3 may not include (e.g., may exclude) any pigment or any dye. The third filter CF3 may include a polymer photosensitive resin and not include a pigment or a dye. The third filter CF3 may be transparent. The third filter CF3 may be formed using a transparent photosensitive resin.

In one or more embodiments, the first filter CF1 and the second filter CF2 may each be a yellow filter. The first filter CF1 and the second filter CF2 may be provided in one body. The first to third filters CF1, CF2, and CF3 may be arranged corresponding to the red luminous area PXA-R, the green luminous area PXA-G, and the blue luminous area PXA-B, respectively.

In one or more embodiments, the color filter layer CFL may further include a light blocking part. The color filter layer CFL may include a light blocking part arranged at the boundaries between adjacent filters CF1, CF2, and CF3. The light blocking part may be a black matrix. The light blocking part may be formed by including an organic light blocking material and/or an inorganic light blocking material, including a black pigment and/or a black dye. The light blocking part may divide the boundaries among adjacent filters CF1, CF2, and CF3. In one or more embodiments, the light blocking part may be formed as a blue filter.

On the color filter layer CFL, a base substrate BL may be arranged. The base substrate BL may be a member providing a base surface on which the color filter layer CFL, the light controlling layer CCL, and/or the like, are arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In addition, in one or more embodiments, the base substrate BL may not be provided.

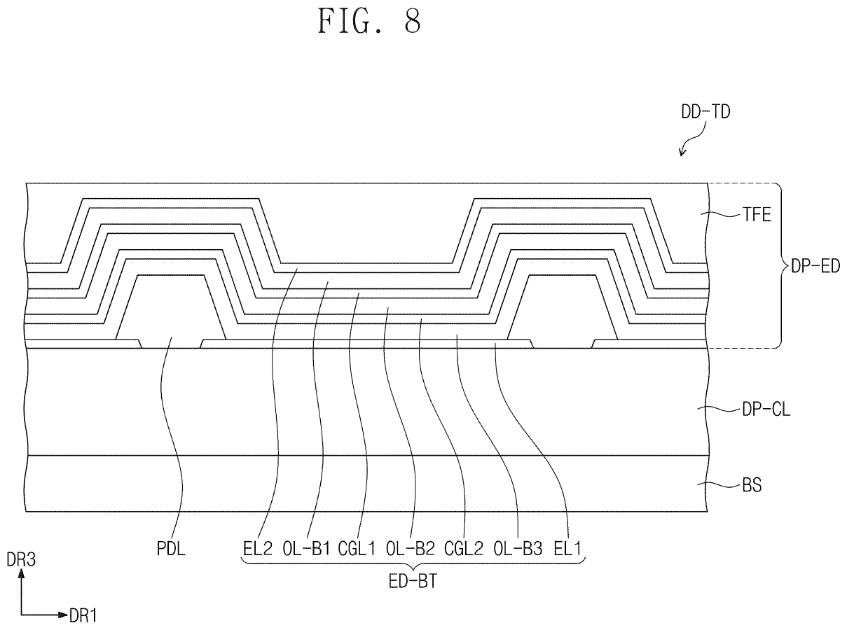

FIG. 8 is a cross-sectional view showing a part of the display apparatus according to one or more embodiments. In a display apparatus DD-TD of one or more embodiments, the light emitting element ED-BT may include multiple light emitting structures OL-B1, OL-B2, and OL-B3. The light emitting element ED-BT may include oppositely arranged first electrode EL1 and second electrode EL2, and the multiple light emitting structures OL-B1, OL-B2, and OL-B3 stacked in a thickness direction and provided between the first electrode EL1 and the second electrode EL2. Each of the light emitting structures OL-B1, OL-B2, and OL-B3 may include an emission layer EML (FIG. 7), and a hole transport region HTR and an electron transport region ETR, arranged with the emission layer (FIG. 7) therebetween.

For example, the light emitting element ED-BT included in the display device DD-TD of one or more embodiments may be a light emitting element of a tandem structure including multiple emission layers.

In one or more embodiments shown in FIG. 8, light emitted from the light emitting structures OL-B1, OL-B2, and OL-B3 may be all blue light. However, embodiments of the present disclosure are not limited thereto, for example, in one or more embodiments, the wavelength regions of light emitted from the light emitting structures OL-B1, OL-B2, and OL-B3 may be different from one another. For example, in one or more embodiments, the light emitting element ED-BT including the multiple light emitting structures OL-B1, OL-B2, and OL-B3 each emitting light in different wavelength regions may be to emit white light (e.g., combined white light).

Between neighboring light emitting structures OL-B1, OL-B2, and OL-B3, charge generating layers CGL1 and CGL2 may be respectively arranged. The charge generating layers CGL1 and CGL2 may independently include a p-type or kind charge (e.g., P-charge) generating layer and/or an n-type or kind charge (e.g., N-charge) generating layer.

At least one of the light emitting structures OL-B1, OL-B2, or OL-B3 included in the display device DD-TD of one or more embodiments may include the above-described amine compound of one or more embodiments.

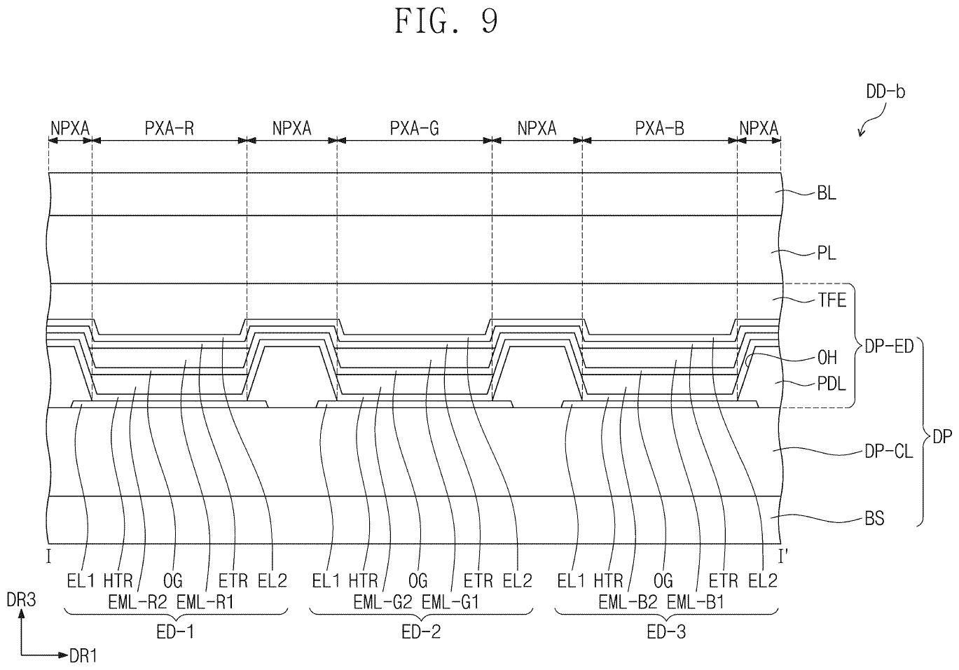

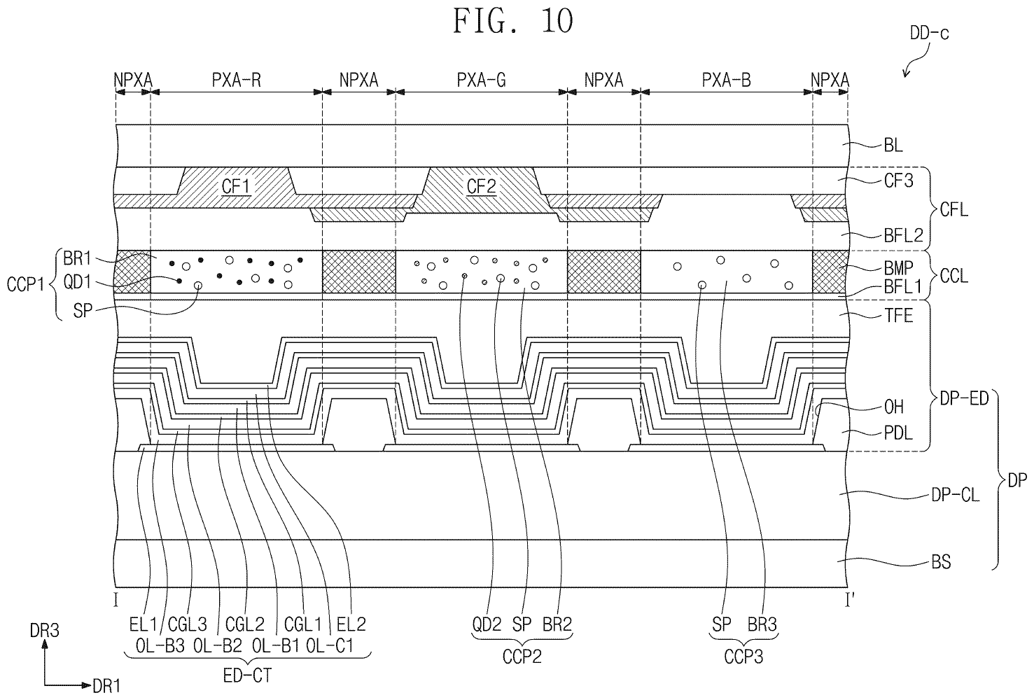

Referring to FIG. 9, a display apparatus DD-b according to one or more embodiments may include light emitting elements ED-1, ED-2, and ED-3, each formed by stacking two emission layers. Compared to the display device DD shown in FIG. 2, the display apparatus DD-b shown in FIG. 9 is different in that first to third light emitting elements ED-1, ED-2, and ED-3 each include two emission layers stacked in a thickness direction. In each of the first to third light emitting elements ED-1, ED-2, and ED-3, two emission layers may be to emit light in substantially the same wavelength region.

In one or more embodiments, the first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be arranged.

The emission auxiliary part OG may include a single layer or a multilayer. The emission auxiliary part OG may include a charge generating layer. In one or more embodiments, the emission auxiliary part OG may include an electron transport region, a charge generating layer, and a hole transport region stacked in order (e.g., in the stated order). The emission auxiliary part OG may be provided as a common layer in all of the first to third light emitting elements ED-1, ED-2, and ED-3. However, embodiments of the present disclosure are not limited thereto, and the emission auxiliary part OG may be patterned and provided in an opening part OH defined in a pixel definition layer PDL.

The first red emission layer EML-R1, the first green emission layer EML-G1, and the first blue emission layer EML-B1 may each be arranged between the electron transport region ETR and the emission auxiliary part OG. The second red emission layer EML-R2, the second green emission layer EML-G2, and the second blue emission layer EML-B2 may each be arranged between the emission auxiliary part OG and the hole transport region HTR.

For example, in one or more embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order (e.g., in the stated order). The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order (e.g., in the stated order). The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order (e.g., in the stated order).

In one or more embodiments, an optical auxiliary layer PL may be arranged on the display device layer DP-ED. The optical auxiliary layer PL may include a polarization layer. The optical auxiliary layer PL may be arranged on the display panel DP and may control reflected light at the display panel DP by external light. In one or more embodiments, the optical auxiliary layer PL may not be provided in the display apparatus.

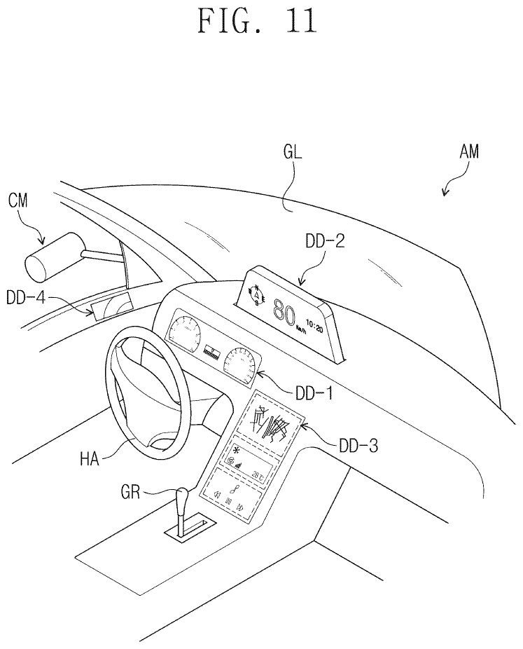

Different from FIG. 8 and FIG. 9, a display apparatus DD-c in FIG. 10 is shown to include four light emitting structures OL-B1, OL-B2, OL-B3, and OL-C1. A light emitting element ED-CT may include oppositely arranged first electrode EL1 and second electrode EL2, and first to fourth light emitting structures OL-B1, OL-B2, OL-B3, and OL-C1 stacked in a thickness direction between the first electrode EL1 and the second electrode EL2. The third light emitting structures OL-B3, the second light emitting structures OL-B2, the first light emitting structures OL-B1, and the fourth light emitting structures OL-C1 are stacked in order (e.g., in the stated order) in the thickness direction. Between the first to fourth light emitting structures OL-B1, OL-B2, OL-B3, and OL-C1, charge generating layers CGL1, CGL2, and CGL3 may each be separately arranged. For example, A first charge generating layer CGL1 is arranged between the first light emitting structures OL-B1 and the fourth light emitting structures OL-C1. A second charge generating layer CGL2 is arranged between the first light emitting structures OL-B1 and the second light emitting structures OL-B2. A third charge generating layer CGL3 is arranged between the second light emitting structures OL-B2 and the third light emitting structures OL-B3.

In one or more embodiments, among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3 and OL-C1 may each be to emit different wavelengths of light.

The charge generating layers CGL1, CGL2, and CGL3 arranged among neighboring light emitting structures OL-B1, OL-B2, OL-B3, and OL-C1 may include a p-type or kind charge (e.g., P-charge) generating layer and/or an n-type or kind charge (e.g., N-charge) generating layer.

At least one selected from among the light emitting structures OL-B1, OL-B2, OL-B3, and OL-C1 included in the display apparatus DD-c of one or more embodiments may include the amine compound of one or more embodiments described above.

The light emitting element ED according to one or more embodiments of the present disclosure includes the amine compound of one or more embodiments described above in at least one functional layer arranged between the first electrode EL1 and the second electrode EL2, and may thus exhibit improved light emitting efficiency and improved lifespan. The light emitting element ED according to one or more embodiments may include the amine compound of one or more embodiments described above in at least one of the hole transport region HTR, the emission layer EML, or the electron transport region ETR arranged between the first electrode EL1 and the second electrode EL2, or in a capping layer CPL. For example, the amine compound according to one or more embodiments may be included in the hole transport region HTR of the light emitting element ED of one or more embodiments, and thus the light emitting element of one or more embodiments may exhibit high efficiency and long lifespan.

The amine compound of one or more embodiments described above includes an amine group, the first substituent, the second substituent, and the third substituent, and may thus increase stability of the amine compound and improve hole transport properties thereof. Accordingly, a light emitting element including the amine compound of one or more embodiments may have increased lifespan and efficiency. In addition, the light emitting element of one or more embodiments includes the amine compound according to one or more embodiments in a hole transport layer, may thus exhibit increased efficiency and lifetime.

FIG. 11 is a diagram showing an automobile AM in which first to fourth display apparatuses DD-1, DD-2, DD-3, and DD-4 are arranged. At least one selected from among the first to fourth display apparatuses DD-1, DD-2, DD-3, and DD-4 may include the same configuration as one of the display apparatuses DD, DD-TD, DD-a, DD-b, and DD-c explained referring to FIGS. 1, 2, and 7 to 10.

In FIG. 11, a vehicle is shown as an automobile AM, but this is a mere example, for example, the first to fourth display apparatuses DD-1, DD-2, DD-3 and DD-4 may be arranged on other transport apparatuses such as bicycles, motorcycles, trains, ships, and/or airplanes. In addition, at least one selected from among the first to fourth display apparatuses DD-1, DD-2, DD-3, and DD-4 including the same configuration as one of the display apparatuses DD, DD-TD, DD-a, DD-b, and DD-c may be introduced in personal computers, laptop computers, personal digital terminals, game consoles, portable electronic devices, televisions, monitors, external billboards, and/or the like. These are suggested as mere examples, and the display apparatus may be introduced in other electronic devices as long as not deviated from the disclosure.

In one or more embodiments, at least one selected from among the first to fourth display apparatuses DD-1, DD-2, DD-3, and DD-4 may include the light emitting element ED of one or more embodiments described with reference to FIGS. 3 to 6. The light emitting element ED of one or more embodiments may include the amine compound of one or more embodiments. At least one selected from among the first to fourth display apparatuses DD-1, DD-2, DD-3, and DD-4 may include the light emitting element ED including the amine compound of one or more embodiments, thereby improving display service life.

Referring to FIG. 11, an automobile AM may include a steering wheel HA for the operation of the automobile AM and a gear GR. In addition, the automobile AM may further include a front window GL arranged to face a driver.

A first display apparatus DD-1 may be arranged in a first region overlapping with the steering wheel HA. For example, the first display apparatus DD-1 may be a digital cluster displaying first information of the automobile AM. The first information may include a first graduation showing a driving speed of the automobile AM, a second graduation showing the number of revolution of an engine (i.e., revolutions per minute (RPM)), and an image showing a fuel state. The first graduation and the second graduation may each be represented by a digital image.

A second display apparatus DD-2 may be arranged in a second region opposite to (e.g., facing) a driver seat and overlapping with the front window GL. The driver seat may be a seat where the steering wheel HA faces. For example, the second display apparatus DD-2 may be a head up display (HUD) showing second information of the automobile AM. The second display apparatus DD-2 may be optically clear. The second information may include digital numbers showing the driving speed of the automobile AM and may further include information including the current time. In one or more embodiments, the second information of the second display apparatus DD-2 may be projected and displayed on the front window GL.

A third display apparatus DD-3 may be arranged in a third region adjacent to the gear GR. For example, the third display apparatus DD-3 may be a center information display (CID) for the automobile, arranged between the driver seat and a passenger seat and showing third information. The passenger seat may be a seat separated from the driver seat with the gear GR therebetween. The third information may include information on road conditions (for example, navigation information), on playing music or radio, on playing a dynamic image (or image), on the temperature in the automobile AM, and/or the like.

A fourth display apparatus DD-4 may be arranged in a fourth region separated from the steering wheel HA and the gear GR and adjacent to a side of the automobile AM. For example, the fourth display apparatus DD-4 may be a digital wing mirror displaying fourth information. The fourth display apparatus DD-4 may display an external image of the automobile AM, taken by a camera module CM arranged at the outside of the automobile AM. The fourth information may include the external image of the automobile AM.

The above-described first to fourth information is for illustration, and the first to fourth display apparatuses DD-1, DD-2, DD-3, and DD-4 may further display information on the inside and outside of the automobile. The first to fourth information may include different information from one another. However, embodiments of the present disclosure are not limited thereto, and a portion of the first to fourth information may include the same information as one another.

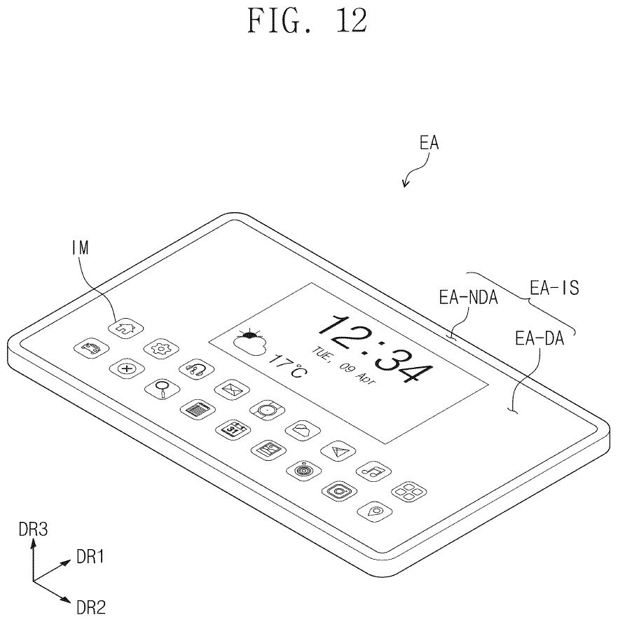

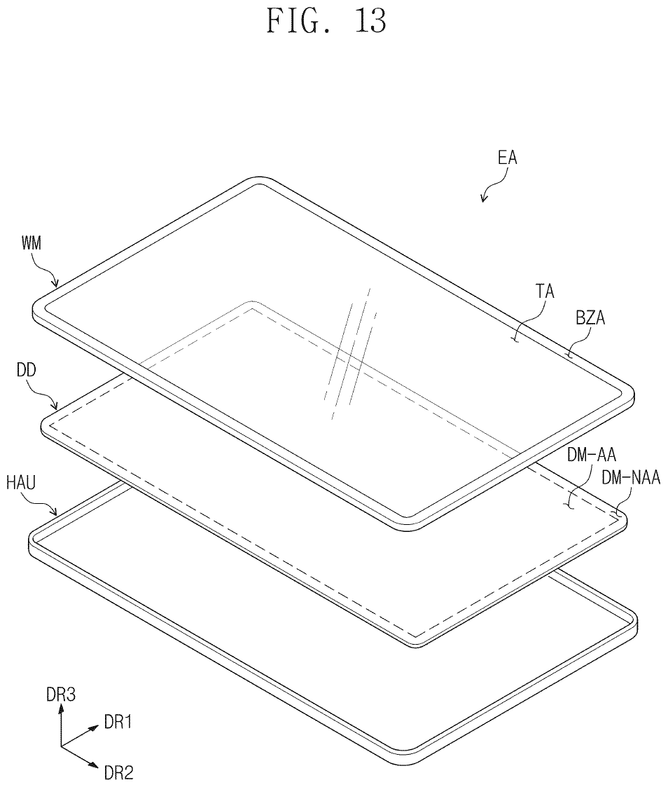

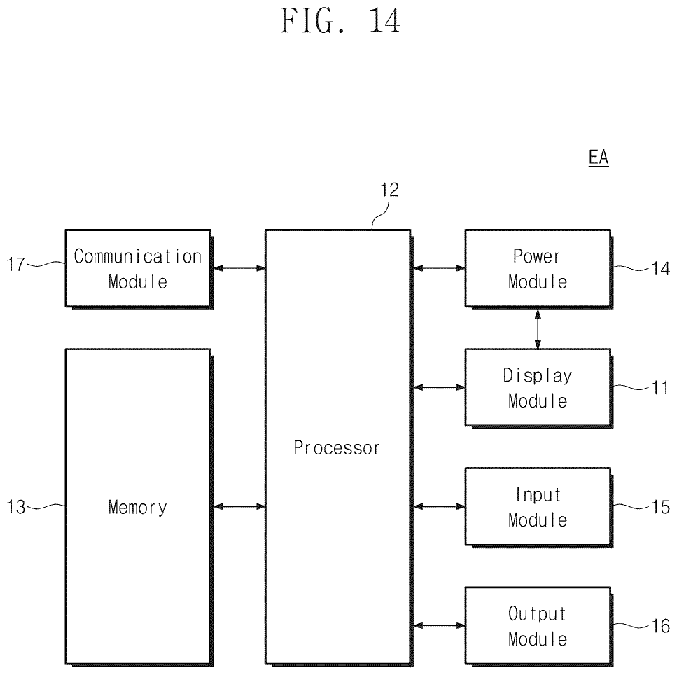

FIG. 12 is a perspective view showing an electronic apparatus of one or more embodiments. FIG. 13 is an exploded perspective view showing an electronic apparatus of one or more embodiments.

An electronic apparatus EA may display an image IM through a display surface EA-IS. The image IM may include a dynamic image as well as a static image. The display surface EA-IS may be parallel to a plane defined by a first direction axis DR1 and a second direction axis DR2. FIG. 12 illustrates the electronic apparatus EA having a flat display surface EA-IS, but an embodiment of the inventive concept is not limited thereto. For example, in one or more embodiments, the electronic apparatus EA may include a curved display surface or a three-dimensional display surface. The three-dimensional display surface may include a plurality of display areas indicating different directions from each other.

The display surface EA-IS may include a display area EA-DA and a non-display area EA-NDA. The electronic apparatus EA may display an image IM through the display area EA-DA.

The non-display area EA-NDA may have a predetermined color. The non-display area EA-NDA may be adjacent to the display area EA-DA. The non-display area EA-NDA may surround the display area EA-DA. Accordingly, a shape of the display area EA-DA may be substantially defined by the non-display area EA-NDA. However, FIG. 12 is an example, and the non-display area EA-NDA may be disposed adjacent to only one side of the display area EA-DA or may not be provided.

Referring to FIG. 13, the electronic apparatus EA may include a display device DD. In addition, the electronic apparatus EA may further include a window member WM and a housing HAU.

The window member WM may cover the entire outer side of the electronic apparatus EA. The window member WM may include a transparent area TA and a bezel area BZA. A front surface of the window member WM including the transparent area TA and the bezel area BZA may correspond to a front surface of the electronic apparatus EA. The transparent area TA may correspond to the display area EA-DA of the electronic apparatus EA illustrated in FIG. 12, and the bezel area BZA may correspond to the non-display area EA-NDA of the electronic apparatus EA illustrated in FIG. 12.

The transparent area TA may be an optically transparent area. The bezel area BZA may be an area having a relatively low light transmittance compared to the transparent area TA. The bezel area BZA may have a predetermined color. The bezel area BZA may be adjacent to the transparent area TA and may surround the transparent area TA. The bezel area BZA may define the shape of the transparent area TA. However, embodiments of the present disclosure are not limited thereto, for example, in one or more embodiments, the bezel area BZA may be disposed adjacent to only one side of the transparent area TA, or a portion thereof may not be provided.

The housing HAU may include a material having relatively high rigidity. For example, the housing HAU may include a frame and/or a plate made of glass, plastic, or metal. The frames and/or plates may be provided in multiple pieces. The housing HAU may provide a predetermined receiving space. The display device DD may be accommodated in the receiving space and protected from external impact.

The display device DD may include substantially the same configurations as those of at least one of the display devices DD, DD-TD, DD-a, DD-b, or DD-c of the embodiments described with reference to FIGS. 1, 2, and 7 to 10. The display device DD may include the light emitting element ED described with reference to FIGS. 3 to 6. Accordingly, the electronic apparatus EA including the display device DD according to one or more embodiments may exhibit excellent reliability.

An active area DM-AA and a peripheral area DM-NAA may be defined in the display device DD. The active area DM-AA may overlap the display area EA-DA illustrated in FIG. 12, and the peripheral area DM-NAA may overlap the non-display area EA-NDA illustrated in FIG. 12.

The active area DM-AA may be an area activated according to an electrical signal. The peripheral area DM-NAA may be an area positioned adjacent to at least one side of the active area DM-AA. The active area DM-AA may include the non-light emitting area NPXA and light emitting areas PXA-R, PXA-G, and PXA-B, illustrated in FIG. 1. The peripheral area DM-NAA may be arranged to surround the active area DM-AA. However, embodiments of the present disclosure are not limited thereto, for example, in one or more embodiments, some of the peripheral areas DM-NAA may not be provided. A driving circuit or driving wirings for driving the active area DM-AA may be arranged in the peripheral area DM-NAA.

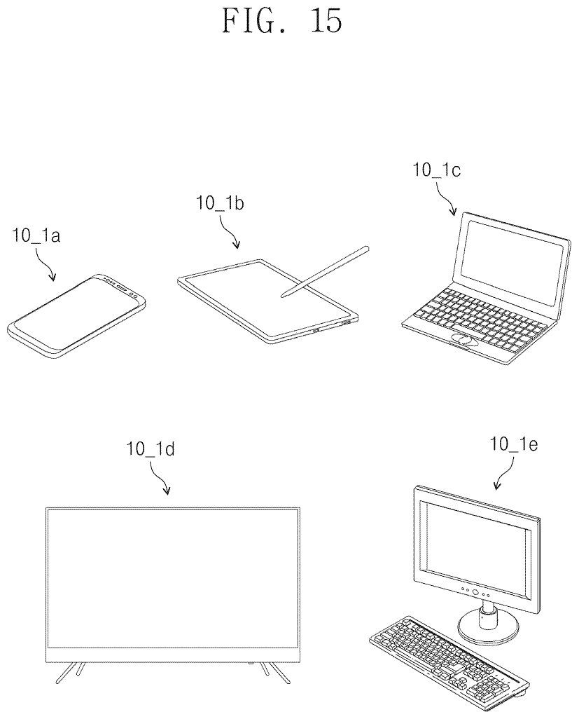

The electronic apparatus EA according to one or more embodiments includes the display device described above, and may further include a module or device having an additional function, in addition to the display device. FIG. 14 is a block diagram of an electronic apparatus according to one or more embodiments of the present disclosure. Referring to FIG. 14, an electronic apparatus EA according to one or more embodiments may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), or a controller. Data information necessary for the operation of the processor 12 or the display module 11 may be stored in the memory 13. If (e.g., when) the processor 12 executes an application stored in the memory 13, an image data signal and/or an input control signal are transmitted to the display module 11, and the display module 11 may process the received signal and output image information through a display screen.

The power module 14 may include a power supply module such as a power adapter or a battery device, and a power conversion module that converts the power supplied by the power supply module to generate power necessary for the operation of the electronic apparatus EA.

The display module 11 may include at least some configurations of the display devices DD, DD-TD, DD-a, DD-b, and/or DD-c, described with reference to FIGS. 1, 2, and 7 to 10. For example, the display module 11 may include a base layer BS, a circuit layer DP-CL, and a display element layer DP-ED among the configurations of the display devices DD, DD-TD, DD-a, DD-b, and/or DD-c, described with reference to FIGS. 1, 2, and 7 to 10. In addition, the display module 11 may further include at least one of an optical layer PP (FIG. 2), a light controlling layer CCL (FIGS. 7 and 10), a color filter layer CFL (FIGS. 7 and 10), and an optical auxiliary layer PL (FIG. 9).

The electronic apparatus EA may further include an input module 15, a non-image output module 16, and/or a communication module 17.

The input module 15 may provide input information to the processor 12 and/or the display module 11. The input module 15 may include various sensor modules as well as physical buttons, a keyboard, and a microphone. Non-limiting examples of sensor module include touch sensors, pressure sensors, distance sensors, position sensors, digitizers, motion recognition sensors, camera sensors, photodetector, photoelectric conversion sensors, temperature sensors, and biosensors such as blood pressure sensors, blood sugar sensors, electrocardiogram sensors, and heart rate sensors.

The non-image output module 16 may receive information other than images transmitted from the processor 12 and provide the information to a user. Non-limiting examples of the non-image output module 16 include an audio module, a haptic module, a light emitting module, and the like, and may include other electronic device-specific functional modules (e.g., a cooling module of a refrigerator, and the like).

The communication module 17 is a module responsible for transmitting and receiving information between the electronic apparatus EA and an external device, and may include a receiving part and a transmitting part. The communication module 17 may include various wireless communication modules such as a mobile communication module, a Wi-Fi module, and a Bluetooth module, or various wired communication modules.

At least one of the configurations of the electronic apparatus EA described above may be included in the above-described display device (at least one of DD, DD-TD, DD-a, DD-b, and DD-c, FIGS. 1, 2, and 7 to 10) according to one or more embodiments. In addition, some of the individual modules functionally included in one module may be included in the display device, and other modules may be provided separately from the display device. For example, the display device may include the display module 11, and the processor 12, the memory 13, and the power module 14 may be provided in the form of other devices within the electronic apparatus EA other than the display device.

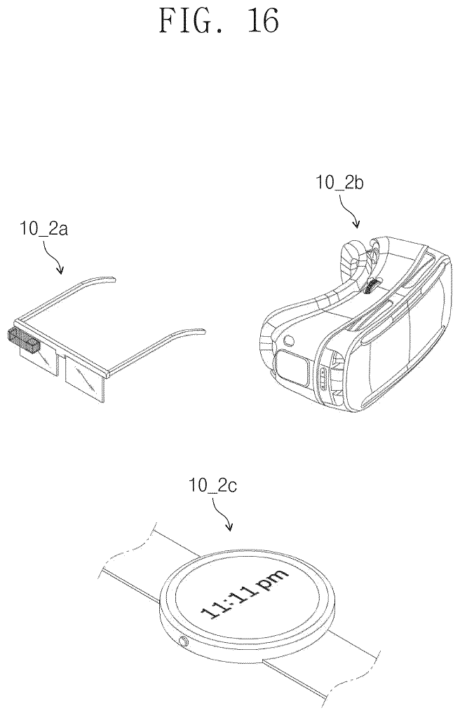

FIG. 15 and FIG. 16 are schematic diagrams showing electronic apparatuses according to one or more embodiments. Referring to FIG. 15 and FIG. 16, various electronic apparatuses to which a display device (at least one of DD, DD-TD, DD-a, DD-b, or DD-c, FIGS. 1, 2, and 7 to 10) according to one or more embodiments is applied may include not only image display electronic apparatuses such as a smart phone 10_1a, a tablet PC 10_1b, a laptop 10_1c, a TV 10_1d, and a monitor for a desk 10_1e, but also wearable electronic apparatuses including display modules such as smart glasses 10_2a, a head-mounted display 10_2b, and a smart watch 10_2c. However, these are embodiments, and the electronic apparatus according to one or more embodiments is not limited thereto.

Hereinafter, with reference to Examples and Comparative Examples, an amine compound according to one or more embodiments of the present disclosure and a light emitting element of one or more embodiments will be described in more detail. In addition, Examples described are shown only for the understanding of the disclosure, and the scope of the disclosure is not limited thereto.

### EXAMPLES

### 1. Synthesis of Amine Compounds

First, a process of synthesizing amine compounds according to one or more embodiments of the disclosure will be described in more detail by presenting a process of synthesizing Compounds 45, 44, 43, 163, 199, 224, 255, 87, 48, 46, 38, 263, 286, 168, and 641 as an example. In addition, the process of synthesizing an amine compound, which will be described hereinafter, is merely provided as an example, and thus the process of synthesizing an amine compound according to one or more embodiments of the disclosure is not limited to Examples described herein.

The amine compound of one or more embodiments may be synthesized by Reaction Formula 1.

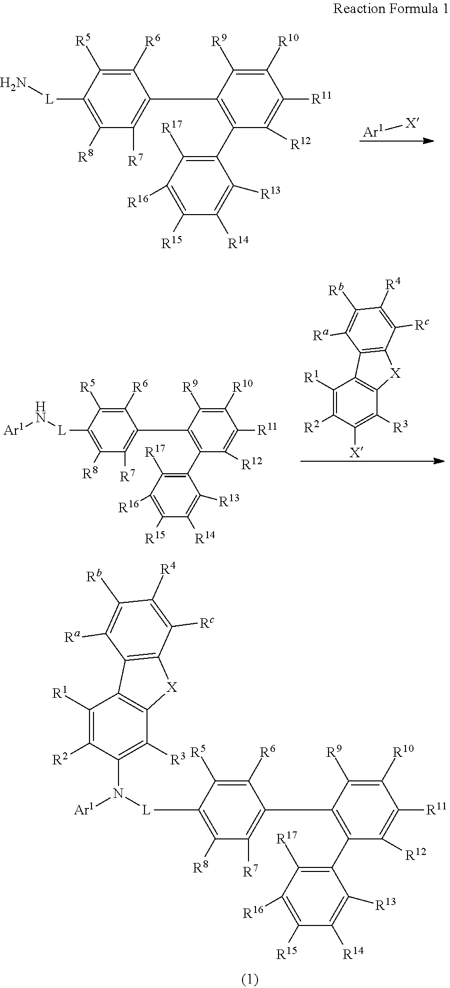

Further, the Reaction Formula 1 may be simplified into Reaction Formulas 2 and 3 to show the synthesis of Example compounds 45, 44, 43, 163, 199, 224, 255, 87, 48, 46, 38, 263, 286, 168, and 641.

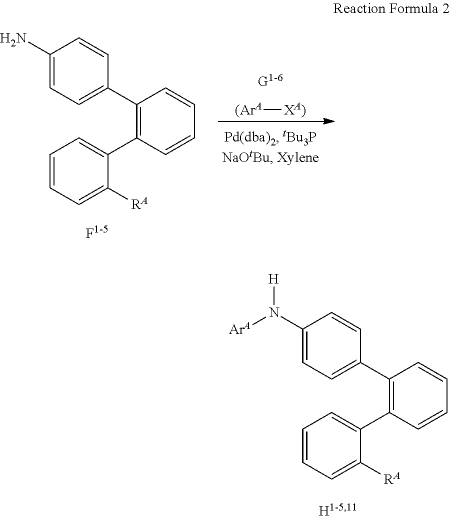

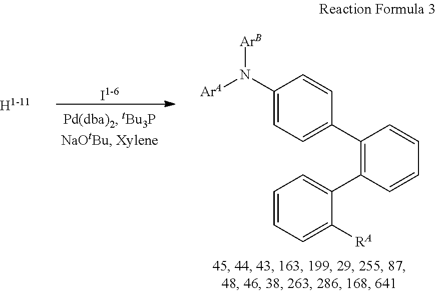

Structures of Intermediates F¹ to F⁵, G¹ to G⁶, H¹ to H⁵, and H¹¹ used in Reaction Formulas 2 and 3 are shown in Tables 1 and 2.

TABLE 1

| Inter- | Sub- | | Inter- | | Sub- | |
|---|---|---|---|---|---|---|
| mediate | stituent | Specific | mediate | Substituent | stituent | Specific |
| F | R^{A} | structure | G | Ar^{A} | X^{A} | structure |
| 1 | 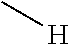 | 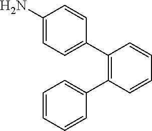 [SMILES: Nc1ccc(-c2ccccc2-c2ccccc2)cc1] | 1 | 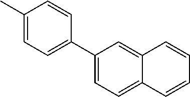 | 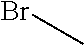 | 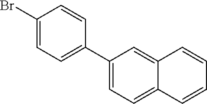 [SMILES: Brc1ccc(-c2ccc3ccccc3c2)cc1] |
| 2 | 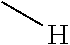 | 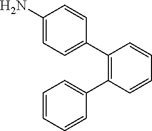 [SMILES: Nc1ccc(-c2ccccc2-c2ccccc2)cc1] | 2 | 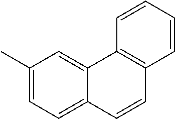 | 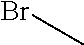 | 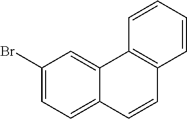 [SMILES: Brc1ccc2ccc3ccccc3c2c1] |
| 3 | 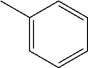 | 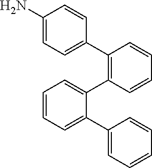 [SMILES: Nc1ccc(-c2ccccc2-c2ccccc2-c2ccccc2)cc1] | 3 | 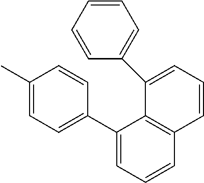 | 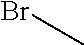 | 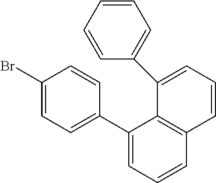 [SMILES: Brc1ccc(-c2cccc3cccc(-c4ccccc4)c23)cc1] |
| 4 | 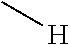 | 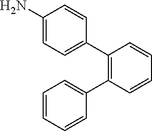 [SMILES: Nc1ccc(-c2ccccc2-c2ccccc2)cc1] | 4 | 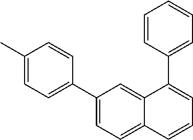 | 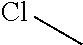 | 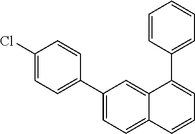 [SMILES: Clc1ccc(-c2ccc3cccc(-c4ccccc4)c3c2)cc1] |
| 5 | 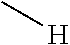 | 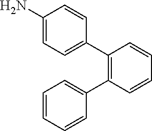 [SMILES: Nc1ccc(-c2ccccc2-c2ccccc2)cc1] | 5 | 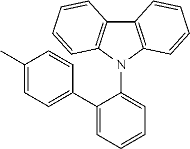 | 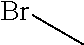 | 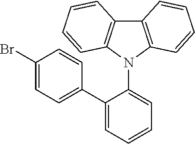 [SMILES: Brc1ccc(-c2ccccc2-n2c3ccccc3c3ccccc32)cc1] |
| | | | 6 | 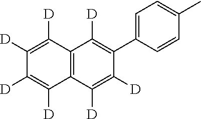 | 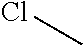 | 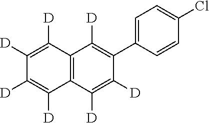 [SMILES: [2H]c1c([2H])c([2H])c2c([2H])c(-c3ccc(Cl)cc3)c([2H])c([2H])c2c1[2H]] |

TABLE 2

| | Substituent | Substituent | Specific |
|---|---|---|---|
| Intermediate H | Ar^{A} | X^{A} | structure |
| 1 | 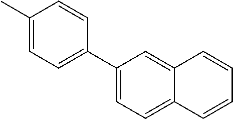 | 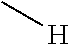 | 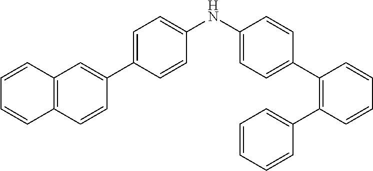 [SMILES: c1ccc(-c2ccccc2-c2ccc(Nc3ccc(-c4ccc5ccccc5c4)cc3)cc2)cc1] |
| 2 | 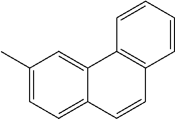 | 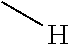 | 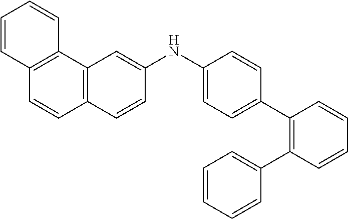 [SMILES: c1ccc(-c2ccccc2-c2ccc(Nc3ccc4ccc5ccccc5c4c3)cc2)cc1] |
| 3 | 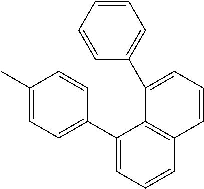 | 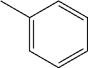 | 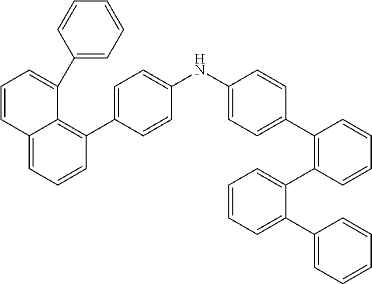 [SMILES: c1ccc(-c2ccccc2-c2ccccc2-c2ccc(Nc3ccc(-c4cccc5cccc(-c6ccccc6)c45)cc3)cc2)cc1] |
| 4 | 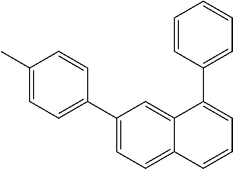 | 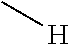 | 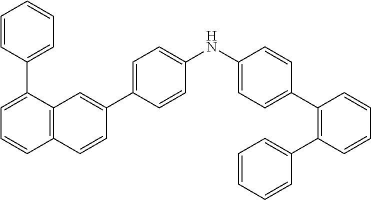 [SMILES: c1ccc(-c2ccccc2-c2ccc(Nc3ccc(-c4ccc5cccc(-c6ccccc6)c5c4)cc3)cc2)cc1] |
| 5 | 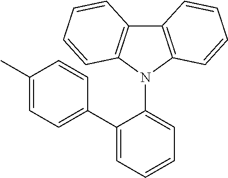 | 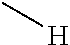 | 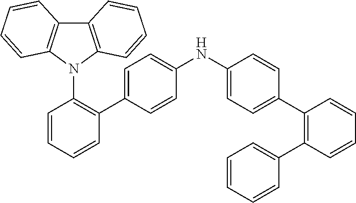 [SMILES: c1ccc(-c2ccccc2-c2ccc(Nc3ccc(-c4ccccc4-n4c5ccccc5c5ccccc54)cc3)cc2)cc1] |
| 11 | 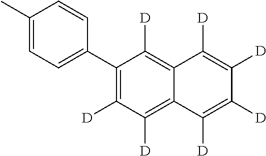 | 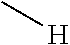 | 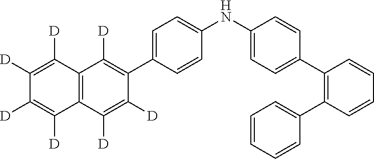 [SMILES: [2H]c1c([2H])c([2H])c2c([2H])c(-c3ccc(Nc4ccc(-c5ccccc5-c5ccccc5)cc4)cc3)c([2H])c([2H])c2c1[2H]] |

Synthesis of Intermediates H¹ to H⁵

### Synthesis of Intermediate H¹

Intermediate H¹ was synthesized by reacting Intermediate F¹ with Intermediate G¹ according to Reaction Formula 2.

A mixture of Intermediate F¹ (4.06 g, 16.6 mmol), Intermediate G¹ (4.92 g, 17.4 mmol), di(benzilideneacetone)palladium(0) (Pd(dba)₂, 476 mg, 0.828 mmol), tri-tertiary-butylphosphine (^{t}Bu₃P, 0.828 mL (2.0 M solution), 1.66 mmol), sodium tertiary-butoxide (NaO^{t}Bu, (4.77 g, 49.7 mmol), and xylene (300 mL) was stirred for 8 hours while being heated at 120° C. in an argon atmosphere. The reaction product was cooled and filtered through Celite, toluene was added thereto, and the mixture was washed with water and a saturated saline solution, dried over anhydrous sodium sulfate, and concentrated. The residue was purified through column chromatography (eluent: hexane/toluene) to obtain Intermediate H¹ (9.50 g, yield: 78%). The production was determined through Fast atom bombardment-Mass spectroscopy (FAB-MS) measurement (m/z=447.2). In addition, in the synthesis of Intermediate H¹, as described above, in Intermediate F¹, R^{A} is H, in Intermediate G¹, Ar^{A} is 4-(2-naphthyl)phenyl, and X^{A} is Br.

### Synthesis of Intermediate H²

Intermediate H² was synthesized in substantially the same manner as H¹ through Reaction Formula 2.

However, in this case, Intermediate F² and Intermediate G² were used instead of Intermediate F¹ and Intermediate G¹ to obtain Intermediate H² (8.77 g, yield: 75%). The production was determined through FAB-MS measurement (m/z=421.2).

### Synthesis of Intermediate H³

Intermediate H³ was synthesized in substantially the same manner as H¹ through Reaction Formula 2.

However, in this case, Intermediate F³ and Intermediate G³ were used instead of Intermediate F¹ and Intermediate G¹ to obtain Intermediate H³ (9.66 g, yield: 72%). The production was determined through FAB-MS measurement (m/z=599.3).

### Synthesis of Intermediate H⁴

Intermediate H⁴ was synthesized in substantially the same manner as H¹ through Reaction Formula 2.

However, in this case, Intermediate F⁴ and Intermediate G⁴ were used instead of Intermediate F¹ and Intermediate G¹ to obtain Intermediate H⁴ (9.08 g, yield: 78%). The production was determined through FAB-MS measurement (m/z=523.2).

### Synthesis of Intermediate H⁵

Intermediate H⁵ was synthesized in substantially the same manner as H¹ through Reaction Formula 2.

However, in this case, Intermediate F⁵ and Intermediate G⁵ were used instead of Intermediate F¹ and Intermediate G¹ to obtain Intermediate H⁵ (9.63 g, yield: 73%). The production was determined through FAB-MS measurement (m/z=562.2).

### Synthesis of Intermediate H¹¹

Intermediate H¹¹ was synthesized in substantially the same manner as H¹ through Reaction Formula 2 above.

However, in this case, G⁶ was used instead of G¹ to obtain Intermediate H¹¹ (7.55 g, yield: 76%). The production was determined through FAB-MS measurement (m/z=454.2).

Intermediates H⁶ to H¹⁰ and Intermediates I¹ to I⁶ used in the synthesis of Example compounds are disclosed below. As shown below, suitable compounds provided with CAS No. were used as Intermediates H⁶ to H¹⁰ and Intermediates I¹ to I⁶.

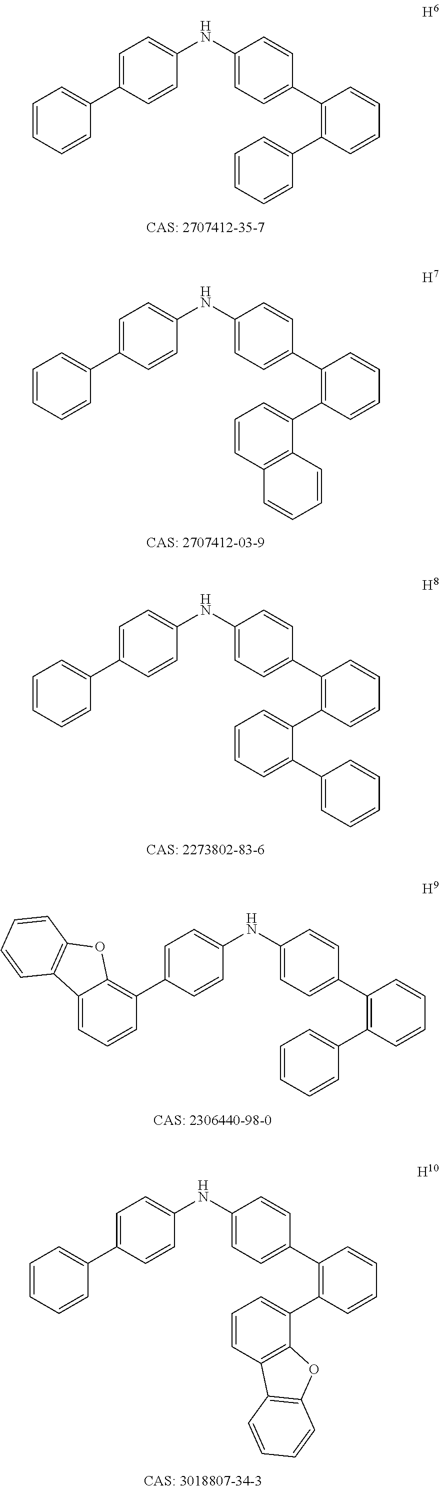 [SMILES: c1ccc(-c2ccc(Nc3ccc(-c4ccccc4-c4ccccc4)cc3)cc2)cc1 ; c1ccc(-c2ccc(Nc3ccc(-c4ccccc4-c4cccc5ccccc45)cc3)cc2)cc1 ; c1ccc(-c2ccc(Nc3ccc(-c4ccccc4-c4ccccc4-c4ccccc4)cc3)cc2)cc1 ; c1ccc(-c2ccccc2-c2ccc(Nc3ccc(-c4cccc5c4oc4ccccc45)cc3)cc2)cc1 ; c1ccc(-c2ccc(Nc3ccc(-c4ccccc4-c4cccc5c4oc4ccccc45)cc3)cc2)cc1]

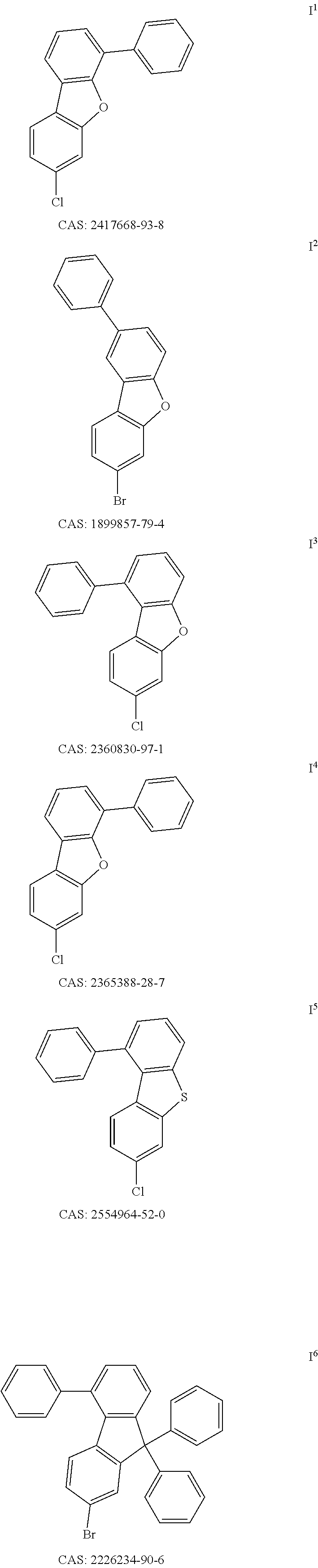 [SMILES: Clc1ccc2c(c1)oc1c(-c3ccccc3)cccc12 ; Brc1ccc2c(c1)oc1ccc(-c3ccccc3)cc12 ; Clc1ccc2c(c1)oc1cccc(-c3ccccc3)c12 ; Clc1ccc2c(c1)oc1c(-c3ccccc3)cccc12 ; Clc1ccc2c(c1)sc1cccc(-c3ccccc3)c12 ; Brc1ccc2c(c1)C(c1ccccc1)(c1ccccc1)c1cccc(-c3ccccc3)c1-2]

### Synthesis of Compound 45

Compound 45 was synthesized by reacting Intermediate H¹ with Intermediate I¹ according to Reaction Formula 3.

A mixture of Intermediate H¹ (4.19 g, 9.35 mmol), Intermediate I¹ (2.74 g, 9.82 mmol), di(benzilideneacetone)palladium(0) (Pd(dba)₂, 268 mg, 0.468 mmol), tri-tertiary-butylphosphine (^{t}Bu₃P, 0.468 mL (2.0 M solution), 0.935 mmol), sodium tertiary-butoxide (2.69 g, 28.1 mmol), and xylene (300 mL) was stirred for 8 hours while being heated at 120° C. in an argon atmosphere. The reaction product was cooled and filtered through Celite, toluene was added thereto, and the mixture was washed with water and a saturated saline solution, dried over anhydrous sodium sulfate, and concentrated. The residue was purified through column chromatography (eluent: hexane/toluene) to obtain Compound 45 (8.72 g, yield: 74%). The production was determined through FAB-MS measurement (m/z=689.3).

### Synthesis of Compound 44

Compound 44 was synthesized in substantially the same manner as Compound 45 through Reaction Formula 3. However, in this case, Intermediate I² was used instead of Intermediate I¹ to obtain Compound 44 (8.82 g, yield: 82%). The production was determined through FAB-MS measurement (m/z=689.3).

### Synthesis of Compound 43

Compound 43 was synthesized in substantially the same manner as Compound 45 through Reaction Formula 3. However, in this case, Intermediate I³ was used instead of Intermediate I¹ to obtain Compound 43 (7.55 g, yield: 71%). The production was determined through FAB-MS measurement (m/z=689.3).

### Synthesis of Compound 163

Compound 163 was synthesized in substantially the same manner as Compound 45 through Reaction Formula 3. However, in this case, Intermediate H⁹ and Intermediate I³ were used instead of Intermediate H¹ and Intermediate I¹ to obtain Compound 163 (7.70 g, yield: 72%). The production was determined through FAB-MS measurement (m/z=729.3).

### Synthesis of Compound 199

Compound 199 was synthesized in substantially the same manner as Compound 45 through Reaction Formula 3. However, in this case, Intermediate H¹⁰ and Intermediate I³ were used instead of Intermediate H¹ and Intermediate I¹ to obtain Example Compound 199 (7.05 g, yield: 73%). The production was determined through FAB-MS measurement (m/z=729.3).

### Synthesis of Compound 224

Compound 224 was synthesized in substantially the same manner as Compound 45 through Reaction Formula 3. However, in this case, Intermediate H⁶ and Intermediate I³ were used instead of Intermediate H¹ and Intermediate I¹ to obtain Example compound 224 (6.68 g, yield: 70%). The production was determined through FAB-MS measurement (m/z=689.3).

### Synthesis of Compound 255

Compound 255 was synthesized in substantially the same manner as Compound 45 through Reaction Formula 3. However, in this case, Intermediate H² was used instead of Intermediate H¹ to obtain Example Compound 255 (6.55 g, yield: 75%). The production was determined through FAB-MS measurement (m/z=663.3).

### Synthesis of Compound 87

Compound 87 was synthesized in substantially the same manner as Compound 45 through Reaction Formula 3. However, in this case, Intermediate H⁸ was used instead of Intermediate H¹ to obtain Example Compound 87 (7.12 g, yield: 82%). The production was determined through FAB-MS measurement (m/z=715.3).

### Synthesis of Compound 48

Compound 48 was synthesized in substantially the same manner as Compound 45 through Reaction Formula 3. However, in this case, Intermediate I⁴ was used instead of Intermediate I¹ to obtain Compound 48 (4.25 g, yield: 65%). The production was determined through FAB-MS measurement (m/z=705.3).

### Synthesis of Compound 46

Compound 46 was synthesized in substantially the same manner as Compound 45 through Reaction Formula 3. However, in this case, Intermediate I⁵ was used instead of Intermediate I¹ to obtain Compound 46 (5.85 g, yield: 68%). The production was determined through FAB-MS measurement (m/z=705.3).

### Synthesis of Compound 38

Compound 38 was synthesized in substantially the same manner as Compound 45 through Reaction Formula 3. However, in this case, Intermediate H⁶ and Intermediate I⁶ were used instead of Intermediate H¹ and Intermediate I¹ to obtain Compound 38 (6.83 g, yield: 72%). The production was determined through FAB-MS measurement (m/z=789.3).

### Synthesis of Compound 263

Compound 263 was synthesized in substantially the same manner as Compound 45 through Reaction Formula 3. However, in this case, Intermediate H⁴ was used instead of Intermediate H¹ to obtain Compound 263 (6.78 g, yield: 75%). The production was determined through FAB-MS measurement (m/z=765.3).

### Synthesis of Compound 286

Compound 286 was synthesized in substantially the same manner as Compound 45 through Reaction Formula 3. However, in this case, Intermediate H³ was used instead of Intermediate H¹ to obtain Compound 286 (6.60 g, yield: 74%). The production was determined through FAB-MS measurement (m/z=841.3).

### Synthesis of Compound 168

Compound 168 was synthesized in substantially the same manner as Compound 45 through Reaction Formula 3. However, in this case, Intermediate H⁵ and Intermediate I³ were used instead of Intermediate H¹ and Intermediate I¹ to obtain Compound 168 (4.85 g, yield: 66%). The production was determined through FAB-MS measurement (m/z=804.3).

### Synthesis of Compound 641

Compound 641 was synthesized in substantially the same manner as Compound 45 through Reaction Formula 3. However, in this case, H¹¹ was used instead of H¹ to obtain Example Compound 641 (8.82 g, yield: 72%). The production was determined through FAB-MS measurement (m/z=696.3).

### 2. Preparation and Evaluation of Light Emitting Elements

Light emitting elements of one or more embodiments, including an amine compound of one or more embodiments in a hole transport layer, were each prepared using a method described herein. Light emitting elements of Examples 1 to Example 15 were prepared using amine compounds 45, 44, 43, 163, 199, 224, 255, 87, 48, 46, 38, 263, 286, 168, and 641, respectively, which are Example Compounds, as materials for the hole transport layer. Comparative Examples 1 to 13 correspond to light emitting elements prepared using Comparative Example Compound C1 to C13, respectively, as materials for the hole transport layer.

### Example Compound

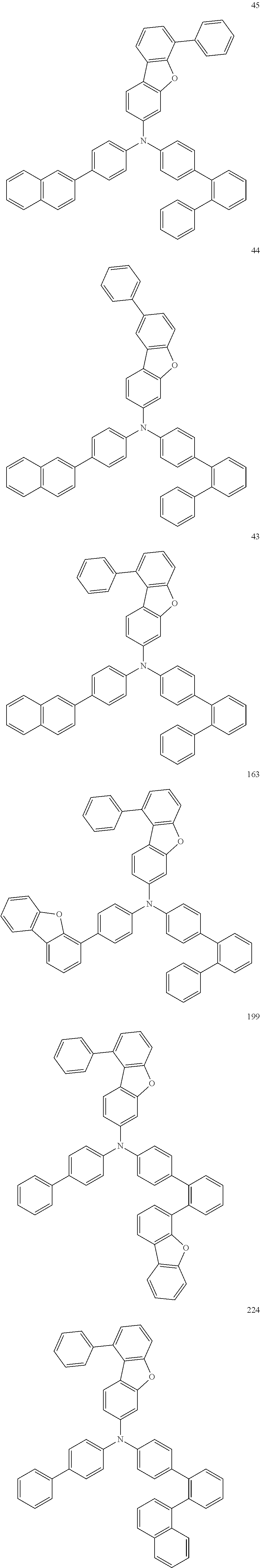

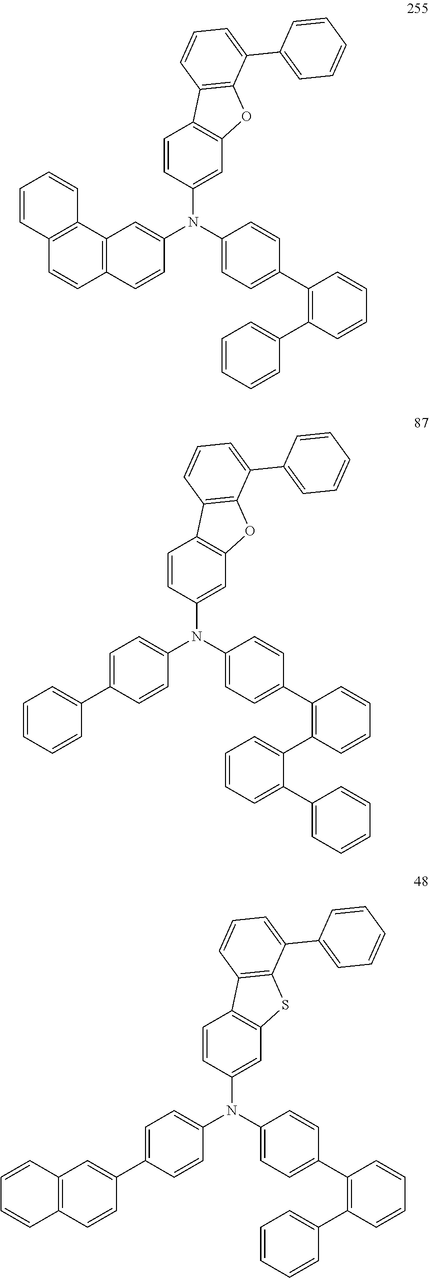 [SMILES: c1ccc(-c2ccccc2-c2ccc(N(c3ccc4c(c3)oc3c(-c5ccccc5)cccc34)c3ccc4ccc5ccccc5c4c3)cc2)cc1 ; c1ccc(-c2ccc(N(c3ccc(-c4ccccc4-c4ccccc4-c4ccccc4)cc3)c3ccc4c(c3)oc3c(-c5ccccc5)cccc34)cc2)cc1 ; c1ccc(-c2ccccc2-c2ccc(N(c3ccc(-c4ccc5ccccc5c4)cc3)c3ccc4c(c3)sc3c(-c5ccccc5)cccc34)cc2)cc1]

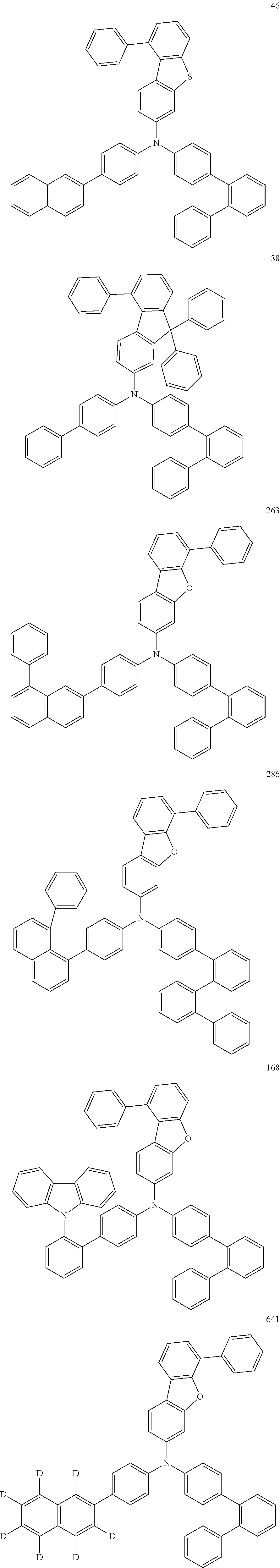

### Comparative Example Compound

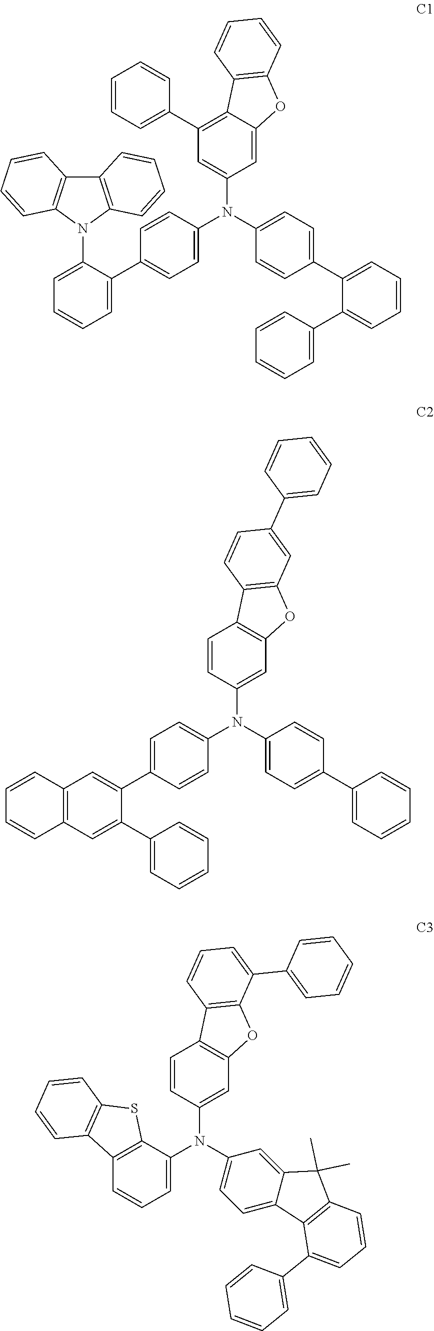 [SMILES: c1ccc(-c2ccccc2-c2ccc(N(c3ccc(-c4ccccc4-n4c5ccccc5c5ccccc54)cc3)c3cc(-c4ccccc4)c4c(c3)oc3ccccc34)cc2)cc1 ; c1ccc(-c2ccc(N(c3ccc(-c4cc5ccccc5cc4-c4ccccc4)cc3)c3ccc4c(c3)oc3cc(-c5ccccc5)ccc34)cc2)cc1 ; CC1(C)c2cc(N(c3ccc4c(c3)oc3c(-c5ccccc5)cccc34)c3cccc4c3sc3ccccc34)ccc2-c2c(-c3ccccc3)cccc21]

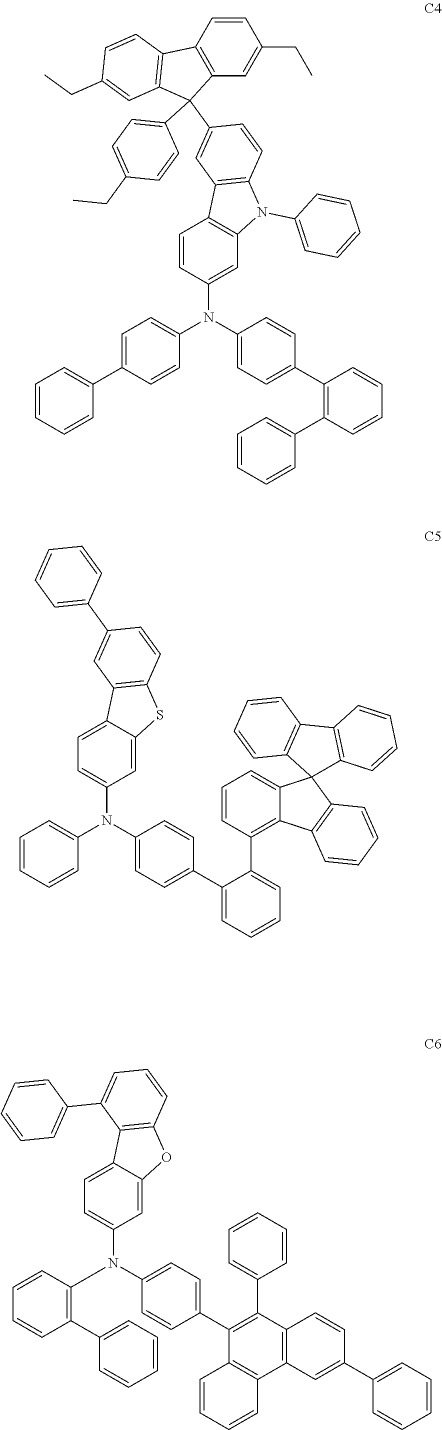 [SMILES: CCc1ccc(C2(c3ccc4c(c3)c3ccc(N(c5ccc(-c6ccccc6)cc5)c5ccc(-c6ccccc6-c6ccccc6)cc5)cc3n4-c3ccccc3)c3cc(CC)ccc3-c3ccc(CC)cc32)cc1 ; c1ccc(-c2ccc3sc4cc(N(c5ccccc5)c5ccc(-c6ccccc6-c6cccc7c6-c6ccccc6C76c7ccccc7-c7ccccc76)cc5)ccc4c3c2)cc1 ; c1ccc(-c2ccc3c(-c4ccccc4)c(-c4ccc(N(c5ccc6c(c5)oc5cccc(-c7ccccc7)c56)c5ccccc5-c5ccccc5)cc4)c4ccccc4c3c2)cc1]

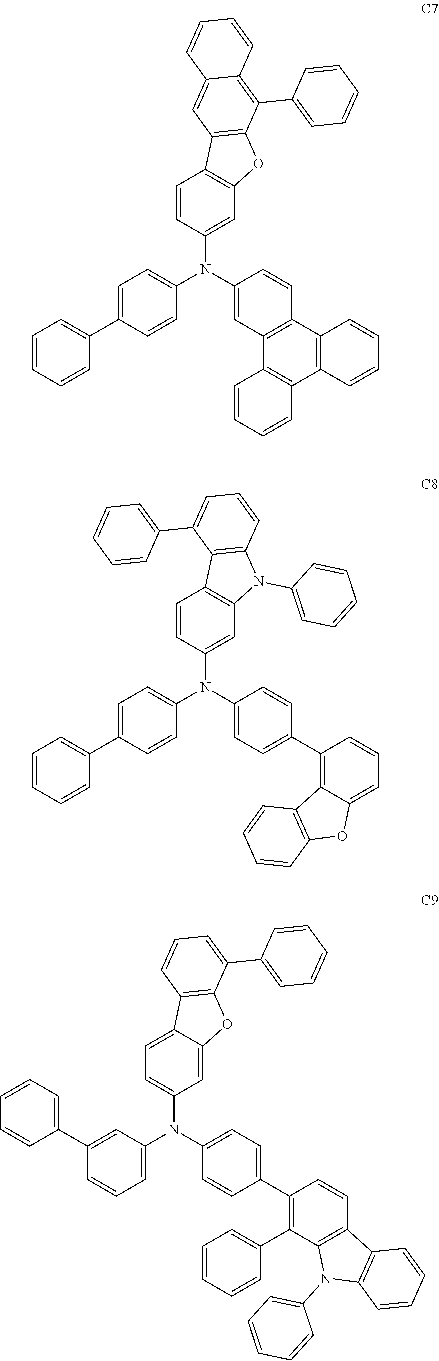 [SMILES: c1ccc(-c2ccc(N(c3ccc4c(c3)oc3c(-c5ccccc5)c5ccccc5cc34)c3ccc4c5ccccc5c5ccccc5c4c3)cc2)cc1 ; c1ccc(-c2ccc(N(c3ccc(-c4cccc5oc6ccccc6c45)cc3)c3ccc4c5c(-c6ccccc6)cccc5n(-c5ccccc5)c4c3)cc2)cc1 ; c1ccc(-c2cccc(N(c3ccc(-c4ccc5c6ccccc6n(-c6ccccc6)c5c4-c4ccccc4)cc3)c3ccc4c(c3)oc3c(-c5ccccc5)cccc34)c2)cc1]

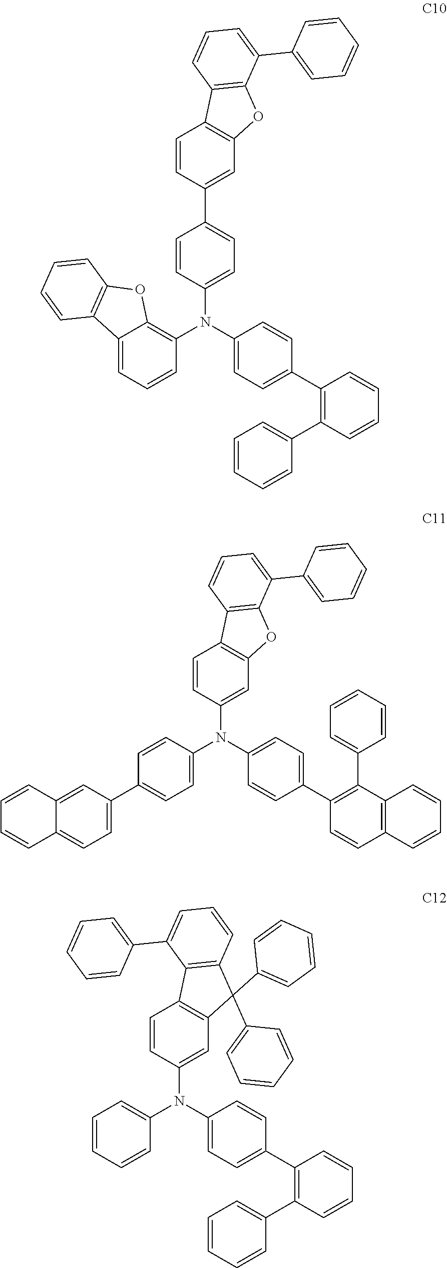 [SMILES: c1ccc(-c2ccccc2-c2ccc(N(c3ccc(-c4ccc5c(c4)oc4c(-c6ccccc6)cccc45)cc3)c3cccc4c3oc3ccccc34)cc2)cc1 ; c1ccc(-c2c(-c3ccc(N(c4ccc(-c5ccc6ccccc6c5)cc4)c4ccc5c(c4)oc4c(-c6ccccc6)cccc45)cc3)ccc3ccccc23)cc1 ; c1ccc(-c2ccccc2-c2ccc(N(c3ccccc3)c3ccc4c(c3)C(c3ccccc3)(c3ccccc3)c3cccc(-c5ccccc5)c3-4)cc2)cc1]

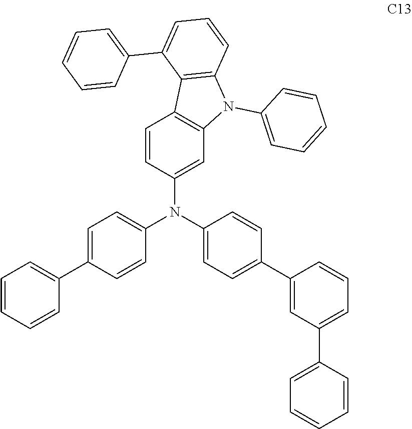 [SMILES: c1ccc(-c2ccc(N(c3ccc(-c4cccc(-c5ccccc5)c4)cc3)c3ccc4c5c(-c6ccccc6)cccc5n(-c5ccccc5)c4c3)cc2)cc1]

An ITO glass substrate (Corning, 15 Ω/cm² (1500 Å)) was cut to a size of 50 mm×50 mm×0.7 mm, washed with isopropyl alcohol and pure water, subjected to ultrasonic cleaning for 5 minutes, and then irradiated with UV for 30 minutes, and ozone-treated. Thereafter, 2-TNATA (4,4′,4″-tris{N,-(2-naphthyl)-N-phenylamino}-triphenylamine) was vacuum deposited to be 600 Å thick to form a hole injection layer. Subsequently one selected from among Example Compounds or one selected from among Comparative Example Compounds was vacuum deposited to be 300 Å thick to form a hole transport layer.

On the hole transport layer, a blue fluorescent host, 9,10-di(naphthalen-2-yl)anthracene (ADN) and a fluorescent dopant, 2, 5, 8, 11-Tetra-t-butylperylene (TBP) were co-deposited at a weight ratio of 97:3 to form an emission layer having a thickness of 250 Å.

On the emission layer, tris(8-hydroxyquinolinato)aluminum (Alq3) was used to form an electron transport layer having a thickness of 250 Å, and then LiF (10 Å) was deposited to form an electron injection layer. On the electron injection layer, aluminum (Al) was used to form a second electrode having a thickness of 1000 Å.

The compounds of each functional layer used for the preparation of the light emitting elements are as follows.

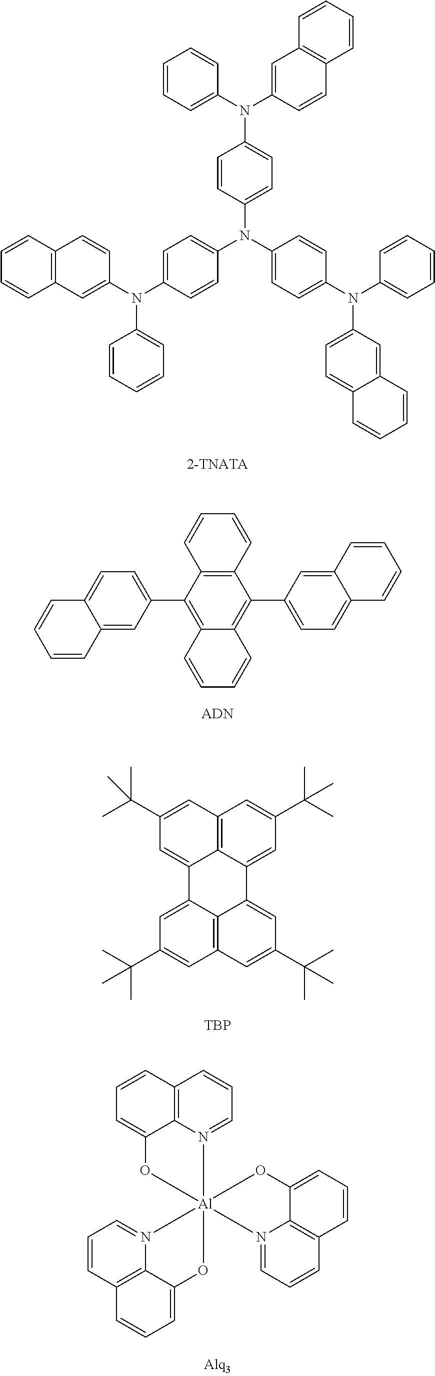

### Evaluation of Light Emitting Elements

Table 3 shows results of evaluation on each of light emitting elements for Examples 1 to 15 and Comparative Examples 1 to 13. In Table 3, luminous efficiency and lifetime of each of the prepared light emitting elements are shown.

In the results of evaluating properties of each of Examples and Comparative Examples shown in Table 3, luminous efficiency indicates an efficiency value at a current density of 10 mA/cm², and element lifetime indicates luminance half-life at a current density of 10 mA/cm². Degradation in purity was indicated as a percentage of the difference in purity of material before and after deposition.

Evaluation of element current density and luminous efficiency was performed in a dark room using a source meter of 2400 Series from Keithley Instruments, a color luminance meter C-200 from Konica Minolta, and PC Program LabVIEW8.2 for measuring products from National Instruments Co., Ltd. of Japan.

In addition, the luminous efficiency and the element service life were shown as comparative values when the luminous efficiency and the element service life of Comparative Example 2 were set to 100%.

TABLE 3

| | | Effi- | | Purity |
|---|---|---|---|---|
| | | ciency | Lifetime | degradation |
| | Hole transport material | (%) | (%) | (%) |
| Example 1 | Compound 45 | 125 | 130 | 0.0 |
| Example 2 | Compound 44 | 115 | 120 | 0.0 |
| Example 3 | Compound 43 | 125 | 125 | 0.0 |
| Example 4 | Compound 163 | 125 | 120 | 0.0 |
| Example 5 | Compound 199 | 125 | 120 | 0.0 |
| Example 6 | Compound 224 | 120 | 115 | 0.0 |
| Example 7 | Compound 255 | 120 | 125 | 0.0 |
| Example 8 | Compound 87 | 125 | 120 | 0.0 |
| Example 9 | Compound 48 | 125 | 120 | 0.0 |
| Example 10 | Compound 46 | 125 | 115 | 0.0 |
| Example 11 | Compound 38 | 120 | 110 | 0.0 |
| Example 12 | Compound 163 | 125 | 130 | 0.0 |
| Example 13 | Compound 286 | 125 | 115 | 0.0 |
| Example 14 | Compound 168 | 120 | 110 | 0.0 |
| Example 15 | Compound 641 | 125 | 135 | 0.0 |
| Comparative | Comparative Example | 105 | 80 | 0.2 |
| Example 1 | Compound C1 | | | |
| Comparative | Comparative Example | 100 | 100 | 0.0 |
| Example 2 | Compound C2 | | | |
| Comparative | Comparative Example | 105 | 80 | 0.2 |
| Example 3 | Compound C3 | | | |
| Comparative | Comparative Example | 95 | 60 | 0.4 |
| Example 4 | Compound C4 | | | |
| Comparative | Comparative Example | 95 | 70 | 0.2 |
| Example 5 | Compound C5 | | | |
| Comparative | Comparative Example | 100 | 60 | 0.4 |
| Example 6 | Compound C6 | | | |
| Comparative | Comparative Example | 95 | 60 | 0.4 |
| Example 7 | Compound C7 | | | |
| Comparative | Comparative Example | 100 | 90 | 0.2 |
| Example 8 | Compound C8 | | | |
| Comparative | Comparative Example | 100 | 60 | 0.5 |
| Example 9 | Compound C9 | | | |
| Comparative | Comparative Example | 95 | 90 | 0.0 |
| Example 10 | Compound C10 | | | |
| Comparative | Comparative Example | 100 | 80 | 0.4 |
| Example 11 | Compound C11 | | | |
| Comparative | Comparative Example | 110 | 90 | 0.1 |
| Example 12 | Compound C12 | | | |
| Comparative | Comparative Example | 95 | 95 | 0.0 |
| Example 13 | Compound C13 | | | |

Referring to the results of Table 3, it is seen that each of the light emitting elements of Examples using the amine compound according to one or more embodiments of the disclosure as a hole transport layer material exhibits greater luminous efficiency and longer element service life than the light emitting elements of Comparative Examples. Comparative Example compounds are found to have reduced luminous efficiency and lifetime and degraded purity when applied to light emitting elements compared to Example compounds. That is, with reference to Table 3, it is determined that a light emitting element using the amine compound according to one or more embodiments of the disclosure showed improvements in luminous efficiency and/or element service life and no degradation in purity compared to Comparative Examples.

The amine compound according to one or more embodiments includes the first substituent, the second substituent, and the third substituent linked to the core nitrogen atom, and thus allow a light emitting element to obtain high efficiency and long service life. The first substituent includes a dibenzofuran moiety, a dibenzothiophene moiety, a carbazole moiety, or a fluorene moiety in which an aryl group is substituted at a specific position. The 3rd carbon of the dibenzofuran moiety, the dibenzothiophene moiety, the carbazole moiety, or the fluorene moiety in the first substituent may be linked to the core nitrogen atom, and at least one of the 5th carbon, the 7th carbon, or the 8th carbon among the other carbons may be linked to the aryl group. The second substituent may include an ortho-terphenyl moiety and may be bonded to the core nitrogen atom through an arylene linker or a heteroarylene linker, or may be bonded directly to the core nitrogen atom without a separate linker. The third substituent is an aryl group or a heteroaryl group and is directly bonded to the core nitrogen atom. As a result, the amine compound of one or more embodiments may have excellent or suitable electrical stability and high charge transport properties due to the introduction of such substituents and the specific substitution positions. Accordingly, the amine compound of one or more embodiments may have improved lifetime and stability. In addition, the light emitting element of one or more embodiments including the amine compound of one or more embodiments may have improved luminous efficiency and lifetime.

Comparative Example Compound C1 included in Comparative Example 1 has an aryl group at the position of R¹ instead of the positions of R^{a} to R^{c} in Formula 1 according to one or more embodiments of the disclosure, and has reduced stability of molecules due to increased molecular distortion near the central nitrogen of an amine group. Accordingly, it is observed that Comparative Example 1 including Comparative Example Compound C1 has reduced luminous efficiency and element service life compared to Examples.

Comparative Example Compound C2 included in Comparative Example 2 corresponds to a compound in which an aryl group is substituted at the position of R⁴ instead of the positions of R^{a} to R^{c} in Formula 1 according to one or more embodiments of the disclosure. Because the distance from the central nitrogen to the position of R⁴ is relatively large, the high-efficiency effect of the substituent is insignificant. In addition, as in Comparative Example Compound C2, when an aryl group is substituted only at the position of R⁴ and not at the positions of R^{a} to R^{c}, a structure is formed in which a benzene ring is arranged linearly from the central nitrogen atom, requiring high temperature when preparing an element (e.g., hole transport layer) through vacuum deposition, making the compound prone to decomposition. Accordingly, it is observed that Comparative Example 2 including Comparative Example Compound C2 has reduced luminous efficiency and element service life compared to Examples.

Comparative Example Compound C3 included in Comparative Example 3 corresponds to a compound including a 9,9-dimethylfluorene moiety in molecules. In the fluorene moiety in which an alkyl group is substituted at position 9, the substituent itself is not chemically stable enough. Accordingly, it is observed that Comparative Example 3 including Comparative Example Compound C3 has reduced luminous efficiency and element service life compared to Examples.

Comparative Example Compound C4 included in Comparative Example 4 corresponds to a compound including a 9-fluorene moiety in molecules. The 9-fluorene moiety itself is not chemically stable enough. Accordingly, it is observed that Comparative Example 4 including Comparative Example Compound C4 has reduced luminous efficiency and element service life compared to Examples.

Comparative Example Compound C5 included in Comparative Example 5 corresponds to a compound including a spirobifluorene moiety in molecules. In the spirobifluorene moiety, the substituent itself is not chemically stable enough. Accordingly, it is observed that Comparative Example 5 including Comparative Example Compound C5 has reduced luminous efficiency and element service life compared to Examples.

Comparative Example Compound C6 included in Comparative Example 6 corresponds to a case in which L is a direct linkage, and R⁹ and R¹⁰, and R¹¹ and R¹² are each bonded together to form a ring in Formula 1 according to one or more embodiments of the disclosure. When both (e.g., simultaneously) R⁹ and R¹⁰, and R¹¹ and R¹² are subjected to fused cyclization as in Comparative Example Compound C6, intramolecular distortion may be caused and thus the thermal stability of material may be reduced. Accordingly, it is observed that Comparative Example 6 including Comparative Example Compound C6 has reduced luminous efficiency and element service life compared to Examples.

Comparative Example Compound C7 included in Comparative Example 7 corresponds to a compound in which one adjacent pair among R¹ to R⁴, and R^{a} to R^{c} is bonded together to form a fused ring structure in Formula 1 according to one or more embodiments of the disclosure. When any one pair among R¹ to R⁴, and R^{a} to R^{c} is bonded together to form an additional fused ring structure as in Comparative Example Compound C7, stacking of molecules becomes excessively (or substantially) high, and thus deposition temperature may increase significantly, which may cause greater decomposition of molecules upon deposition. Accordingly, it is observed that Comparative Example 7 including Comparative Example Compound C7 has reduced luminous efficiency and element service life compared to Examples.

Comparative Example Compound C8 included in Comparative Example 8 corresponds to a case in which L is a direct linkage and any of the pairs R¹² and R¹³, and R¹² and R¹⁷ are bonded together to form a ring in Formula 1 according to one or more embodiments of the disclosure. When R¹² and R¹³, or R¹² and R¹⁷ are bonded together to form a heterocycle such as a dibenzofuran ring, as in Comparative Example Compound C8, a benzene ring arranged between the central nitrogen atom and the heterocycle, and the heterocycle may be greatly distorted sterically, thereby reducing thermal stability. Accordingly, it is observed that Comparative Example 8 including Comparative Example Compound C8 has reduced luminous efficiency and element service life compared to Examples.

Comparative Example Compound C9 included in Comparative Example 9 corresponds to a case in which L is a direct linkage, and R¹¹ and R¹² are bonded together to form a nitrogen-containing heterocycle in Formula 1 according to one or more embodiments of the disclosure. When R¹¹ and R¹² are bonded together to form a nitrogen-containing heterocycle such as a carbazole ring as in Comparative Example Compound C9, intramolecular distortion is caused, resulting in poor chemical stability. Accordingly, it is observed that Comparative Example 9 including Comparative Example Compound C9 has reduced luminous efficiency and element service life compared to Examples.

Compared to the amine compound according to one or more embodiments of the disclosure, Comparative Example Compound C10 included in Comparative Example 10 corresponds to a case in which the dibenzofuran moiety of the first substituent is linked to the central nitrogen atom through a linker. When the dibenzofuran moiety, the dibenzothiophene moiety, or the carbazole moiety included in the first substituent is not directly bonded to an amine group but is linked to the central nitrogen atom through a linker, the high efficiency and long life effects from the first substituent may be reduced. Accordingly, it is observed that Comparative Example 10 including Comparative Example Compound C10 has reduced luminous efficiency and element service life compared to Examples.

Comparative Example Compound C11 included in Comparative Example 11 corresponds to a compound including an N,N-bis(4-(2-naphthyl)phenyl)amine moiety in molecules. When the N,N-bis(4-(2-naphthyl)phenyl)amine moiety is included in molecules, stacking of molecules becomes excessively (or substantially) high, and thus deposition temperature may increase significantly, which may cause greater decomposition of molecules upon deposition. Accordingly, it is observed that Comparative Example 11 including Comparative Example Compound C11 has reduced luminous efficiency and element service life compared to Examples.

Comparative Example Compound C12 included in Comparative Example 12 corresponds to a case in which the compound has a similar structure to Example Compound 38, but the substituent corresponding to Ar¹ according to one or more embodiments of the disclosure is an unsubstituted phenyl group. Herein, in Formula 1, when X is CAr³Ar⁴, the fused ring structure containing X has greater steric hindrance to cause an increase in distortion of an amine skeleton at the center of molecules, making the molecules unstable. Accordingly, when Ar¹ is an unsubstituted phenyl group, sufficient spread of HOMO orbital in the substituent Ar¹ is not secured, and thus due to the incapability of making up for the destabilization, the overall stability of Comparative Example Compound C12 is less than that of Example Compound 38, resulting in a slight decrease in efficiency and reduced lifetime.

Comparative Example Compound C13 included in Comparative Example 13 has a similar structure to Comparative Example Compound C8, but does not have an o-terphenylyl group moiety ([1,1′:2′,1″-terphenyl]-4-yl group moiety) corresponding to the second substituent according to one or more embodiments of the disclosure. Because this o-terphenylyl group moiety is considered to be an essential part in ensuring sufficient efficiency herein, the element efficiency has failed to reach the level of one or more embodiments of the disclosure.

A light emitting element according to one or more embodiments includes an amine compound according to one or more embodiments, and may thus exhibit high efficiency and long life characteristics.

An amine compound of one or more embodiments may exhibit high efficiency and long life characteristics if (e.g., when) applied to light emitting elements. For example, the amine compound in one or more embodiments includes three substituents linked to a core nitrogen atom, enhancing the efficiency and longevity of light-emitting elements. The first substituent features a dibenzofuran, dibenzothiophene, carbazole, or fluorene moiety with an aryl group at a specific position. The second substituent may include an ortho-terphenyl moiety, bonded to the core nitrogen atom either directly or through a linker. The third substituent is an aryl or heteroaryl group directly bonded to the core nitrogen atom. These specific substituents and their positions contribute to the compound's electrical stability and charge transport properties, resulting in improved lifetime and stability. Consequently, light-emitting elements incorporating this amine compound exhibit enhanced luminous efficiency and longevity.

A display device/apparatus of one or more embodiments may exhibit high display quality by including the light emitting element of one or more embodiments of the disclosure.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

In the context of the present application and unless otherwise defined, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element, the display device, the display apparatus, or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, each suitable feature of the various embodiments of the disclosure may be combined or combined with each other, partially or entirely, and may be technically interlocked and operated in various suitable ways, and each embodiment may be implemented independently of each other or in conjunction with each other in any suitable manner unless otherwise stated or implied.

Although the disclosure has been described with reference to example embodiments of the disclosure, it will be understood that the disclosure should not be limited to these embodiments but one or more suitable changes and modifications may be made by those skilled in the art without departing from the spirit and scope of the disclosure. Accordingly, the technical scope of the disclosure is not intended to be limited to the contents set forth in the detailed description of the disclosure, but is intended to be defined by the appended claims and equivalents thereof.

What is claimed is:

1. A light emitting element comprising:
a first electrode;
a second electrode on the first electrode; and
at least one functional layer between the first electrode and the second electrode and comprising an amine compound represented by Formula 1: 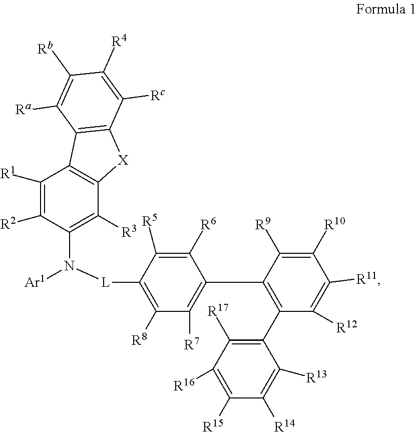
wherein in Formula 1,
X is O, S, NAr², or CAr³Ar⁴,
R^{a} to R^{c} are each independently hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one selected from among R^{a} to R^{c} is a substituted or unsubstituted aryl group having 6 to 30 ring-forming carbon atoms,
R¹ to R⁴ are each independently hydrogen, deuterium, a substituted or unsubstituted alkyl group having 1 to 20 carbon atoms, or a substituted or unsubstituted aryl group having 6 to 30 ring-forming carbon atoms,
Ar¹ is a substituted or unsubstituted aryl group having 6 to 30 ring-forming carbon atoms or a substituted or unsubstituted heteroaryl group having 2 to 30 ring-forming carbon atoms,
Ar² to Ar⁴ are each independently a substituted or unsubstituted aryl group having 6 to 30 ring-forming carbon atoms,
L is a direct linkage, a substituted or unsubstituted arylene group having 6 to 30 ring-forming carbon atoms, or a substituted or unsubstituted heteroarylene group having 2 to 30 ring-forming carbon atoms,
R⁵ to R¹⁷ are each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring,
in Formula 1, a case in which L is a direct linkage and R⁹ and R¹⁰, and R¹¹ and R¹² are each bonded together to form a ring is excluded,
in Formula 1, a case in which L is a direct linkage and any of the pairs R¹² and R¹³, and R¹² and R¹⁷ are bonded together to form a ring is excluded,
in Formula 1, a case in which L is a direct linkage and R¹¹ and R¹² are bonded together to form a nitrogen-containing heterocycle is excluded,
in Formula 1, a case in which when X is CAr³Ar⁴, Ar¹ is an unsubstituted phenyl group is excluded,
a case in which the amine compound represented by Formula 1 contains a fluorene moiety substituted with at least one alkyl group at position 9 or unsubstituted, a substituted or unsubstituted 9-fluorene moiety, a substituted or unsubstituted spirobifluorene moiety, or a substituted or unsubstituted N,N-bis(4-(2-naphthyl)phenyl)amine moiety is excluded, and
at least one hydrogen of the amine compound is optionally substituted with deuterium.

2. The light emitting element of claim 1, wherein the at least one functional layer comprises:
an emission layer;
a hole transport region between the first electrode and the emission layer; and
an electron transport region between the emission layer and the second electrode, and
wherein the hole transport region comprises the amine compound represented by Formula 1.

3. The light emitting element of claim 2, wherein the hole transport region comprises:
a hole injection layer on the first electrode; and
a hole transport layer on the hole injection layer, and
wherein the hole transport layer comprising the amine compound represented by Formula 1.

4. The light emitting element of claim 1, wherein the amine compound represented by Formula 1 is represented by any one selected from among Formulas 2-1 to 2-3: 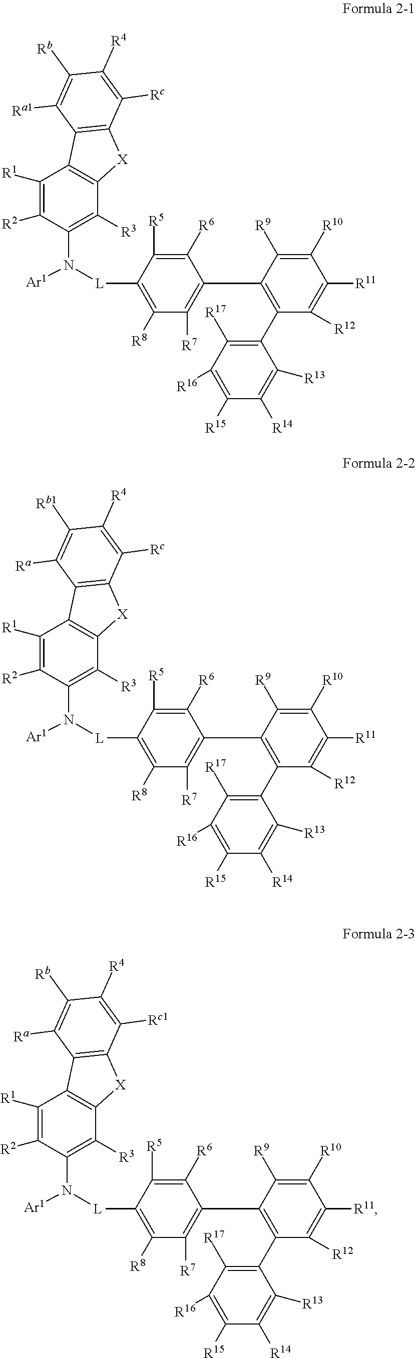
in Formulas 2-1 to 2-3,
R^{a1}, R^{b1}, and R^{c1} being each independently a substituted or unsubstituted aryl group having 6 to 30 ring-forming carbon atoms, and
Ar¹, L, X, R^{a}, R^{b}, R^{c}, and R¹ to R¹⁷ each being the same as defined in Formula 1.

5. The light emitting element of claim 1, wherein the amine compound represented by Formula 1 is represented by Formula 3: 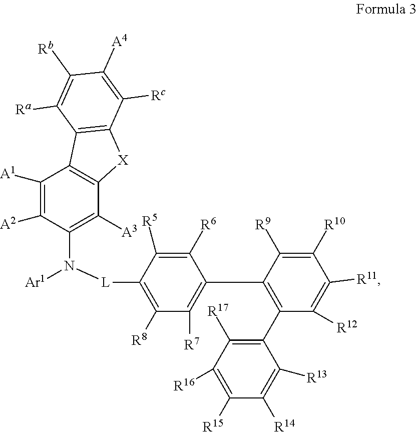
in Formula 3,
A¹ to A⁴ being each independently hydrogen or deuterium, and
Ar¹, L, X, R^{a}, R^{b}, R^{c}, and R⁵ to R¹⁷ each being the same as defined in Formula 1.

6. The light emitting element of claim 1, wherein Ar¹ is a substituted or unsubstituted phenyl group, a substituted or unsubstituted biphenylyl group, a substituted or unsubstituted naphthyl group, a substituted or unsubstituted naphthylphenyl group, a substituted or unsubstituted phenanthrenyl group, a substituted or unsubstituted dibenzofuranyl group, or a substituted or unsubstituted dibenzothiophenyl group.

7. The light emitting element of claim 1, wherein when Ar¹ is substituted, then a substituent of Ar¹ is deuterium, a substituted or unsubstituted phenyl group, or a substituted or unsubstituted naphthyl group.

8. The light emitting element of claim 1, wherein the amine compound represented by Formula 1 is represented by Formula 4-1 or Formula 4-2: 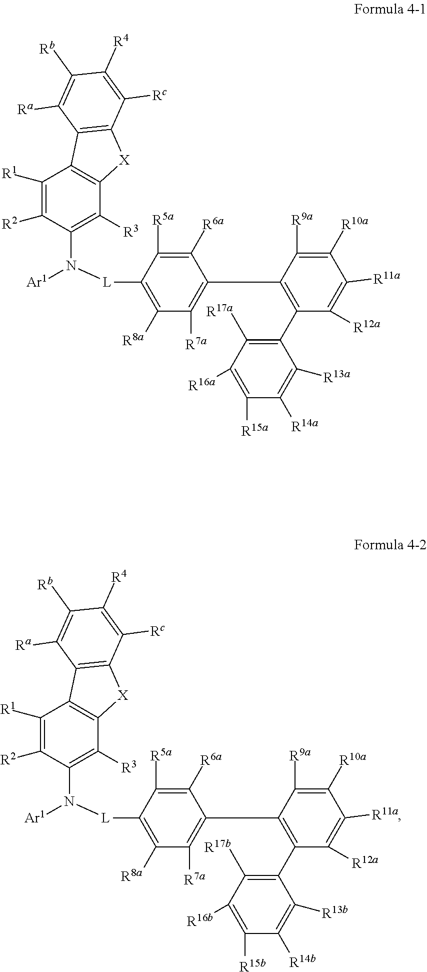
wherein in Formula 4-1,
R^{5a} to R^{17a} being each independently hydrogen, deuterium, a substituted or unsubstituted alkyl group having 1 to 20 carbon atoms, or a substituted or unsubstituted aryl group having 6 to 30 ring-forming carbon atoms,
in Formula 4-2,
R^{5a} to R^{12a} being each independently hydrogen, deuterium, a substituted or unsubstituted alkyl group having 1 to 20 carbon atoms, or a substituted or unsubstituted aryl group having 6 to 30 ring-forming carbon atoms,
R^{13b} to R^{17b} being each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring, and
in Formulas 4-1 and 4-2,
Ar¹, L, X, R^{a}, R^{b}, R^{c}, and R¹ to R⁴ each being the same as defined in Formula 1.

9. The light emitting element of claim 1, wherein the amine compound represented by Formula 1 is represented by Formula 5: 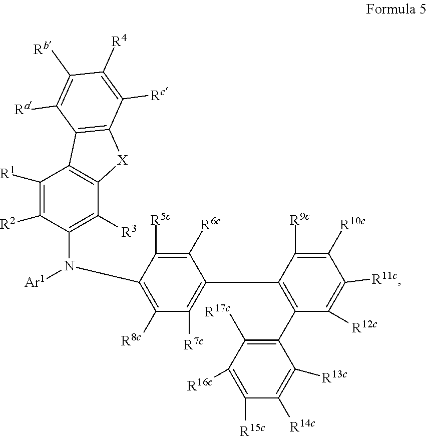
in Formula 5,
R^{a}′ to R^{c}′ being each independently hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one selected from among R^{a}′ to R^{c}′ being a substituted or unsubstituted phenyl group,
Ar^{1a} being a substituted or unsubstituted aryl group having 6 to 30 ring-forming carbon atoms,
R^{5c} to R^{17c} being each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring,
in Formula 5, a case in which when R^{11c} and R^{12c} are bonded together to form a ring, Ar^{1a} is a substituted or unsubstituted naphthylphenyl group being excluded,
in Formula 5, a case in which L is a direct linkage and R^{9c} and R^{10c}, and R^{11c} and R^{12c} are each bonded together to form a ring being excluded,
in Formula 5, a case in which L is a direct linkage and any of the pairs R^{12c} and R^{13c}, and R^{12c} and R^{17c} are bonded together to form a ring being excluded,
in Formula 5, a case in which L is a direct linkage and R^{11c} and R^{12c} are bonded together to form a nitrogen-containing heterocycle being excluded,
in Formula 5, a case in which L is a direct linkage and R^{6c} and R^{9c} are bonded together to form a ring being excluded,
when Ar^{1a} is substituted, a substituent of Ar^{1a} being deuterium or a substituted or unsubstituted aryl group having 6 to 15 ring-forming carbon atoms, and
R¹ to R⁴, X, and L each being the same as defined in Formula 1.

10. The light emitting element of claim 1, wherein the amine compound represented by Formula 1 is represented by Formula 6: 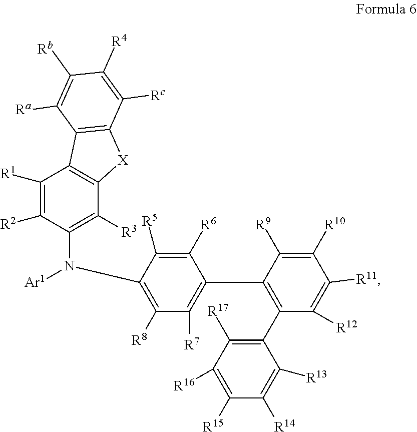
in Formula 6,
Ar¹, X, R^{a}, R^{b}, R^{c}, and R¹ to R¹⁷ each being the same as defined in Formula 1.

11. The light emitting element of claim 1, wherein the amine compound represented by Formula 1 is represented by any one selected from among Formulas 7-1 to 7-3: 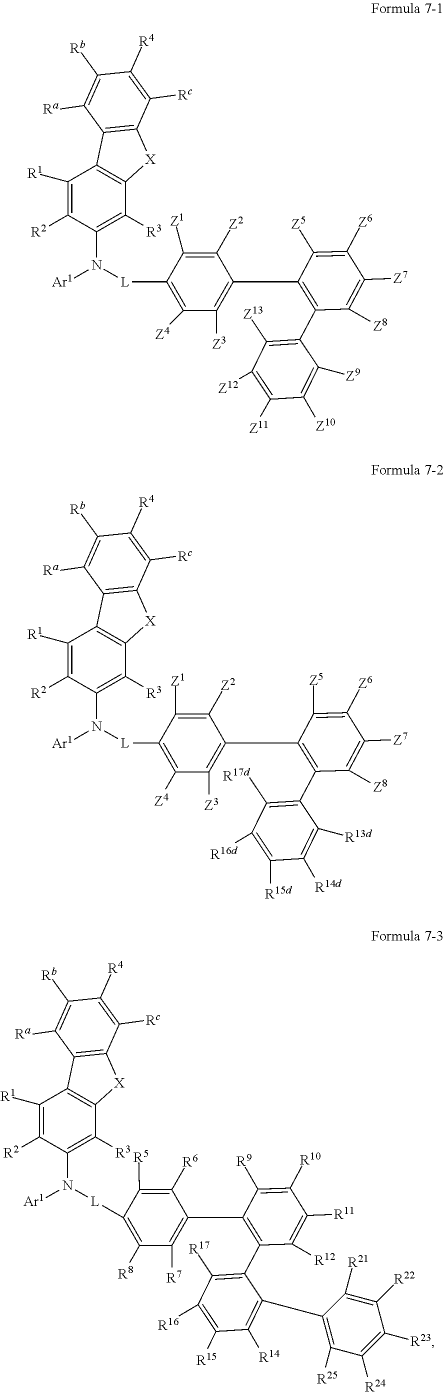
wherein in Formula 7-1,
Z¹ to Z¹³ are each independently hydrogen or deuterium,
wherein in Formula 7-2,
Z¹ to Z⁸ are each independently hydrogen or deuterium,
R^{13d} to R^{17d} are each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring,
one adjacent pair among R^{13d} to R^{17d} is bonded together to form a ring,
wherein in Formula 7-3,
R²¹ to R²⁵ are each independently hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, and/or bonded to an adjacent group to form a ring, and
wherein in Formulas 7-1 to 7-3,
Ar¹, L, X, R^{a}, R^{b}, R^{c}, R¹ to R¹², and R¹⁴ to R¹⁷ are each the same as defined in Formula 1.

12. The light emitting element of claim 1, wherein the amine compound represented by Formula 1 is represented by Formula 8, and
the amine compound is a compound satisfying any one selected from among combinations shown in Compound Combination Table 1: 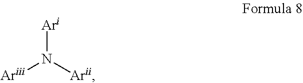
in Formula 8,
Arⁱ being any one selected from Substituent Group A,
Arⁱⁱ being any one selected from Substituent Group B, and
Arⁱⁱⁱ being any one selected from Substituent Group C:r 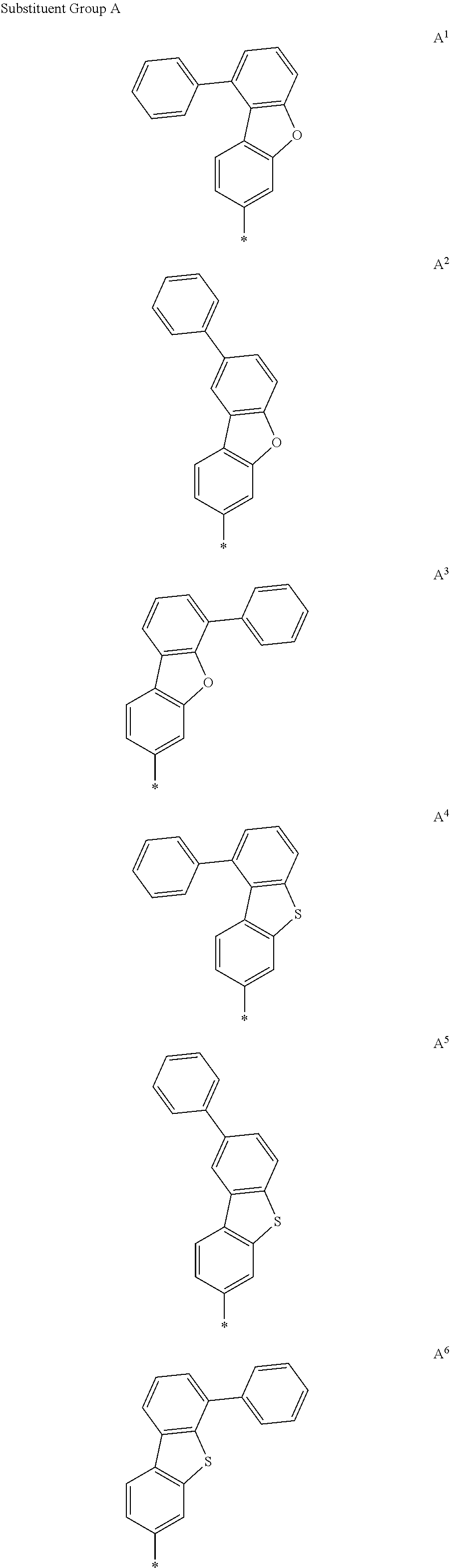 [SMILES: *c1ccc2c(c1)oc1cccc(-c3ccccc3)c12 ; *c1ccc2c(c1)oc1ccc(-c3ccccc3)cc12 ; *c1ccc2c(c1)oc1c(-c3ccccc3)cccc12 ; *c1ccc2c(c1)sc1cccc(-c3ccccc3)c12 ; *c1ccc2c(c1)sc1ccc(-c3ccccc3)cc12 ; *c1ccc2c(c1)sc1c(-c3ccccc3)cccc12] 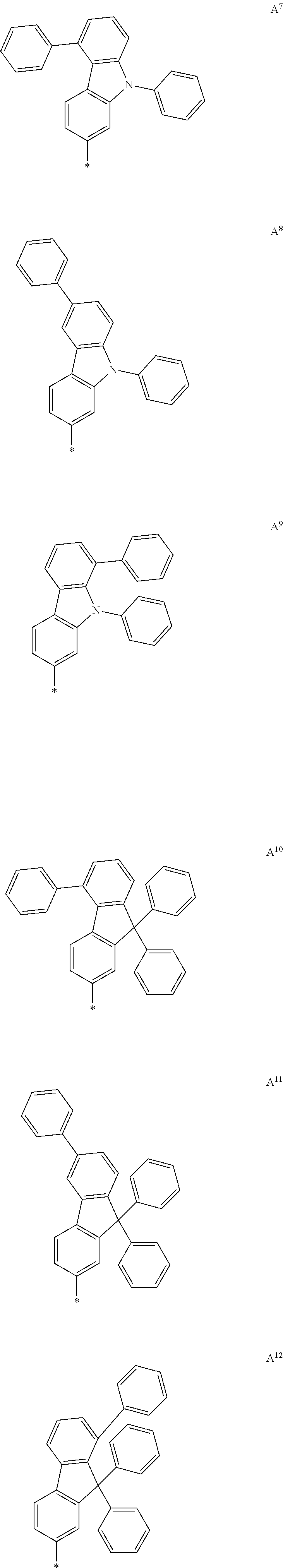 [SMILES: *c1ccc2c3c(-c4ccccc4)cccc3n(-c3ccccc3)c2c1 ; *c1ccc2c3cc(-c4ccccc4)ccc3n(-c3ccccc3)c2c1 ; *c1ccc2c3cccc(-c4ccccc4)c3n(-c3ccccc3)c2c1 ; *c1ccc2c(c1)C(c1ccccc1)(c1ccccc1)c1cccc(-c3ccccc3)c1-2 ; *c1ccc2c(c1)C(c1ccccc1)(c1ccccc1)c1ccc(-c3ccccc3)cc1-2 ; *c1ccc2c(c1)C(c1ccccc1)(c1ccccc1)c1c(-c3ccccc3)cccc1-2] 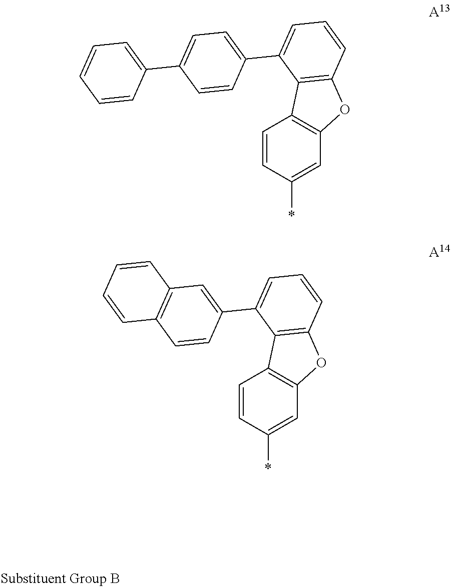 [SMILES: *c1ccc2c(c1)oc1cccc(-c3ccc(-c4ccccc4)cc3)c12 ; *c1ccc2c(c1)oc1cccc(-c3ccc4ccccc4c3)c12] 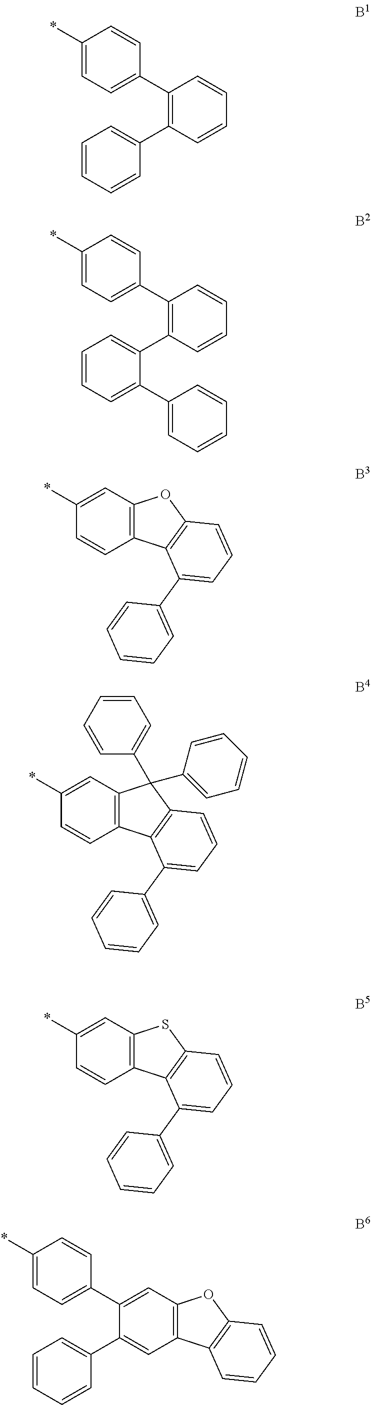 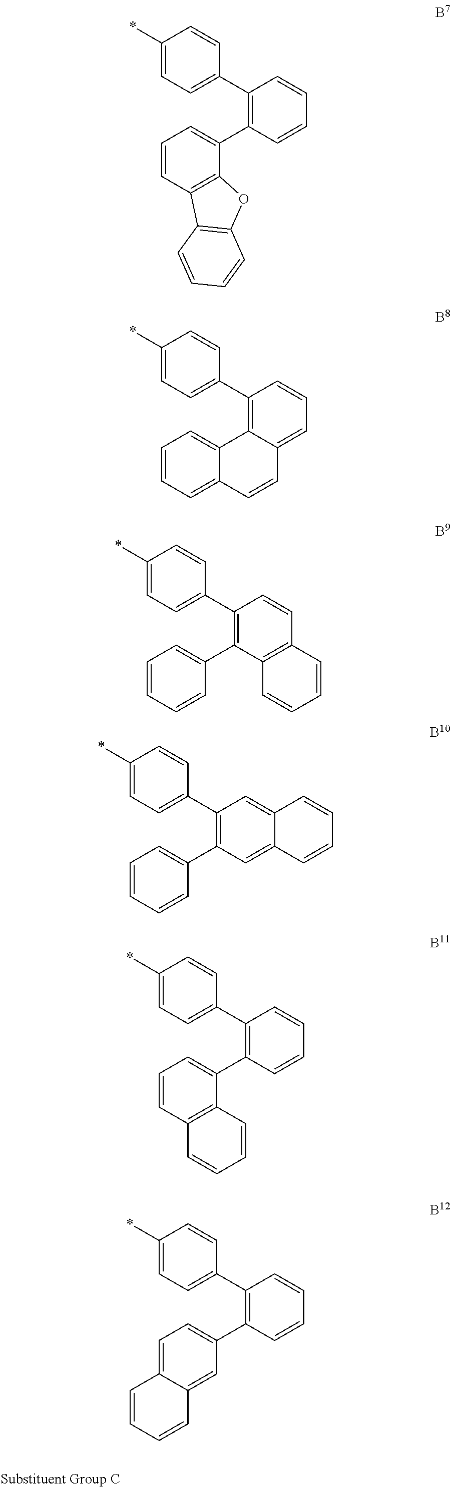 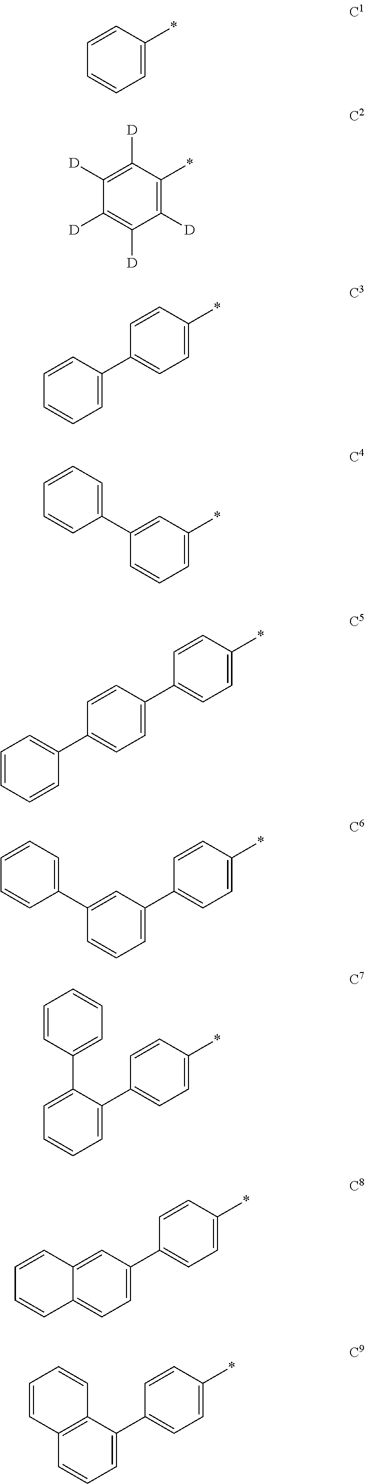 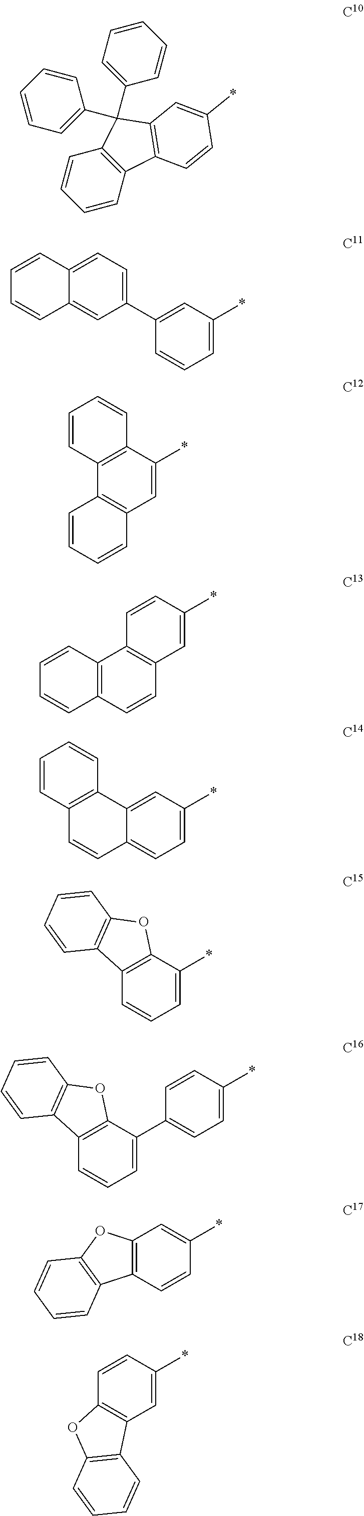 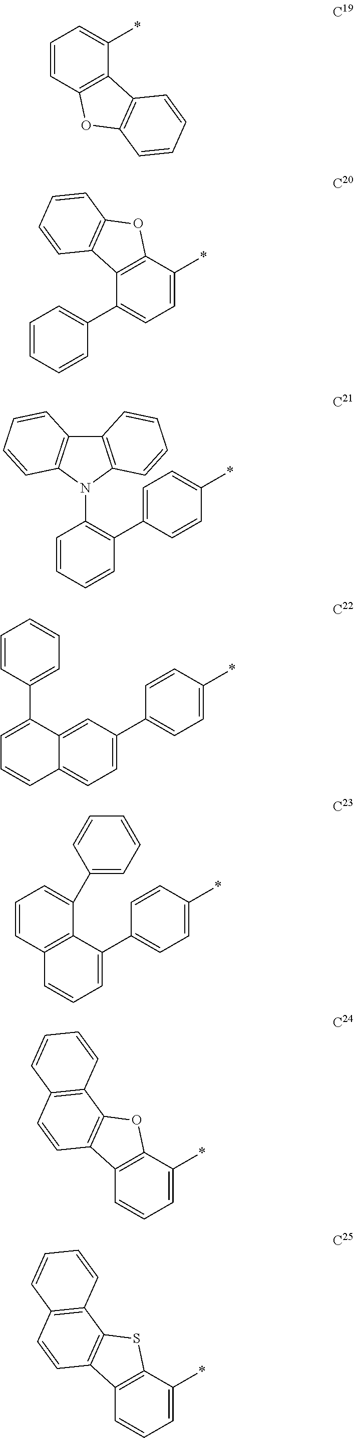 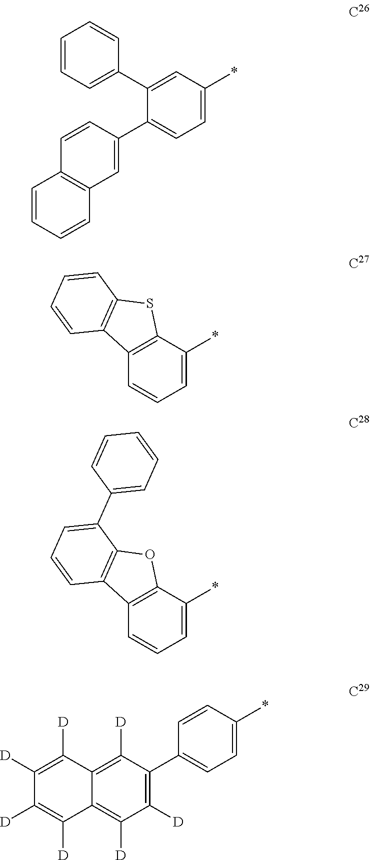
TABLE 1
Compound Combination

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 5 | 1 | 1 |
| 6 | 6 | 1 | 1 |
| 7 | 7 | 1 | 1 |
| 8 | 8 | 1 | 1 |
| 9 | 9 | 1 | 1 |
| 10 | 1 | 1 | 28 |
| 11 | 1 | 2 | 28 |
| 12 | 12 | 1 | 1 |
| 13 | 13 | 1 | 1 |
| 14 | 14 | 1 | 1 |
| 15 | 1 | 1 | 2 |
| 16 | 2 | 1 | 2 |
| 17 | 3 | 1 | 2 |
| 18 | 4 | 1 | 2 |
| 19 | 5 | 1 | 2 |
| 20 | 6 | 1 | 2 |
| 21 | 7 | 1 | 2 |
| 22 | 8 | 1 | 2 |
| 23 | 9 | 1 | 2 |
| 24 | 10 | 1 | 2 |
| 25 | 11 | 1 | 2 |
| 26 | 12 | 1 | 2 |
| 27 | 13 | 1 | 2 |
| 28 | 14 | 1 | 2 |
| 29 | 1 | 1 | 3 |
| 30 | 2 | 1 | 3 |
| 31 | 3 | 1 | 3 |
| 32 | 4 | 1 | 3 |
| 33 | 5 | 1 | 3 |
| 34 | 6 | 1 | 3 |
| 35 | 7 | 1 | 3 |
| 36 | 8 | 1 | 3 |
| 37 | 9 | 1 | 3 |
| 38 | 10 | 1 | 3 |
| 39 | 11 | 1 | 3 |
| 40 | 12 | 1 | 3 |
| 41 | 13 | 1 | 3 |
| 42 | 14 | 1 | 3 |
| 43 | 1 | 1 | 8 |
| 44 | 2 | 1 | 8 |
| 45 | 3 | 1 | 8 |
| 46 | 4 | 1 | 8 |
| 47 | 5 | 1 | 8 |
| 48 | 6 | 1 | 8 |
| 49 | 7 | 1 | 8 |
| 50 | 8 | 1 | 8 |
| 51 | 9 | 1 | 8 |
| 52 | 10 | 1 | 8 |
| 53 | 11 | 1 | 8 |
| 54 | 12 | 1 | 8 |
| 55 | 13 | 1 | 8 |
| 56 | 14 | 1 | 8 |
| 57 | 1 | 2 | 1 |
| 58 | 2 | 2 | 1 |
| 59 | 3 | 2 | 1 |
| 60 | 4 | 2 | 1 |
| 61 | 5 | 2 | 1 |
| 62 | 6 | 2 | 1 |
| 63 | 7 | 2 | 1 |
| 64 | 8 | 2 | 1 |
| 65 | 9 | 2 | 1 |
| 66 | 10 | 2 | 1 |
| 67 | 11 | 2 | 1 |
| 68 | 12 | 2 | 1 |
| 69 | 13 | 2 | 1 |
| 70 | 14 | 2 | 1 |
| 71 | 1 | 2 | 2 |
| 72 | 2 | 2 | 2 |
| 73 | 3 | 2 | 2 |
| 74 | 4 | 2 | 2 |
| 75 | 5 | 2 | 2 |
| 76 | 6 | 2 | 2 |
| 77 | 7 | 2 | 2 |
| 78 | 8 | 2 | 2 |
| 79 | 9 | 2 | 2 |
| 80 | 10 | 2 | 2 |
| 81 | 11 | 2 | 2 |
| 82 | 12 | 2 | 2 |
| 83 | 13 | 2 | 2 |
| 84 | 14 | 2 | 2 |
| 85 | 1 | 2 | 3 |
| 86 | 2 | 2 | 3 |
| 87 | 3 | 2 | 3 |
| 88 | 4 | 2 | 3 |
| 89 | 5 | 2 | 3 |
| 90 | 6 | 2 | 3 |
| 91 | 7 | 2 | 3 |
| 92 | 8 | 2 | 3 |
| 93 | 9 | 2 | 3 |
| 94 | 10 | 2 | 3 |
| 95 | 11 | 2 | 3 |
| 96 | 12 | 2 | 3 |
| 97 | 13 | 2 | 3 |
| 98 | 14 | 2 | 3 |
| 99 | 1 | 2 | 8 |
| 100 | 2 | 2 | 8 |
| 101 | 3 | 2 | 8 |
| 102 | 4 | 2 | 8 |
| 103 | 5 | 2 | 8 |
| 104 | 6 | 2 | 8 |
| 105 | 7 | 2 | 8 |
| 106 | 8 | 2 | 8 |
| 107 | 9 | 2 | 8 |
| 108 | 10 | 2 | 8 |
| 109 | 11 | 2 | 8 |
| 110 | 12 | 2 | 8 |
| 111 | 13 | 2 | 8 |
| 112 | 14 | 2 | 8 |
| 113 | 1 | 3 | 8 |
| 114 | 1 | 4 | 8 |
| 115 | 1 | 5 | 8 |
| 116 | 1 | 6 | 8 |
| 117 | 1 | 7 | 8 |
| 118 | 1 | 8 | 8 |
| 119 | 1 | 11 | 8 |
| 120 | 1 | 12 | 8 |
| 121 | 1 | 1 | 13 |
| 122 | 1 | 2 | 13 |
| 123 | 1 | 3 | 13 |
| 124 | 1 | 4 | 13 |
| 125 | 1 | 5 | 13 |
| 126 | 1 | 6 | 13 |
| 127 | 1 | 7 | 13 |
| 128 | 1 | 8 | 13 |
| 129 | 1 | 9 | 13 |
| 130 | 1 | 10 | 13 |
| 131 | 1 | 11 | 13 |
| 132 | 1 | 12 | 13 |
| 133 | 3 | 3 | 8 |
| 134 | 3 | 4 | 8 |
| 135 | 3 | 5 | 8 |
| 136 | 3 | 6 | 8 |
| 137 | 3 | 7 | 8 |
| 138 | 3 | 8 | 8 |
| 139 | 3 | 11 | 8 |
| 140 | 3 | 12 | 8 |
| 141 | 3 | 1 | 13 |
| 142 | 3 | 2 | 13 |
| 143 | 3 | 3 | 13 |
| 144 | 3 | 4 | 13 |
| 145 | 3 | 5 | 13 |
| 146 | 3 | 6 | 13 |
| 147 | 3 | 7 | 13 |
| 148 | 3 | 8 | 13 |
| 149 | 3 | 9 | 13 |
| 150 | 3 | 10 | 13 |
| 151 | 3 | 11 | 13 |
| 152 | 3 | 12 | 13 |
| 153 | 1 | 1 | 4 |
| 154 | 1 | 1 | 5 |
| 155 | 1 | 1 | 6 |
| 156 | 1 | 1 | 7 |
| 157 | 1 | 1 | 9 |
| 158 | 1 | 1 | 10 |
| 159 | 1 | 1 | 11 |
| 160 | 1 | 1 | 12 |
| 161 | 1 | 1 | 14 |
| 162 | 1 | 1 | 15 |
| 163 | 1 | 1 | 16 |
| 164 | 1 | 1 | 17 |
| 165 | 1 | 1 | 18 |
| 166 | 1 | 1 | 19 |
| 167 | 1 | 1 | 20 |
| 168 | 1 | 1 | 21 |
| 169 | 1 | 1 | 22 |
| 170 | 1 | 1 | 23 |
| 171 | 1 | 1 | 24 |
| 172 | 1 | 1 | 25 |
| 173 | 1 | 1 | 26 |
| 174 | 1 | 1 | 27 |
| 175 | 1 | 2 | 4 |
| 176 | 1 | 2 | 5 |
| 177 | 1 | 2 | 6 |
| 178 | 1 | 2 | 7 |
| 179 | 1 | 2 | 9 |
| 180 | 1 | 2 | 10 |
| 181 | 1 | 2 | 11 |
| 182 | 1 | 2 | 12 |
| 183 | 1 | 2 | 14 |
| 184 | 1 | 2 | 15 |
| 185 | 1 | 2 | 16 |
| 186 | 1 | 2 | 17 |
| 187 | 1 | 2 | 18 |
| 188 | 1 | 2 | 19 |
| 189 | 1 | 2 | 20 |
| 190 | 1 | 2 | 21 |
| 191 | 1 | 2 | 22 |
| 192 | 1 | 2 | 23 |
| 193 | 1 | 2 | 24 |
| 194 | 1 | 2 | 25 |
| 195 | 1 | 2 | 26 |
| 196 | 1 | 2 | 27 |
| 197 | 1 | 7 | 1 |
| 198 | 1 | 7 | 2 |
| 199 | 1 | 7 | 3 |
| 200 | 1 | 7 | 4 |
| 201 | 1 | 7 | 5 |
| 202 | 1 | 7 | 6 |
| 203 | 1 | 7 | 7 |
| 204 | 1 | 7 | 9 |
| 205 | 1 | 7 | 10 |
| 206 | 1 | 7 | 11 |
| 207 | 1 | 7 | 12 |
| 208 | 1 | 7 | 14 |
| 209 | 1 | 7 | 15 |
| 210 | 1 | 7 | 16 |
| 211 | 1 | 7 | 17 |
| 212 | 1 | 7 | 18 |
| 213 | 1 | 7 | 19 |
| 214 | 1 | 7 | 20 |
| 215 | 1 | 7 | 21 |
| 216 | 1 | 7 | 22 |
| 217 | 1 | 7 | 23 |
| 218 | 1 | 7 | 24 |
| 219 | 1 | 7 | 25 |
| 220 | 1 | 7 | 26 |
| 221 | 1 | 7 | 27 |
| 222 | 1 | 11 | 1 |
| 223 | 1 | 11 | 2 |
| 224 | 1 | 11 | 3 |
| 225 | 1 | 11 | 4 |
| 226 | 1 | 11 | 5 |
| 227 | 1 | 11 | 6 |
| 228 | 1 | 11 | 7 |
| 229 | 1 | 11 | 9 |
| 230 | 1 | 11 | 10 |
| 231 | 1 | 11 | 11 |
| 232 | 1 | 11 | 12 |
| 233 | 1 | 11 | 14 |
| 234 | 1 | 11 | 15 |
| 235 | 1 | 11 | 16 |
| 236 | 1 | 11 | 17 |
| 237 | 1 | 11 | 18 |
| 238 | 1 | 11 | 19 |
| 239 | 1 | 11 | 20 |
| 240 | 1 | 11 | 21 |
| 241 | 1 | 11 | 22 |
| 242 | 1 | 11 | 23 |
| 243 | 1 | 11 | 24 |
| 244 | 1 | 11 | 25 |
| 245 | 1 | 11 | 26 |
| 246 | 1 | 11 | 27 |
| 247 | 3 | 1 | 4 |
| 248 | 3 | 1 | 5 |
| 249 | 3 | 1 | 6 |
| 250 | 3 | 1 | 7 |
| 251 | 3 | 1 | 9 |
| 252 | 3 | 1 | 10 |
| 253 | 3 | 1 | 11 |
| 254 | 3 | 1 | 12 |
| 255 | 3 | 1 | 14 |
| 256 | 3 | 1 | 15 |
| 257 | 3 | 1 | 16 |
| 258 | 3 | 1 | 17 |
| 259 | 3 | 1 | 18 |
| 260 | 3 | 1 | 19 |
| 261 | 3 | 1 | 20 |
| 262 | 3 | 1 | 21 |
| 263 | 3 | 1 | 22 |
| 264 | 3 | 1 | 23 |
| 265 | 3 | 1 | 24 |
| 266 | 3 | 1 | 25 |
| 267 | 3 | 1 | 26 |
| 268 | 3 | 1 | 27 |
| 269 | 3 | 2 | 4 |
| 270 | 3 | 2 | 5 |
| 271 | 3 | 2 | 6 |
| 272 | 3 | 2 | 7 |
| 273 | 3 | 2 | 9 |
| 274 | 3 | 2 | 10 |
| 275 | 3 | 2 | 11 |
| 276 | 3 | 2 | 12 |
| 277 | 3 | 2 | 14 |
| 278 | 3 | 2 | 15 |
| 279 | 3 | 2 | 16 |
| 280 | 3 | 2 | 17 |
| 281 | 3 | 2 | 18 |
| 282 | 3 | 2 | 19 |
| 283 | 3 | 2 | 20 |
| 284 | 3 | 2 | 21 |
| 285 | 3 | 2 | 22 |
| 286 | 3 | 2 | 23 |
| 287 | 3 | 2 | 24 |
| 288 | 3 | 2 | 25 |
| 289 | 3 | 2 | 26 |
| 290 | 3 | 2 | 27 |
| 291 | 3 | 7 | 1 |
| 292 | 3 | 7 | 2 |
| 293 | 3 | 7 | 3 |
| 294 | 3 | 7 | 4 |
| 295 | 3 | 7 | 5 |
| 296 | 3 | 7 | 6 |
| 297 | 3 | 7 | 7 |
| 298 | 3 | 7 | 9 |
| 299 | 3 | 7 | 10 |
| 300 | 3 | 7 | 11 |
| 301 | 3 | 7 | 12 |
| 302 | 3 | 7 | 14 |
| 303 | 3 | 7 | 15 |
| 304 | 3 | 7 | 16 |
| 305 | 3 | 7 | 17 |
| 306 | 3 | 7 | 18 |
| 307 | 3 | 7 | 19 |
| 308 | 3 | 7 | 20 |
| 309 | 3 | 7 | 21 |
| 310 | 3 | 7 | 22 |
| 311 | 3 | 7 | 23 |
| 312 | 3 | 7 | 24 |
| 313 | 3 | 7 | 25 |
| 314 | 3 | 7 | 26 |
| 315 | 3 | 7 | 27 |
| 316 | 3 | 11 | 1 |
| 317 | 3 | 11 | 2 |
| 318 | 3 | 11 | 3 |
| 319 | 3 | 11 | 4 |
| 320 | 3 | 11 | 5 |
| 321 | 3 | 11 | 6 |
| 322 | 3 | 11 | 7 |
| 323 | 3 | 11 | 9 |
| 324 | 3 | 11 | 10 |
| 325 | 3 | 11 | 11 |
| 326 | 3 | 11 | 12 |
| 327 | 3 | 11 | 14 |
| 328 | 3 | 11 | 15 |
| 329 | 3 | 11 | 16 |
| 330 | 3 | 11 | 17 |
| 331 | 3 | 11 | 18 |
| 332 | 3 | 11 | 19 |
| 333 | 3 | 11 | 20 |
| 334 | 3 | 11 | 21 |
| 335 | 3 | 11 | 22 |
| 336 | 3 | 11 | 23 |
| 337 | 3 | 11 | 24 |
| 338 | 3 | 11 | 25 |
| 339 | 3 | 11 | 26 |
| 340 | 3 | 11 | 27 |
| 341 | 4 | 1 | 4 |
| 342 | 4 | 1 | 5 |
| 343 | 4 | 1 | 6 |
| 344 | 4 | 1 | 7 |
| 345 | 4 | 1 | 9 |
| 346 | 4 | 1 | 10 |
| 347 | 4 | 1 | 11 |
| 348 | 4 | 1 | 12 |
| 349 | 4 | 1 | 13 |
| 350 | 4 | 1 | 14 |
| 351 | 4 | 1 | 15 |
| 352 | 4 | 1 | 16 |
| 353 | 4 | 1 | 17 |
| 354 | 4 | 1 | 18 |
| 355 | 4 | 1 | 19 |
| 356 | 4 | 1 | 20 |
| 357 | 4 | 1 | 21 |
| 358 | 4 | 1 | 22 |
| 359 | 4 | 1 | 23 |
| 360 | 4 | 1 | 24 |
| 361 | 4 | 1 | 25 |
| 362 | 4 | 1 | 26 |
| 363 | 4 | 1 | 27 |
| 364 | 4 | 2 | 4 |
| 365 | 4 | 2 | 5 |
| 366 | 4 | 2 | 6 |
| 367 | 4 | 2 | 7 |
| 368 | 4 | 2 | 9 |
| 369 | 4 | 2 | 10 |
| 370 | 4 | 2 | 11 |
| 371 | 4 | 2 | 12 |
| 372 | 4 | 2 | 13 |
| 373 | 4 | 2 | 14 |
| 374 | 4 | 2 | 15 |
| 375 | 4 | 2 | 16 |
| 376 | 4 | 2 | 17 |
| 377 | 4 | 2 | 18 |
| 378 | 4 | 2 | 19 |
| 379 | 4 | 2 | 20 |
| 380 | 4 | 2 | 21 |
| 381 | 4 | 2 | 22 |
| 382 | 4 | 2 | 23 |
| 383 | 4 | 2 | 24 |
| 384 | 4 | 2 | 25 |
| 385 | 4 | 2 | 26 |
| 386 | 4 | 2 | 27 |
| 387 | 4 | 7 | 1 |
| 388 | 4 | 7 | 2 |
| 389 | 4 | 7 | 3 |
| 390 | 4 | 7 | 4 |
| 391 | 4 | 7 | 5 |
| 392 | 4 | 7 | 6 |
| 393 | 4 | 7 | 7 |
| 394 | 4 | 7 | 8 |
| 395 | 4 | 7 | 9 |
| 396 | 4 | 7 | 10 |
| 397 | 4 | 7 | 11 |
| 398 | 4 | 7 | 12 |
| 399 | 4 | 7 | 13 |
| 400 | 4 | 7 | 14 |
| 401 | 4 | 7 | 15 |
| 402 | 4 | 7 | 16 |
| 403 | 4 | 7 | 17 |
| 404 | 4 | 7 | 18 |
| 405 | 4 | 7 | 19 |
| 406 | 4 | 7 | 20 |
| 407 | 4 | 7 | 21 |
| 408 | 4 | 7 | 22 |
| 409 | 4 | 7 | 23 |
| 410 | 4 | 7 | 24 |
| 411 | 4 | 7 | 25 |
| 412 | 4 | 7 | 26 |
| 413 | 4 | 7 | 27 |
| 414 | 4 | 11 | 1 |
| 415 | 4 | 11 | 2 |
| 416 | 4 | 11 | 3 |
| 417 | 4 | 11 | 4 |
| 418 | 4 | 11 | 5 |
| 419 | 4 | 11 | 6 |
| 420 | 4 | 11 | 7 |
| 421 | 4 | 11 | 8 |
| 422 | 4 | 11 | 9 |
| 423 | 4 | 11 | 10 |
| 424 | 4 | 11 | 11 |
| 425 | 4 | 11 | 12 |
| 426 | 4 | 11 | 13 |
| 427 | 4 | 11 | 14 |
| 428 | 4 | 11 | 15 |
| 429 | 4 | 11 | 16 |
| 430 | 4 | 11 | 17 |
| 431 | 4 | 11 | 18 |
| 432 | 4 | 11 | 19 |
| 433 | 4 | 11 | 20 |
| 434 | 4 | 11 | 21 |
| 435 | 4 | 11 | 22 |
| 436 | 4 | 11 | 23 |
| 437 | 4 | 11 | 24 |
| 438 | 4 | 11 | 25 |
| 439 | 4 | 11 | 26 |
| 440 | 4 | 11 | 27 |
| 441 | 7 | 1 | 4 |
| 442 | 7 | 1 | 5 |
| 443 | 7 | 1 | 6 |
| 444 | 7 | 1 | 7 |
| 445 | 7 | 1 | 9 |
| 446 | 7 | 1 | 10 |
| 447 | 7 | 1 | 11 |
| 448 | 7 | 1 | 12 |
| 449 | 7 | 1 | 13 |
| 450 | 7 | 1 | 14 |
| 451 | 7 | 1 | 15 |
| 452 | 7 | 1 | 16 |
| 453 | 7 | 1 | 17 |
| 454 | 7 | 1 | 18 |
| 455 | 7 | 1 | 19 |
| 456 | 7 | 1 | 20 |
| 457 | 7 | 1 | 21 |
| 458 | 7 | 1 | 22 |
| 459 | 7 | 1 | 23 |
| 460 | 7 | 1 | 24 |
| 461 | 7 | 1 | 25 |
| 462 | 7 | 1 | 26 |
| 463 | 7 | 1 | 27 |
| 464 | 7 | 2 | 4 |
| 465 | 7 | 2 | 5 |
| 466 | 7 | 2 | 6 |
| 467 | 7 | 2 | 7 |
| 468 | 7 | 2 | 9 |
| 469 | 7 | 2 | 10 |
| 470 | 7 | 2 | 11 |
| 471 | 7 | 2 | 12 |
| 472 | 7 | 2 | 13 |
| 473 | 7 | 2 | 14 |
| 474 | 7 | 2 | 15 |
| 475 | 7 | 2 | 16 |
| 476 | 7 | 2 | 17 |
| 477 | 7 | 2 | 18 |
| 478 | 7 | 2 | 19 |
| 479 | 7 | 2 | 20 |
| 480 | 7 | 2 | 21 |
| 481 | 7 | 2 | 22 |
| 482 | 7 | 2 | 23 |
| 483 | 7 | 2 | 24 |
| 484 | 7 | 2 | 25 |
| 485 | 7 | 2 | 26 |
| 486 | 7 | 2 | 27 |
| 487 | 7 | 7 | 1 |
| 488 | 7 | 7 | 2 |
| 489 | 7 | 7 | 3 |
| 490 | 7 | 7 | 4 |
| 491 | 7 | 7 | 5 |
| 492 | 7 | 7 | 6 |
| 493 | 7 | 7 | 7 |
| 494 | 7 | 7 | 8 |
| 495 | 7 | 7 | 9 |
| 496 | 7 | 7 | 10 |
| 497 | 7 | 7 | 11 |
| 498 | 7 | 7 | 12 |
| 499 | 7 | 7 | 13 |
| 500 | 7 | 7 | 14 |
| 501 | 7 | 7 | 15 |
| 502 | 7 | 7 | 16 |
| 503 | 7 | 7 | 17 |
| 504 | 7 | 7 | 18 |
| 505 | 7 | 7 | 19 |
| 506 | 7 | 7 | 20 |
| 507 | 7 | 7 | 21 |
| 508 | 7 | 7 | 22 |
| 509 | 7 | 7 | 23 |
| 510 | 7 | 7 | 24 |
| 511 | 7 | 7 | 25 |
| 512 | 7 | 7 | 26 |
| 513 | 7 | 7 | 27 |
| 514 | 7 | 11 | 1 |
| 515 | 7 | 11 | 2 |
| 516 | 7 | 11 | 3 |
| 517 | 7 | 11 | 4 |
| 518 | 7 | 11 | 5 |
| 519 | 7 | 11 | 6 |
| 520 | 7 | 11 | 7 |
| 521 | 7 | 11 | 8 |
| 522 | 7 | 11 | 9 |
| 523 | 7 | 11 | 10 |
| 524 | 7 | 11 | 11 |
| 525 | 7 | 11 | 12 |
| 526 | 7 | 11 | 13 |
| 527 | 7 | 11 | 14 |
| 528 | 7 | 11 | 15 |
| 529 | 7 | 11 | 16 |
| 530 | 7 | 11 | 17 |
| 531 | 7 | 11 | 18 |
| 532 | 7 | 11 | 19 |
| 533 | 7 | 11 | 20 |
| 534 | 7 | 11 | 21 |
| 535 | 7 | 11 | 22 |
| 536 | 7 | 11 | 23 |
| 537 | 7 | 11 | 24 |
| 538 | 7 | 11 | 25 |
| 539 | 7 | 11 | 26 |
| 540 | 7 | 11 | 27 |
| 541 | 10 | 1 | 4 |
| 542 | 10 | 1 | 5 |
| 543 | 10 | 1 | 6 |
| 544 | 10 | 1 | 7 |
| 545 | 10 | 1 | 9 |
| 546 | 10 | 1 | 10 |
| 547 | 10 | 1 | 11 |
| 548 | 10 | 1 | 12 |
| 549 | 10 | 1 | 13 |
| 550 | 10 | 1 | 14 |
| 551 | 10 | 1 | 15 |
| 552 | 10 | 1 | 16 |
| 553 | 10 | 1 | 17 |
| 554 | 10 | 1 | 18 |
| 555 | 10 | 1 | 19 |
| 556 | 10 | 1 | 20 |
| 557 | 10 | 1 | 21 |
| 558 | 10 | 1 | 22 |
| 559 | 10 | 1 | 23 |
| 560 | 10 | 1 | 24 |
| 561 | 10 | 1 | 25 |
| 562 | 10 | 1 | 26 |
| 563 | 10 | 1 | 27 |
| 564 | 10 | 2 | 4 |
| 565 | 10 | 2 | 5 |
| 566 | 10 | 2 | 6 |
| 567 | 10 | 2 | 7 |
| 568 | 10 | 2 | 9 |
| 569 | 10 | 2 | 10 |
| 570 | 10 | 2 | 11 |
| 571 | 10 | 2 | 12 |
| 572 | 10 | 2 | 13 |
| 573 | 10 | 2 | 14 |
| 574 | 10 | 2 | 15 |
| 575 | 10 | 2 | 16 |
| 576 | 10 | 2 | 17 |
| 577 | 10 | 2 | 18 |
| 578 | 10 | 2 | 19 |
| 579 | 10 | 2 | 20 |
| 580 | 10 | 2 | 21 |
| 581 | 10 | 2 | 22 |
| 582 | 10 | 2 | 23 |
| 583 | 10 | 2 | 24 |
| 584 | 10 | 2 | 25 |
| 585 | 10 | 2 | 26 |
| 586 | 10 | 2 | 27 |
| 587 | 10 | 7 | 1 |
| 588 | 10 | 7 | 2 |
| 589 | 10 | 7 | 3 |
| 590 | 10 | 7 | 4 |
| 591 | 10 | 7 | 5 |
| 592 | 10 | 7 | 6 |
| 593 | 10 | 7 | 7 |
| 594 | 10 | 7 | 8 |
| 595 | 10 | 7 | 9 |
| 596 | 10 | 7 | 10 |
| 597 | 10 | 7 | 11 |
| 598 | 10 | 7 | 12 |
| 599 | 10 | 7 | 13 |
| 600 | 10 | 7 | 14 |
| 601 | 10 | 7 | 15 |
| 602 | 10 | 7 | 16 |
| 603 | 10 | 7 | 17 |
| 604 | 10 | 7 | 18 |
| 605 | 10 | 7 | 19 |
| 606 | 10 | 7 | 20 |
| 607 | 10 | 7 | 21 |
| 608 | 10 | 7 | 22 |
| 609 | 10 | 7 | 23 |
| 610 | 10 | 7 | 24 |
| 611 | 10 | 7 | 25 |
| 612 | 10 | 7 | 26 |
| 613 | 10 | 7 | 27 |
| 614 | 10 | 11 | 1 |
| 615 | 10 | 11 | 2 |
| 616 | 10 | 11 | 3 |
| 617 | 10 | 11 | 4 |
| 618 | 10 | 11 | 5 |
| 619 | 10 | 11 | 6 |
| 620 | 10 | 11 | 7 |
| 621 | 10 | 11 | 8 |
| 622 | 10 | 11 | 9 |
| 623 | 10 | 11 | 10 |
| 624 | 10 | 11 | 11 |
| 625 | 10 | 11 | 12 |
| 626 | 10 | 11 | 13 |
| 627 | 10 | 11 | 14 |
| 628 | 10 | 11 | 15 |
| 629 | 10 | 11 | 16 |
| 630 | 10 | 11 | 17 |
| 631 | 10 | 11 | 18 |
| 632 | 10 | 11 | 19 |
| 633 | 10 | 11 | 20 |
| 634 | 10 | 11 | 21 |
| 635 | 10 | 11 | 22 |
| 636 | 10 | 11 | 23 |
| 637 | 10 | 11 | 24 |
| 638 | 10 | 11 | 25 |
| 639 | 10 | 11 | 26 |
| 640 | 10 | 11 | 27 |
| 641 | 3 | 1 | 29. |

13. An electronic apparatus comprising:
a base layer;
a circuit layer on the base layer; and
a display element layer on the circuit layer and comprising a light emitting element,
wherein the light emitting element comprises a first electrode, a second electrode on the first electrode, and at least one functional layer between the first electrode and the second electrode and containing an amine compound represented by Formula 1: 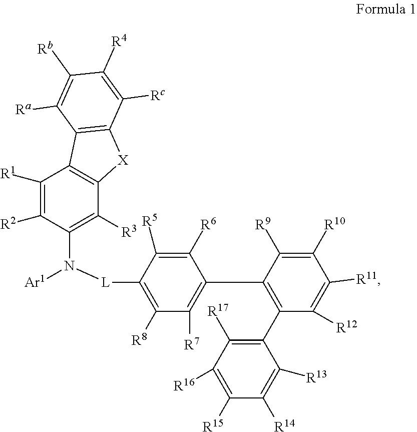
wherein in Formula 1,
X is O, S, NAr², or CAr³Ar⁴,
R^{a} to R^{c} are each independently hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one selected from among R^{a} to R^{c} is a substituted or unsubstituted aryl group having 6 to 30 ring-forming carbon atoms,
R¹ to R⁴ are each independently hydrogen, deuterium, a substituted or unsubstituted alkyl group having 1 to 20 carbon atoms, or a substituted or unsubstituted aryl group having 6 to 30 ring-forming carbon atoms,
Ar¹ is a substituted or unsubstituted aryl group having 6 to 30 ring-forming carbon atoms or a substituted or unsubstituted heteroaryl group having 2 to 30 ring-forming carbon atoms,
Ar² to Ar⁴ are each independently a substituted or unsubstituted aryl group having 6 to 30 ring-forming carbon atoms,
L is a direct linkage, a substituted or unsubstituted arylene group having 6 to 30 ring-forming carbon atoms, or a substituted or unsubstituted heteroarylene group having 2 to 30 ring-forming carbon atoms,
R⁵ to R¹⁷ are each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring,
in Formula 1, a case in which L is a direct linkage and R⁹ and R¹⁰, and R¹¹ and R¹² are each bonded together to form a ring is excluded,
in Formula 1, a case in which L is a direct linkage and any of the pairs R¹² and R¹³, and R¹² and R¹⁷ are bonded together to form a ring is excluded,
in Formula 1, a case in which L is a direct linkage and R¹¹ and R¹² are bonded together to form a nitrogen-containing heterocycle is excluded,
in Formula 1, a case in which when X is CAr³Ar⁴, Ar¹ is an unsubstituted phenyl group is excluded,
a case in which the amine compound represented by Formula 1 contains a fluorene moiety substituted with at least one alkyl group at position 9 or unsubstituted, a substituted or unsubstituted 9-fluorene moiety, a substituted or unsubstituted spirobifluorene moiety, or a substituted or unsubstituted N,N-bis(4-(2-naphthyl)phenyl)amine moiety is excluded, and
at least one hydrogen of the amine compound represented by Formula 1 is optionally substituted with deuterium.

14. The electronic apparatus of claim 13, wherein the light emitting element further comprises a capping layer on the second electrode, and
wherein the capping layer has a refractive index of about 1.6 or greater in a wavelength range of about 550 nm to about 660 nm.

15. The electronic apparatus of claim 13, further comprising a light control layer on the display element layer and comprising quantum dots,
wherein the light emitting element is to emit first color light,
the light control layer comprises:
a first light control portion comprising a first quantum dot that converts the first color light into second color light having a longer wavelength than the first color light;
a second light control portion comprising a second quantum dot that converts the first color light into third color light having a longer wavelength than the first color light and the second color light; and
a third light control portion that transmits the first color light.

16. The electronic apparatus of claim 15, further comprising a color filter layer on the light control layer,
wherein the color filter layer comprises:
a first filter that transmits the second color light;
a second filter that transmits the third color light; and
a third filter that transmits the first color light.

17. An amine compound represented by Formula 1: 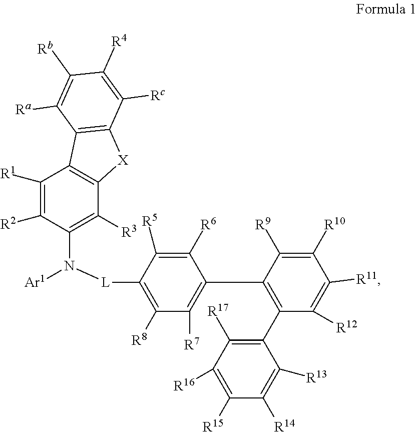
wherein in Formula 1,
X is O, S, NAr², or CAr³Ar⁴,
R^{a} to R^{c} are each independently hydrogen, deuterium, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms,
at least one selected from among R^{a} to R^{c} is a substituted or unsubstituted aryl group having 6 to 30 ring-forming carbon atoms,
R¹ to R⁴ are each independently hydrogen, deuterium, a substituted or unsubstituted alkyl group having 1 to 20 carbon atoms, or a substituted or unsubstituted aryl group having 6 to 30 ring-forming carbon atoms,
Ar¹ is a substituted or unsubstituted aryl group having 6 to 30 ring-forming carbon atoms or a substituted or unsubstituted heteroaryl group having 2 to 30 ring-forming carbon atoms,
Ar² to Ar⁴ are each independently a substituted or unsubstituted aryl group having 6 to 30 ring-forming carbon atoms,
L is a direct linkage, a substituted or unsubstituted arylene group having 6 to 30 ring-forming carbon atoms, or a substituted or unsubstituted heteroarylene group having 2 to 30 ring-forming carbon atoms,
R⁵ to R¹⁷ are each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring,
in Formula 1, a case in which L is a direct linkage and R⁹ and R¹⁰, and R¹¹ and R¹² are each bonded together to form a ring is excluded,
in Formula 1, a case in which L is a direct linkage and any of the pairs R¹² and R¹³, and R¹² and R¹⁷ are bonded together to form a ring is excluded,
in Formula 1, a case in which L is a direct linkage and R¹¹ and R¹² are bonded together to form a nitrogen-containing heterocycle is excluded,
in Formula 1, a case in which when X is CAr³Ar⁴, Ar¹ is an unsubstituted phenyl group is excluded,
a case in which the amine compound represented by Formula 1 contains a fluorene moiety substituted with at least one alkyl group at position 9 or unsubstituted, a substituted or unsubstituted 9-fluorene moiety, a substituted or unsubstituted spirobifluorene moiety, or a substituted or unsubstituted N,N-bis(4-(2-naphthyl)phenyl)amine moiety is excluded, and
at least one hydrogen of the amine compound represented by Formula 1 is optionally substituted with deuterium.

18. The amine compound of claim 17, wherein the amine compound represented by Formula 1 is represented by Formula 3: 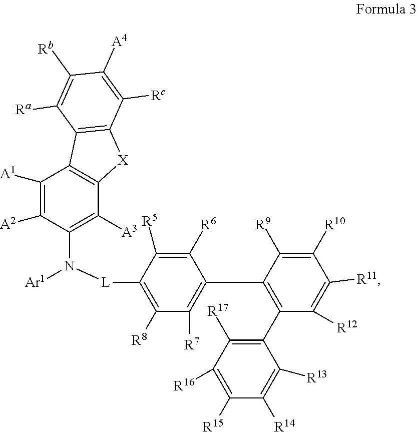
in Formula 3,
A¹ to A⁴ being each independently hydrogen or deuterium, and
Ar¹, L, X, R^{a}, R^{b}, R^{c}, and R⁵ to R¹⁷ each being the same as defined in Formula 1.

19. The amine compound of claim 17, wherein the amine compound represented by Formula 1 is represented by Formula 4-1 or Formula 4-2: 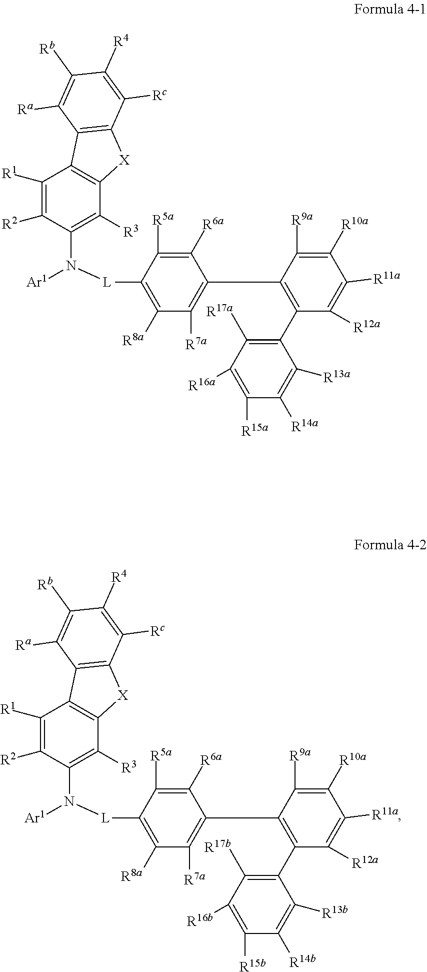
in Formula 4-1,
R^{5a} to R^{17a} being each independently hydrogen, deuterium, a substituted or unsubstituted alkyl group having 1 to 20 carbon atoms, or a substituted or unsubstituted aryl group having 6 to 30 ring-forming carbon atoms,
in Formula 4-2,
R^{5a} to R^{12a} being each independently hydrogen, deuterium, a substituted or unsubstituted alkyl group having 1 to 20 carbon atoms, or a substituted or unsubstituted aryl group having 6 to 30 ring-forming carbon atoms,
R^{13b} to R^{17b} being each independently hydrogen, deuterium, a substituted or unsubstituted alkyl group having 1 to 20 carbon atoms, a substituted or unsubstituted alkenyl group having 2 to 20 carbon atoms, or a substituted or unsubstituted aryl group having 6 to 30 ring-forming carbon atoms, and/or bonded to an adjacent group to form a ring, and
in Formulas 4-1 and 4-2,
Ar¹, L, X, R^{a}, R^{b}, R^{c}, and R¹ to R⁴ each being the same as defined in Formula 1.

20. The amine compound of claim 17, wherein the amine compound represented by Formula 1 is represented by Formula 8, and
the amine compound is a compound satisfying any one selected from among combinations shown in Compound Combination Table 1: 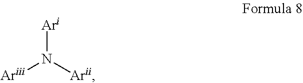
in Formula 8,
Arⁱ being any one selected from Substituent Group A,
Arⁱⁱ being any one selected from Substituent Group B, and
Arⁱⁱⁱ being any one selected from Substituent Group C: 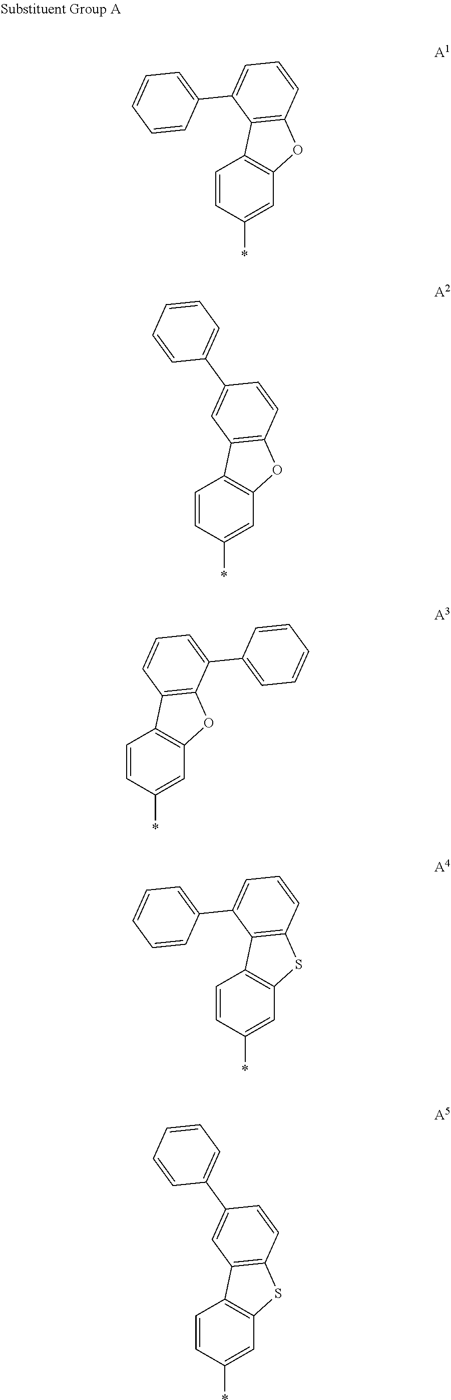 [SMILES: *c1ccc2c(c1)oc1cccc(-c3ccccc3)c12 ; *c1ccc2c(c1)oc1ccc(-c3ccccc3)cc12 ; *c1ccc2c(c1)oc1c(-c3ccccc3)cccc12 ; *c1ccc2c(c1)sc1cccc(-c3ccccc3)c12 ; *c1ccc2c(c1)sc1ccc(-c3ccccc3)cc12] 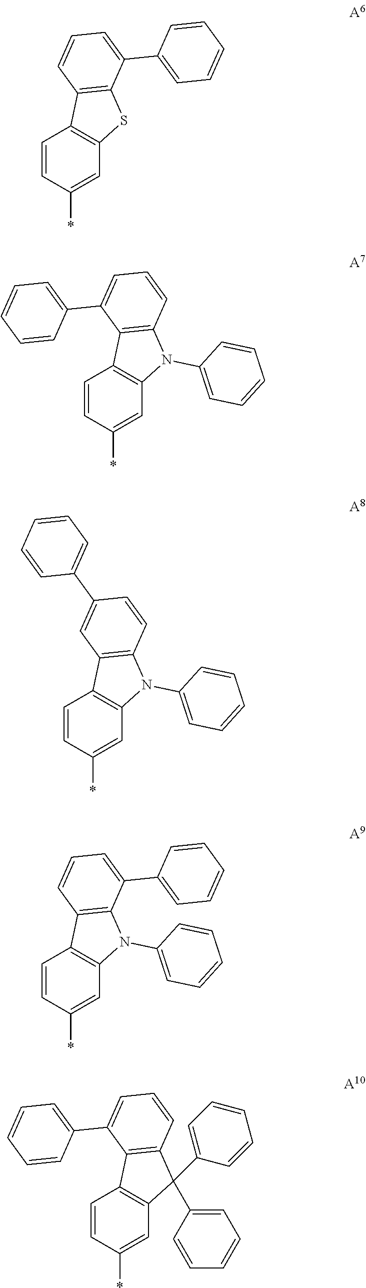 [SMILES: *c1ccc2c(c1)sc1c(-c3ccccc3)cccc12 ; *c1ccc2c3c(-c4ccccc4)cccc3n(-c3ccccc3)c2c1 ; *c1ccc2c3cc(-c4ccccc4)ccc3n(-c3ccccc3)c2c1 ; *c1ccc2c3cccc(-c4ccccc4)c3n(-c3ccccc3)c2c1 ; *c1ccc2c(c1)C(c1ccccc1)(c1ccccc1)c1cccc(-c3ccccc3)c1-2] 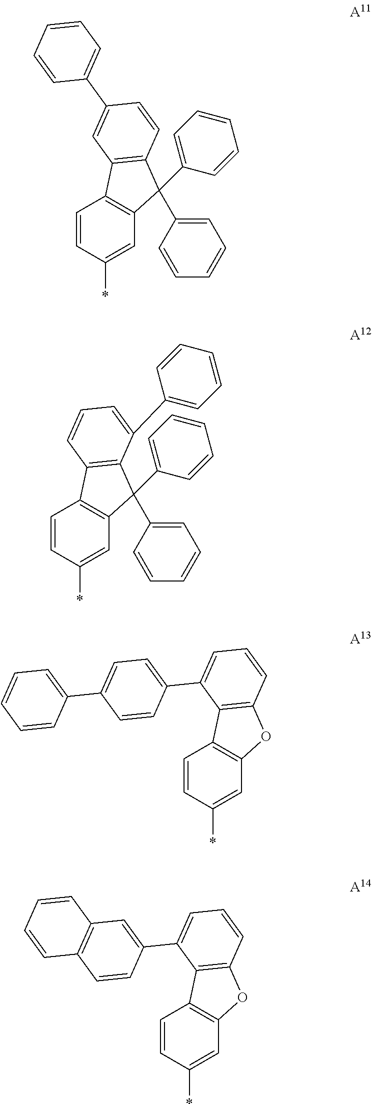 [SMILES: *c1ccc2c(c1)C(c1ccccc1)(c1ccccc1)c1ccc(-c3ccccc3)cc1-2 ; *c1ccc2c(c1)C(c1ccccc1)(c1ccccc1)c1c(-c3ccccc3)cccc1-2 ; *c1ccc2c(c1)oc1cccc(-c3ccc(-c4ccccc4)cc3)c12 ; *c1ccc2c(c1)oc1cccc(-c3ccc4ccccc4c3)c12] 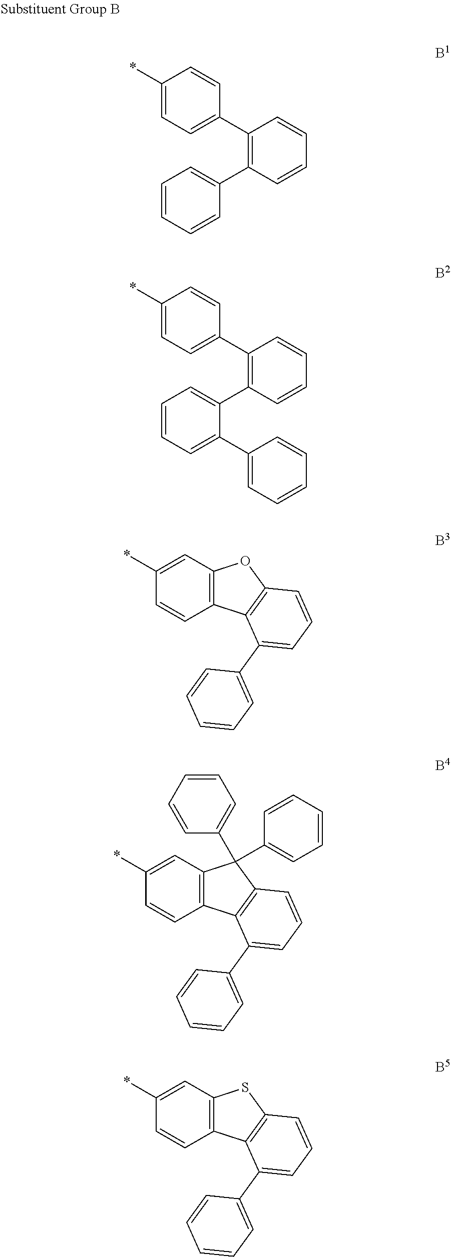 [SMILES: *c1ccc(-c2ccccc2-c2ccccc2)cc1 ; *c1ccc(-c2ccccc2-c2ccccc2-c2ccccc2)cc1 ; *c1ccc2c(c1)oc1cccc(-c3ccccc3)c12 ; *c1ccc2c(c1)C(c1ccccc1)(c1ccccc1)c1cccc(-c3ccccc3)c1-2 ; *c1ccc2c(c1)sc1cccc(-c3ccccc3)c12] 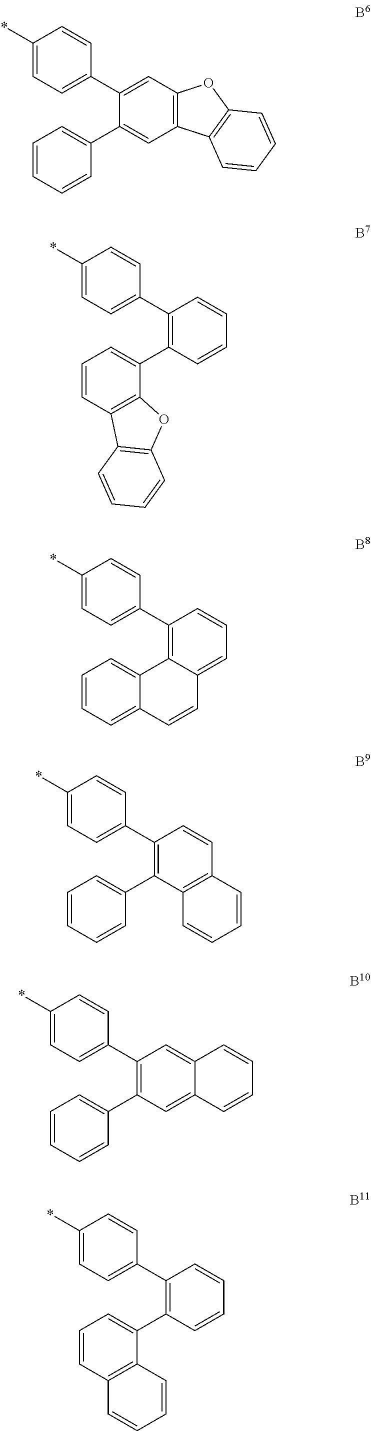 [SMILES: *c1ccc(-c2cc3oc4ccccc4c3cc2-c2ccccc2)cc1 ; *c1ccc(-c2ccccc2-c2cccc3c2oc2ccccc23)cc1 ; *c1ccc(-c2cccc3ccc4ccccc4c23)cc1 ; *c1ccc(-c2ccc3ccccc3c2-c2ccccc2)cc1 ; *c1ccc(-c2cc3ccccc3cc2-c2ccccc2)cc1 ; *c1ccc(-c2ccccc2-c2cccc3ccccc23)cc1] 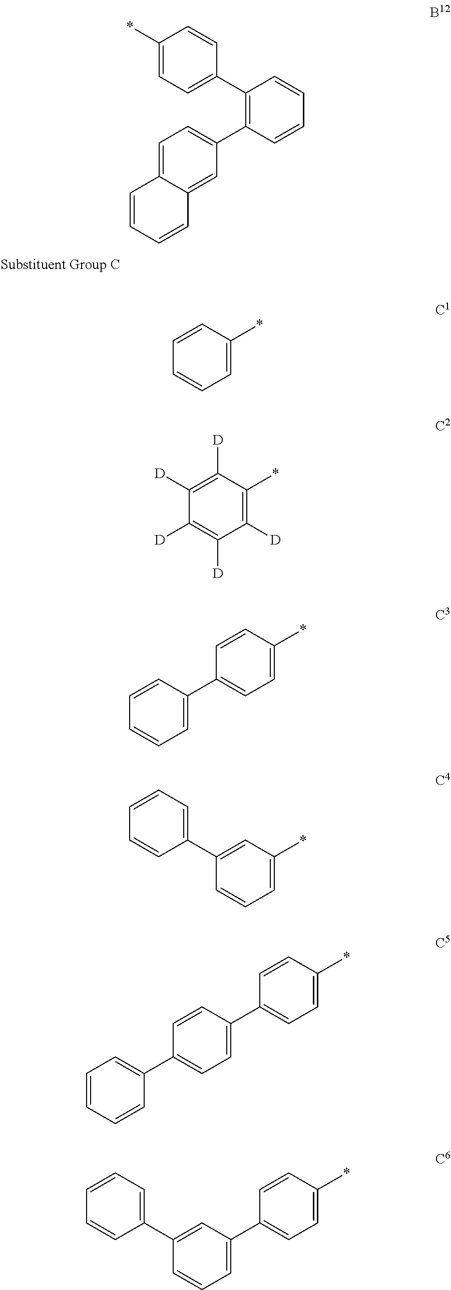 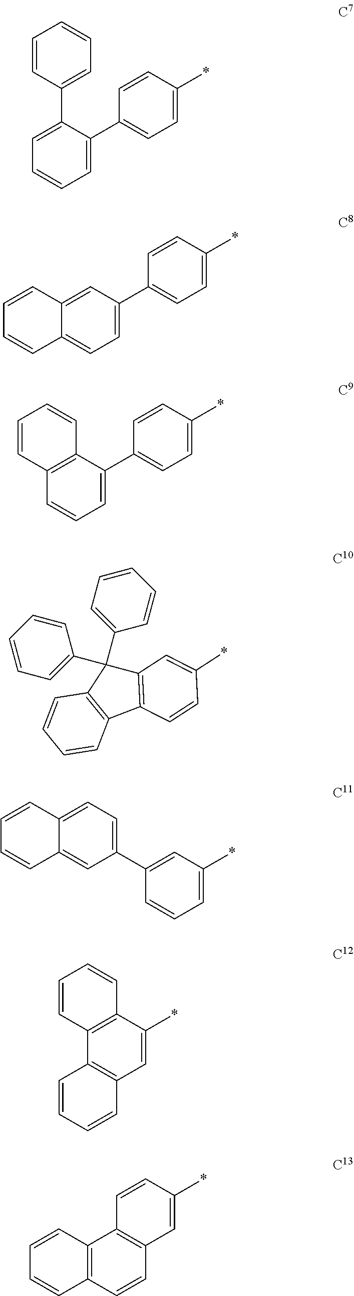 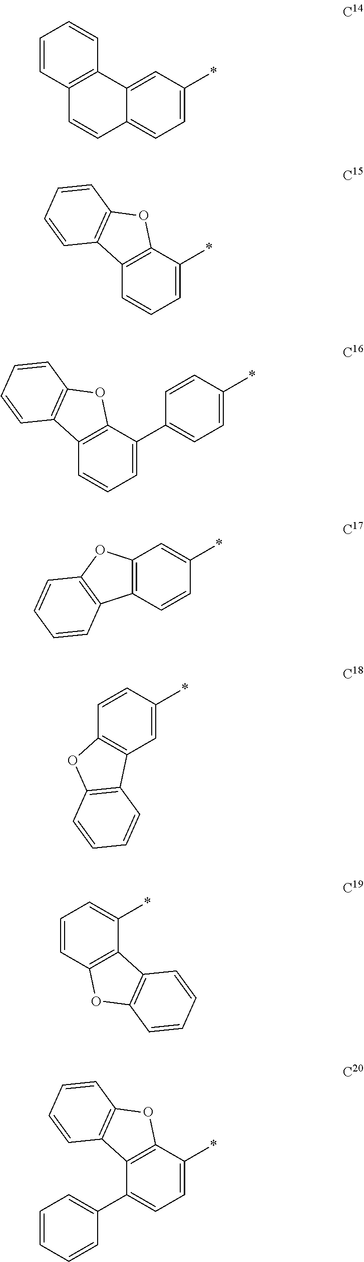 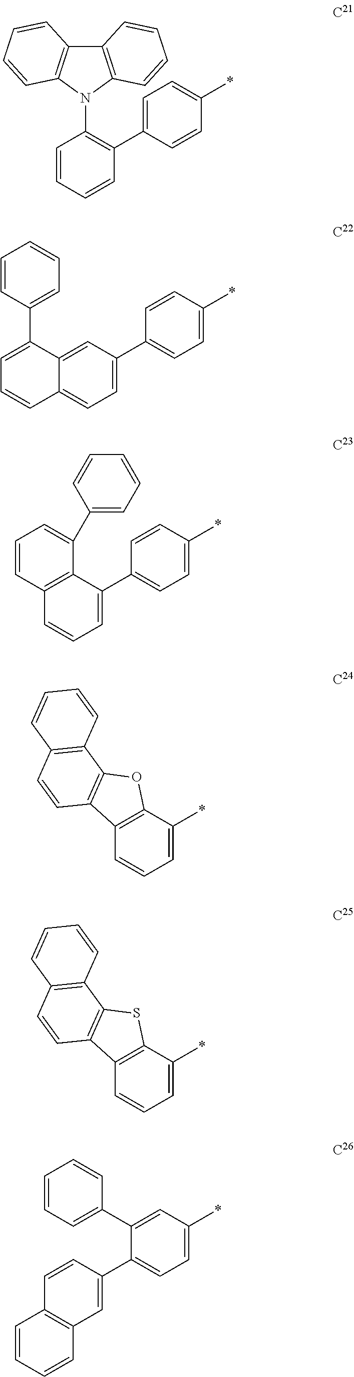 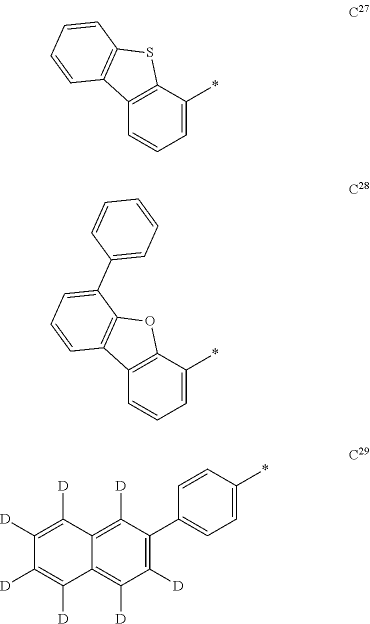 [SMILES: *c1cccc2c1sc1ccccc12 ; *c1cccc2c1oc1c(-c3ccccc3)cccc12 ; *c1ccc(-c2c([2H])c([2H])c3c([2H])c([2H])c([2H])c([2H])c3c2[2H])cc1]
TABLE 1
Compound Combination

| No. | A | B | C |
|---|---|---|---|
| 1 | 1 | 1 | 1 |
| 2 | 2 | 1 | 1 |
| 3 | 3 | 1 | 1 |
| 4 | 4 | 1 | 1 |
| 5 | 5 | 1 | 1 |
| 6 | 6 | 1 | 1 |
| 7 | 7 | 1 | 1 |
| 8 | 8 | 1 | 1 |
| 9 | 9 | 1 | 1 |
| 10 | 1 | 1 | 28 |
| 11 | 1 | 2 | 28 |
| 12 | 12 | 1 | 1 |
| 13 | 13 | 1 | 1 |
| 14 | 14 | 1 | 1 |
| 15 | 1 | 1 | 2 |
| 16 | 2 | 1 | 2 |
| 17 | 3 | 1 | 2 |
| 18 | 4 | 1 | 2 |
| 19 | 5 | 1 | 2 |
| 20 | 6 | 1 | 2 |
| 21 | 7 | 1 | 2 |
| 22 | 8 | 1 | 2 |
| 23 | 9 | 1 | 2 |
| 24 | 10 | 1 | 2 |
| 25 | 11 | 1 | 2 |
| 26 | 12 | 1 | 2 |
| 27 | 13 | 1 | 2 |
| 28 | 14 | 1 | 2 |
| 29 | 1 | 1 | 3 |
| 30 | 2 | 1 | 3 |
| 31 | 3 | 1 | 3 |
| 32 | 4 | 1 | 3 |
| 33 | 5 | 1 | 3 |
| 34 | 6 | 1 | 3 |
| 35 | 7 | 1 | 3 |
| 36 | 8 | 1 | 3 |
| 37 | 9 | 1 | 3 |
| 38 | 10 | 1 | 3 |
| 39 | 11 | 1 | 3 |
| 40 | 12 | 1 | 3 |
| 41 | 13 | 1 | 3 |
| 42 | 14 | 1 | 3 |
| 43 | 1 | 1 | 8 |
| 44 | 2 | 1 | 8 |
| 45 | 3 | 1 | 8 |
| 46 | 4 | 1 | 8 |
| 47 | 5 | 1 | 8 |
| 48 | 6 | 1 | 8 |
| 49 | 7 | 1 | 8 |
| 50 | 8 | 1 | 8 |
| 51 | 9 | 1 | 8 |
| 52 | 10 | 1 | 8 |
| 53 | 11 | 1 | 8 |
| 54 | 12 | 1 | 8 |
| 55 | 13 | 1 | 8 |
| 56 | 14 | 1 | 8 |
| 57 | 1 | 2 | 1 |
| 58 | 2 | 2 | 1 |
| 59 | 3 | 2 | 1 |
| 60 | 4 | 2 | 1 |
| 61 | 5 | 2 | 1 |
| 62 | 6 | 2 | 1 |
| 63 | 7 | 2 | 1 |
| 64 | 8 | 2 | 1 |
| 65 | 9 | 2 | 1 |
| 66 | 10 | 2 | 1 |
| 67 | 11 | 2 | 1 |
| 68 | 12 | 2 | 1 |
| 69 | 13 | 2 | 1 |
| 70 | 14 | 2 | 1 |
| 71 | 1 | 2 | 2 |
| 72 | 2 | 2 | 2 |
| 73 | 3 | 2 | 2 |
| 74 | 4 | 2 | 2 |
| 75 | 5 | 2 | 2 |
| 76 | 6 | 2 | 2 |
| 77 | 7 | 2 | 2 |
| 78 | 8 | 2 | 2 |
| 79 | 9 | 2 | 2 |
| 80 | 10 | 2 | 2 |
| 81 | 11 | 2 | 2 |
| 82 | 12 | 2 | 2 |
| 83 | 13 | 2 | 2 |
| 84 | 14 | 2 | 2 |
| 85 | 1 | 2 | 3 |
| 86 | 2 | 2 | 3 |
| 87 | 3 | 2 | 3 |
| 88 | 4 | 2 | 3 |
| 89 | 5 | 2 | 3 |
| 90 | 6 | 2 | 3 |
| 91 | 7 | 2 | 3 |
| 92 | 8 | 2 | 3 |
| 93 | 9 | 2 | 3 |
| 94 | 10 | 2 | 3 |
| 95 | 11 | 2 | 3 |
| 96 | 12 | 2 | 3 |
| 97 | 13 | 2 | 3 |
| 98 | 14 | 2 | 3 |
| 99 | 1 | 2 | 8 |
| 100 | 2 | 2 | 8 |
| 101 | 3 | 2 | 8 |
| 102 | 4 | 2 | 8 |
| 103 | 5 | 2 | 8 |
| 104 | 6 | 2 | 8 |
| 105 | 7 | 2 | 8 |
| 106 | 8 | 2 | 8 |
| 107 | 9 | 2 | 8 |
| 108 | 10 | 2 | 8 |
| 109 | 11 | 2 | 8 |
| 110 | 12 | 2 | 8 |
| 111 | 13 | 2 | 8 |
| 112 | 14 | 2 | 8 |
| 113 | 1 | 3 | 8 |
| 114 | 1 | 4 | 8 |
| 115 | 1 | 5 | 8 |
| 116 | 1 | 6 | 8 |
| 117 | 1 | 7 | 8 |
| 118 | 1 | 8 | 8 |
| 119 | 1 | 11 | 8 |
| 120 | 1 | 12 | 8 |
| 121 | 1 | 1 | 13 |
| 122 | 1 | 2 | 13 |
| 123 | 1 | 3 | 13 |
| 124 | 1 | 4 | 13 |
| 125 | 1 | 5 | 13 |
| 126 | 1 | 6 | 13 |
| 127 | 1 | 7 | 13 |
| 128 | 1 | 8 | 13 |
| 129 | 1 | 9 | 13 |
| 130 | 1 | 10 | 13 |
| 131 | 1 | 11 | 13 |
| 132 | 1 | 12 | 13 |
| 133 | 3 | 3 | 8 |
| 134 | 3 | 4 | 8 |
| 135 | 3 | 5 | 8 |
| 136 | 3 | 6 | 8 |
| 137 | 3 | 7 | 8 |
| 138 | 3 | 8 | 8 |
| 139 | 3 | 11 | 8 |
| 140 | 3 | 12 | 8 |
| 141 | 3 | 1 | 13 |
| 142 | 3 | 2 | 13 |
| 143 | 3 | 3 | 13 |
| 144 | 3 | 4 | 13 |
| 145 | 3 | 5 | 13 |
| 146 | 3 | 6 | 13 |
| 147 | 3 | 7 | 13 |
| 148 | 3 | 8 | 13 |
| 149 | 3 | 9 | 13 |
| 150 | 3 | 10 | 13 |
| 151 | 3 | 11 | 13 |
| 152 | 3 | 12 | 13 |
| 153 | 1 | 1 | 4 |
| 154 | 1 | 1 | 5 |
| 155 | 1 | 1 | 6 |
| 156 | 1 | 1 | 7 |
| 157 | 1 | 1 | 9 |
| 158 | 1 | 1 | 10 |
| 159 | 1 | 1 | 11 |
| 160 | 1 | 1 | 12 |
| 161 | 1 | 1 | 14 |
| 162 | 1 | 1 | 15 |
| 163 | 1 | 1 | 16 |
| 164 | 1 | 1 | 17 |
| 165 | 1 | 1 | 18 |
| 166 | 1 | 1 | 19 |
| 167 | 1 | 1 | 20 |
| 168 | 1 | 1 | 21 |
| 169 | 1 | 1 | 22 |
| 170 | 1 | 1 | 23 |
| 171 | 1 | 1 | 24 |
| 172 | 1 | 1 | 25 |
| 173 | 1 | 1 | 26 |
| 174 | 1 | 1 | 27 |
| 175 | 1 | 2 | 4 |
| 176 | 1 | 2 | 5 |
| 177 | 1 | 2 | 6 |
| 178 | 1 | 2 | 7 |
| 179 | 1 | 2 | 9 |
| 180 | 1 | 2 | 10 |
| 181 | 1 | 2 | 11 |
| 182 | 1 | 2 | 12 |
| 183 | 1 | 2 | 14 |
| 184 | 1 | 2 | 15 |
| 185 | 1 | 2 | 16 |
| 186 | 1 | 2 | 17 |
| 187 | 1 | 2 | 18 |
| 188 | 1 | 2 | 19 |
| 189 | 1 | 2 | 20 |
| 190 | 1 | 2 | 21 |
| 191 | 1 | 2 | 22 |
| 192 | 1 | 2 | 23 |
| 193 | 1 | 2 | 24 |
| 194 | 1 | 2 | 25 |
| 195 | 1 | 2 | 26 |
| 196 | 1 | 2 | 27 |
| 197 | 1 | 7 | 1 |
| 198 | 1 | 7 | 2 |
| 199 | 1 | 7 | 3 |
| 200 | 1 | 7 | 4 |
| 201 | 1 | 7 | 5 |
| 202 | 1 | 7 | 6 |
| 203 | 1 | 7 | 7 |
| 204 | 1 | 7 | 9 |
| 205 | 1 | 7 | 10 |
| 206 | 1 | 7 | 11 |
| 207 | 1 | 7 | 12 |
| 208 | 1 | 7 | 14 |
| 209 | 1 | 7 | 15 |
| 210 | 1 | 7 | 16 |
| 211 | 1 | 7 | 17 |
| 212 | 1 | 7 | 18 |
| 213 | 1 | 7 | 19 |
| 214 | 1 | 7 | 20 |
| 215 | 1 | 7 | 21 |
| 216 | 1 | 7 | 22 |
| 217 | 1 | 7 | 23 |
| 218 | 1 | 7 | 24 |
| 219 | 1 | 7 | 25 |
| 220 | 1 | 7 | 26 |
| 221 | 1 | 7 | 27 |
| 222 | 1 | 11 | 1 |
| 223 | 1 | 11 | 2 |
| 224 | 1 | 11 | 3 |
| 225 | 1 | 11 | 4 |
| 226 | 1 | 11 | 5 |
| 227 | 1 | 11 | 6 |
| 228 | 1 | 11 | 7 |
| 229 | 1 | 11 | 9 |
| 230 | 1 | 11 | 10 |
| 231 | 1 | 11 | 11 |
| 232 | 1 | 11 | 12 |
| 233 | 1 | 11 | 14 |
| 234 | 1 | 11 | 15 |
| 235 | 1 | 11 | 16 |
| 236 | 1 | 11 | 17 |
| 237 | 1 | 11 | 18 |
| 238 | 1 | 11 | 19 |
| 239 | 1 | 11 | 20 |
| 240 | 1 | 11 | 21 |
| 241 | 1 | 11 | 22 |
| 242 | 1 | 11 | 23 |
| 243 | 1 | 11 | 24 |
| 244 | 1 | 11 | 25 |
| 245 | 1 | 11 | 26 |
| 246 | 1 | 11 | 27 |
| 247 | 3 | 1 | 4 |
| 248 | 3 | 1 | 5 |
| 249 | 3 | 1 | 6 |
| 250 | 3 | 1 | 7 |
| 251 | 3 | 1 | 9 |
| 252 | 3 | 1 | 10 |
| 253 | 3 | 1 | 11 |
| 254 | 3 | 1 | 12 |
| 255 | 3 | 1 | 14 |
| 256 | 3 | 1 | 15 |
| 257 | 3 | 1 | 16 |
| 258 | 3 | 1 | 17 |
| 259 | 3 | 1 | 18 |
| 260 | 3 | 1 | 19 |
| 261 | 3 | 1 | 20 |
| 262 | 3 | 1 | 21 |
| 263 | 3 | 1 | 22 |
| 264 | 3 | 1 | 23 |
| 265 | 3 | 1 | 24 |
| 266 | 3 | 1 | 25 |
| 267 | 3 | 1 | 26 |
| 268 | 3 | 1 | 27 |
| 269 | 3 | 2 | 4 |
| 270 | 3 | 2 | 5 |
| 271 | 3 | 2 | 6 |
| 272 | 3 | 2 | 7 |
| 273 | 3 | 2 | 9 |
| 274 | 3 | 2 | 10 |
| 275 | 3 | 2 | 11 |
| 276 | 3 | 2 | 12 |
| 277 | 3 | 2 | 14 |
| 278 | 3 | 2 | 15 |
| 279 | 3 | 2 | 16 |
| 280 | 3 | 2 | 17 |
| 281 | 3 | 2 | 18 |
| 282 | 3 | 2 | 19 |
| 283 | 3 | 2 | 20 |
| 284 | 3 | 2 | 21 |
| 285 | 3 | 2 | 22 |
| 286 | 3 | 2 | 23 |
| 287 | 3 | 2 | 24 |
| 288 | 3 | 2 | 25 |
| 289 | 3 | 2 | 26 |
| 290 | 3 | 2 | 27 |
| 291 | 3 | 7 | 1 |
| 292 | 3 | 7 | 2 |
| 293 | 3 | 7 | 3 |
| 294 | 3 | 7 | 4 |
| 295 | 3 | 7 | 5 |
| 296 | 3 | 7 | 6 |
| 297 | 3 | 7 | 7 |
| 298 | 3 | 7 | 9 |
| 299 | 3 | 7 | 10 |
| 300 | 3 | 7 | 11 |
| 301 | 3 | 7 | 12 |
| 302 | 3 | 7 | 14 |
| 303 | 3 | 7 | 15 |
| 304 | 3 | 7 | 16 |
| 305 | 3 | 7 | 17 |
| 306 | 3 | 7 | 18 |
| 307 | 3 | 7 | 19 |
| 308 | 3 | 7 | 20 |
| 309 | 3 | 7 | 21 |
| 310 | 3 | 7 | 22 |
| 311 | 3 | 7 | 23 |
| 312 | 3 | 7 | 24 |
| 313 | 3 | 7 | 25 |
| 314 | 3 | 7 | 26 |
| 315 | 3 | 7 | 27 |
| 316 | 3 | 11 | 1 |
| 317 | 3 | 11 | 2 |
| 318 | 3 | 11 | 3 |
| 319 | 3 | 11 | 4 |
| 320 | 3 | 11 | 5 |
| 321 | 3 | 11 | 6 |
| 322 | 3 | 11 | 7 |
| 323 | 3 | 11 | 9 |
| 324 | 3 | 11 | 10 |
| 325 | 3 | 11 | 11 |
| 326 | 3 | 11 | 12 |
| 327 | 3 | 11 | 14 |
| 328 | 3 | 11 | 15 |
| 329 | 3 | 11 | 16 |
| 330 | 3 | 11 | 17 |
| 331 | 3 | 11 | 18 |
| 332 | 3 | 11 | 19 |
| 333 | 3 | 11 | 20 |
| 334 | 3 | 11 | 21 |
| 335 | 3 | 11 | 22 |
| 336 | 3 | 11 | 23 |
| 337 | 3 | 11 | 24 |
| 338 | 3 | 11 | 25 |
| 339 | 3 | 11 | 26 |
| 340 | 3 | 11 | 27 |
| 341 | 4 | 1 | 4 |
| 342 | 4 | 1 | 5 |
| 343 | 4 | 1 | 6 |
| 344 | 4 | 1 | 7 |
| 345 | 4 | 1 | 9 |
| 346 | 4 | 1 | 10 |
| 347 | 4 | 1 | 11 |
| 348 | 4 | 1 | 12 |
| 349 | 4 | 1 | 13 |
| 350 | 4 | 1 | 14 |
| 351 | 4 | 1 | 15 |
| 352 | 4 | 1 | 16 |
| 353 | 4 | 1 | 17 |
| 354 | 4 | 1 | 18 |
| 355 | 4 | 1 | 19 |
| 356 | 4 | 1 | 20 |
| 357 | 4 | 1 | 21 |
| 358 | 4 | 1 | 22 |
| 359 | 4 | 1 | 23 |
| 360 | 4 | 1 | 24 |
| 361 | 4 | 1 | 25 |
| 362 | 4 | 1 | 26 |
| 363 | 4 | 1 | 27 |
| 364 | 4 | 2 | 4 |
| 365 | 4 | 2 | 5 |
| 366 | 4 | 2 | 6 |
| 367 | 4 | 2 | 7 |
| 368 | 4 | 2 | 9 |
| 369 | 4 | 2 | 10 |
| 370 | 4 | 2 | 11 |
| 371 | 4 | 2 | 12 |
| 372 | 4 | 2 | 13 |
| 373 | 4 | 2 | 14 |
| 374 | 4 | 2 | 15 |
| 375 | 4 | 2 | 16 |
| 376 | 4 | 2 | 17 |
| 377 | 4 | 2 | 18 |
| 378 | 4 | 2 | 19 |
| 379 | 4 | 2 | 20 |
| 380 | 4 | 2 | 21 |
| 381 | 4 | 2 | 22 |
| 382 | 4 | 2 | 23 |
| 383 | 4 | 2 | 24 |
| 384 | 4 | 2 | 25 |
| 385 | 4 | 2 | 26 |
| 386 | 4 | 2 | 27 |
| 387 | 4 | 7 | 1 |
| 388 | 4 | 7 | 2 |
| 389 | 4 | 7 | 3 |
| 390 | 4 | 7 | 4 |
| 391 | 4 | 7 | 5 |
| 392 | 4 | 7 | 6 |
| 393 | 4 | 7 | 7 |
| 394 | 4 | 7 | 8 |
| 395 | 4 | 7 | 9 |
| 396 | 4 | 7 | 10 |
| 397 | 4 | 7 | 11 |
| 398 | 4 | 7 | 12 |
| 399 | 4 | 7 | 13 |
| 400 | 4 | 7 | 14 |
| 401 | 4 | 7 | 15 |
| 402 | 4 | 7 | 16 |
| 403 | 4 | 7 | 17 |
| 404 | 4 | 7 | 18 |
| 405 | 4 | 7 | 19 |
| 406 | 4 | 7 | 20 |
| 407 | 4 | 7 | 21 |
| 408 | 4 | 7 | 22 |
| 409 | 4 | 7 | 23 |
| 410 | 4 | 7 | 24 |
| 411 | 4 | 7 | 25 |
| 412 | 4 | 7 | 26 |
| 413 | 4 | 7 | 27 |
| 414 | 4 | 11 | 1 |
| 415 | 4 | 11 | 2 |
| 416 | 4 | 11 | 3 |
| 417 | 4 | 11 | 4 |
| 418 | 4 | 11 | 5 |
| 419 | 4 | 11 | 6 |
| 420 | 4 | 11 | 7 |
| 421 | 4 | 11 | 8 |
| 422 | 4 | 11 | 9 |
| 423 | 4 | 11 | 10 |
| 424 | 4 | 11 | 11 |
| 425 | 4 | 11 | 12 |
| 426 | 4 | 11 | 13 |
| 427 | 4 | 11 | 14 |
| 428 | 4 | 11 | 15 |
| 429 | 4 | 11 | 16 |
| 430 | 4 | 11 | 17 |
| 431 | 4 | 11 | 18 |
| 432 | 4 | 11 | 19 |
| 433 | 4 | 11 | 20 |
| 434 | 4 | 11 | 21 |
| 435 | 4 | 11 | 22 |
| 436 | 4 | 11 | 23 |
| 437 | 4 | 11 | 24 |
| 438 | 4 | 11 | 25 |
| 439 | 4 | 11 | 26 |
| 440 | 4 | 11 | 27 |
| 441 | 7 | 1 | 4 |
| 442 | 7 | 1 | 5 |
| 443 | 7 | 1 | 6 |
| 444 | 7 | 1 | 7 |
| 445 | 7 | 1 | 9 |
| 446 | 7 | 1 | 10 |
| 447 | 7 | 1 | 11 |
| 448 | 7 | 1 | 12 |
| 449 | 7 | 1 | 13 |
| 450 | 7 | 1 | 14 |
| 451 | 7 | 1 | 15 |
| 452 | 7 | 1 | 16 |
| 453 | 7 | 1 | 17 |
| 454 | 7 | 1 | 18 |
| 455 | 7 | 1 | 19 |
| 456 | 7 | 1 | 20 |
| 457 | 7 | 1 | 21 |
| 458 | 7 | 1 | 22 |
| 459 | 7 | 1 | 23 |
| 460 | 7 | 1 | 24 |
| 461 | 7 | 1 | 25 |
| 462 | 7 | 1 | 26 |
| 463 | 7 | 1 | 27 |
| 464 | 7 | 2 | 4 |
| 465 | 7 | 2 | 5 |
| 466 | 7 | 2 | 6 |
| 467 | 7 | 2 | 7 |
| 468 | 7 | 2 | 9 |
| 469 | 7 | 2 | 10 |
| 470 | 7 | 2 | 11 |
| 471 | 7 | 2 | 12 |
| 472 | 7 | 2 | 13 |
| 473 | 7 | 2 | 14 |
| 474 | 7 | 2 | 15 |
| 475 | 7 | 2 | 16 |
| 476 | 7 | 2 | 17 |
| 477 | 7 | 2 | 18 |
| 478 | 7 | 2 | 19 |
| 479 | 7 | 2 | 20 |
| 480 | 7 | 2 | 21 |
| 481 | 7 | 2 | 22 |
| 482 | 7 | 2 | 23 |
| 483 | 7 | 2 | 24 |
| 484 | 7 | 2 | 25 |
| 485 | 7 | 2 | 26 |
| 486 | 7 | 2 | 27 |
| 487 | 7 | 7 | 1 |
| 488 | 7 | 7 | 2 |
| 489 | 7 | 7 | 3 |
| 490 | 7 | 7 | 4 |
| 491 | 7 | 7 | 5 |
| 492 | 7 | 7 | 6 |
| 493 | 7 | 7 | 7 |
| 494 | 7 | 7 | 8 |
| 495 | 7 | 7 | 9 |
| 496 | 7 | 7 | 10 |
| 497 | 7 | 7 | 11 |
| 498 | 7 | 7 | 12 |
| 499 | 7 | 7 | 13 |
| 500 | 7 | 7 | 14 |
| 501 | 7 | 7 | 15 |
| 502 | 7 | 7 | 16 |
| 503 | 7 | 7 | 17 |
| 504 | 7 | 7 | 18 |
| 505 | 7 | 7 | 19 |
| 506 | 7 | 7 | 20 |
| 507 | 7 | 7 | 21 |
| 508 | 7 | 7 | 22 |
| 509 | 7 | 7 | 23 |
| 510 | 7 | 7 | 24 |
| 511 | 7 | 7 | 25 |
| 512 | 7 | 7 | 26 |
| 513 | 7 | 7 | 27 |
| 514 | 7 | 11 | 1 |
| 515 | 7 | 11 | 2 |
| 516 | 7 | 11 | 3 |
| 517 | 7 | 11 | 4 |
| 518 | 7 | 11 | 5 |
| 519 | 7 | 11 | 6 |
| 520 | 7 | 11 | 7 |
| 521 | 7 | 11 | 8 |
| 522 | 7 | 11 | 9 |
| 523 | 7 | 11 | 10 |
| 524 | 7 | 11 | 11 |
| 525 | 7 | 11 | 12 |
| 526 | 7 | 11 | 13 |
| 527 | 7 | 11 | 14 |
| 528 | 7 | 11 | 15 |
| 529 | 7 | 11 | 16 |
| 530 | 7 | 11 | 17 |
| 531 | 7 | 11 | 18 |
| 532 | 7 | 11 | 19 |
| 533 | 7 | 11 | 20 |
| 534 | 7 | 11 | 21 |
| 535 | 7 | 11 | 22 |
| 536 | 7 | 11 | 23 |
| 537 | 7 | 11 | 24 |
| 538 | 7 | 11 | 25 |
| 539 | 7 | 11 | 26 |
| 540 | 7 | 11 | 27 |
| 541 | 10 | 1 | 4 |
| 542 | 10 | 1 | 5 |
| 543 | 10 | 1 | 6 |
| 544 | 10 | 1 | 7 |
| 545 | 10 | 1 | 9 |
| 546 | 10 | 1 | 10 |
| 547 | 10 | 1 | 11 |
| 548 | 10 | 1 | 12 |
| 549 | 10 | 1 | 13 |
| 550 | 10 | 1 | 14 |
| 551 | 10 | 1 | 15 |
| 552 | 10 | 1 | 16 |
| 553 | 10 | 1 | 17 |
| 554 | 10 | 1 | 18 |
| 555 | 10 | 1 | 19 |
| 556 | 10 | 1 | 20 |
| 557 | 10 | 1 | 21 |
| 558 | 10 | 1 | 22 |
| 559 | 10 | 1 | 23 |
| 560 | 10 | 1 | 24 |
| 561 | 10 | 1 | 25 |
| 562 | 10 | 1 | 26 |
| 563 | 10 | 1 | 27 |
| 564 | 10 | 2 | 4 |
| 565 | 10 | 2 | 5 |
| 566 | 10 | 2 | 6 |
| 567 | 10 | 2 | 7 |
| 568 | 10 | 2 | 9 |
| 569 | 10 | 2 | 10 |
| 570 | 10 | 2 | 11 |
| 571 | 10 | 2 | 12 |
| 572 | 10 | 2 | 13 |
| 573 | 10 | 2 | 14 |
| 574 | 10 | 2 | 15 |
| 575 | 10 | 2 | 16 |
| 576 | 10 | 2 | 17 |
| 577 | 10 | 2 | 18 |
| 578 | 10 | 2 | 19 |
| 579 | 10 | 2 | 20 |
| 580 | 10 | 2 | 21 |
| 581 | 10 | 2 | 22 |
| 582 | 10 | 2 | 23 |
| 583 | 10 | 2 | 24 |
| 584 | 10 | 2 | 25 |
| 585 | 10 | 2 | 26 |
| 586 | 10 | 2 | 27 |
| 587 | 10 | 7 | 1 |
| 588 | 10 | 7 | 2 |
| 589 | 10 | 7 | 3 |
| 590 | 10 | 7 | 4 |
| 591 | 10 | 7 | 5 |
| 592 | 10 | 7 | 6 |
| 593 | 10 | 7 | 7 |
| 594 | 10 | 7 | 8 |
| 595 | 10 | 7 | 9 |
| 596 | 10 | 7 | 10 |
| 597 | 10 | 7 | 11 |
| 598 | 10 | 7 | 12 |
| 599 | 10 | 7 | 13 |
| 600 | 10 | 7 | 14 |
| 601 | 10 | 7 | 15 |
| 602 | 10 | 7 | 16 |
| 603 | 10 | 7 | 17 |
| 604 | 10 | 7 | 18 |
| 605 | 10 | 7 | 19 |
| 606 | 10 | 7 | 20 |
| 607 | 10 | 7 | 21 |
| 608 | 10 | 7 | 22 |
| 609 | 10 | 7 | 23 |
| 610 | 10 | 7 | 24 |
| 611 | 10 | 7 | 25 |
| 612 | 10 | 7 | 26 |
| 613 | 10 | 7 | 27 |
| 614 | 10 | 11 | 1 |
| 615 | 10 | 11 | 2 |
| 616 | 10 | 11 | 3 |
| 617 | 10 | 11 | 4 |
| 618 | 10 | 11 | 5 |
| 619 | 10 | 11 | 6 |
| 620 | 10 | 11 | 7 |
| 621 | 10 | 11 | 8 |
| 622 | 10 | 11 | 9 |
| 623 | 10 | 11 | 10 |
| 624 | 10 | 11 | 11 |
| 625 | 10 | 11 | 12 |
| 626 | 10 | 11 | 13 |
| 627 | 10 | 11 | 14 |
| 628 | 10 | 11 | 15 |
| 629 | 10 | 11 | 16 |
| 630 | 10 | 11 | 17 |
| 631 | 10 | 11 | 18 |
| 632 | 10 | 11 | 19 |
| 633 | 10 | 11 | 20 |
| 634 | 10 | 11 | 21 |
| 635 | 10 | 11 | 22 |
| 636 | 10 | 11 | 23 |
| 637 | 10 | 11 | 24 |
| 638 | 10 | 11 | 25 |
| 639 | 10 | 11 | 26 |
| 640 | 10 | 11 | 27 |
| 641 | 3 | 1 | 29. |
